SEQUENCE LISTING (112 sequences)

SEQ ID NO: 1 - protein, 299 aa, Photorhabdus
MMREYSNEDDFIKEKTNLVKSENVEADNYLETEYLTYLAKLIGMTERENHHLNSIKLIDDIIELHNDRKGNKLLWNDNWQDKIIDRDLQSIFKKIDEMVSEFGGLEAYKDIVGENPYDPTEPVCGYSAQNIFKLMTEGEYAVDPVKMAKTGKINGNQFAEKLEHLNSSNNYVALINDHRLGHMFLVDIPSTNRERVGYIYQSDLGDGALPALKIADWLKSRGKESINVNKLKKFLNDEFTMLPDNEQKGLIAEIFDLNKDIDSVKSGKIKKDKAVDIYLREYDINDFISNIEKLKTKLA

SEQ ID NO: 2 - protein, 309 aa, Photorhabdus
MFQNRIRNEKTTQSGKGKTLDRMTDSLYLEIPNVEAVTLAYQKLTSKYRKFDNKTKLILDSSDEFSQLKSEKQRKGFSKSGLKNNGVSDRKFIYTKNALKNFAAHAGYEHNGHYEDEFVNFKDNNKNLAKGKLFPGISLIERRKLSIVKNKEGKWEHKETDEAEAYKVTDIEKFISGVRSMYLQGNTFLHAKTEALIRKHIANNENILPTMAGIAGLHAEVQALNNLFISGDKGTKKREKWKYIRNMLESSIFTQRLTTGQAGKDFAACHNCSGILSSPVNVITGKVESAGDNFLSTLSRYKTSQESPI

SEQ ID NO: 3 - protein, 278 aa, Photorhabdus
MEREYSEKQKNPSKLSRKTAISERIAALERSGLSNSNQPVPQFARPYTSNRPVVNINPGRSSIAVATANSTSPVNIPTPAPASPDKLLPSTSCDTTSSILIVGKYNLELTSQGKIVVFRGDNRTPEQIVAAGGFYPWSKQDVGKIKKELIDEFIEIGPSAHMMGHVRSPNKNYVSTGMNMDSGGFGEQSNYLYKMEIPGLKPQDMNERTLGEKIRQDKRGINYPHFLMSHLTLAESEFVAMIPARSEELTFITPIPLSYITSYRKRGTNTWLPMPLKK

SEQ ID NO: 4 - protein, 1633 aa, Photorhabdus
MSNYEYDIVTQHDTYQIKDNEYTVVNGKYWQYEQEGNKNNNKVSISLMKENQNDPVWITSDIKEISLYIIENLFSYHKFSAELQHTLKNAVKAVFNEYSEIKYSELLHNINNIFNLFFIKIYNTSDIDTAINILTAKIEIYDKLEKINQDKTDSNNTNVDIWEELGINAEEPLLKIYRQAFSTGDIDDEVYSDALLTFMSDGNLELGDKEKSDYNQRIKDKTDLFESYKKGIEKVASLITTNNINPGIPITYPETEKSINIGDDLLLAQLAKEEIALKKQNRTEYSQQDIFELQTLQAAKYHLLILSSLGALLYQIAPNVEKMTKGHGDYRDIIFSQEQAESLFKKHNIQYDTNHVLSQESKHIEMEGCIILTAAIIYRMRKENATVEQALNYSTLETIKLFENDKKKLNPFNTNNVKPAGYFSFIDFKKRDKFDSQYNFNEQFNVYKNKYSHYESISFSKLILSSPAAQLTAEEIVNPPEEAFLYSVEQGMGNVAMIKMYQGNWLVISTIQGGVKAKKYSRQQVDSNPTLRAMSKPNALFLIERKMETGMGILMPNMMVNTGKRLFPTGYERAKTLSGFAETSRYKNSYNAFWNDYYGITSGMNVGISFTGSPKFNFYKEENLLSVTATIIQQGLNDIAIKSKQALDITSGWHIAATILIPFYNVIYKSTTDSEYELTGEDIGSIVFDTANVLLVVATLGMSLTESMAAKVTQTTLRLRQAGLTGRALITAVVRTLPEHGIITLRQSSGIILGGLIDLIEPLPIRSTLTLTYRGVINAVGAMRNSIKLEKSFADIFGKSTRGLGKLKNEWKVSNLPLEEIVPHSNGGEIYKGIYSIRPTNPETAVKQNFYIKEAGANYQVKWDDANHTWRVVNPTYPEQFSYWPAVKLDKNGHWVTHADVSNKFLILEQSKRIDQELEAAHSNINNDNILDAFIHINTAFKDCERYDIDKLSDITDTLTHFFEKSLKPGDKKAIFSTEIMSIQQAWIREVILPLQNNSSISIEKINAIKTELPYLLRKTFPIESQLPNQLVANKIALAIEEIPNTRIPKYTSGNISKTVQYTSLLENNHVDIPPVGITITGNDTFINQVTRVLSEIDEIPSGNIVIQELEKQGLNIQPPTMNDIVREKNGQFYANNSAGSHIAFDPENHLIGTEEKLIDEPWRTREPAIALYHEMLHIYYNRYPTWFTSIDNKVIDQKVSGGFSLLEESRIVGTKYYVNDKNTLFDFNDSDYLLENNSALLTENRFRAEYAIFKNKSEYVIRPYSGKGDSQIPLTKTKININESHRNVMGVGSGKPEKMPNESATDYRNRVREWRKANKQPEADIGTGDMRKTKAEARVKLLKENYPQFEPQKIELGGAFQLWTVPNEPANKLMLSSHGYFFSDSAATQVPAGKTIQFLGPHGKTLLEAPENPLYSPFDVTLGNSGFTVQPYATIESGNKAGLGSVKIGDKTFTVNDIQNIATDDVENYLLATGVEANASNHGKVRNYGIKYYEKMPDEEVKAAIWKNRADETSTHKYDALLVSPEAGNRKKLSDIFALMKTDERMSKYDEITFVACREELNRINMKSIHDTGLGGGYEPKLEPTVILSRRRREATFTADGAIIYSIIAVNLHHNFITEEIVGIAPFLFINN

SEQ ID NO: 5 - protein, 324 aa, Photorhabdus
MEHEYNEKEKQRNSAIKLNDAIRNNEENMDMTSPLELNFQNTNRKSRGLRERFSATLQRNLPGHSMLDRELTTDGQKNQESRFSPGMIMDRLMHFGVRTRLGKVRNSASKYGGQVTFKFAQTKGTFLDQIMKHKDTSGGVCESISAHWISAHAKGDSIFNQLYVGGKKGKFHIDTLFSIKQLQMDGYLDDEQSTMTEYWLGTQGMQPNIQRNDDTDEHSSKVVGETGNRGTKDLLHAILDTGDKGSGYKKISFLGKMAGHTVAAYVDDQKGVTFFDPNFGEFSFPDKTSFSHWFTDDFWPKSWYSLEIGLGQEFEVFNYAPEAP

SEQ ID NO: 6 - protein, 287 aa, Photorhabdus
MPNKKYSENTHQGKKPLMKSEANNEHDIQNSSLGIGLDLNSMMGNSSTSLSHIQDYSFWKENISEYYKWMVVVKAHLKQLDWTLKSMDSPESAGTNIAKNTGTTALQTLLNTGGSIAGAAIGGAIGSAIAPGVGTIAGMGIGALAGTGLNYLNDTVIEKLNEKLEIAYPYPKTRNMIFDINNYDKNPIIKAIKKKTNKDNLKVTAGSSLTSQLVGKVTSPIKFPAYKLADLAIALAGLSSDKARHILDFTDSIREVLNESHSDAVAFMRKNYGDNAMGLAGLSSRIK

SEQ ID NO: 7 - protein, 322 aa, Photorhabdus
MEREYSEKEKHKKRPIQLRNSIEQHEEETANNSLGLGLDLNQATNPPKVPKDNYNEENGDLFYGLANQRGRYIKSVNPNFDPDKINSSPMIIDVYNNNVSNTILNKYPLDKLVKLSGNPQKYANNIKVENSLQQDVASSKRGWYPLWNDYFKTGNENKKFNIADIYKETRNQYGSDYYHTWHTPTGAAPKLLWKRGSKLGIEMAASNEKTKIHFVLDGLNIQEVVNKQKGSTPLEQGRGESITASELRYAYRNRERLAGKIHFYENDQETVAPWEKSPELWQNYIPKNKNQNESSTPQRNNGTLYRLGGPFRKLRASLRKRS

SEQ ID NO: 8 - protein, 308 aa, Photorhabdus
MMEHEYSKEEEKKRQQSKPNNATHDESNLPLELEKHFNARTPATAHSKWFTYENDTEVELTTERIKEIFSNKQPKIIIAGDGHNKPPFQYAKNIPDVNSSFDAGTLQLYIEATDEQINENNPEYIPKEFMAKPGLFTNKNRRAEIVGWEDSELSNAMKEMFELSDKSTREKLTPEETSSFYKLHETAIRHFFRPEFNQLRDEFFEILAKAGSNRELDKIALEMIGFTSGTWRDEYINPTLAEKIAKHAAEKENHTFVVSIGDAHLSENPMQEYLNKRRNGGEFKHQIIFTRDKRPILPDNMKTGNKNS

SEQ ID NO: 9 - protein, 298 aa, Photorhabdus
  misc_feature (148)..(148): Xaa can be any naturally occurring amino acid
MLKYANPQAVPTQRTKNTAKKPSSSSSFDGQLELSNGEWSKHSEMGLKRGGLINSIRRRIARNGNIGRFNELIDSEAKKWPSEPVDKNIHMIWIGTRNISEKNIKLSIDTAKKNPDYNTSIIYDSGISGHEGARNFMLEKFEGSNVNXSLAFPKGIGVMREYAPEAGKATAFPNTPIAVTKNNPIINKTLDLAVGNYQRGEKNVLKLAGPDVFTQALYQEIPGLNSKVLNAQLDQFELAKRQALGLPLEKPKSFADEKLTSVEKEKINRPYQSMRGLSGHVMNGADHSWAVDTEVLGH

SEQ ID NO: 10 - protein, 299 aa, Photorhabdus
MMREYSNEDDCTKEKTNLVKSENVEADNYLEMEHLTYLAKLISMTERENHHLNSIKLIDDIIELHNDRKGNKLLWNDNWQDKIIDRDLQSIFKKIDEMVSEFGGLEAYKDIVGESPYDPTEPVCGYSAQNIFKLMTEGEYAVDPVKMAKTGKINGNQFAEKLEHLNSSNNYVALINDHRLGHMFLVDIPSTNRERVGYIYQSDLGDGALPALKIADWLKSRGKESINVNKLKKFLNDEFTMLPENEQKGLIAEIFDLNKDIDSVKSGKIKKDKAVDIYLREYDINDFISNVEKLKTKLA

SEQ ID NO: 11 - protein, 148 aa, Photorhabdus
MIFKMLNLAVFYLLGNIFHYLICQKFICYFCSVLKSVTMFLTKVAVQIALYLNILPTMAGIAGLHAEVQALNNLFISGDRGTEKRENWKYIRNMLESTIFTQRLTAGQAGKDFAACHNCSGILSSPVNVITGKVESAGGNFFINIISI

SEQ ID NO: 12 - protein, 170 aa, Photorhabdus
MEREYSEKPKNLSQLSRKTAISERRAMFERNASSNNEQPVPQFARSYTSNRSVVNINPGRSSIAVVTANSTSPVNISTPAAASPDKLLPSTSCDTTSSTLTVGKYKLELTSQGKVVVFRGDNRTPEQIVAAGGFGEQSNYLYKMEIPGLKPQDMNERTLGEKIRQDSRGN

SEQ ID NO: 13 - protein, 298 aa, Photorhabdus
MKYDPRLRTWVEDDFDYEKNFKKQTDYINYKDLEKQLKENVDYYALLDENEAIIFLKELGCDIKSFLNDTAFPVTDVLSNFAGNIKDALGVFKVAKNFKPINIGIFTYIINELKGKGIKAIEYLGKNGERYIKLTDRPGIRKYLNATRYLINNKKIMEVGIGSVAMEGSIVKGARFGVIYSAAYRSVELMFKSEYDLTNFFVNLSMDMAKIIVATIIAKSTVAAATSFVVTAALSTTAIAIGVFIIGALVVWGLMWLDDEFKISETIIRRLKEHKVKTPISTYHSDQIFNAWGRYYRG

SEQ ID NO: 14 - protein, 328 aa, Photorhabdus
MPNKKHSENTHQGRKPLIKSEANNEHDIENSSLGIGLDLNSTIGNNSASLSQIQDYSFWKENISEYYKWMVVVKAHLKQLDWTLKSMDSSESAGTNIAKNIGTTALQTLLNTGGSIAGGAIGGAIGSAIAPGVGTIAGMGIGALAGTGLNYLNDTVIEKLNEKLEIAYPYPKTRNMIFDINNYDKNPIIKAIKKKTNKDNLKVTAGSSLTSQLVGKVTSPIKFPAYKLSDLAISHNRALAGLSSDKARHILDFTDSIREVLNESHSDAVAFMRKNYGDNAMGLSGLSSRIKGEKLTLATLARTRNKIENRINSINKQTLKLSSKNSNE

SEQ ID NO: 15 - protein, 322 aa, Photorhabdus
MEREYSEKEKHKKRPIQLRNSIEQHEEETANNSLGLGLDLNQATNPPKVPKDNYNEENGDLFYGLATQRGRYIKSVNPNFDPDKINSSPMIIDVYNNNVSNTILNKYPLDKLVKLSGNPQKYANNIKVENNLQQDVASSKRGWYPLWNDYFKIGNENKKFNIADIYKETRNQYGSDYYHTWHTPTGAAPKLLWKRGSKLGIEMAASNEKTKIHFVLDGLNIQEVVNKQKGSTPLEQGRGESITASELRYAYRNRERLAGKIHFYENDQETVAPWEKSPELWQNYIPKNKNQNESSTPQRNNGALYRLGGPFRKLRASLRKRS

SEQ ID NO: 16 - protein, 308 aa, Photorhabdus
MMEHEYSKEEEKKRQQSKPNNATHDESNLPLELEKHSNARTSATAYSKWFTYENDMEVELTTERVREIFSNKQPKIIIAGDGHNKPPFQYTKNIPDVNSSFDAGTLQLYIEATDEQINENNPEYIPKEFMAKPGLFTNKNRRAEIVGWEDSELSNAMKEMFELSDKSTREKLTPEETSSFYKLHETAIRHFFRPEFNQLRDEFFEILAKAGSNRELDKIALEMIGFTSGTWRDEYINPTLAEKIAKHAAEKENHTFVVSIGDAHLSENPMQEYLNKRRNGGEFKHQIIFTRDKRPILPDNMKTGKKNS

SEQ ID NO: 17 - protein, 1633 aa, Photorhabdus
MSNYEYDIVTQHDTYQIKDNEYTVVNGKYWQYEQEGNKNNNKISISLMKDNQNDPVWITSDIKEISLYIIENLFSYHKFSAELQHTLKNAVKAVFNEYSEIKYSELLHNINNIFNLFFIKTYNTSDINTAINILTAKIEIYDKLEKINQDKTDLNNTKVDIWEELGINAEEPLLKIYRQAFSTGDIDDEVYSDALLTFMSDGNLKLGDKEKSDYNQRIKDKTDLFESYKKGIEKVASLITTNNINPGIPITYPETEKSINIGDDLLLAQLAKEEIALKKQNRTEYSQQDIFELQTLQAAKYHLLILSSLGALLYQIAPNVEKMTKGHGDYRDIIFSQEQAESLFKKHNIQYDTNHVLSQESKHIEMEGCIILTAAIIYRMRKENATVEQALNYSTLETIKLFENDKKKLNPFNTNNVKPAGYFSFIDFKKRDKFDSQYNFNEQFNVYKNKYSHYESISFSKLILSSPAAQLTAEEIVNPPEETFLYSVEQGMGNVAMIKMYQGNWLVVSTIQGGVKARKYSQQQVDSQPTLRAMSRPNALFLIERKIMIGIGIFMENQIVNTGKRLFPTGYERAKTLSGFAETSRYKNSYNAFWNDYYGITSGMNVGISFTGSPKFNFYKEENLLSVTATIIQQGLNDIAIKSKQALDITSGWHIAATILIPFYNVIYKSTTDSEYELTGEDIGSIVFDTANVLLVVATLGMSLTESMAAKVTQTTLRLRQAGLTGRALITAVVRTLPEHGIITLRQSSGIILGGLIDLIEPLPIRSTLTLTYRGVISAVGAMRNSIKLEKSFADIFGKSTRGLGKLKHEWKVSNLPLEEIVPHSNGGEIYKGIYSIRHTNPETAVKQNFYIKEAGANYQVKWDDANHTWRVVNPTYPEQFSYWPAVKLDKNGHWVTHADISNKFLILEKSKRIDQELEAAHSNINNDNILDAFIHINTAFKDCERYDIDKLSDITDTLTHFFEKSLKPGDKKAIFSTEIMSIQQAWIREVILPLQNNSSISIEKINAIKTELPYLLRKTFPIESQLPNQLVANKIALAIEEIPNTRIPKYTSGNISKTVQYTSLLENNHVDIPPVGITITGNDTFINQVTRVLSEIDEIPSGNIVIQELEKQGLNIQPPTMNDIVREKNGQFYANNSAGSHIAFDPENHLIGTEEKLIDEPWRTREPAIALYHEMLHIYYNRYPTWFTSIDNKVIDQKVSGGFSLLEESRIVGTKYYVNDKDTLFDFNDSDYLLENNSALLTENRFRAEYAIFKNKSEYVIRPYSGKGDSQIPLTKTKININESHRNVMGVGSGKPEKMPNESATDYRNRVREWRKANKQPEADIGTGDMRKTKAEARVKLLKENYPQFEPQKIELGGAFQLWTVPNEPANKLMLSSHGYFFSDSAATQVPAGKTIQFLGPHGKTLLEAPENPLNSPFDVTLGNSGFTVQPYATIESGNKAGLGSVKIGDKTFTVNDIQNIATDDVENYLLATGVEANASNHGKVRNYGIKYYEKMPDEEVKAAIWKNRADETSTHKYDALLVSPEAGNRKKLSDIFALMKTDERMSKYDEITFVACREELNRINMKSIHDTGLGGGYEPKLEPTVILSRRRREATFTADGAIIYSIIAVNLHHNFITEEIVGIAPFLFIDN

SEQ ID NO: 18 - skipped

SEQ ID NO: 19 - protein, 293 aa, Photorhabdus
MFKYDTSEKMAKFGKGKTSDGMLLDTLYLEIPDEKAVMSAYKSQILDELRNFSEKTHSFFSGKKPLYSKKYLANLAAHAGYVHVTDYNSIGNYKDGFVNFKDNSRNLAEGKLFPGIRLIKRPKLSIVRDKETERWKKQESDEADAYEITDIESFISGVRDMYSRANVDLHPVIESLIRNHIVNNDHVLPTMAGIAGLHAEVQALNNLLILADGRAGKIVGGRKIEEYMQDMLKSFIFTQRLTTKQAGNDFAACHNCSGILSVPANVITGKVASAGSNFSLILSRYKNSQESPI

SEQ ID NO: 20 - protein, 340 aa, Photorhabdus
MLKHANPQTVSTQRTKSTAKKPSSSSSFDRQFELSNSENQPGEGNKDWTIKGWRQRFADRSLNKGHISPLMNKGLLVGSEEALINVPVVAHRYDSSHQLTDAGPLKADSHSNNLDPFYGVVTGFRGDQVTSSESGSGSIGGHWGKNTLDSNITGINVVNGASGTVGIRIALKDIQHGAPVIVTSGALSGCTMVYAVKNGYFFAYHTGQKPGDKEWKTGRQGVVATYRSHQALSPDSEPMAVGEQNNDLVNIFASYDQGIITYMGKPGVIIDNTAENVGVFNYDEVKLEKPDIRAGYSYALLAKDDKGKVNVKVLSEDVIVPLGNKGKTIKAINSLKKRLL

SEQ ID NO: 21 - protein, 336 aa, Photorhabdus
MPRYANYQINPKQNTKNSHGKSSSSNFSSGYFSSSNNSLDDSLIRQQVKREFIWEGHMKEIEEASRLGNFAVSFRAAGGPTLRALGKGAAAKGHDILEKTIKPGSINKAYPKDEASNVIKKVQEAGIEGYVGHWDKKTGRLLGIYMSSGHGLSDEQVNGKIYPIDLNNLEASLSALKTKENWAALPFTGDYDMHDMISFTGQPHSVPSNSSEERKIIDRINRLVARSDPNRPFGDIEHNVIRHGAQVSYPAFAMDKEKEEIKKHGGIVKAVAEPGEFPVAIVSKGKWTIANNIDELNQFYNSIGAKMKVSWKPGAENPGFVSNPQRPGMARFSRKR

SEQ ID NO: 22 - protein, 299 aa, Photorhabdus
MMREYSKEDDCVKEKTNLAESENVEADNYLEMDCLNYLAKLNGMPERKDHSLNSTKLIDDIIKLHNDRKGNKLLWNDNWQDKIIDRDLESIFKKIDEMVSEFGGIEIYKDIVGENPYDPTEPVCGYSAQNIFKLMTEGEHAVDPVKMAQTGKINGNEFAEKLEQLNSSNNYVALINDHRLGHMFLVDIPSTNREKVGYIYQSDLGDGALPALKIADWLKSRGKESINVNKLKKFLSNEFTMLSESEQKELIAEIFDINKDIANVKLGKIKKDKAVDVYLREYDLNDFISNIEKLKTKLV

SEQ ID NO: 23 - protein, 327 aa, Photorhabdus
MPIIGHKEDLIRTERSSVDLTRSSNNRQTDNLELNIPQHKRDNKDIEHAVIYGFSQHRGPEMQKAFADNKNPVTIDEYNAGLGIMGELSLSDYFRISQDLKENRLPELNEKNIQNHSLKYFDAMGVNMKSADPNVKEEAKEQQRAYTRSWGFYMMENKEKLDIQSKINNLIPKKKSFFSKSPGEDEYKKLDEFILKNSNGSNLTIPKQRKILMKFASAKNAVDVTKNLSGEEQTWLKDIIATAFFRQTSKLGMSWFIEQLASPDFRFVIVGFNGEELTTDQIRSNKPWKHGNRRKEGASEYAEPITFSEIRHAHRKGYDSKINFIKK

SEQ ID NO: 24 - protein, 926 aa, Photorhabdus
MISTFDPAICAGTPTVTVLDNRNLTVREIVFHRAKAGGDTDTLITRHQYDLRGNLTQSLDPRLYDLMQKDNTVQPNFYWQHDLLGRVLHTVSIDAGGTVTLSDIEDRPALNVNAMGVVKTWQYEANSLPGRLLSVSEQSANEAVPRVIEHFIWAGNSQAEKDLNLAGQYMRHYDTAGLDQLNSLSLTGAHLSQSLQLLKDDQMPDWAGDNESVWQNKLKNEVHTTQSTTDATGAPLTQTDAKENMQRLAYNVTGQLKSSWLTLNGQLEQIIVKSLAYSESGQKIREEHGNGVVTKYSYEPDTQRLINITTQRSKGHVFSEKLLQDLLYEYDPVGNIVSILNRAEATHFWRNQKVSPRNTYTYDSLYQLIQSTGREMADIGQQNNKMPTPLVPLSSDDKVYTTYTRTYSYDRGNNLTKIQHRAPASHNIYTTEITVSNRSNRAVLSHNGLTPREVDAQFDASGHQISLPTGQNLSWNQRGELQQATTINRDNSATDREWYRYNAGSARILKVSEQQTGNSTQQQQVTYLPGLELRTTKSGTNTTEDLQVITMVETERTQVRILHWSAGKPNDIANNQVRYSYDNLIESNVMELDTKGKIISQEEYYPYGGTAIWTARNQIEASYKTVRYSGKERDKTGLYYYRHRYYQPWLGRWLSADPAGTVDGLNLYRMVKNNPIRYQDESGTNANDKAQAIFKEGKKIAINQLKIASNFLKDSKNSENALEIYRIFFGGHQDIEQLPQWKKRIDSVIYGLDKLKTTKHVHYQQDKSGSSSTVADLNVDEYKKWSEGNKSIYVNVYADALKRVYEDPLLGREHVAHIAIHELSHGVLRTQDHKYIGVLSSPGSHDLTDLLSILMPPANEQDRTEKQRRATGARKALENADSFTLSARYLYYTAQDPNFLSSLRKAHRDFNNKKTDRLIIRPPERR

SEQ ID NO: 25 - protein, 324 aa, Photorhabdus
MEREYNKKEKQKKSAIKLDDAVGNNEENMDMTSPLELNSQYTNRKRPGLRERFSATLQRNLPGHSMLDRELTTDGQKNQESRFSPGMIMDRIMHLGVRTRLGKVRNSASKYGGQVTFKFAQTKGTFLDQIMKHKDTSGGVCESISAHWISAHAKGESIFDQLYVGGQKGKFHIDTLFSIKQLQMDGYLDDEQSTMTEYWLGTQGIQPNRQKNDNMNEHSSKIVGETGTRGTKDLLRAILDTGDKGSGYKKISFLGKMAGHTVAAYVDDQKGVTFFDPNFGEFNFPDKVSFSHWFTDDFWPKSWYSLEIGLGQEFEVFNYEPKEP

SEQ ID NO: 26 - protein, 304 aa, Photorhabdus
MVYEYAKTNDRKRKLSTQSDNYEEKSFSPVLDLSRNNQNTPNMEDEYETPQNFINRTGREKLFRAIRMVASNKRDPITKDQVSVPPDGNLFTELKDKHLDRAAEYKKLKTWPTHASIIATSPSANTPIAQHVSGDDALSPYISTGDKPGAVQNTVRNWNGIGPASERRLRPEKTWSPIIEIDVNKLPDTTKIFDLNKPNNTFFSTTNSDIAQNAFADKEVLISPEIPGLAITRVINDPEEIKQIANLNPSQSLIEKKNTIPEEKIIFEEKKSVPIHDSDADIPSSSFVFPKRKKPRNIRSRTDS

SEQ ID NO: 27 - protein, 542 aa, Photorhabdus
MVFEHDKTVERKRKPSIQLGNDKEKSSEQALELPQSKQNNPLLHDLITSNNLRKEAAVFAKQIGPSYQGILDGLEHLHNLSGNEQLTAGFELHRRITRYLEEHPDSKRNAALRRTQTQLGDLMFTGTLQEVRHPLLEMAETRPAMASQIYQIARDEAKGNTPGLTDLMVRWVKEDPYLAAKSGYQGKIPNDLPFEPKFHVELGDQFGEFKTWLDTAQNQGLLTHTRLDEQNKQVHLGYSYNELLDMTGGVESVKMAVYFLKEAAKQAEPGSAKSQEAILLNRFANPAYLTQLEQGRLAQMEAIYHSSHNTDVAAWDQQFSPDALTQFNHQLDNSVDLNSQLSFLLKDRQGLLIGESHGSDLNGLRFVEEQMDALKAHGVTVIGLEHLRSDLAQPLIDKFLTSENEPMPAELAAMLKTKHLSVNLFEQARSKQMKIIALDNNSTTRPAEGEHSLMYRAGAANNVAVERLQQLPAEEKFVAIYGNAHLQSHEGIDHFLPGITHRLGLPALKVDENNRFTAQADNINQRKCYDDVVEVSRIQLTS

SEQ ID NO: 28 - protein, 2957 aa, Photorhabdus
MKGIEGVIMLSHDILPEKLLVSEKKHENVGSYFSDDIGEQSEQTEVSHFNLSLDDAFDIYADISIENQQELKNKDNNTNIWSSLGRGDDDHNLKKIINDAFKEKLPQLMEYRRKGYNVIGLDKEGIKKLEGMLKAVPPEIQQPTMKNLYSAAQELLNTLKQHPLLPENQDMIQQSNLVIRNLSDALEAINAVSKVNQVEWWEEVHKTNKAQSDRLIAATLEELFFKVKDKRLPGSNDDYCQQEREETERKIKDLLLYDGYQLTAEHFKFGRLRKSLLAESRVTRLKLAEYLEKKSVGILTAARDAKMYAMKILLAQTRNNGFNAKDLINAGQVNDRLLSFQQYARHIRAVDGEIDGIILSNPLVVACIKETNDEPAHIKIARAILPVSEELGTVSKVLRETKEKVQPSKPKEELNHPHQDWWNRGDELWKYIKKTSWNIKETSVHVTQMVGYEASKTASRAKHKLKESSYSESINGAVKGTALLLLDEIQQAENRIRQIPQFAWDVQEAVEQHSSVIQRTAYPDELPELSELLNEQLKHEEARWQAVKKQSRDKLQELIAPITRLAQEKWAQDLYFQLGEELRKERQDRWKDIQQFDEIMAEAVGQFAEMARELDSEAVRLAEHGHSGGKELQEKVAKWLRDLSKLKGKVKAGVAKITGTSLDNFSRSGMLARGMSEWAEDLKQSYLQETLQEGSAVAAELFERTLMEVVEENRTHFAKESDPEAERFLKRLALALKHAAENTTVYPPTPEEILAGSRSLPEDIRHWAEKKVVSGAISAAFRGGFKLVTGTFSLPVRVVIRGAKTGGTLYRGVRAINRSVRLGQGPATQVKSKFINQELSKTAFRLTLSLSPLVAWGMAASITAGRLYNEKDYPEKIIKNIVIDLPEELLWIGGYAGINAAIRAHAEKAIQQAIQHALDEQADKLALRINKEIAGKSADVNVEIIPQETSVSPAETAQSTPEPLSDFASTSQLTMPELIDIQDNNSAQQPKVRRKRDVSVESEISIDNLNIINANTREDKVNSEIKSELRSELKRFENSDANSPMSDVERAIFIDLFLYKNKYEVSESQQDYKNTWLKFRRELESQENKEIKEYLRFRSIIEAYEIYDKKRLDDDTIPEAGTIIKEVIDFFQKLKKENPITFMKLAEAMVKFQYYYEEEDENEDRYFKMAEIYYFLNKTENEKKSKTFHLDIIDKYPNENNRLLDEFFLNKNNNNPDLDEIIYKLQSMQEKYRESYEMLSKVENIHQVLSDDSKNEENIFLDNRIIAAQVFDGSINISLQDKKKWLNRYDQIRNEEGSDGWKLMHIESILINLRRINTAINLTAMKSESALLLIDKLLNFQKKARENILHISETPHEDFTSYSQFKTRKELGNDDSKYYAQFDNYKDNHDAEKEAKEILSQVVARASLSFSELFDKVESIKLFSFVYKNRDGGAPLAAPGRTVVIKFPGKDTGGLVISNLFLRNHVKRISTKEMEDLKPLTEGMYTRATQHRSLGSYYHIGSQSEHTNALEILSGMNKEELKTHLKKQGIWFGEPALFSNEYPKQENTGHLENTTLKNAIIGVSTIQNNAAANYLRSTMYESTGWEKLGDRFIPFYEIGRRKHYDREYEINSEQLTLDIITSIAIAYPAARGIVATIRSSAIPSILKSGLRGSALFKSLSLELGKMGFNASKVFGGAVYELIEPYPINSHLNRHNVFNKVKDTAWEFHTDVGLKGGGLKDFIDRFTKEPKEITISGYKFKRIKYNQENFDTMQRMALDYAYNPDSKGKIAQAQQAYKTGKEDYNAPQYDNFNGLSLDKKIERYISPDTDATTKGVLAGKMNESIKDINAFQTAKDAQSWKKSANKANKVVLTPQNLYLKGKPSECLPESVLMGWALQSSQDAKLSKMLMGIYSSNDITSNPLYKSLKELHANGNASKFNASATSISNINVSNLATSETKLFPTEISSVRVDAPKHTMLISKIKNRENKIKYVFYDPNYGMAYFDKHSDMAAFFQKKMQQYDFPDDSVSFHPLDYSNVSDIKISGRNLNEIIDGEIPLLYKQEGVQLEGITPRDGIYRVPPKNTLGVQETKHYIIVNNDIYQVEWDQTNNTWRVFDPSNTNRSRPTVPVKQDTNGEWFKHSETGLKGGGPIDDIRKYIARKSAIKIFNQSINYSATKWPPEPIDKNIHMIWIGTKNISEKNIKLSIDTAKKNPDYNTSIIYDSGISGHEGAKKFMLEKFQDSNVNIIDFRKKSYFSQLKQEPSFAYYEQVIAENKYAQASDILRLLVLKYEGGIYKDIDDIQVKGFGSLTFPKGIGVMREYAPEAGKATAFPNTPIAVTKNNPIINKTLDLAVSNYQRGEKNVLKLAGPDVFTQALYQEIPGLDSKVLNAQLYQLELAKRQALGVPLEKPKNFADEQLTSAEKEKINRPYQSIRGLSGYVENGADHSWAVDTNIPSTSTQTSTIVTPLAPKTEMLPPVPSSSTKSSTSAPVLQEKISYNLATDIDATDYLNQLKQKTNINNKISSPAGQCESLMKPVSDFMRENGFTDIRYRGMFIWNNATEQIPMNHFVVVGKKVGKDYVFDVSAHQFENKGMPDLNGPLILAAEDWAKKYRGATTRKLIYYSDFKNASTATNTYNALPRELVLESMEGKTFITSPNWYQTFKRTHNIHPEVTVSDPATFSLNYSVNPTAENLSPPPPPPIPSHGQVPKTVTPPPPPMRSPLSLSQPLERLPANKTKPIGFNPGENKASFSKLEEAGKHYYKDDKSRQAAPVNTMSDFDNRYLSHTTEAPAPSNVAHLAPGNIYNTKVTAKGAEKPAYDIYISKDGESLITSSSYKVDDITTDSKFGKPLPYSEIMFNSLKKSGVDPKNLKRSVQASIENKVTQDVISAIGTRIQRGQVIRVSPTENPDAFYTLLGTDNCKATLHMLNQHAEEFGHKVVTSIEFKGTGYLVMNIGTSTQTSTIVTPPPMPGTSQLVQ

SEQ ID NO: 29 - protein, 327 aa, Photorhabdus
MPNKKYSENTHQGKKPLIKSEANNEHAIDNSPLGIGLDLNSILGNNSASLSQIHDYSFWKENISEYYKWMVVVKAHLKQLDWTLKSMDSPESAGANIAKNIGTTTLQTLLNTGGSIAGGAIGGAIGSAIAPGVGTIAGMGIGALAGTGLNYLNDTAIEKLNEKLEIAYPYPKTRNMIFDINNYDKNPLIKAIKKKTKKDNLKVMAGSSLTSQLLGRITPIKIPAYKLADLAVSHHRALAGLSSDKARHILDFTNSIREVLNESHSDAVAFMRKNYGDNAMGLSGLSSKIKGDKLTLDTLARTRNKIENRINSINKQTLKLSSKNSNE

SEQ ID NO: 30 - protein, 322 aa, Photorhabdus
MEREYSEKEKHKKHPIQLRDAIEQHAEETANNSLGLGLDLHQAINTPKVPKDNYNEENGDLFYGLAAQRGRYIKSVNPNFDPDKTNSSPMVIDVYNNHVSNTILNKYPLDKLGKLYGNPQKYAKDIKVTNSLQQDVAASKRGWYPLWNDYFKAGNENKKFNIADIYKETRNQYGSDYYHTWHEPTGAAPKLLWKRGSKLGIAMAASNEKTKIHFVLDGLNIQEVVNKQKGSTPLEQGRGESITASELRYAYRNRERLAGKIHFYENDQETIAPWEKSPELWQNYIPKNKSQNESSTPQRNNGALYRLGGPFRKLRASLRKRS

SEQ ID NO: 31 - protein, 297 aa, Photorhabdus
MVHEYSINDRQKRHSFSSANPIDPEVTNRENSRHRFPKDNYNKGHGDLFYGLAPERGKYIKEANPKFDPNNPENAAMIIDVYNDEISRVILNNNANKISTNRLLNFIYNFRKNRLENLMKNPEKYAKDIKVKDNLRENISPKKIEKYPLWNDYFEAGIRNKKFNIAEIFKETASQYNSDYYHAWHIGGNSAPRLLWKRGSKLGIEIAASNQRTKIHFILDGLKIEDVVNKTKGPAPLKAGPGESITASELRYAYRNRARLAGRIHFYENGKETIAPWDKDPELWQKYTPKNRSGMEL

SEQ ID NO: 32 - protein, 340 aa, Photorhabdus
MLKYANPQTVATQRTKNTAKKPPSSTSFDGHLELSNGENQPYEGHKIRKIKGLRQHLADRSLNKGHISPLMNKGLLVGSKDVSIDIPVIAHRYDSSHQLTDAEPLKADSHSNHLDPFYGVIAGFRGDQVTSSESGSGSIGVHWGKNTLDSNIMGVNVVNGASGTVGIRIALKDIQHGSPVIVTSGALSGCTMVYSVKNGYFFAYHTGQKPGNNEWKTGRQGVVATYLSHQALSPDSEPMTVGEQNNDLVNIFANYDQSVITYMGKPGVLIDKMAENVGVFNYDEIKPEKPAIRAGYSYALLAKDDKGKVNVKVLSEDVIVSSGKQGNTVKAINSLKKRLL

SEQ ID NO: 33 - protein, 336 aa, Photorhabdus
MPRYANYQINPKQNIKNSHGKSSSSDFSSGYLSFSNNSLDDPFIRQQVKREFIWEGHMKEIEEASRLGNFAVSFRAAGGPTLRALGKGAAAKGHDILEKTIKPGSINKAYPKDEASDVIKKVQEAGIEGYVGHWDKKTGRLLGIYMSSGHGLSDEQVNGKIYPIDLNNLEASLSALKAKENWAALPFTGDYDMHDMISFTGQPHSVPSNSSEERKIIDRINRLVARSDSNRPFGDIEHNVIRHGAQVSYPAFAMDKEKEEIKKHGGIVKAVAEPGEFPVAIVSKGKWTIANNIDELNQFYNSIGAKMKVSWKPGAENPGFVSNPQRPGMARFSRKR

SEQ ID NO: 34 - protein, 328 aa, Photorhabdus
MPNKKYSENTHQGKNPLMKSGANNEHDLQDSPLGIGLDLNSMLVNSSTSLSQIQDYSFWKENISEYYKWMVVVESHLKQLDWTLKSMDSPESAGTNVAKNMGVTALQSLLNTGSSIAGGAIGGAIGSAIAPGVGTIAGAGIGALAGTGLNYLNDTAMSKLSKKLEIAHPYPKTRNMILDINNYDKNPIIKAIKKNVNKDNLKVTAGSSLTSKLVGTVTSPIKFPAYKFAELAVSHHRALEGLSDDKARHILDFTNSIREVLKESHSDAVAFMRKNYGDNAMGLSGFSSKIKREKLTLNTLAKTKNEIENRINSINKQTLKVSSRSRNE

SEQ ID NO: 35 - protein, 324 aa, Photorhabdus
MLSTEKHNKDTKHPRNREKKFSIQPENSTQDDEDIKNNSLGVGLDLDQMIRNTSSTLTNAPQKPEDGYYYHISRGNNLQSFLQNGFKPQGSPGPTLSEEDFSRRKIGIIKLIYSIIATTINKNRKAKKISKDNFLMPQEFWHEFKNFYQNIPTQTNIDDQLLKKSITESIDKLDQNKFMEKHSDRKQTIINNEREAILQQDERINEIISSRAKMIQQREAENTEGYIYLAPHKNTLLEYMKHLQEEKNLFLILAVKEDIFTEKGLEQDPQEPHGAVRYKGALSTEELNFVNQEGQICAIPASIGEMDYGDFILNQQQVIDFCKK

SEQ ID NO: 36 - protein, 336 aa, Photorhabdus
MPINDLKKKFEISPQAAQAIGAPARSNSSKQAEHQTEHLELDTSKNRRDRKDLNAQATPNQQHTKKLETEVNNGGNKSKAQAHTPDLVMKKESSVTPNTRKSPNEKIKAEDIFHRYKDRFSPSDRELPFEIMNEITNNGIAFSSEKAPESHLDKVKDKKFTLRHYTSGNGQEKPTFNEIGSNFNLVNEGIKTLKRTQGSNTNEDDWNRLGNTAFTFFLLAIDGEVSDRKFLSNTTHFAEIDIENPAELKELGLDETEFFASPDLLHEKNLSQAPAVKGKLSDLKSLLLKQSGIKPVQLQSLGAKGILERIDSKFNGSLEIKIPGNVKVKEWKKVEK

SEQ ID NO: 37 - protein, 315 aa, Photorhabdus
MPNSKYSEKVNHSANGAEKCSIHSNQYNINNCTLGLGLDLNKKLRTGNERNIEGAQPFIPFPSKQKQYSTSPIAMADILNESALTSQPIITDLINPQKIKMSDGVKNILNNKEGGGDLVFKALQIKPSDETLPFNALKIVDTYQEEMPNKDMSISAYWAPQGGYVDIPAQPDISRHPQYVFTPNFSGCSFVVDKMNEDTLRVRHVQGGQEDVEYNNQNIDHGMGMITAMEFRDYGYHEADDKVIENTYGFAFLKFNQEKKQWQLHYQKIAAAPNIINIKTKSSWLPFSKPSIEADTFTFKNMKVPGYSRKNINNN

SEQ ID NO: 38 - protein, 309 aa, Photorhabdus
MPKLTELLSRFENPIQNQPNHISKKNPISNSKVLNNSEEKTAPLELKHDDSKIKSQVSIPNLVKKNEKPAASNTPNNSHEKVKAEDIFNRFKSKFDPYDRELPFDIMNKITNNEIKFSSEKSKDDYLAKVKDKKFTLRHYTAGTGQEKPTFDEISSNFNLVNKGIKTLNRTQGSNTNEDDWNRLGNTAFTFYLLAIDGEVSNRKFLSNTTHFAEINIEDSEELKELGLDQAEFFASPDLLHEKNLSQAPAVKGKLSDLKSLLLKRSGISSVQLGRLDAKAILKSIDNEFGNSLEIKIPGNVKVNKWNKI

SEQ ID NO: 39 - protein, 340 aa, Photorhabdus
MPRYSNSQRTPTQSTKNTRRTSPSSNSSTEHLSLSNAPTNDSSVRQEVKEKFIWEGHWEGHMEAIEKASILGNFAVSFRAAGKPTLEALGKGAAAKGHDILEKTIKPGSIEKAYPENEASDVIKKVREAGIEGYVGHWNKETGRLEGIYMSSGHGLPNGQVNGKIYPIDLNNLEASLAPLKEKKNWAALPFTGDYDMHDMISFTTQPHSVPSNSSEEKKIIDRINEYIAKSDSNRPFEDIEHNVIRHGPQVSYPAFAMDKEKKEIKERGGIVKAVAEPGEFPVAIVSKGKWTIANNINELEQFYNSIGAKMKASWKPGAGNPGFVSNPQKPGMARFSRKK

SEQ ID NO: 40 - protein, 280 aa, Photorhabdus
MFSTYSSKNDNQTINKINTEEKHENTETDNHLEINLEHTGKSKPDIEPKDVTTGTINAGTLLYKTTAIPEFLDNAKSLGLAEYEKRHKDIQDYLNLGKAEDAEKLKNKSQWAGQYFALEKSYDEYANEAPDSYNNLLKNAGKDLLENTEEVKVFLYTFKVTKDIKVLKPHNNSNSYYVGDTEGWEKAKEIMNDVQSQSEKNDNPFPELKNLEDKNFLLEELGEKGYAWMGPLHAKEGAEKGTEFSYELAISPNLLRQHLTLESEELLGTYKNRYGYWDKK

SEQ ID NO: 41 - protein, 138 aa, Photorhabdus
MKKTDEKYGQYEYKDEDITSYPIAWTNPDNGKIYIGINSPEYSHLNNKGESELNLAKIISTIIHESLHASSHQHKGLQSQTDTGADNLNYDEYVTDYFAREVYKQILPDKDYVANCFTKGLGGENKIWGGNIVEFMIQ

SEQ ID NO: 42 - protein, 539 aa, Photorhabdus
MVYEYDKTIERRRNPSIQLNNNEKSSEQALELSQNNPLLHDLITSNNLRKEAAVFAKRIGPSYQEILDELEHLHHLSGNEQLAAGFELHRRITHYLEEHPDSKRNTALRRTQTQFGDLMFTGTLQKIRHSLLEMAETRPEMASHIYQIAREEVKGNTPGLTDLMVRWVKEDPYLAAKTGYQGKIPNDLPFEPKFHVELGAQFDDFKKWLDTAQSKELLTHTRLDEQNKQVHLGYSYNELLDMTGVESVQMAVYFLKEAAKQAEPGSTKSQEDILLHRFANPTYLAQLEHSRLAQIEAIYHSSHDTDVTAWDQQFASDALTQFNHQLNNTVDLNSQLSLLLKDRQGLLIGESHGSDLNGLRFVEEQMEVLKAHGVTVIGLEHLRSDLAQPLIDKFLASGNEPMPAELAALLKTKHLSANLFEQARSKQMKIIALDNNSTTRPTVEGTQHGLMYRAGAANNVAVERLRQLPAGEKFVAIYGNAHLQSHEGIDHFLPGITHRLGLPALKVDENNRFTAQVDNINQRKRYDDVVELPRIQLTS

SEQ ID NO: 43 - protein, 323 aa, Photorhabdus
MEHEYSEKEKPQKCPIQLRDSIEHDKEDINTTTPLELNSQYTNRKRAGLRERFSTTLQRNLPGHSMLDRELTTDGMKNQESRFSPAMIMDRMMHFGVRTRLGKVRNSASKHGGQVTFKFAQTKGTFLDQIMKHKDTSGGVCESISAHWISAHAKGESIFDQLYVGGQKGKFHIDSLVSIKQLQMDSYLDDEQSTMTEYWLGTQGIQPIMQKNDVDEHSSKVVGQTGNKGTTDLLRAILDTGDKGSGYKKISFLGKMAGHTVAAYVDDQKGVIFFDPNFGEFSFPSITSFSRWFTDDFWPKSWYNLEIGLGQQFEVFNYELKKS

SEQ ID NO: 44 - protein, 381 aa, Photorhabdus
MYDSKKKNSEPTTKKKFERSNYSQWDDSINHYEDMNRARIKNRNDILTTVDYFGEKKKTMHTFEYQSDIKHDTNFNNKNKSLFESFAASFVLQNPSFFSGVIDKLSKKLFNIISKIDERNNFQKKLYDFIEKDTSPEGQFGRFTLGKNEILNVLQVKSDTPQLFVKKMLLIKSLGAFIIDFSSKDIGNYDFIFDGKGREVNDIIEKNRPTNLFKVRGRTNIKSSQHRSDIGILDTPTFDSLTEEQKSFLTIPELTKRRPLFRTFTHELDAEDKRVVESVFVNRTFDCDSPLIGSVSGSTSCVLVAADILFPDMTMVERKKLAIATFAFLVGGGYHSATEVFDVAYPGLDLNKEIEELIENNPIQENAGVATLRQLIGNSGF

SEQ ID NO: 45 - protein, 308 aa, Photorhabdus
MPISNLAKESEVRAVKDIPCKNIETDNHLEIGLSSGLSRSKDTSKFKKNSINTIKLIDDIIALHNDPKGNKLLWNDNWQDKIINRDLANIFEKIDESVSELGGLEMYQEMVGVNPYDPTEPVCGLSAQNIFKLMTEGEHAVDPVEMAQTGKIDGNEFAESVDQLSSAKNYVALVNDRRLGHMFLIDIPSNDQETVGYIYQSDLGQGALPPLKIADWLNSRGKDAVSLNKLKKLLSREFNLLSDDEKRALISETLDIHKDVSNVELDRIKRDRGVDIYLTEYDVNNFYENIETLKSKLSNYDKKLSKPK

SEQ ID NO: 46 - protein, 295 aa, Photorhabdus
MLANVLPNLASFLKYEKETPLFFIEDGFNFQNLNPGRVPLIKTPEQRKAGDTQSPAFLCSGVILRGTIHSNDYKFWQPSPSSIKSGGVSFSYLRKDAKFKRLAYGYKNGFIIFPEHIAPEDRVDFSVLCAFPIDGYTNERANQGCGENITKAKDKGKSCQEQNVTNSDDWIKNYRKVNSQDFFQCGFNVTKDVNNPAIAFYQMLESIKKLPRTPNTPPKQNEIRISTWEESDPNKLPIEALFYSENSGLADAQKDQRDYKNATGKFLPIVKMLLPRTLNEDALFKFNIKDQVINP

SEQ ID NO: 47 - protein, 50 aa, Photorhabdus
MMREYSNEDDFIKEKTNLVKSENVEADNYLETEYLTYLAKLIGMTERENH

SEQ ID NO: 48 - protein, 50 aa, Photorhabdus
MFQNRIRNEKTTQSGKGKTLDRMTDSLYLEIPNVEAVTLAYQKLTSKYRK

SEQ ID NO: 49 - protein, 50 aa, Photorhabdus
MEREYSEKQKNPSKLSRKTAISERIAALERSGLSNSNQPVPQFARPYTSN

SEQ ID NO: 50 - protein, 50 aa, Photorhabdus
MSNYEYDIVTQHDTYQIKDNEYTVVNGKYWQYEQEGNKNNNKVSISLMKE

SEQ ID NO: 51 - protein, 50 aa, Photorhabdus
MEHEYNEKEKQRNSAIKLNDAIRNNEENMDMTSPLELNFQNTNRKSRGLR

SEQ ID NO: 52 - protein, 50 aa, Photorhabdus
MPNKKYSENTHQGKKPLMKSEANNEHDIQNSSLGIGLDLNSMMGNSSTSL

SEQ ID NO: 53 - protein, 50 aa, Photorhabdus
MEREYSEKEKHKKRPIQLRNSIEQHEEETANNSLGLGLDLNQATNPPKVP

SEQ ID NO: 54 - protein, 50 aa, Photorhabdus
MMEHEYSKEEEKKRQQSKPNNATHDESNLPLELEKHFNARTPATAHSKWF

SEQ ID NO: 55 - protein, 50 aa, Photorhabdus
MLKYANPQAVPTQRTKNTAKKPSSSSSFDGQLELSNGEWSKHSEMGLKRG

SEQ ID NO: 56 - protein, 50 aa, Photorhabdus
MMREYSNEDDCTKEKTNLVKSENVEADNYLEMEHLTYLAKLISMTERENH

SEQ ID NO: 57 - protein, 50 aa, Photorhabdus
MIFKMLNLAVFYLLGNIFHYLICQKFICYFCSVLKSVTMFLTKVAVQIAL

SEQ ID NO: 58 - protein, 50 aa, Photorhabdus
MEREYSEKPKNLSQLSRKTAISERRAMFERNASSNNEQPVPQFARSYTSN

SEQ ID NO: 59 - protein, 50 aa, Photorhabdus
MKYDPRLRTWVEDDFDYEKNFKKQTDYINYKDLEKQLKENVDYYALLDEN

SEQ ID NO: 60 - protein, 50 aa, Photorhabdus
MPNKKHSENTHQGRKPLIKSEANNEHDIENSSLGIGLDLNSTIGNNSASL

SEQ ID NO: 61 - protein, 50 aa, Photorhabdus; identical to SEQ ID NO: 53

SEQ ID NO: 62 - protein, 50 aa, Photorhabdus
MMEHEYSKEEEKKRQQSKPNNATHDESNLPLELEKHSNARTSATAYSKWF

SEQ ID NO: 63 - protein, 50 aa, Photorhabdus
MSNYEYDIVTQHDTYQIKDNEYTVVNGKYWQYEQEGNKNNNKISISLMKD

SEQ ID NO: 64 - protein, 50 aa, Photorhabdus
MEHEYNEKEKQRNSAIKLNDAIRNNEENMDMTSPLELNSQNTNRKSRGLR

SEQ ID NO: 65 - protein, 50 aa, Photorhabdus
MFKYDTSEKMAKFGKGKTSDGMLLDTLYLEIPDEKAVMSAYKSQILDELR

SEQ ID NO: 66 - protein, 50 aa, Photorhabdus
MLKHANPQTVSTQRTKSTAKKPSSSSSFDRQFELSNSENQPGEGNKDWTI

SEQ ID NO: 67 - protein, 50 aa, Photorhabdus
MPRYANYQINPKQNTKNSHGKSSSSNFSSGYFSSSNNSLDDSLIRQQVKR

SEQ ID NO: 68 - protein, 50 aa, Photorhabdus
MREYSKEDDCVKEKTNLAESENVEADNYLEMDCLNYLAKLNGMPERKDHS

SEQ ID NO: 69 - protein, 50 aa, Photorhabdus
MPIIGHKEDLIRTERSSVDLTRSSNNRQTDNLELNIPQHKRDNKDIEHAV

SEQ ID NO: 70 - protein, 50 aa, Photorhabdus
MISTFDPAICAGTPTVTVLDNRNLTVREIVFHRAKAGGDTDTLITRHQYD

SEQ ID NO: 71 - protein, 50 aa, Photorhabdus
MEREYNKKEKQKKSAIKLDDAVGNNEENMDMTSPLELNSQYTNRKRPGLR

SEQ ID NO: 72 - protein, 50 aa, Photorhabdus
MVYEYAKTNDRKRKLSTQSDNYEEKSFSPVLDLSRNNQNTPNMEDEYETP

SEQ ID NO: 73 - protein, 50 aa, Photorhabdus
MVFEHDKTVERKRKPSIQLGNDKEKSSEQALELPQSKQNNPLLHDLITSN

SEQ ID NO: 74 - protein, 50 aa, Photorhabdus
MKGIEGVIMLSHDILPEKLLVSEKKHENVGSYFSDDIGEQSEQTEVSHFN

SEQ ID NO: 75 - protein, 50 aa, Photorhabdus
MPNKKYSENTHQGKKPLIKSEANNEHAIDNSPLGIGLDLNSILGNNSASL

SEQ ID NO: 76 - protein, 50 aa, Photorhabdus
MEREYSEKEKHKKHPIQLRDAIEQHAEETANNSLGLGLDLHQAINTPKVP

SEQ ID NO: 77 - protein, 50 aa, Photorhabdus
MVHEYSINDRQKRHSFSSANPIDPEVTNRENSRHRFPKDNYNKGHGDLFY

SEQ ID NO: 78 - protein, 50 aa, Photorhabdus
MLKYANPQTVATQRTKNTAKKPPSSTSFDGHLELSNGENQPYEGHKIRKI

SEQ ID NO: 79 - protein, 50 aa, Photorhabdus
MPRYANYQINPKQNIKNSHGKSSSSDFSSGYLSFSNNSLDDPFIRQQVKR

SEQ ID NO: 80 - protein, 50 aa, Photorhabdus
MPNKKYSENTHQGKNPLMKSGANNEHDLQDSPLGIGLDLNSMLVNSSTSL

SEQ ID NO: 81 - protein, 50 aa, Photorhabdus
MLSTEKHNKDTKHPRNREKKFSIQPENSTQDDEDIKNNSLGVGLDLDQMI

SEQ ID NO: 82 - protein, 50 aa, Photorhabdus
MPINDLKKKFEISPQAAQAIGAPARSNSSKQAEHQTEHLELDTSKNRRDR

SEQ ID NO: 83 - protein, 50 aa, Photorhabdus
MPNSKYSEKVNHSANGAEKCSIHSNQYNINNCTLGLGLDLNKKLRTGNER

SEQ ID NO: 84 - protein, 50 aa, Photorhabdus
MPKLTELLSRFENPIQNQPNHISKKNPISNSKVLNNSEEKTAPLELKHDD

SEQ ID NO: 85 - protein, 50 aa, Photorhabdus
MPRYSNSQRTPTQSTKNTRRTSPSSNSSTEHLSLSNAPTNDSSVRQEVKE

SEQ ID NO: 86 - protein, 50 aa, Photorhabdus
MFSTYSSKNDNQTINKINTEEKHENTETDNHLEINLEHTGKSKPDIEPKD

SEQ ID NO: 87 - protein, 50 aa, Photorhabdus
MKKTDEKYGQYEYKDEDITSYPIAWTNPDNGKIYIGINSPEYSHLNNKGE

SEQ ID NO: 88 - protein, 50 aa, Photorhabdus
MVYEYDKTIERRRNPSIQLNNNEKSSEQALELSQNNPLLHDLITSNNLRK

SEQ ID NO: 89 - protein, 50 aa, Photorhabdus
MEHEYSEKEKPQKCPIQLRDSIEHDKEDINTTTPLELNSQYTNRKRAGLR

SEQ ID NO: 90 - protein, 50 aa, Photorhabdus
MYDSKKKNSEPTTKKKFERSNYSQWDDSINHYEDMNRARIKNRNDILTTV

SEQ ID NO: 91 - protein, 50 aa, Photorhabdus
MPISNLAKESEVRAVKDIPCKNIETDNHLEIGLSSGLSRSKDTSKFKKNS

SEQ ID NO: 92 - protein, 50 aa, Photorhabdus
MLANVLPNLASFLKYEKETPLFFIEDGFNFQNLNPGRVPLIKTPEQRKAG

SEQ ID NO: 93 - DNA, 19592 bp, Photorhabdus
atgtctacaagtacatctcaaattgcggttgaatatcctattcctgtctatcgctttattgtttctgtcggagatgagaaaattccatttaatagtgtttcaggattagatattagttatgacaccattgaataccgagatggtgttggtaattggttcaaaatgccgggtcagagtcagagcactaatatcaccttgcgtaaaggcgttttcccggggaaaacagaactgtttgattggattaactctattcagcttaatcaggtagagaaaaaggatattaccatcagtttaactaatgatgcaggtaccgaattattaatgacctggaatgtttctaatgcttttcccacttcattgacttcaccttcatttgatgccaccagtaatgatattgcagtacaggaaattacgctgatggcagatcgggtgattatgcaggctgtttgaagcattgatatttaatcatctcatataagggaacttttatgacaaccgttaccagttatcctggcgtttatattgaagaattaaatagcctggccttgtcagtttcaaatagcgccacagcggttcctgtttttgctgtggacgaacaaaaccaatatattagtgaagataatgcaatccgtattaattcgtggatggattatcttaatctgattggcaattttaataatgaagacaaattagatgtttctgtgcgtgcttattttgccaatggaggtggatattgttatctcgtcaaaacaacgagtttagaaaaaattattccaaccttggatgatgtaaccttattggttgctgcgggcgaagatattaaaacgacagtagatgttttatgtcagccaggaaaagggttattcgcagtctttgatggccctgaaacagagttgactatcaacggtgcggaagaggcaaaacaagcctataccgccacaccattcgctgcggtttattatccttggttgaaagcggattgggctaacatagatattccacccagtgcagtgatggcgggagtttatgcatcggtggatttatcccgtggtgtatggaaagcgcctgccaatgttgcgttgaaagggggcctggaacctaaatttttagtcacggatgaattgcagggtgaatataacactggccgcgctatcaatatgattcgtaatttcagtaacacaggtactacggtttggggtgcaagaaccctggaagataaagacaattggcgttatgttccagtgcgacgcttgtttaattctgtggagcgggatatcaagcgtgccatgagctttgctatgttcgagcctaataatcagcctacttgggagcgggtacgggcggcgattagcaactacctttatagcctgtggcaacaggggggattagctggcagcaaagaagaagacgcttattttgtgcaaattggtaaaggtataacgatgacacaggagcagattgatgcagggcaaatgattgttaaagtcggtttggctgctgtacggcctgcggaatttatcattctccagtttacgcaagatgtagaacagcgttaatcatatgattatgaggagttatcatgtctgctattctgaaagcgcctggcgtttatattgaagaagacgcttccctagcgttgtctgtcagtaacagcgcgactgccgtgcctgtttttatcggaaaatttactccgacagtggttgattcaatccaagtctgtacccgtatcagcaactggcttgaattcacttcctctttttccctagctccaacagttgagattgttgtccaatctaacactgaatctgaatctgaatctgaaacttaccactatattgagacaatcaatttatctccagctgtggaagcattgcgactctattttcaaaatggcggaggagcttgctatatctacccattaaatgatgctgaagatgaattggttctggcggccataccagaagtcattgaacagaaaggtgatattactctgttggtttgcccggaactcgatctggattacaaaactaagatctatggcgcagtgagctcactgttgaatgataacaaagtgggctatttcctgattgcggatagcaatgatggagaatctgtgtcaggagtatggaatagtgctaaggccgccgcctattatccccagttggaaactaacctaaaattttccacgttgcctggggataaggacattcgtatcagcggttatcaggatgatgatgaaacacataaaccgaaaaacttggatgagctcaggacaatcaacgaggcgttggcacaggatattgatgcaagattgctcgaggagaaacaacgtgctgtcatcattccgccaagtgctgccattgcgggcatttattgccaaacggataatcgtcgcggtgtttggaaagcgccagccaacgttgcgctcacagggatcgggagtttgcttgataaggtagacgatgaacggcagggagagatgaatgacaagggaatcaatgtcatccgttcatttaccgaccgtggttttatggtctggggagcccgtacttgtgtggacgctgccaacatcagctggcgttatattcctgttcgtcgcctgttcaattccgttgaacgagatatccgccaggcgctgcgcgctgtgttgtttgaaactaatagtcagcctacctgggtacgtgctaaggctgccgttgatcaatatctttataccctttggcagaaaaatgcattgatgggtgctcgcccggaagaagcttattttgtgcaaattggtcaggatatcaccatgtccgaggctgatattaaacagggtaagatgatcatgactgttggtttggcagcagtgcggccagctgagttcatcattctgcaatttacgcaggatgttgttcagtaatctccatgactaaacgccaggcactgtattgacagtgcctactctaaccatcttggaggaggtgatgatgatggagagactccaaccgggtgtgactttaacagaaagtataatcacgatgggtcagcaagagatacccagtgctgtgccggtgtttattggttacaccgttcgttatccggaacaatcggaagcatcagtccgtatcgacagtttggccgagtataccagcctgtttggtgacgaccatgtgatgatgtttgctgtcaggcactattttgataatggcgggcaacaggcatttgttttacccctgaaggacaatatgccatcagtggagatgaccacagctgaagcggaaaatctgatagccgcattgcgctctgctacggttagcgaagccattggtgggcatagtcagattacactgattttggtaccggatatggctcggcttaatgacagtgatattgatgactcctcaacccaggtaagcctgtggtcccaaggctgggaggcgctgctgcaattgagtcaggttaggcccaacctctttgtgctgttagatgcgccggataatgttgaacaggcgcagaagtgtatgacaacgctatcgtcagattatcgtcaatggggggcagcatattggcctcgtctggaaactacctatcagaaagaaatatctggcaaggacaatgaatctcagggaattttccaggggactgttctgtcacccacagccgcggtcgcagcggtaattcaacgcacggataacgacgcgggtgtttggaaagcaccggccaatattgccttatcccaggttattcgacctgttaaatcttatcttcagggaagtgtactgtttaacagcagcggcacttcgctcaatgtgatccgcagtttcccaggtaagggcatacgggtatggggatgccgcactctggaaaacacggataatacgcagtggcgctatctgcaaacacgtcggctggtttcctatgtaacagcgcatttgacccaattggctcgcatgtatgtctttgagccaaataatgaacttacctggatgaagttaaaaggacaaagttacaactggttacggcaattatggttgcagggtggcttgtatggttcacaggaggatgaggcatttaacattctgttaggcgtaaacgagacgatgactgaggatgatgttcgtgcaggaaaaatgatcatgaaagttgagttggctgtgttgtttcctgccgaatttattgagatcagtttggtgtttaatacccaaacagaggcgctgtcttaagaaggaaaaagtacgatgaacgattattacacacccgtggtatcccatcgttttatggcgagttttatttttaaccgcattcccgatccgctggatattcgttttcagcgtatctctggccttagtcgggaactacaggtgactcagtacagtgagggaggagaaaatgcccgtaataactatttagctgagaaaatccaacacggtacgttgactttggaacggggcgtgatgacagtctcgccattgacctggatgtttgatcgggtattgagtggtgaaaaaatcgcttatgccgatgtggtggtgatgctactgaatgaaaattcactgccattgtccagttggacgttgagcaatgcgctgccggtacgctggcaaaccagcgactttgacgctaacagcaatgccatattggtgaatacccttgaattgcgttaccaggatatgcgctggcttggagtcaaaatatgacagtagaaatcagagagttacttatccaggcaaaggtagtgccatcaacacgaccgactgaatcagaacggcaaaaccattctttgatacaggaaagtctggatgaggcgacttgggtggaaacgataaaacgcgaagtgttggccgcattacgcgatgaggaagggtggcgtccatgagtctgattgaacgtggtttagctaagctgacaattaatgcttataaggatagggaagggaagatacgggcaggaacgttgcaggccatgtataaccctgactccttgcaactggattaccaaacggattatcagcaatcccaagcgattaatagcgaaaagcaaagtagcatttatgtacaggccaagcccgcagggttatcacttgaattaatttttgatgccacgatgccgggtaacaaaacccccattgaagagcagctcatgcagctcaagcaactgtgcagtgtggatgcaaccagtaacgagacgcgattcctgcaagttaaatggggcaaaatgcgttgggaaagtcggggttactttgctggcagggccaagagtttgtctgtgaattacactttgtttgatcgtgatgcgactcccttgagggtacgggtaatattggcattagtggctgatgaaagtctggtgttgcaggagactgaacaaaatctgcaatctccggcaaaaatcgcattacgcatacaggatggggtatctctggctctgatggcagccagtacggcatcaacattgtcaggcggtgtggattatctgacgctggcctggcaaaacggtctggataatctcaatgggttcgttccgggtgaaatattgcaggccaccaggggagacgaatcatgagccaccaactgaaaattattgcagatggtaaggcactgtcacttttggccgcggtagatgtggacacctgttatcgggttaacagtataccttctgcgacattgaaactgagcgtaccggataggccactctcttctttcagtcagacggatgttcagacagaactggcccactgtcaggtagggaaaaccctgcgtctggaattgattgatggtagcaaaaaatgggtgctgtttaatggtcttattacccgtaaggctctgagaattaagaataagcaattattgctcactctggttgtcaagcatcggttgcaactgatggtggatacccagcattcacagctgtttaaagacaaaagcgaaaaagcgatcttaagcacgctattgaatcagaccggaatcaatgctcgcttcggaaagatagcggcgttagatcaaaagcatgaacagatggtgcaatttcgttgttcagactggcattttctgttgtgccgactgtcggcaaccggtgcatggttgttacctgccatagaagacgttcagtttgttcaacctgatgctctgaaatcaaactcagcctataccttgaagagcaggggggatgagaacaaagacatcgttgtcaaggatgcttactggcagtttgacaatcaaatcaaccccgctttgctggaagtcagtggctgggatatcagtaagcagcaggtacaatcaggcggtcgctacggaaaaatcgcgttgggtaaggcggcactctctcctgatggattggcatcccttaataaaacgggttgggacatttgttatagcagtccgttaacaacccaggaaagcggttatctggcacagggattattgcttaaccagcgcatttctggggtgacaggagaatttttgctcaaaggagatgggcgttaccagttgggagacaacattcagctgactggatttggttcacagttagatggtacggcaagcattactgaggttcgccaccgtcttaatcggcgaattgattgggaaaccacggtgagcattggtttacaacatgaatatttgccgatattacctgatgctcccgaactacatattgcgacagtagcgaaatatcagcaggacagtgcggtgttaaaccgtatccccattattctgccggtactgaatcgtcccaatgaatttttgtgggccagattggggaaaccttatgctagccatgaaagcggtttctgtttttacccagagccaggtgacgaagttattattggtttttttgaaaatgatccgcgttatccagttattttaggtgctatgcataatccgaaaaataaggccccttttgaaccaacccaagataatagggaaaaagtattgatcgttaaaaaaggtgaagcgcaacaacaattagtcattgatggcaaagagaaaatgatccgaattaatgcgggtgaaaatcaaataatgcttcagcaagataaagacatttctctgtcaacgaaaaaagaattaacactgaaagcgcagacaatgaatgccacgatggataaatcattggcaatgtccgggaaaaacagtgttgaaatcaaaggcgcaaaaattaatcttacccaatgaaaggtgacgatgaatggaaaatcaaatactgacacaactctatggtcgtggttgggcttttcctccggtcttttcccttgaaaagggggtagagatggctgaaggggcggaagatgtgagacaaagtttgcagattctgtttagtactgagccgggggaacgtcttatgcgtgaaaattatggctgcggattaaatgattttatgtttgaaaatatccgcaatgaacttattgctgaaattgaatcccatatccatgacaacgtattacgatatgaaccccgggctgatatgactgatattcaggttcgtcaatcccctggcatggggaatactttgcaagtgcaggtcatgtatcgcctgagagggagtgatatcaatcaacaaatccagggagtacttgcactgagtgaaggccgggtgacggaggtagtatgagtgaagcgattgtggtggatggtgacgtgttacagtttgatcccaactttggcaatcggcaggtgacggttcccagcccaggaaaaattagcggcacaggacatgcgcaggtaagtggaaaaaaagtgtgtattctgggggatgagaaacaggtcagggtttctgcaacctatattacaacaacacatactacgccgggaacaggaaccattactatcagtgctctggatgctggccagcaggcccttcagtgtaccagtggggcggctttaattatcaaggggcagcaatttacggcgatgtttacgcctgaattgccagccatgaataatacagtgactccgccacaaccggatgttacgacaccttcatcaggaaaaggacgttttatcactcaacaaaattttgctaccgtaaattagagtattgactgaattaaatagaattaacgaaggtgtaaataattatttatttgctgacgaatcgctgtgacaaataaacacaggtgatgttatggaattaaatgagttaactaacaaattgtcaaatttggtgccaatgaccgattttaaattagataatcgagccagtttgcaattgcttaaatatattgaagcgtatacgaagataataccctttaattctggcgataaatattggaatgactttttctttatgtcaggaaatacgccagagaaacttgcaaaattatatcagaaagaaatagaacccaatggggagttattacctcagcaggcttttttgttggcggttttgcgtttattggaaacaccaatatccttattaaatgtattacctgctgctcatcgtgagctctattatcgggagcttttaggcttgtcttcccatgcggcacagcctgatcaggttgctttatctatggaactgaattcgacagtgatggaacagctgctccctgaaggaaccctgtttgaggctggtcaggatgaacaaggcaatgcattgcaatatgccctggatgccagtttgctggctaatcgtggatatatcagtgacttgcgctggttacggaatgacggggaaaagcaatgggttacttctgctccatgggatttacaggcacaggtgtcactgccgtctgatgggatacgattatttggtaagacaaatagtgatcagcaggtatttggtggggtgttgataacgtcatcacttctggcgatggaagcggggataaggaagatcattgttacttttgagcaggagatgaacacccaagaactggtggcacaggtcagcagtggaaatcaatggctaacattgacgtctgaggtaaataagaaagaggtcacactgacactgtcagacaaagaaccggcaatcagtgcgccagaggatctggataatctctttttcacgcaaccggtactcaggctacagggaaaggatagtcaggcactgccggaggtgacgggtatcagcgtttcggaaaaggatgatactaaggatacctcttttgagatgtatcacttaacaccatttggttatagcagtgatatagagccattggaggaaaatccagcgttatatttaggctttactgatgtaaagccagggcaaacactggcgctgtattggaaattaaaatccccgcagcaaccaaccgtttcctggtattacctggatcaacataatcaatgggctgaattggattcatgggtcagtgatggaacccagaatctgtatcaggatggtacttggcacgttgagttgcctgtggatgcatccaatcaggcagagcagatgccagttggacgctattggttgcgggcagtggtggaggtacccgctcatgagggggcgttggggaaggctccttggctatatggtctaatctataacgccatgacggcaaccttggttaatgtagatagcatcagtgacagccatttcttaacccctttgcctgccagcagcatacagcggcccgttgaacccatcattgtgttggcatcggtcaaccagccttgggcatcatggggtggacgtatacctgaatcctacagtgccttttttgaacggatagctcaaaacctgtctcatcgaaaccggtccttaacctggggaaatatggtgacattactcaaagagcgttatgtcagcatctttgatgttaagtatccaggtaatgatgaactcaccagagtgccagcattggagcagcagcaactaacagtgattccagcaaaccggtacaacgatagcgatgattctctgcgtccggtactgaatcctgctcgtctgcaagagatggctgattggttgcagcagaaagactctccctgggcctctattgaggtcaggaatccagaatacttggatgtgaaaatccattacgaggtgatttttaaacctgatgtgaacgaagattttggctatcgccagctacagcagcaactgtgtgaggtgtatatgccttggagcatagatgagcagcggcccgttgtattgaataacagcattaattatttccagttgttagccactattcaacagcaaccgctggttgagcgagtcactcgtctgacactacatcgggctgattcttctgatgagagtgatggtacagcatctgtggaagccaaagataatgaagtgcttattttagtctgggaagaggacgataatctgcaataccgaggaaatgactatgagtaatcaggatgcactgtttcatagcgttaaagacgatattcactttgataccttgctggaacaagctcatcaggtgattgaaaaacaggctgaaaaactgtggagtgatacggcagagcatgatccgggtatcacatttttgcagggaatcagttacggtgtgtcagatttggcttaccgacatacattacccctgaaagatttactgactccggcgccggatgagcagcagcaagagggaatttttcctgccgaatttggcccgcataatacactgacttgtgggccggtgacagcggatgattatcgcaaggcattgttagatctacacagcagcgacagcctggatggtactcagcaggatgagggggattttctgttccggagtgtgcaactggtgcgtgaaccggaaaaacagcgttatacctattggtatgatgcaaccaagagggaatatagctttgtcaacagtgaaggggctaaagagtttaccttgcgggggaattactggttgtatctggaaccaacccgttggactcagggtaatattgccgctgctaccagacaactgacagaatttttgactaaaaatcgcaatattggtgaatctgtcagcaacattatctggctacaaccggttgatctgccactgttgctggatgttgaactggatgatgatgtaggtgcacaggatgtccccggtatttttgcggcggtgtatagcaccgcagagcagtatctgatgcctggagcacagcgttaccgtacggaagtactgcaaaatgctgggatgagcaatgatcaaatcttcgaaggtccattattggaacatggctggataccagagctgccggcagcccgtgattatactcaaaggctcactctcaatcttagccggttggtaaatagtctgcttgagattgagggcattaaacatgtgaatcgtcttcgtctggatgatagcttcgataaaactgctattgaacccgttaagggggatacctggtcgtggtcgatcaaagagggctattatccacgtctttggggagaagacccacttaaccaattggcgcaacaaaatggcccgcttagggtgatagccaaaggagggattagcgtcagtgtgagtaaagagcaaatccaggccagtttacccagtcaatcactgattcaaaatgagccggtaatattggcttacggccagcaccgtgacgttggcagctattatcccgtcagtgatactttgccgccttgctatggactacaacattctttgtctgaaagtgaacacttattgccacttcatcaatttatgttgccatttgaacaattattggcctgtggttgtcaacagatagccatgctcccgcggttactggcttttcagcgcgaaggttatgaggtttggggtgatcagtggccctttaagtcaggctcagtgaatgatgacgcccatcaagattatgcccctgcattaaaggatttgttaggacagattgcgctggatagtgatcatgaattggatattattaattacttgctgggttactttggcacacagcgggcaccgcgtacctttacgacacaactcgatgattttcgtgcggtccaacagggttatctggcccagcaaccgacattgacttaccaccgctccaatattcgtatcgatcaggtatcgtcgctacaaaaacgtattgctgctcgcatggggctgggcggtgagttgtttaaacctcaaccggatctgagccaactgcctttttatttgattgaacatcgagcgttgctgccagtcaaacccaatagtcagtttgataaggaacagaaaccagcctcggtgacagaggaggggggcagccaaacaggtcaacattatgtggtcattgaacagaagggcattgatggcaagctgacacaggggcaagtgatcaatttaattctgtatgaaggagagcagggagaaacccaatttacgatacgcggtcagatggtattcaaaaccgagggggataagttttggttggatgtgaataatagtgcgcaactggaatataatctggcgcgggtaatgacagcagccaaggcgagtaaactcttttggcaaaacagcccggtatggatggaggatatgggctatcgtctggcctatgctagtgaccaatcctcattgcctgtgaatcaacggcgcttgacccgcacagtgcaaactccattcccgccgatggttgttgtaggtagcgaaatcaccctgttaaagcaggtggggatagtcaatttaaaaaaagcggagtcagaaaaactttatgcaaaagttgttagctttgatcgcattgaagggaccttgattattgagcgtttgggtaattccactctggcttttcctacctcggaagaggcgtggcggtatagttggtatttttcgggggagaaatatgaaaggactgaccgcttttcatttgtgattagcgtagtagtgaacagtgacttaattaaattgcccggtgttgatccctataaattggaagaatgggtgaaagaaacgattcttaccgaatttccagctcatatttctatgattatccattggatggatcgggaagcctttttaaatttcgccaatacctatcagcgttggcaaaataatggtacgccactgggggatgcggcttattccattctagaaagtttgacacttggtaaattgccatctgccttaaaaggtgttggcacaatgcgtattgccacatctagtcaaagagaagaagtggtgggtagtaatggtgatcaatggaatacagatggaataacccagaatgaattattctatgttcctaaagagagctaggaaaaataaatatctgccactaatgatgttgaattaaatatgttttctggagttaatcatgaacgaaactcgttataatgcaactgtacaagaacaacaaacattatctaatccaaaagctgttggacctgacatcgataaattaaaggataaatttaaagagggcagtattcccctgcaaaccgatttcaatgagttaattgatattgccgatattggacgtaaagcctgtggtcaagcgccacaacaaaatggcccaggagaaggattgaaattggctgatgacggtacgcttaatttaaaaataggcactttttccaataaagacttttctccattaatattaaaagatgatgttttatctgtagatcttggtagtggtctgactaatgaaaccaatggaatctgtgtcggtcagggcgatggtattacagttaacactagcaatgtagctgtaaaacaaggtaacggaattagcgttactagtagtggtggtgttgccgttaaagttagtgctaataagggacttagcgttgatagtagtggtgttgcagttaaagttaatactgataagggaattagcgttgatggtaatggtgttgcagttaaagttaatactagtaaaggaattagcgttgataatacaggtgttgcagttatagctaatgctagtaagggaattagcgttgatggtagtggtgttgcagttatagctaatactagtaaaggaattagcgttgatggtagtggtgttgcagttatagctaatactagtaaaggaattagcgttgataatacaggtgttgcagttatagctaatgctagtaagggaattagcgttgatggtagtggtgttgcagttatagctaatactagtaaaggaattagcgttgatggtagtggtgttgcagttatagctaatactagtaaaggaattagcgttgatagtagtggtgttgcagttaaagttaaagctaatggcggaattaaagtagatgctaatggtgttgcaattgatcctaataatgtactccccaagggagtgattgtaatgttctctggcagtactgcaccaactggttgggcgttatgtgatggcaataatggtacaccaaatttaatcgatcgatttattttaggtgggaaagggactgatattaatggagtgagtactaatacagcttcaggtactaaaaatagtaagttattcgatttcagttctgatgaagctacattaactattgatggtaaaacactggggagagcattatcgttacagcaaatacctaatcatgcacactttagtggaataattatggatacagagaaagttaattattatggaagtaaaaaaatcacaacaaatgtgtggggtgtaacaacaggagataatacttcagtacgatatatttataagtcatcaggtgtacttgactctaacaataatgtctccaacagtaccttaggcggaaacagtctgcagacgcacgatcatgatattaagataacgggcacaggaaaacattctcacaaaaacaaagtaacagtcccttattatattctggctttcatcataaagctttaatatatatgaaaaattgaaaatataaattatccattaataataaagaggatattagcatgacttcggagccaaatctgttaaaccggattacaattactattgaagctaataatcaacaagtagctagaaaagtattgcatggctccttgcttaatcaagctaatataaataaattatttaattcatactttaatgaatatgaaattaataggggtgtttatttagaaacattaatcctgaatcttggtacgataaatttccatgattttaattcattgtttcctactctcctaaaagctgcattgaataaagaattcagtcaatatcagataaacaaccatagggaagaaatgctatttaatgagacaatatcaaatcaagctactgataagtcttacatatttggcgataacaaattaattgatgcagagaatttcattcactttttatatcaaaagcattccacattaaatctagtagaagcaatgggaaataatggtattgaaaaattaacaaatcagttaacacaaatagaaaataaatttgcgttattattggcaaaaagttgtttgtctgaggaaggcttaaaacgactcttggctatcaaacaacccgatttattaatcgctatcaatcgcagattatctgaaagaataaatagaccacaatatcaggagaagcttgtttcctgcggacaactgatatttagtgctctgggatatatacaacagtacaatatacaggaaattcctaaaccggatgaaaaagttattgcacgcataacaactgaacttaataataatggtttgcttaatacaatacctattattacactatttcgtcagagtgggattaacgattcatcactaaatgattggctaaagaaaatctggcaggtgagatcaatttcacagttatgcagaaagtatctttctgctaaggaataccaatatctgtcagaacattttgtttcaaagagcgtcgataaaaatagatatgatgaagagcccgtaaatcagagcatattatcaaggttgaataataattccattaaagaaggaaataatcacagtcaactctgtactctcagtagactatattctgaacccgttgtattacctgaacaaaccattctacgtcaggttagtaatacagtagatcagagcatattatcaaggttgaataatgcctccattaaagaaggaaataaccaaagtcaacttcgcactctcagtagactatattctgagcccgttgcattacctgaacaaaccattccacgtcaggttagtaatacaggtatattaattctatggccaatgctacctacactatttaaccagcttggtctacttgagaaaaagaaatttatccatcgtcaggcccagtttaatgccgttgattttcttgattacctgatttggggaaccgaagatgtgaaagtggaacgaaaggttttgaataatgttctatgtgggttaatggctgatgaaattactgaaccaatgcctattgaaccagaaaaacaatggataataattcaatggctggacgctattatctcccaactttctggctggaaaaagttaagtcgtaatgacgtccgtcaattatttctacaacgaccaggagaattactgatcaatgaacaggaaattaaaatcacaatacagcaacaaccatttgatgctctgttaactgattggccgtggccaatgaatatggcttgttttagctggttgagtcaaccattaaccattacgtggttataaccattgaccacaatgacttagtctgagtaaaaaatatgaatatatcgcctgttttttatgattcattgaatcaggataacgaccgtgatctatcgtttttatttagcgaactggaacgaatagatctcgctcttcaacaccatttttattgtgtagaaagtcagcgaagtgagctcctggatgagtttctgctcactgaggcggaagtggtgaccaggctggataagccacttggtaaacctcattggataaatgatgattatctggcgatatcgcaaaagggcaatgtaagcctaatggcagcgtccagattaatggatctgatcgaacgctttgaactgactgattttgagcgcgatgttttactattaggcttattgccccattttgatagccgctattatcgactgttttcgctgattcaagggggacaacagggtcgattaccttcttttgcgctggcattggaactgttttgccactcggcgctggagaaacaggtacagcaagcgagttttctgcaccgggcacctttgatgggttgccagctattatccatcgatactagtcaaaaaacgctggcctggctccagactccctttattactgacagcggggtatatcactttttactggggcatcactacattatgccggctttagaacattgtgctgagtggttaacaccgacagggattggctgttatcctgaaggattaaaacaagtactgggtaacgtattgttatctgacaacgataatattagaccgattgtcttattacggggaatggccggcagtgccagagcttataccattactaatatgatggcttcagaagggaagcaaacactgctggtagatatatccaaacttgctgatagcgatgaaaaaaacattattcttcagataaagcatattttgcgggaaacccgcatgcatggagcatgtttattattacggaatttttgcttgttagtggaacagaataaacaactattggactccctgtcagagttattgaatcaacctgaattaagaattgtttgcctgattgagccttattccccattggtatggctgaaaaagataccggtattactgattgagatgccacttttaacgcctgcggaaaaagccagattgttaattgccagcttaccggataattgttccgaggatattgatacgataactttaagccagcgttacacttttaacccagaaaccctgccattgattttgcaagaggcccagctttatcaacagcagcgagatccgctggatatcttgcagcaatgcgatatacgccaggcattaaatttgcgtgctcaacaaaatttcggtcaattggcacagcggattattcctaagcgctcattaaaggatttattggtatccgatgagattgctcagcagttacgggaaatactcatagcaattaagtatcgggaacaggttctggcgggagggtttaaagataaaattgcctatggcactggtatcagcgccctgttttatggtgattcaggcactggaaaaaccatggcagcagaagtgattgctgaccacattggcgttgacttaataaaagtggatttatctacagtagtgaataaatacatcggtgaaacagaaaaaaacttatcccgtattttcgatttggcggaacaggatgcaggggtattattctttgatgaagctgacgcactgtttggtaaacgcagtgaaactaaagattcccaggacagacatgccaatattgaagtttcttacttattacagcgcctggagaattacccgggtctggtcattttatccaccaataatcgtggtcatttagacagtgcttttaatcgtcgttttactttcattacccgttttacttacccggatgaaaaaatccgtaaaaaaatgtggcaggaaatttggcctagaaatataaaaatatcggaagatatcgattttaacgaattagctcaacgaacaagcgtgactggcgcgaatatccgcaatattgctttattgtcttcattctttgcttcagagcaggggaatgatgaagtcagtaatgaaaatattgaaattgcattgaagcgtgaattagctaaagtcggacgattaacattttaaaagttatcacaatgaaagtattgaaatattaaataaatttattaccaaaaagttatcacgatataatttaagagaggttttttatgttaaacacgcaaactattattgatgtcaataaggcaatggatgccatgctgcgcgcatatctgaatcaagatattgccattcgttttgatctacctgaattggatactatgcaatctgatgcgatggtaagtatctttctttatgacattcatgaagatttacagcttcgctcggcagaatcaagagggtttgatgtttatgccgggaggttattgcctggttgggtaaatattaaatgtaactatctgattacctattgggaagcttctaagccagcgactgatgccagcagtccggatagccaacctgataaccaggcaatacaagtgatgtcacaagtattaaatgccttgattaataatcgtcaattggcaggtattcctggtgcttatactcaggttgtaccgcctaaagagagtttaaatagcctggggaatttctggcaatcactgggtaatcgcccacggctttctctcaattattcagtgacagtacctgttagcctaaacgatggtcaggatagcgcgactccggttaccgcggtttcttctacagtggaacaaacggcatcgctcagtcaagaagtggttagtcatgctttacgcgaattactcattacggaattaggaggaggagaggataaccggttggtactgagtaaagttgaattatccgcagtgaaagagacgatgactcaagacagtccggctcagatgattatattgttgtctgtttcaggcattacacgacaggaatatttgaaggaaattgataatatctttgatcgttgggtaaataatgctgaagttattaccactattgatgattgtgggattagaattgaaagtataacgaaagataatcttgtaggaatttaa

SEQ ID NO: 94 - DNA, 18490 bp, Photorhabdus
atggccacaaccacagttgactatccaataccggcttatcgatttgttgtctccgttggtgatgaacaaatcccttttaacagcgtttcggggctggatattacttatgatgtcatcgagtataaagatggcaccggtaattattataaaatgccgggtcaacgtcagttaatcaatattacactgcgtaaaggggtattccctggcgacactaaactttttgattggcttaattccattcagcttaatcaggttgagaaaaaagatgtttcaattagcttgaccaacgaagttggaactgaaattttaatgacctggagcgtagccaatgcattcccaacctcattaacatctccttcttttgatgccaccagcaatgatatcgctgttcaagaaataaaactgactgccgatcgagtcactattcaggcagcttaaagcatcacgatgattgatatatcagacgggacaaaatgatcctcaaaatttggcacaacggctacccgtccaactaaatttaccctcttacagttcacgcaaaatatcgcacaatacaattggaggcaatatgccaacaacaacttatcccggcgtttatattgaagaagacgcctcactgtcactttccgttcgctcaagtgcaacggcggtgcccgtttttaccgttgaagatgacagtcaacttcatactcctaccagagtgaatagttggttagaatatctgacaaaaaaagcagataaaaaattcaattctaccgacaaacttgatatcgcattgcgcgcttattttattaacggcggcggatatggttatctcgtcaaagcgggtgaattaacaaatcaaattccaaaacttaacgatgtcacattactggtcgcggctggagaaaatatcaaagatgctgtgagtacactttgtcaaccgggcaaaggcttatttgccattctggatggcccaaccgaagagttaaagtctgatggcaaatccagagatccgtatgatcaaagcccttttgccgccgtttattacccctggctagttgctgattgggcagacaatattccgccaagcgcggccattgccggtatctattgttcagttgaccgtacccgcggtgtctggaaagccccagcaaatgtcatattacaaggcggggtgaaaccgaagtttaaagtcaccgatgacttacaaggtatttacaacaccggtaaagccatcaatatgatccgtgaatttccgaataccggtgtcaccatctggggcgcccgcacacttaaggacgaagataactggcgttacatcccagttcgccgcctgtttaacagtgcagagcgagacattaaaaatgccatgagtttcgcggtctttgaacctaacagccaacccacctggaaagctgtacaccgagctattgataattatctctatgccctttggcaacaaggagggctagcaggaaacaaagctgaacaagcttactttgtgcaaattggtaaagggataaccatgaccgatgatgatatcaagcaagggaaaatgattgttaaagtgggtatggccgcagtgcgcccggctgaatttatcatccttcaattttcacaaaatgtagcacagtaaccgtactgaggcgcggtttaacaccgcgtccattcagtctattgaatggaggagacaataatgataacggagataaaacagccgggcgtcaccatcacggaaaattcgatatccccgaaatcagataatgaatttatcggcgtccccgtttttattggccataccgaaaaaaattcaagccataaaacggctgttaaactaaatagcctgatggactttacccaagctttcggtgcatcaggattaacctattattcagtacgccacttttttgaaaatggtggacagcaagcttatatcttgtcactggggattaatcaacagctaaaagattttcaatcattgattaccgccctgcaatggaactgggtaaaacaagccattgccgcagaaaacgaaatcacattgattgttgtgcctgatattacccgttttaatgatctcagcgctcaaaaaagcctttggctacaactctggcaatcaatacttgaactgtgtaaaagtcggcgtggcatcatgggattactggacgcgcctgatgatccaacattagcaactgagtgtttaaaacaattctcttccactgatcgccaatggggcgccgtatactggccaaggctaaaaagtacctaccaagaaaacggtacatacattgtactttcacctactgctgcggtcgccgccgttatgcaacgcaatgacagtcagaaaggcatatggactgctcccgccaatgtggctttagccaacgtcatcggtccggtacgttcttacattgaagctggaaccttgctgaatcaagaaggcacttcgttgaatctggtgcgtagcttccccggcaaaggcattaaaatctggggctgccgcactctggataacatacctcattctccctggcgttatatccaaattcgccgtttggtttcctatatcgaagctcatataacccaacttggccgcgcctttgtctttgaacccaacaacgccatcacctggatgaaatttaaaggtcaggcccacaactggctacgtcaattatggctaaaaggtggattacggggcactcaggaagatcaagcatttgaggtgttactgggtgttaatgaatccatgagtgaaacggatatcttggccggaaaaatgatcatgaaaatcaggctggcgctgttaattccggcagaatttattgagctgagtctgacgtttgatatccgtaacaataccgtacctagctaatctaaacaggggaaaaacatgtacaacttatacaccccgtcagtatctcaccgttttatcgccagttttctgtttaacaacattcccagcccacttgatatcgcctttcagcgtatatctggcctgagccgagaactgcaaaccacccaacatagccaaggtggagaaaacgccagaaacgtctggttatccgagaagatccaacatggcagcctggtgctggagcgcggtgttatgaccatcactcccctcaccttggtttttgatcgcgtgctgcgcggtgaaaaagccgtgtatgccgatgttgtcatcatgctactgaatgaaaatgcgttacccgtggcgagctggacagtcagtaacgcgctaccggttcgttggtccaccagcgactttgatgctaatagcaacaccgtactggtgagttctctggaattacgttatcaggatatgcgctggttaggagtaaaagcatgacggtagaaattaaagaactgattattcaggctaaagtcaccgattctacgagtgatcaactcgccccaagaacattagcccaagaaaagctggataacgcccgtttgattgacatagtgaaacgggaagtgttagaggcattacgtgaaggaggccatcatgagtttaattgaacgtggtttatccagactcaccctaaccgcttttaaagaccgagaaggtaaagtttccgtgggtcgcttacaagccatgtataaccccgatacgatccagcttgactaccaaacccgctaccaacaggatgaaagtgttaatcgtgccagccaaagcagccgttatgtattatcccaacccgccggattatccttagttctgctgtttgatgcctcgatgcccgataataacatgccgatagaaacccagcttgcgaccctgaaatccctgtgtgcgattgatgccagcaccaaagtaccccacttccttaaaatcaaatggggcaaaatgcgctgggaaaacaaaggttatttcgcctgccgagccagtagcctggccgtcaactataccctgtttgaccgggatgccacaccattgcgggccagcgccactctatctctggtagcggacgaaagctttattattcaagctaccgaacggcagttaaaatcaccgccggccactgcggttagcgtaactgatatgctctccctgcctttgattgctttagatgctggagcgtctctggctggtggcattgattatctctcgctggcctggcaaaacggtctggataatcttgatgactttacccccggacaaacactgcaagcgcggggggatgcatgaagatacccatgataaccctcaaaataggtggcaaaacgctcaatcaattgactgtcatcagtctgacaataaaccatcaaatcaatggcattccctcgaccaacatcaccttggggatcgctggcgatgcgagccatattttcgacaccaaagcccaagctgaactggcaagttgtcgccccaataatgaactcaccctacagatccaaaaaaccgtggtgtttaaagggagcatcgttcgacaagcacttgaactgaaaggtcaagacagcatcattaccctgacagcaaaacatccactacaaaagttaactcatagcctccattcacaattattcagtcaacagagtgatgaagcgattatcaggaaattattcaatcaggcgggtatccaaacaacgataaagcaggctcctcaacttaaaaccgttcatgaacaaatggtgcaatttcgttgcaatgactgggcattcctaaaaagccgattgattgccactaatacctggctgttgcccggcaatgaatcggttactttgataacacctaaggccctgaatcaatcgacagtgcatactcttcatcgacaggccagtgctgaagatattgtgttatttgcagcggatctccaatggaataaccaatatagccctaaaacggtgagtgtacgtgcctgggatattgctcaacaaaagctttccccagcaattaatacccaaaacagtcagcttggcagtcataaattggccgtggacagtatcgccgcactggctgataaagagtggcaatgggcttacagctatccattagataatgaacaagccaaacaccttgctcaaggcattatgaataacctgcgaagccataatatatctggcagttttgaaatcgaaggtaatcaccgttatcaaccgggggatgtcttggcgttaaatggttttggtcaggggatggacggtcaagggattatcaccggagtcagtcagataattaatcagcggcaaggctggcacaccctattaaccttaggcatgttacccgatgtagaaccgccggtgcctcaggtgaaagagttgcatatcggtatcgtggaaaaataccagcaagaccgccaatcactaagccgtatcccagtcagaatacccgcattaaacttgaccaaaggtgtcctttttgcccggctaggtaaaccttatgccagtcatgaaagcggattttgcttttatcccgaaccgggagatgaagtgattatcggattctttgaatgtgatcctcgttttccagtgatattaggttccatgcataatccgaaaaataaaccaccgttagaacccagtgaaaaaaatccggtgaaaactttagttatcaagcaaggggataaacaacaagcattaatattcgataataaagaaaacacggtggcacttaatagcggcgaaaataaagtctctctgcaacaggataaaaacattacgctcaattcaactaaaaatctcatcactcaggcccaagaaattaatatacaagcggaaaaatctctgtcagccacaggaaaatctggcgtcgatattaagggcgcgaaaattaacttaacccagtaatgaggtattgaaatgacaagccaaatattagccaatatttacggttgcggctggaaatttccgccacagttttctattgaaactggcgtagaaatggccgaaggtgccgaaaacgttcgccaaagtatgaaaatcctttttttaactgaacccggtgaacgaattatgcgtgaagattatggttgtggtctgaatgattacatgtttgaaaatatcagtgatgaattattatcggagattcaaacccgcattgaagaacgagtattgcgctatgaaccccgtgctgaaatcacagatatccaagtaactcagaaaacagactcaccgaatactttacatattcaagtgacctatgccctgagaggcagccaaatcagtcaacagcttgaaggggttcttgagatcaacgaaggtcaggcaaaggtgagtctatgagcaaacaactcattattgatggcgacagcctgctattcgagccattattcggcaaccggcaggtcactattttgatgccagcgaccatcagaggcagcggacacgcgcaaatccaaggcagaaagatagcgattgtcggcgatgaaaaaaaggtacaacttcaagcgcaatacattaccccaagccacccggtacctggcataggcacagttaccattgctcaattagataccagccagcaagtcaacttttgccacagccctgccacagtgatagttgtcgggcagcaatttaccgctcgatttaccccatcacagccggcaattaatccgtcaaccgggccagatgtcacaacacccagtatgggcaaaggccgttttattgccagtcaacatactatcaacgccggataaataactctgcaaaatcattattcaataacgttcctattctgcaatagctatcagcaatatattcaaataacaggtggtataatatgggactcaccgaattaaaaaataaactctctgctatcgtactcgatacggattttaaacttgatgaaagaagtacactggatattttaaactggctacaagaatatgctaaaaaaatccctttcaatcaagagaaaaaacagttctgggatagtttctattttattcaggaaaatagtcctgagaaattagccgatctttaccaaaacgttaataaaacgaatggccatttaccggcccatcaagcttttgttttagcctttttaaaacttttagaaaccaccaaagtattatttaatacttttccggcacgacatcgtgatctttattaccgggaattattaggtctaaaacccagaaatgcccaagcagatagtgttgctttaggcattaccttaaatacagataacacagaacatcttattcctaaaggaaccttgttcgatgccgggcaggacagggccggaaatccgctacaatacgcatcaaatgcagatttactggcgaatcaaggaaaattgagcgatctgcgttggtgtcgaaaagataatgatagctggcaatctgcaatactactgaaccactcagataatattgaattacctgaaaacagtattcgactttttagtccaacgccggatgatattcccgttttatccggttatttgataacttcgtctttatttgctatgccaacgggggaacgcagtattacattgactttagcagataattggcatggtgatattaagcacatcaccgctaaaatcagttcgggagatcactggctttcactatcagtaaaaaaagaacaagacaatagtattcactatcttaaactttatttatcaaccaatgatgaccccatcggtcctcctgatgctttggataatatagcgtttgatgtaccggtattaaagctgggcactgttcagggacctatactacccaagattacgggtattgaaattagcattaacggcaacagtaatgtacattattcctctgataacggtattgaaaaaatagatgcagctagttttccctttggacaatcaccgtcaccaggttccggttttaatctgattgcccctgaatggtatggtacagaaagcgccaaaattactcttactcctcaatggactggattacccaaagaggggtttaaagagtggtatcaaggatatagttctacccccgaaaataatgcatttaaagtacaggcttatttaatcacacctcaaaagagagaaaaatttaatgaagctcagtcattatttaatgaaagtaaagacaagaaaccacaaggaaaaagcctaacttttaccttacctgcaatggattattcctttgcaaacagcccatcatctaataactggcccgcatcaatacgcatagaactaaccgaacaggattttatgcatgcccaatattggcaaaatcctacgggtaaaaaacagccctatacccccaaaatgaacacattacaaattcagttcagtgccaaagttaaacccgaacaattttccgtttattctctcacgccttttggttggggaaaaacaggagaaaatagaacatcattaacccatgatacattctatttaggttttaccgatgtattaccaggacaaactttatccctgtactggcagttagaaggtattaaaaagctccctttatcctggtcttatctgaatcaagaaaatacctggagtccattggataatcaggtgcatgaccaaacccacaacctatttgatcgaggaatctggcgtacctcattgccacatgatgcttcaaaccaagcctctcaaatgccaaaaggacaatattgggtgaaggcacacattttacaaacgaatcaagcaaccctgactgatctgtattggtatcgaaaagataatgatgtctggaaatccgcaacacctcttagcctttcaaataacatgaaattacccgcaaacggtattcagatttttagcccaacatctcatgatgttccagttcgatacggctacctaattacttcatctttattctcattcctcaagaaaggacgcaatatcacattaattttagcaggagatagctgggagggtaatcctgaaaacatcaccgctaaaatcagttcaggaaatcactggttaacactatccgtcgaatatctgagtaatactaatagtcttaagttgcaattatcagataataataatgatcccatcagcccccctaatgctctggataatatgacgtttgacacgccattgttaaaactagaagccactcaggatttcactttgccctggatttataaggtatgcgttaatagcaacaatatactctctacctctgacagctcagatgcagcgattactcgtttcccctttggccaatcaccatcgttgggttccagctttagtccgaaaatcgttttcccggaatggtttgaatctgaatacgcatcagacaccacgatcacgattacccctcaatgggttaacctgcccacagaaaacttttcatcgtggtatgacggatatattaataaacctgccgataatagcgtatttaaaatagagggttatttacttactcattatcagggaaaaatcaaactcacagaagctgagacaggaagcgaaacccaagcattattcaatggaaacaatgcaccacaaggaaaaagcctgactttcactttacctaataggtataacttctatccgcgcaaccatcagtcaatgaagatagaaataaaactcgttaaacaagactttatgcacactcaacataagagcaatcccacaggcaaaaaaccaccctataccccgcaaatcagtgccttacaggtggaattcaatgctacagctttccatcgaaaattctccgtttatcctctcacgccttttggctggggcaaaacaggagaaaatagcacaccattaattcatgatacattttatttaggcttgaccgatatatcaccagagcaaactttttctctgtattggcagctaaagggccttaaagagctacctttgtcttggttttatctaagtgaagaaaatagctggaaatcattaaatagatcaacttacaaccaaacccacaacctgtttgaatcagcagaacaaagtatcctattaccacgggatgcttcaaaccaagcctctcaaatgccattaggacggtattggctgaaagcacagatagaacaggagaaaaaacagataaagatagcgcttcctgattattatccaagaatcagggggctgttgtataacgctaccatcgccactttaatcaacgctgaagctgttgagcaatctcaccttatcaacggattggctgctaacaacattaaacaaccggttaactcatccgttgccatcaacgaagttattcaaccctggacatcctggaacggtcgcccaaaagaaaccgagtcagcattcctggcacgagttcctgcccggctctctcatcgtaaccgagtgctaagctggggtaacattgccactttattaaaagagaattttagtagcttattcgatgtcaaatacccttctgtcagtgaattaaccaaaattccagcgccagaaaagcgacaattaaccatcatccccgacaaccgctataaagataatgatgattcactacgcccagtattgaaccaagccagactgaccgagatggtcgaatggttagatcgattaagtagcccttggacaactattgaaattaaaaatcccacatatgttaacgttctgatccactatgaactgatatttacctcggatgttaaccccgattatggcctccatcagctacaacaagaactcagtcgaaaatatatgccgtggggagaaaatgcagctattggcgtaacacccggtaatcgtattgactacttccagttattagcctcaattcaacaatcaccgctggttgaacgggtcaccaacttaacgttaaaaaaaggcagccagcctaccgtaagtgaaagtatagaagccgccgatgatgaagtactgattttagtctggtcataaaaacttccccaacctaaggaattaacaaatgaataatcgagatatgctatttcctatcattaaagacgatattacctttgattctttattcgcccaggcaaaagccgttattgaacaacaatcggggcagctctggaataatacaggtgaaaatgatcccggcattactttattagaagcctgttgttatggcgcatccgatctggcctatcgccacacattgccactgcgagatttgcttactcctcaagaaaatgaacgaatagatgatggcatttttcccaaagaatttggtccacaacaaatactgacctgcggcccaattaccgcggaagattaccgtcgagctttgttagatttgcgtagtgataacaccgttgaaggttattttttctttaatgatgcacagctcattcgtgaaccggaaaatcaacgctattcatattggtataacaaagaaaaacgcgaatacagttttactcaagaccaatacagcgaacaattacagttaacactgagaggaaactattggctctatttacttcccagtcggaaaacccagctcgataacaccctggctgaagaaagactcaacatttttctgaaagataaccgaaacttaggagaatcggtcagtaaaattatttggctagaacccattaaactgtcattgaaaattgatattcagcttgatgatgacgccaaagatattgctgatatatttgctaaagtttatatgattgcagaacaaatggtgcttgaaaaaccattacgttataccactcaagcgatgaaagaactgggttacagtcaggaacaaatatttgaaggcccttatttacaccacggttggataccgaaattacctcaaaccaaagattatactcaccctaccgtattaaatctcagtcctttaattaatcagttactggctatcaaaggggtgaaacatattacccaatttacattggataagcctgataaaaaaatttctaagttaccaaatgataattggtcttgggaaatcgctccgggatattacccaaaactatggggagatactccattagaattaattacctcaccaacaagcccactcaccatcacggcaaaagggggaattaaaattgctattactaaacaacagatagaaaaaaacataatgacagaaccactaattaatacacagccagaattattgaactggggtaaacatcgcaaagtcctggattactatccgataagcaataaattacccgcttgctatggattacaaactaatacccaacaacagctacagttgcatcaatttatgctgccttttgaacaaatgctagcgaataactgcgctgaacttgctttattgccaagactattagcttttaaacaacgaggaaatacggtacatggcattcaatggccttttaaagaaaatacggttggtcaacatgttcataaggacatagtatctaatttaaacaataatgctacgaaaatcgataataatgccgatgactacgacaaggaactcgttattctagattatttgttaagatattttggggctcaatgtgcaatcccacgactatcaccagacccaccacaatcatcattaacagaacctcagactaaaaaagattttctatctactcagcgcgaatatctggctcaacagccaaaactgacttatcagcgtaacaatattcggattgataaagtatcagcactgcaaaaacgtatcgctgcccgattaggtctgggaggagaatgtttcaaagcagagcctgacttagctcaccttcctttctacctcattgaacatcgtaggctcttaccagtaaaacctgatataaaattctatattgagcaacaacctaattctctggaaattgaaaatgataaattaaaaatcacacagaaagattcagcgggtcggttactgcaaggtcaagttattaacctggaatttcgtgagggctatgatgaatttacattgctaaacttaatgataactgaagtgacaagagatacattcaccattagcattaataatagccgtgatctcagagacaatctggacaaagtgcaacacgcgtttgaacaaacgaataatctgagctggcacaatagcttaatatggatggaagatatggattatcaattggtttatgccaatggagaacaactggaaaaagcggaaaatgaacgatggattaccattaacaatcaaagtgctttccctgctatgatcggagagaatgatgaaatcacactaaaaattcaatccgattatgaacttaaaaccaaagtcgtgcggcttgattataacaacaaaaaaattctgattataaaagatgcgacatcaataaataattttccgccaaaaagagaagcatcatattattcttgctcttctctaaaagacaatgggtacggatattcggatgaatataaatatgaacttacttatattgatacagattctacaaaagaaaatgagtgctggattactatcagcgatccaaataatttgttttctcctgatatcatcgcagagaatgacgaaattatattgaaagctaaccctaattatgagtttaaaacgcacgtagtaaaatttgatcgtattaatagacaaatattacttaggaaaaatacagacctggaaaataattttccatcagaaaacaacacatcgcactatcgctggcatttctctggtgaaaaatatgcccaaactgaccatttttcatttgttgtcagtgcagtactgaatcgagaattaattgagaggggcacagtcgatctctataaattagagtcttgggtaaaaactgagattttatctgaattacccgcgcatatctcactcgttattcattggctatcatcggaagaattcgaaaaatttgccagtacttataaagtttggcaaaataatggcgctcctttaggtgatcacgcatataaaattctagaaacattaacacttgggaaaaaaccttctacttcagcaagaaggtccagcagctatatagaagcacagtaataattcttacagaacattaacccatatttatcttataatatcaaacatcataaaaacaatcttcagctcattataatgacatatttcatactcaggtttcttcatatctgttaattacaaagagaatattaatatgatctcagcaccaaatctgttaaatcggattatcattactattgaagcgaataacgcacaggcagctaaaaaagtattgcatggctccctgcttaatcaatccagtataaacaaactctttgattcatactttaaccaatatgttgttaatcagactatctacctgaagacactcaccctgaatcttggcgaaatacgattaaatagttttaattcacagtttgttattcggcttaatactattctgagtcaagcattgagccaatatcaggtaaataatcaaactgatattgagaaatttatttattacttatatcgaaaagattctatattaaacccaatagaggaaatcaataatcgtgaaattactgacatcaatattaagcaattaattaaccaattaccccagatacaaaacaattggacactattattggcaaaaagctgtttatccacacatagcctgaaaaaactcctggctatcaaaaaaacagctttattaaccgccattaatcgtaaattatctgaaaagatcaatatatcaccctatcagcaggaatcggtttccacctggcaattgatactgaatgcgctgaaatatatacagcgacataatacacaggaaatacctgaacccgatgcgaaagtcatatcactcattacaacggaactcaatgacaatgccattaatacagcaccaattattgcattatttcgccaagttataaccaaccattccccactgaataagtggctggaacaactgtggcaaacaaagcgaatttcacagttatgtaaaaaacagctgtcaattgaagaataccaacatctatcggagcgctttattgccaaacacgggaataaaaataaatctgataaaaaatcatccatgacttccgaaccgctgttattacctgaacaccctccaccacgtcaggtcaataatgctggaatattagttctgtggccgatgttacctactctatttaaccaattcggcctgtttgaaaaacaaaaatttattcatcgtcaagctcaatttagggctgttaatctacttgattatctcatttggggaaacgaagaaacacagacagaacgaaaaatattgaattgcgttctgtgtgggttaattgccgatgaggacacggaatcaatccctattgagccagaaaaacaacaggtaatagaacaatggttagatgcagttatcagtcaacttcctgcctggaaaaaattaagccgcaatgatagccgccaattgtttttacaacgcccgggggaattgctgacaaatgagcaggaaatcaaaattacggtacaacctcaaccattcgatgcactgttaaataactggccctggccgttaaatatcgccaaacttccctggctggatcgccctttattaatcaactggtaaaacattgacaaggtttatatgaaagaacatcaatatagaatagtcgatctacgctggatttattcccatttggagcgcatcgatctgctgttacaacgtcactattaccaaaagagagacaaatacgattcattgccagaaagttttttgcttgaagaagatgaattagaacaacgtctagcaaaaccgttgggtattcctcattggctaacagcaaataccggcgctggtgatacagaaacagaaaatcattctgcttccggcacattatcactgctagtcacgcgttttaaactcactgaatttgaacgtgatgtgttattgctaggtttattaccgcattttgacaaccgctatcatgcgttatttgctactctgcacggtaacagtaaaaaacagtggcccagttttgatttagcgattgaattatttagccaacatcaaagtaactggcaattatttcaacaccactttttaccgcaagctccattaatcaatcaccatttattacgactcaataaccaagaggaacccatttggctacaaactcaatttttaactcacaatgcagtctggtcttttttatccggtcagcgcgtcattttacctcccttaatatcctgcgcttactggcatattccaacctcacagacttggtatccaccaatccttggtcatgcatttgaaaaaatattgctgaatgaaacggacgaaatacgcccgctggtggttcttaaaggaaaacaggacagcgccagagaactggcagtcagtaatattatgggaattcacggcattaacactttaacgttcgatttatttcacctgccagatgaagagtgcaccacctcaatactcaatctgctaatagatgcaatacgagaaacccggctacataatgcctgtttattaatccgtaacttttctttgctggcagaggaaaagagaatatcgcatagagaattatcagctctactgaatcaacccaaattacgtgtggtttgtctggcagagtcagaagaatcattagcatgggttaaacacctgccgatagtgcaaattaatatgccaccggcgacgctggcagataaaaaaacgatgctggaagccagtttgccagataatgtcactaaaggaattaatataactcaattatgtcaacgtttttcatttacagcagaaacattaccgttaattatcaaggaagctcatcaataccaaatcctccgacaaccggaagatcaattgaaagaatctgatctacgtaaggcattaaattgccgcgcccaacaaaatttcggtaaattagcccagcgtatgacaccaaaacgaagttttaatgatttggttatttccgctgacttaactcaacagttgaaagaaatcatcgcagcaattaattaccgtgaccaaattctgggcgcaggttttcgggaaaaaatcagctatggtactggtattagcgccctattttacggtgaatccgggacggggaaaaccatggccgcagaagtgattgccagctatcttggtgttgatctgattaaggtagatctttctaccgtggtgaataaatacatcggtgaaaccgaaaaaaatatctcccgtattttcgatctggccgaagcggattccggggtgctgtttttcgatgaagccgatgccttattcggtaaacgcagtgaaaccaaagatgcccaagatagacatgccaatattgaagtttcttatttattacagcgactagaaaattatccgggattagtgattttagcgactaacaatcgcaaccatttggatagtgcgtttaatcgccgctttacctttattacccgctttacttatcccgatgaagcattacgcaaagcaatgtggcaggcaatttggcctgaacaacttaagttatcagatcaacttgattttgagcatttggctaaacaggcaaatctgaccggtgctaatatcagaaatattgccttattatcatcaatattagctacagataataatagtgatcaaattgaaaataaacatatagcgcgagcattgatacttgaattaaataaaacgggccgattgattttttaatcatttatacccaataaatttcgagttgcagcgcggcggcaagtgaacgaatccccaggagcatagataactatgtgactggggtgagtgaaagcagccaacaaagcagcaacttgaaggatgaagggtatatagaattggagtgaatatgacaaatataattaaccctaataatgcgattcttgaagttaataacgcattaaatgatattttatctcagtatttaactaatattgatatccgctttgatctaccagaaataaattcaatcccatcaacccctacagtgagtatatttctttatgatatacatgaagacctacaattacgttctgctgaaccaagaagttatcatcctaccaccagctcattattgccgggatgggtaaatattaattataactatttaattacttactggcattcaagtaatccatcaagcgacagttctacccctgatagtcaacccaataatcaagcggcacaagtcatgactgctattttaaatgcattggttaacaaccgacaattacctaaaattcctggcgcatataccagagtcattccacctcaagaaaatctaaatagcttaggtaacttttggcaagcgcttggcaatcgccctcgcctttctttattatattcaattaccgcaccggtaaaactgcaaaatattaaagatgtcataaagcccattagccaaatttccacttctgtggatcaaaaatcaaatctggataattcgcaaatcaaccaagccttatttagcaaattgggtgccgatttaggtggcacacaagatgttcgtcttgctcttgcgaaagtgaatctgacaaccaaacctgctaaagaaaataatgaaaatcaaaataataaaaatgtaattattgaagtttctggcattacccatttggattatttacccagaataaaaggtattctttcaacatgggtaaatagtcatagtgctgttgttaggataaatgatattggtattattgtttcagaatataaatatgataaattaacaggcgtttaa

SEQ ID NO: 95 - DNA, 22492 bp, Photorhabdus
atgaatacagctcaagaaattattaaccgtttatcggggagagccgttacgcttggttgggatgttgttattgcttatgaccgaaaaaaaattaacactctgttagagcaacaatatgttgaaaaggtaaaaaacggggagaacttcccgcttatcaactgggagaaccagagaaaaacacttcaatttaaagatcttcaattaggtgttccacttatttcttttgagaattcaacactggaaaattcaagggcgcttgccacgatagaatttatttcaggagctattattgaatttagtgactccgggcaaataatcaactataagaagattgaacctagtcatggttatggcatggtgctgactatcgatctcatggctggtacaggttcagtagaagaacaaggtcgggtgataataaatcttaacgaaggcgccatactcgatttgcatgttatccaacaaccgccagcagaagtggtagaatttttccgcacttggttgatggctaataaaatgacttatgaattaggtaagctggatctgagtagtcaagctggtctagtgcctcgttcttttcgtattcgtactcagcgggcgcctgaaaaaattcgtaaagcgacgagcgatgaaggaaatggcgctgttttgttgtttgttgccactaactataaccctacaagtggaactttacctgccaaggattatccgtggctaatccctgaggaatattcaggcgcattgcttatcggtaataaatgcttatttaaagacattctgaaaccgaatctggatcagttgtttgataaaggggaatggacattaaaagttcagcaaacggattctgatcaactgctgcattatctggaggcaaactctgcatatataacagataagccttatatggcagactttgaaggaactcaggatggagtctggacaggacgttataaatttgagactggccggggacattatggggtgtatgaaaatgtacgctttcctatcaatggaatgttgatgaaaccggctaaaactggattacagttatcaatagattcaccacaaagccatcaatttaatgttgatttcggaatgaagtggttccattgtgctaatataatgtgtggttattcctggtttaacgagacttacccattttatcttgatggaaaatcattttatcaagttcatattgaccctgataaagaggtgatttattttactgggccagatgaagatattaatattgtaggaaattacagcccgcctgcgtggtggcaatctaaatggcaaaaacatatcagtgatgattttacggatatttcctcggaaaaatttaagcgactcagtcaaataaaattgccagaaatatgcatgtttgccgtgaaccatttattatttcctggtcataatactttgctgttgaaagacgtttatttaccgggtgatatggtgattttcggtgatattaacccatcacttaccgcttttcgggttacgccattaaaagcaacagtggtggcaaagggaacccaacaatttaaagccatagaaactaattgatgattatacccttcatccttcaagttgctgctttgttggctacgttcactcaccccagtcacatagttagctatgctcccggggattcgctccctggccgtcgcgatgcatcttgaaatccatagggtatatatttaattggataagtcttttttattttaacattataacctgattctttttggataaaattaaaggattattaacatgtctattacacaagaacaaatcgctgctgaatatcctattcctagttaccgttttatggtttctataggagatgtgcaagtcccttttaatagtgtttcgggattagataggaaatatgaggttattgaatataaagatggcattggtaattattataaaatgccaggacaaatacagagggttgatattacacttcggaaaggcatattctctgggaaaaatgatttatttaattggattaattccattgaactcaatcgggtagaaaaaaaggatattacaattagtttaactaatgatactggcagtaaagtcttaatgagttgggttgtttcgaacgcctttccgagctcactgacggccccttcatttgatgcttcaagtaatgaaattgcagtacaagaaatttcattagttgctgatcgggtaacaattcaggttccctgataactaaaaactttaaggaaaaataatgtctgtacaaacaacttatcccggaatttatattgaagaagatgcatcattgtctctatctatcaataatagtccaacagcaatccctgtttttatcggtaaattttacaacttggatggttccttacctaaagtgggaacatgttctagaattaccagttggttagatttcactaaaaaattttcggtagctcctcctcaaaccatttcattgatcgcgtcgccaattgctgacacacaagaaagtgtacccaaagcagttcaatatacttataaggccgagtttgaaacctcagaaaatctggcaaatggtgcctatgcggtacaacattatttccagaatggcggtggtatttgctatatcatacctttagttagcgtgaaaaaagaggatgctgcgattgagttaacaaaattacctgaattaattgaaagacaacaagagattacgttaatcgtctgcccggaggacgataagacgctcactgttgatagcagtaaaaaatcggatgtttataacagcatcaatacattattgagtaataaggtaggttattttctcattgcagattcagatgatggcaaagcagttcctgatacgttgccggaaaaaactgcggtctattatcctggtttactaacttcttttacacaacgctatgcccgacctgccgattctgctatcaaagtgaccggtattacaaatatatcaactctggctgatattcacaccaacttggccgatgactactcaacagcaagtcaggttattaatgatgttttggaaaaaaataataagctcgcatcgtctcccattattttacctcccagcgccgctgttgctggtgcttatgccgctgttgatgtgagtcgtggtgtttggaaagcacctgcgaatgtgatgttaagtaatgccacgccaatcattagtatttccgatgcggaacaaggtgtgatgaacccattaggtattaatgctattcgtagttttactggtagaggtactttgatttggggagctcgtactctggataaaacggataactggcgctatgttcctgtacgtcgtttattcaatagcgcagagcgagatattaagttagcaatgcgttttgcagtttttgagcctaactcccaaccaatttgggaaaaggtcaaggctgctatcaatagctatttgcagtcactttggcagcaaggtgcactgcaaggcaataaacccgatgaagcctggtttgtacaaattggtaaaggcgtgaccatgacagatgatgatattaagaatgggagaatgattatcaaaatcggcatggcggcagtacgtccggcagaattcattattttacagtttacgcagaatatcgcccagtaacttaggtctataccctatagatttcaagatgcatcgcggcggcaagggagcgaatccccgggagcatatacccaatagatttcaagttgcagtgcggcggcaagtgaacgcatccccaggagcatagataactatgtgactggggtaagtgaacgcagccaacaaagcagcagcttgaaagatgaagggtatagataacgatgtgaccggggtgagtgagtgcagccaacaaagaggcaacttgaaagataacgggtatatttaatatgggcgatttattgcccatttttgtgaaaggaaatgagttatgtcgccaacgctacccggtgtaacgatgactcaggcgcagataacagcgttcggtgtcagtacattaaatatgcccgtattcatagggtattgtacgagattgcctgccttttcagcgcctgtaaaagtaaacagtttagctgaaacagaacaaataatagggaaagaagggcgtttgtatgctctattgcgccactttttcgataacgatgggatacaagcttttattctgtcgttaggcgcacctgctggggaaaatgctaatagttggcttgaggcattacaacagcccgatttgtatgcggctgttgcagcagagccgctaattacacttttagccgtcgttgaggcaagtgaactgaaccaaaaagaaggtaatgaggctgtggaagcttggcgacagtactggaaagcagtattagcgttatgtcaggcacgcagtgacttgtttgccatattggaggcaccagatgataccgcattaatcaagcgtagtttgcaggattttcatcataaggcacgtcagtttggcgctctctactggccaaggctagaaacatcttatcaatcctctcagttaaaaattttgtctcctattggtgcagtagcagcggttattcaaagtaatgatgtccggcgaggggtaggacatgcacctgccaatatagcgttaaaacagacgattcgcccgataaagtcccgcctggaattagaagagttgtatgaagaatcggatggttcactgaatctgatttgtagttttccagctcgtggtactcgtatttggggatgtcgtacgttggcgggtattgattcaccttggcgttatattcaaacccgattattgacttcacacgtggaaaggcaactcagccagttagggtgcatgttgatgtttgaacctaataacgcagtcacttggatgaagtttaaaggccatgctgggaatctattaaggcagctttggttacaaggggtgctgtatgggcagcgtgaagatgaagccttttccgttgaaatagatgaaaacgaaacgatgactcgccaggatattgatgaaggcagaatgattgctcgtattcatttggcattgttagcaccggcagagtttatcgctgtgactttgaattttgatactcgctcaggcattgcgacgagtacataataaatcggaatatctccatgacactaccagcagagctttataccccagcggtttcacatcgttttattgttaattttctttttaaaggtttacttccttctcccgtagatattcgatttcaacgtgtttctggtttagggcgtgagttacaggttgaacagcgccatcaggggggagaaaacgcacggaatcattggttggctgaacgtatacagcataatagcttgatattagaaagaggggttatggtcgttacccctttaacactgatgtttgatcaggtgatgcggggggaaactctcaattgggcagatgtggtaattattcttctcgatcaggctcaacgtccgataacaagttggaccttgagtcatgcgctaccggttcgctggcaaacaggagatttagatgccaacagtaaccaagtgctgattaacaccttagagctgcgttatgaagatatgcgcattataggggtaaaattatgactatcgaaatccgtgaactcattgttcaagcccgtgttgtcgggactgataccaaaacaacacgaaccgttcctttatctattgtgcaaatggaaacacttatagaacaacgtctggttgaaaaagtgaagcgggagatattagacgtactccgggaagaacaaggtggtgggttatgagcttgcttgaacgaggtctggctaaactcacgattacgggttggaaggagcgtgagcgtaaacatcagattggtaaactagaagcaatgtataacccggaaacacttcaactggattatcaaactgattatctccctgatgttagcaataatcaggtaacagtgagtaaccgctacgttttgtcaaagcccgcagggttaacactatccttgttatttgatgccaatatggctggtcttacgacaaccgtcgagtcccaaatcactaccctcaaatcgctttgtttagttaatgcaagtactgatgaacccaattttttggaaattaattggggggcaatgcgttgggaaaataaaaattattttgttggtcgggctagtggattgtctctgacttatttgcgctttgatcgtaacgcaacaccattgcgtgtgagtgcgcagctcacattagtcgcagatgaaagctttgtgctccaggataaccaagccaagttagatgcgccgccggtatcagtagttaatgtcccggatctgacttcattacctgcactggcgaatatcgctagcgtaaccactatgttgggagtggattatttaatgttagcccgcaccaatgatatggataatttggatgatatgcagccaggtcagacattgcgaacaccggaggcatcatgagttttttagataacagtaacttcaagccatcagatatcaaactgttcgttaacattcagggagtggagaaggaactcaacgaactgatagtaagcgaattgaaaatctcccgacgtatcaatgccattccgcaggcagttgtaaagctaagagcgaaagagagtgaaagtggtgtatatcagtctgatgtacagcggatgttgaagagttgccgtccgggagtaaaggcagagcttcgtattttgaatacccggctattcagtggcgatattgtgcagcaaaaaacagagttagtgtatgcgaaaacacacactatcaaattggtgctacgccatgacttacagcgcatcaccggtaattttcgtaccagagtgtttgcgaatacccgtgatcgtaaagtgatagccgatctattgaataccgcaacattaaagccggcattttcggggacatcacattgggatatagatcatgagcaactggttcagtatcgttgcagtgattggcaatttttgttgcaacggctctatgctacgaatagctggttgttagctgaagaagataaagataacactcaggggaaagtgaccattattgctccaaattctttgcccctgaatgagcgttggacactgcaacatcaggctgatcatcaggctatccggctttacagcacggagctgatgctggataaccggtttgatacagcggaggctgttgttagtgcttgggatattgatgatcaggcattactcgtggcgtggaaagaaacccttagtcaagttgggaaagatgcgttagcgtcagataattttagccagacaaataaagattcgagtgaactgttattaagttgtccgctctctacaaaagaagttcaatttttaacgcgtagccaattagtcatgcggcgcttgacggccgttcgtggttcactgaaggttgaaggcagtactaagtaccgtttagggcatgaactgatgttgtcaggttttggtgaaaatatggatggctcacaaatactgacgggagtggatcatcgaataacggcagaagaaagttggaaaacaaccttacatgtgggattagaactgccgttaaaggcagagtatgtcactcaggttaacggtgttcatatcggcaaggttgctgattatcaatcagatagcaaaaaatgggatcgtattcctgttttgatccctgcatttggaacgaatattcccttgtttgcccgattgggaaaaccctacgccagccaccaaagtggattttgtttctatcctgaaacgggtgatgaagtcattctcagttttttggaaggggaccctcgttatcctgtcattattgattccctgcataatcctaaacaacagactccattgcaaatcagcaaagagaataatctcaaaatgttgatgattaagcagagcgataaagatgagcaacaattgttatttgatagccagcaacaaacagtcgcgttaatcggtaagaaaaatatcgaggttaaaggtgagtatatcaacctgactaaatcaaaggggactcgataatggcaaatacgcttattggccaggtatatggtcaaggatgggcttttcccattaaatttattcctgataataaagaaaccgcagatcaaacagccggtattgttatggctcaagggattgaagatgtcagtcaatcgctggaaatattatttcttaccgagcctggcgaacgaattatgcgtgaagattttggttgtggtttacaagattttgtttttgaaaatattagtgatacgctaatttctgccatcaaaaatcgtattcagcaagcaatattacgttatgaacctcgcgcatatttattgaacgttgatattcaaaccaaagaaaaccaacctggacatctgctcattcagattaattggaaattacgtggtagtgatatatctcagcgtttagacggagtgcttagactccattcaggtcaagcattggaactgttatgaccaattatattattatcgacggggatctcattcaaataaatcccaaatttgagggtgatcgaactcttacgattaatggtattcctaaaataagcgggaatggagatgcgcaaattgaaggaaaaaatatttgtgtgtcaggtgatcacttaactgtctcaattccagccatttatataacctccagacatcctgttgcaggtagtggaaaagtgaaaattacaaatttatctgacgaccaactagcagaattttgtgttagtggggatgttgtgattattgaaggcagtcagtttgaagctcagtttacaccggataagccggccactaatccaagtaaccaagatgcagataatcctgcgccttcgaatgggagtgggagatttatacactcacagaacttcgttaaggcagaaaaataaaaaattttgccgaagcggttaataagtatgaataagcggggcggataaaaacatggatcttgctgaattaaataatacgttgatgaatgacttaccaacgaccaattttaagttagaaacaaaggacccattaacgcaattaaagtggttacaacgttatacagaaaatattcgtttttatgcgaatgatgattatttctggcatcaattctggttcttaaaaaatcacacaccagaagcgctctttgctcgtttgcaaggtgaaacgttggctgatggagaattgcctcctcatcaagcgctattgctggcctttttacaacagcttaagacgccaggaatcatgcttgatactttttcagcccgtcatcggcaattgtactatcaggaattgctagggataacgcagaaagatgcacaacctgatcatgtggcgcttggcgtggtattaagtactggtattgcagaatatttattaccgacaggcacattagtggatggtggacaagacagcagcggaaattcactgcaatatgcgttggataccgatttattggttaatccagggcaattaacagatgttcgctacagctatttggatcataagacctataaaatcttcatcttgcaagatgataaagcgaatatcagttggccctcttcaggcgctcgtttatttgtagcacctgagggcaacggacaggaaaaggcacctgaacaaaagttggcactttacctgggatttgatgatatacagccagggcaaactctttctttattttggcaattcattgcatcaactcccctgacattaaaatggttttatctgaacgagataaataactgggtgaagctagatagtgtcagagataacacggatggcttttttatcagtggattatggcaagcgatattacctgatgatgcggtgaaaatgtattttccagagacaacttctgtaaaacgctactggattaaagctgaggtggaatcgcttactgaatctggcgatttgtggcaaccgctattagaaggcatcttgtataacgctcaaacagcaacgctggttgatgcagacaacacagatgaaaagcactttcatgatgggctgatgccttttagcgtgcagcatttggtcaacaccgtttcagaggtaaaaaaaattgagcagccctggtcttcttgggggggaacgccacaggaagacactactgatttcttccatcgagcggcaacacgtcttcagcatcgccagcgtgcgttaacttgggataaccaaattgccatgttgaaggctgaatttccgcggatttatgatgtcatctcaccaaatatcacgtggatgaaccaacttcagacatcaaatacgcaaacgctgatcgttattcctgatgtgaactacagcgacaacaaggatcgcttacggccacaattcagccctgccagcttgcgacaaatgagtgactggttacagattcacactagcgcatgggcgaatccacaagtggaaaatccaatttatattgatgtctctgtgacctatgaggtgcaatttagtgcgggtgtgaatcctgattatgccctccggcaattacaacaatggttgagttcaatttatatgccatggtatcacgcagataaaaaaggtgttgccgctggcgatcaaatcgatttttaccaactgtttgcagatattcagcgagtaccttacgtggagcatgtcaaaacattgacattgaccacaaaagacacctcattaaccaatggcggggttattaaggcacagcaaaatgaagtgctggtgttggtatggcaacaaggagaacaaattaggcagggagaatcgaaatgaggcagcataatgagttatttcctgtagtaaaagacgcgataagctttgaaaacctgcaagctcagggtgagaaggttattagtgatcagtccggtaacatatggagcgataaagataaacatgatcctggtataacattactagactctttaagttacggtgtttcggatttagcgtatcggcactcattacctttaaccgatttattaaccattgctggaaaagatacgctttttccagccgaattcgggccacagcagacgctaacttgtggccctataacactggatgattaccggcgtgcgttacttgatttacatggtaatgatgcatttaaaatatcagctagtgaccccagagactttttgtttcaggatatacagttaatttgtgagccaaaaagtaagcgttataaatactatttcaatcccgaaacgcttgaatatacattcacgccaccttcaggggataaatttaaaactttaacactacgagggaattattggctttattggataccaacccgttgggcaggtaaatcagctaatttgccgttagttaagcgggtgatggaagattttctccgtgaaaatcgaaatttgggggaaaatgttgttcaagtgacacgggtgatatcaacgcctatttatcctgagctggtcattgagctggcggatgatattacagatgcggcatcagtattagcatcaatctatatgctattagaacagtgggcgatgccgatgcctgctcgctttactaccgaagcattacaggccaagggattaacaaacgaagagatctttgatgggccgtggttgcgtcatggttggatacctcagttaccgacctctcaaaactaccatacaggcatggttctgaagatgaatcatctgattaaccaattgctggcggttgaaggtataaagcgcgtagttagcctgacgttgccagaaacagaatatttgcatcagataaaagatgataattggtcctggcaattagatgttggttattatccattattatggggagctaatccactagaggtaattacagagaaaaataacaattatgtcaaattgttcgcaaaaggtggggtacgattacaacctgatcagaaaagtgttgagcggttattatcacaggaatcactcattaataatgctgcatccacgttaccggctggtaaggtgcgtgatctcaaagcctatacacctataagccgcaggttgcctgcctgttatggtttgcagaatactttgcaaaagttaaaacctgaacaacgacacttatatcagttcctattaccattggagcaaatgcttgctgatggatgtgcgcggcttgcatttttgccacatttgttagcatttagggaccgaagcggaaatatcagtgatacactctggcctttcaagaatacagaggacacaattgcccaacaggttcatcaggaatatgccggtacattaaaagcctttcaacagcaggaaattagcctgtttgatgataaaaatagaccgcatcatggcaatatcaatcgggaattagatattcttgattatctgctagggtattttggtacacaacgtgcaaagcgtccattaacgcaggatattcatgattttctgcaaacccagcgaggttatttggcacagcagccggagttgggttatcagcgtgataatatccgtattgatcgagtttcagctttacaaaaacgtatagcagcccgaattgggctagatggtactattttcaaagaatcggttgatttaagtaagttacctttttatttgattgaacatcgtcagcttttaccaaatttaccccatcttgactttcaacatgatcaaactccccaatcttttgtgatttccgacaacattgttaaagtgaaacaagcgggaatagcagataaaatcgttcgtggacagcttattgattttatagatattgaaagcaaatttaccgttcgtgcccaaatgattgtcgctgtagagggaaatgaattttctctggatacaaaaaatagtattcaacttgaaaagaatctgcagttattacaatcagcgtctgagaaaaacaatttacgatggagaaatagcacggcgtggttagaggatatgacgtatcgtatcaattatactgacgatcaggttatagacgataaaacaaaacaatgtcgtttacaaagtaatactaaatcgccttttccagccttaattgcaccaaaaaataagattacgattattaagcaatcttctccactctccagtattgctgaatttactgatgaaccagaattcaaattagttgcaacggtgacagagattgatcggattgaagggatattgactatcgaacgggatgacaaccaactccctttcccgactaaagaagagagtaatcaatatatatggtacatatctgatgaaaactatatttcaagtgatcgtttctcttttgtggtgagcgtcgtgctgaatcgcggtttggttgaaagggaagatattgatcaatataagctagaggaatggatagagcgtgaaacacttgcagagtttcctgcacatatttcgttaattactcattggctggcatctgaaaatttcgatgattttgcgaagacatatcaacgttggcaaaacaatggggcgcagttaggggatgaatcctacaccattttggaaaaactgacattagggcatttaccaacaggacttactggcattagtaatatgtttattgctacagaagctcagcgtctagaagttgttggcgagagtggtaatgagtggaatacccaggcaattattaacaacgaactattctatgttccctcacagaatagttaataccgagtgttgtgatcaacttttattataagccggaggataaatggacaacaaaaataacaaacctactgatcaagagattctaaaaacatcacgggctgtcggagaaattccttcagcggataatttaaaaaatcgttttaaagctcgttcgattccattagagacggattttactaatctcattgaccttgctgaagttggacgattggctatcggccagtcaccatcgcagcaaagtaaaacgcctggcaccggaatggaattaacttcggatggtaaattacaagtcaaggctggggcaggtgttgatatcgataataataatcgtattactattaagtctggtcatggaattaaggttgatggaaacggcatttccgttaaaccaggttcgggtattaaggttgatagtaatggtgtaaatgtcaatattgatgatttttgggaggaaatacgcaataaaattatgcctaaaggaaccatgctgcctatttatggcacacctaacccctctgcgctgccaacaggatgggaatggtgtgatggtaaagatggcagacctaatttaaaaaaagggaaatataacttactatcaggtcagtcttcaggtactgatactttttgggcagataataagaatggagatacagagatcaacgtgttatttgtttactatatgattaaggttgtgtaatatcttaagtaatatgcattactctaaaatgaatgatttatatttaagtaacataataattaagttgtgttgtagggctgtttttatgagaaatataaaaacggaggtaataattggcttcaaaatatcagtgatgaaatagagttatttcgctttataaaaattttgttttatttcttttaataattatttatagaaggtaatgatatgtgcacacaaaaaaacgtgttagatagactgaaagatagaaatattacattgggttgggatgttgttgttgcatataaccaagaaagtgttaataagttattgaagcaacaatatgttgaaaaagtttactcaaatgaacattttgtttttaaagattggcatgatgataataaaacgaaatttattgagggattaacagtaggcgctccactagtttcatttgaggaggcgtctttatccgatgctaatgtaaaagtgacacttaactttctttctggtagatggagagttatacaagcaaataccggcacaccaattgaatggaaagaaattgttcctggcagtggctataaagcagaattagttgttccgcttaaatcaataactggtagtgtaagtaaaaaagatatcatattaaaattcaaagatgctgtcgtaaaaaaaataaatttatttgacaatcaagagcctgattttattaattatttcaagcaatcgatcagtgagggaaattatactttagggcaactggtgacagacagcacaccgggattaattcctgctgaatttcatattcgtactcaaccccatccaaaaacacgtgagcgtggttctcaatatgtaggaaatggtgcggtactgttgtttattaaaacgcaatatggcggaagtggaacattgcctgtaaatgattttgattggttaattcctgatgatcatactagcgcattagtcatttcgagtaagaccatgatggggcaaatattgccaaaacaatacaaagataaattgcctggtgatcctcagtttagcccaccaaaaagagtcaatgataaacaagactctgcttattatattacgattaccgatggtggatttgatggtaatagccctatagagaagtcatggttacgttctgattatagcaatgggatttggactggtgaacgtggtaatgctattattggtgaaaaaggaaagcggataccaccacgttttccataccaaaattttgttattaaacctcatggtgaatcgttatttcaaggatgggagaataagataaattacactcaaaagtgtgcaagatatttccgacatcatagtaatagtataactttcgaagatactgcattaatggatctcagtattggtggacaaggtagtatcaattgccagattgatggtgaacatttctatttaaaatcagatgatttttcccccaatgtcagctatgaaccaacttcattctgggataaatttatcggtggggtggatgcaaatgtgaaagatgaattcagagatgaattagcacaacaggcagaagcaaagttaaaacaggtatttaatattgaattgcctgaaatcagtctgttttctattaaacatctgctctttcctggcatggatgttatgcaacttaaacagggttatttcccaggagatttgattatctttggggatatttcacctaaattgaccacaattcaggtggctcctttggaagccatggttgcccttaaagaaaatcaaaaattcactgtcgtacctgaaaataaaaatgttagttggaagttggatcataatagtgaggctatcaatgatccgggaaatattgatgataaaggtatttatacggcaccgggcagaatcagatctggttctgaagtcattaaagtcactgcaactgacggcgatggaaatcaggcatcggcggcgctgacgttggttccttcttctgttgcattaacaccttcttttgcttttatctctgaagcagataagaaacctatattattattggcgaatgtcctagacggaaaagcagtaacatggaatgtggaaagctgtacaggcagccaatgtggttctgttgatcagaatgggctttatactccaccagcagggcgttttaacgatggatttacttttgcatccatcaccgcaactgcaaaagatggtagtcaagcacgaaccattatttgtctaatggcatcaatgccaggacatggtttttacaaggttgaacctaatttacgtttgaatgtgaaagtaggggaagaaattatctttaaagcgcaggcagatagctataatggtgatcctgatacttgggaaattttccctcctcgcggaaaattaagtgaacctgagtttgaacccaataatgatcctgaaactaatgatacaatttttggtcattataaggtgacctataccgcgccgactaatgttacctcacctgaattgcttgttgtccatgtatgggagaaaaataggcataatgagaaaaacaaaggtaaggcaggatatgcacttattgaaattatcccagatgataaatagaaaatttatttaaataaaaatcacagcgggtttatctcgctgtgattaaagtcatctttttttatagattgtttatctctaataataattttattttataatataaaggaaattaaaatgaataatgaatataaaaataacaccgtgaattggcgtatttcacctgatacggtaggaagtattgataataacggtttatatacagcacctaatcgggtaaagaatatcgaatttgtccaagtaatggcaagcgatgctaataataatcaatcttctgcgattattactgttattccctcttctgttgcgttaacgccatcgtttacttttatctctgaggcaaaaaaaacatcagtcacttttaaagcgacagaacttgaagggaaaaaagtgacatggagtataaataattataccagtaatcagtatggttccatcgatcaaaatggtatctacacaccaccggaaagtcgttttaacgatggatatacttttgtatctattacagcaaaagcggaaaatggcgctgaagcgcaagcgcttatttgcttgatggccaaaattccagggcatgcctttttcgatgttcagcctaatatatgtttaagtgtgaagcctggagaagaaatcatttttagagctaacgcagatcgttataatggtgatcctgattcctgggaaattttcccgtctcttggtaaattgggtgagcctgagtatataaaaaataacgatccagaaattcctatttatggatattatcaagtgaaatatattgcgccaaccaatataaattcttcccaaatactcgttgtgcgtacttgggaatatgacaaacatgatgagcataatcaaggtaaagcaggatatgcattcattgaaattgtgccagaaaatgagctttaatatatatacccaatagatttcgagccgcagcgcggcggcaagtgaatgaatccccaggagcatagataacgatgtgactggggtgagtgaacgcagccaacaaagaggtaacttgaaagataatgagtataaatgactttagtaagagaaattatggcttcattcagaactatttattagagtaattaactttataaagacatttaatggaaaatataatagaaaaatttaatattaatattgaagtctcatctgaaattattggagagagtttattaaactcccctttattgatgagtagagaaatcagcaatcaattatctgaaatattattagattataaagaatataatattgcattggataagttagtgttaaatataggagaaataccctatgaaatatttgaacaacaattctatggtcgtttgggaaaattattaaatgaaaagttaacaataataataaatgataaattattggtaaaaaacatatcaacctcgttatttcctgaatgttttagtgaaaaaagaaacccattattaaatagagtcataaaaaatttaccttctaatttggtttttgaagttcattcaatggtaaaaatagaatcagtaaataacaaaaaacaagctaatatattgacatcttatctggcttattctttttttaataaaagcaaattacaacaacatttattttccactagtaataataaattaattgagagcttatacgcactttttctaacggatcagaatcgaatacctactgctcataaaataggaaaaggtgcacttatactatctgcccttatttggctttattctaattccaatgattatctgcccaaaccagaaagcactctgttgttacaaatagaacaggatataaaacaaggatatttgcctttaacgttgttaatcactttcttccagaacagaaatggcgggcgtgttttttgcgattggcagtatgcgttatggcaaatcgatatcatcaaaaatcacttaggcattaaaataacatcgaaagaaccccatttacgggagaaaataatgttacaaccagttaatgcttctgatcgatcctctgtgctgatatcagacgaaaaattgacaataccgttaacaattacaggtgcgggattagtgcttctctggccactattaactccactattttcgtcttttgatttgttagataagaaaagtttttcagacaatttggcacaggaaatagcatttaatttattggaatggttagtctggggagatgagatgctgttacatcaggaatcatcattatctttattactctgcggaatagatcaccaaacaatactggagcgccaggttcttattcctgagcacaaggaaaaattaaataactggttgcaaggtatttgtactcaacttttctcttggaaaaagctagggatcgatgatatgcgccaactttttttgcagcgtcaggctgcactttattatgaagatgatggccgttggttattaacggtgcagcgtgaagcttatgatgtattactgactcaaatgccttggccgtggccattgaatattgtgacattaccttggctagctgagccgattagtatcacttgggaaggtatctctgaaccaacggatttgtcattttggtaatccaatatctcattaggaactctatgcatgtacgatttatctgatgatcttgccagacagaatatttcaccggaatatgaattgacggttttgctgtctcagactgctatattggataaacgaattcgtttacgaattcaggaattaatgcaacagcaaacactattgggagaaagtggacagacgtcttttgatgatatttcattttcattcgtttcgagtgaacaacaaaaatcatcttatttggtgtcaccgcatcaaaattggacgaaagaggattttcctcctgagccgatcccatctcgtagccgtctaggacaattagttgaacggtttgacttaactcaatttgaaattgatttgattttattgtgcctgttgcctcatcttgacagacgttatctaacgttattttctcttgttccggtaagtggaggtaataacagcaaaaagcagatgttaacgttgggattggctttggagttgctttgtccgagtgtagtagagcgcaatgcgcaacgtgccagtttattaccacaggcaccgctttgggattatcgtttatttcagttgcgcggtgatatgtctgtttcctacgatgaaataccgttagcaatcgataattctcttatgcattggttattggggcatgatgctctcccgatttctcttctctcccgggctcattggcttcctgttcctgaagtgcctgatattttgcctgatttcaccaaccaattgatagaactctgccaaatggaacaagaggggatgctgacaataatcgccggcggagccggaagtggcagcaaaacaagtgttgcacgcgcagcatcacaagtagggcgctctgtattgttgttatcgttagcatcagtgacactgagtgaacatgaaactattacactgataacactggcattacgtgaagcacaactaagaaatgcctgtcttatgtttgaagctttggatgagttttgtgaagcacgccccgctttgcagctctggctaggaaatcgactggctcgttgttcgattccgctgttttgtcaattacctaagcaagcatcattattgccattggatgcaatttcacaagttgtattgtctatgccaatgccttctttaatggtgaaggctgcagcattagcttcaatgatgacgaattattttccagacaattcattggatgttgaaagtttagtgacatgtttccatccttctccattgatattgaaaaaggcccttagtgaagcagaaatttatcgccgactacggggggaaacggctagtttgagattagatgatgtgcaaatgtccctgcgttttcggttacagcagaattttggacgtttagcacagagaattacaccacaacgaacctttgatgatttgatcatcagtgaatctcaacagcaacaattacaagaaatcctggcggctattcggcaacgagataggatgctagagcaaggatttgctcgtaaagtgagctatgggacgggtatcagcacgctattttttggtgaatctggcacaggaaaaacgatggtagcagaagtgttagctggtgttttaggtgtggatttgatcaaggtagatttgtccactgtggttaacaaatatattggtgaaactgaaaaaaatctggctcgtgtttttgattatgcccaagaagacgccggggtattgttctttgatgaggcagatgcattgtttggcaaacgaagtgaaactaaagatgcaaaagatcgtcatgctaatattgaagtttcctacctattgcaacgccttgaaagttatccagggctggtgatattagccaccaattaccgtaatcatttagactcagcatttagtcgtcgcctgactttttcggtacgattctcttttccagatgtttccttacgggaacggatgtggcggattatctggccatcgggaattcaattagccgacgacatcagtttttcagcgttggcaaaacgggctgaattaacgggggcgaatatccgtaatattgcgctactcgctagttggctggcagtagatgaaggaaatgaaaaaattactatggctcatattgaatgcgcattacgacgtgaactgagtaaagttgggcgcattgatttaccttaatttttctttgtaatcgggagacaactatggttaaaaatatcaaatcagatgaaaccttactgatattaaatagtaaaatagaagatgcattaaaagcgtatttaccgggcgaagatgtcgttattcggttcgatatgtttggtaaaaatgaaaatccagattctcctaccgtgtgcgtttttctttatgatattcaggaagatctgcaattacgcgtgggagaagggcggcaatacctgcctgcgacaggaaattttgtcccgggatgtgtcaatgttcgttgtaattatcttatttcctactgggagccggaacagagcggagggcagggatcgccaaccatacgttctaatagtcaatcaatgaagataatgaactgtgtattgaatgcattaattaatcatcgttcatttcctggtttacccagaacttatacgagagttcttcctcctaatgaacaattaaatagcttaggaaacttttggcaatcattagataataagcctcgactatgtttaagttatatggtgactattcctattcaacttaccccgccgacagagaaggtatctcctgtcattacctcaaaaactgatattactcgaaaaccatcgcttaacttttatcttgaggcagatgaaattatccgtcaggcattagttgatgccttaatatctcaaacaacagaatctatggatacgataactagctggctggcaaaagttgttattatttgtcgaccaccagaaataatgaataaacaaatgattgaacaaactgtgaaattaattattgctggaattacagaagagggattagctggaaatataaagacaatcactcaaaagtgggtggaagagaagacgattattggtgaaatcgacgatgtttctctagttatttcccaagttgacacgacagcgttgtctgctgtaacaataccgacatctgtttaa

SEQ ID NO: 96 - protein, 14 aa, Photorhabdus
TGQKPGNNEWKTGR

SEQ ID NO: 97 - DNA, 34 bp, Artificial Sequence
  Primer
tatcatatgtctacaactccagaacaaattgctg

SEQ ID NO: 98 - DNA, 27 bp, Artificial Sequence
  Primer
atctctagaacagatattccagccagc

SEQ ID NO: 99 - DNA, 19 bp, Artificial Sequence
  Primer
ggcgtcacactttgctatg

SEQ ID NO: 100 - DNA, 20 bp, Artificial Sequence
  Primer
tcggtggcagtaaattgtcc

SEQ ID NO: 101 - DNA, 27 bp, Artificial Sequence
  Primer
atgtctacaagtacatctcaaattgcg

SEQ ID NO: 102 - DNA, 19 bp, Artificial Sequence
  Primer
gactcccttgagggtacgg

SEQ ID NO: 103 - DNA, 21 bp, Artificial Sequence
  Primer
ttctgatgagagtgatggtac

SEQ ID NO: 104 - DNA, 24 bp, Artificial Sequence
  Primer
tgaataaagaattcagtcaatatc

SEQ ID NO: 105 - DNA, 20 bp, Artificial Sequence
  Primer
tagtggctgatgaaagtctg

SEQ ID NO: 106 - DNA, 22 bp, Artificial Sequence
  Primer
ggaagccaaagataatgaagtg

SEQ ID NO: 107 - DNA, 19 bp, Artificial Sequence
  Primer
catttcttccctatggttg

SEQ ID NO: 108 - DNA, 24 bp, Artificial Sequence
  Primer
ttaaattcctacaagattatcttt

SEQ ID NO: 109 - protein, 133 aa, Homo sapiens
RSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 110 - DNA, 399 bp, Artificial Sequence
  Synthetic Polynucleotide
cggtcaagtcactcgcgtctggggagaatcgaggctgatagtgagagccaagaggatatcataagaaacatagcacgccatttggcacaggtaggcgattctatggatcgctccatcccgcctggacttgtcaatggtcttgcgcttcaacttcgtaacacttcccggtccgaggaagacagaaatcgggaccttgcgactgctctggaacaactgcttcaagcatatcctcgtgacatggagaaagaaaagactatgttagtattagctcttcttttagctaaaaaggtagcttcgcacactccaagtttattgcgggacgtttttcacaccactgttaatttcatcaatcagaacctgcgtacttatgtgagatctttggcgagaaatggtatggat

SEQ ID NO: 111 - protein, 15 aa, Homo sapiens
LSESLKRIGDELDSN

SEQ ID NO: 112 - DNA, 45 bp, Artificial Sequence
  Synthetic Polynucleotide
ctgtcggagagtttgaagcgtataggtgacgagctggacagcaat

(19) United States
(12) Patent Application Publication
(10) Pub. No.: US 2023/0076614 A1
(43) Pub. Date: Mar. 9, 2023

(54) LEADER SEQUENCE

(71) Applicant: THE UNIVERSITY OF WARWICK, Coventry (GB)

(72) Inventors: Nicholas R. WATERFIELD, Coventry (GB); Joseph HEALEY, Coventry (GB); Alexia HAPESHI, Coventry (GB)

(73) Assignee: THE UNIVERSITY OF WARWICK, Coventry (GB)

(21) Appl. No.: 17/613,911

(22) PCT Filed: Jun. 5, 2020
(86) PCT No.: PCT/GB2020/051380

§ 371 Date: Nov. 23, 2021

(30) Foreign Application Priority Data
Jun. 7, 2019 (GB) 1908155.3

(51) Int. Cl. C07K 14/195; B82Y 5/00

(52) U.S. Cl. CPC C07K 14/195; B82Y 5/00

(57) ABSTRACT

The present invention provides use of a Photorhabdus Virulence Cassettes (PVC) effector leader sequence, for packaging a payload into a PVC Needle Complex, and related methods for manufacturing a packaged PVC Needle Complex. The payload is one or more selected from a polypeptide, a nucleic acid, or a combination thereof, and the leader sequence and the payload form an effector fusion that is distinct from a wild-type PVC effector protein.

The present invention relates to a leader sequence, and use of a leader sequence for packaging molecules into protein complexes.

Biological molecules (e.g. peptides, proteins and nucleic acids) have great potential as broadly applicable therapeutics. Indeed, there has been a trend in recent years for the pharmaceutical industry to move away from ‘small molecule’ drugs, toward more complex macromolecular therapeutics (aka. “biologics”). Such biologics include protein-based therapeutics (notably antibodies, hormones, growth factors and cytokines) and nucleic acid-based treatments (such as short-interfering RNAs, DNA/RNA vaccines and gene therapies).

While the biologics market has developed significantly in recent years, the low availability of effective delivery systems (and practicable methods for manufacturing such delivery systems) has limited the diversity of molecular targets of such bio-therapeutics, especially when the target is cytosolic. Indeed, the majority of approved peptide therapeutics on the market act by targeting extracellular components, such as membrane receptors or secreted molecules (e.g. present in the interstitial space). For example, humira (the most successful therapeutic monoclonal antibody) targets the extracellularly secreted cytokine TNFα. Insulin acts by binding its cognate receptor present on the cell membrane (the same being true of other hormone peptide therapeutics).

Similar problems exist in the agricultural industry, where protein-based pesticides are typically toxins which must target an extracellular component of a cell of a pest. By way of example, Bacillus thuringiensis toxins are commonly used natural pesticides which must bind membrane receptors to exert their toxic effects.

Methods for cytosolic delivery of biological molecules have been developed for laboratory research, which generally involve delivering the molecules within lipid vehicles which fuse with the plasma membrane of a cell, before emptying their payload into the cytosol. However, such methods find limited use in medicine and veterinary, e.g. due to the non-specific nature in which they deliver molecules to cells.

Bacterial secretion systems have been explored as potential delivery systems, given their natural ability to secrete (or more particularly ‘inject’) molecules into target cells. The most studied of such secretion systems is the Type III secretion system (T3SS), a “protein appendage” found in several Gram-negative bacteria. However, a significant drawback of these systems is that they remain associated with the bacterial membrane at all times, requiring use of actual bacterial cells (comprising the secretion system) as the delivery system. As such, it is difficult to fully control what molecules are transferred from the bacteria to the target cell (even when the biologic of interest is overexpressed), as these secretion systems function by providing a connection (e.g. channel) between the bacteria's cytosol and the target cell's cytosol, through which other components (potentially harmful to the host) may flow.

Therefore, there exists not only a need for improved delivery systems, but also means for producing such systems which find compatibility with molecules (payloads) having a range of sizes and molecular properties.

The present invention solves one or more of the above-mentioned problems.

The present invention is predicated on the surprising finding that toxigenic Photorhabdus Virulence Cassettes (PVC) effector proteins of Photorhabdus bacteria comprise a previously unknown “leader sequence” (or “leader peptide”), which functions to package (or “load”) PVC effectors into a so called PVC Needle Complex (e.g. “nanosyringe”), which subsequently delivers the PVC effector to a target cell where it exerts its toxigenic effect(s) (the PVC effectors representing a payload of such nanosyringes). Moreover, the inventors have found that such leader sequences can be practically utilized to direct a payload linked thereto to be packaged into a PVC Needle Complex (and related/homologous complexes), a well characterized molecular delivery system of Photorhabdus. Thus, the newly discovered leader sequence surprisingly functions to load the PVC Needle Complex with a molecular payload (or “warhead”).

Further to this finding, the inventors have developed an advantageous, practical utility for such leader sequence for packaging/loading ‘heterologous’ payloads (including non-Photorhabdus molecules) into PVC Needle Complexes, independent of the size, molecular properties or provenance of the heterologous payload.

In a first aspect the invention provides use of a Photorhabdus Virulence Cassettes (PVC) effector leader sequence, for packaging a payload into a PVC Needle Complex; wherein the payload is one or more selected from a polypeptide, a nucleic acid, or a combination thereof (preferably a polypeptide); and wherein the leader sequence and the payload form an effector fusion that is distinct from a wild-type PVC effector protein.

In one aspect, an aspect of the invention provides use of a PVC effector leader sequence, for packaging a payload into a PVC Needle Complex;
wherein the payload is one or more selected from a polypeptide, a nucleic acid, or a combination thereof (preferably a polypeptide); and
wherein the leader sequence and the payload form a fusion that is distinct from a PVC effector protein (e.g. wild-type PVC effector protein).

In other words, the invention provides in one aspect a method for packaging a payload into a PVC Needle Complex with a PVC effector leader sequence, comprising contacting an (effector) fusion with a PVC Needle Complex, wherein the payload is one or more selected from a polypeptide, a nucleic acid, or a combination thereof (preferably a polypeptide); and wherein the leader sequence and the payload form the (effector) fusion, that is distinct from a PVC effector protein (e.g. wild-type PVC effector protein).

The terms “fusion” and “effector fusion”, in the context of a (effector) fusion formed by the leader sequence and the payload (and that is distinct from a wild-type PVC effector protein) are used interchangeably herein.

This use (of the leader sequence) was demonstrated, as outlined in the examples, by expressing an effector fusion (tagged with a detection label) and a PVC Needle Complex in a cell (e.g. host bacterial cell) wherein the effector fusion is packaged into the PVC Needle Complex (via the leader sequence), isolating the PVC Needle Complex, then detecting the presence or absence of the payload within the PVC Needle Complex (e.g. a disrupted version thereof) via Western blot detection of the detection label. The presence of the payload is detected when fused to a leader sequence only, but not when the payload lacks a leader sequence.

The term “PVC effector leader sequence” means the leader region (polypeptide region) from a PVC effector polypeptide which is capable of packaging a payload (e.g. effector) into a PVC Needle Complex, and is preferably amino acids 1-50 of a PVC effector, or amino acids 2-50 when omitting the initial methionine. The inventors have demonstrated that the leader sequence is encompassed within (or may consist essentially of) amino acids 1-50 of a multitude of identified PVC effector polypeptide sequences. However, leader sequences having alternative lengths and positioning within a PVC effector are intended to be encompassed (e.g. with the proviso that said leader sequence is capable of packaging a payload into a PVC Needle Complex).

The remaining (non-leader sequence) portion of a PVC effector is referred to an “effector portion” (e.g. payload) herein. The effector portion preferably comprises or consists essentially of amino acids 51-C terminus of a PVC effector protein.

Thus, in one embodiment, a PVC effector leader sequence is encompassed within amino acids 1-50 or 2-50 (preferably 1-50) of a PVC effector polypeptide.

In embodiment, a PVC effector leader sequence comprises (or consists essentially of) amino acids 1-50 or 2-50 (preferably 1-50) of a PVC effector polypeptide.

The term “wild-type PVC effector protein” is used synonymously with the term “endogenous PVC effector protein”, or simply “PVC effector protein”, and refers to an (e.g. intact) PVC effector sequence having an endogenous leader sequence (i.e. endogenous to the given PVC effector, preferably amino acids 1-50 of the PVC effector) associated with the effector portion (e.g. the payload, preferably amino acids 51-C terminus of a PVC effector protein). Examples of wild-type PVC effectors may comprise (or consist essentially of) an amino acid sequence of one or more sequence selected from SEQ ID NO.: 1-SEQ ID NO.: 46. The fusion/effector fusion of the invention described herein is thus distinct from a PVC effector protein (e.g. wild-type PVC effector protein), as the leader sequence is not fused to an effector portion with which it may be fused in the case of a wild-type PVC effector protein. By way of example, the fusion/effector fusion may comprise the leader sequence of the “Pnf” PVC effector protein (e.g. the leader of SEQ ID NO.: 78) fused to the effector portion of the hvnA (gene Plu1649) PVC effector protein (e.g. amino acids 51-295 of SEQ ID NO.: 46), but is not intended to refer to the leader sequence of the “Pnf” PVC effector protein (e.g. the leader of SEQ ID NO.: 78) fused to the effector portion of the Pnf PVC effector protein (e.g. amino acids 51-340 of SEQ ID NO.: 32).

On the other hand, the fusion/effector fusion may comprise the leader sequence of, e.g., the “Pnf” PVC effector protein (e.g. the leader of SEQ ID NO.: 78) fused to a non-effector portion, for example a non-Photorhabdus protein such as Cre recombinase. Thus, the leader sequence finds utility in packaging a range of e.g. heterologous (non-wild-type) agents into a PVC Needle Complex, opening the possibility to use the PVC Needle Complex as a modular, diverse delivery system for delivering not only natural effectors, but also ‘unnatural’ payloads to a cell for the first time. As such, it is possible to manufacture a PVC Needle Complex having a payload of choice.

Another aspect of the invention provides a method for manufacturing a PVC Needle Complex comprising a payload (e.g. in other words, a method for manufacturing a packaged PVC Needle Complex), the method comprising:
a. contacting (e.g. within a host cell) a PVC Needle Complex with an effector fusion comprising a PVC effector leader sequence fused to a payload;
b. wherein the payload is one or more selected from a polypeptide, a nucleic acid or a combination thereof (preferably a polypeptide); and
c. wherein the effector fusion is distinct from a wild-type PVC effector protein.

An aspect of the invention provides a method for manufacturing a PVC Needle Complex comprising a payload (e.g. in other words, a method for manufacturing a packaged PVC Needle Complex), the method comprising:
a. contacting (e.g. within a host cell) a PVC Needle Complex with a fusion, the fusion comprising a PVC effector leader sequence fused to a payload, wherein the leader sequence and the payload form a fusion that is distinct from a PVC effector protein (e.g. wild-type PVC effector protein); and
b. wherein the payload is one or more selected from a polypeptide, a nucleic acid or a combination thereof (preferably a polypeptide).

In one embodiment, said contacting may occur within a cell (e.g. bacterial host cell), in a cell lysate, or in a purified cell lysate (preferably within a cell). In one embodiment, said contacting may occur within a cell free expression system. Similar, a use described herein may comprise a contacting step (between the fusion/effector fusion and PVC Needle Complex) occurring within a cell (e.g. bacterial host cell), in a cell lysate, cell free expression system, or in a purified cell lysate (preferably within a cell, more preferably a bacterial host cell).

A cassette (operon) encoding the PVC Needle Complex may be operably linked to a first promoter, and a gene encoding the fusion/effector fusion (payload) may be operably linked to a second (preferably different) promoter. In one embodiment, said first and/or second promoter is an inducible promoter (e.g. an arabinose inducible promoter such a pBAD, and/or an IPTG inducible promoter). Thus, the invention embraces an expression system wherein an operon encoding the PVC is present within a first vector/plasmid (optionally operably linked to a first promoter), and the sequence encoding the effector fusion (leader sequence fused to payload) is present within a second (preferably different) plasmid (optionally linked to a second promoter).

In one embodiment, the PVC Needle Complex and/or (preferably and) effector fusion may be expressed in one or more host selected from a bacterial cell, a yeast cell, an insect cell and/or a mammalian cell. In a preferable embodiment, the PVC Needle Complex and effector fusion may be expressed together in a host cell selected from a bacterial cell, a yeast cell, an insect cell and a mammalian cell (preferably a bacterial cell). Suitable mammalian cells include a HEK293 cell and/or a CHO cell.

The PVC Needle Complex and/or (preferably and) the effector fusion (payload) may be expressed in a heterologous bacterial expression system (preferably E. coli). In one embodiment, the PVC Needle Complex and/or (preferably and) the PVC effector may be expressed in a Photorhabdus cell, optionally wherein the PVC operon of the Photorhabdus cell is endogenous to the cell (and optionally wherein the PVC operon is operably linked to an inducible promoter which may be incorporated into the genome to be operably linked to the PVC operon via genetic engineering). For example, an inducible promoter may be introduced into the genome of a Photorhabdus cell 5′ to a PVC (operon), preferably by recombineering as described in the examples (e.g. Example 3).

The payload may be, for example, a therapeutic payload, such that a PVC Needle Complex finds utility in medical treatment.

In a further aspect, the invention provides a (packaged) PVC Needle Complex, for use in a method of treatment;
a. wherein the PVC Needle Complex comprises (e.g. is packaged with) an effector fusion which comprises (or consists essentially of) a PVC effector leader sequence fused to a payload;
b. wherein said payload is one or more selected from a polypeptide, a nucleic acid or a combination thereof (preferably a polypeptide); and
c. wherein the effector fusion is distinct from a wild-type PVC effector protein.

A further aspect of the invention provides a (packaged) PVC Needle Complex, for use in a method of treatment;
a. wherein the PVC Needle Complex holds (e.g. is packaged with) a fusion which comprises (or consists essentially of) a PVC effector leader sequence fused to a payload;
b. wherein said payload is one or more selected from a polypeptide, a nucleic acid or a combination thereof (preferably a polypeptide); and
c. wherein the fusion is distinct from a PVC effector protein (e.g. wild-type PVC effector protein).

In one aspect, the invention provides a method of treating a subject, the method comprising administering a (packaged) PVC Needle Complex to a subject (e.g. a patient);
a. wherein the PVC Needle Complex comprises (e.g. is packaged with) an effector fusion which comprises (or consists essentially of) a PVC effector leader sequence fused to a payload;
b. wherein said payload is one or more selected from a polypeptide, a nucleic acid or a combination thereof (preferably a polypeptide); and
c. wherein the effector fusion is distinct from a wild-type PVC effector protein.

In other words, an aspect of the invention provides a method of treating a subject, the method comprising administering a (packaged) PVC Needle Complex to a subject (e.g. a patient);
a. wherein the PVC Needle Complex holds (e.g. is packaged with) a fusion which comprises (or consists essentially of) a PVC effector leader sequence fused to a payload;
b. wherein said payload is one or more selected from a polypeptide, a nucleic acid or a combination thereof (preferably a polypeptide); and
c. wherein the fusion is distinct from a PVC effector protein (e.g. wild-type PVC effector protein).

In a preferable embodiment, the payload is a polypeptide.

The subject may be a mammalian subject, preferably a human subject.

The terms “PVC Needle Complex holds an effector fusion” and “PVC Needle Complex comprising an effector fusion” means a PVC Needle Complex having a packaged effector fusion, or in other words, a PVC Needle Complex that is packaged with an effector fusion.

The term “packaged effector fusion”, “fusion” and “effector fusion” (e.g. wherein the fusion/effector fusion is distinct from a wild-type PVC effector protein) embraces a combination of a PVC effector leader sequence and a payload which remains in contact (e.g. fused) subsequent to packaging into PVC Needle Complex (e.g. the leader sequence has not been cleaved off the payload), as well as combination of a PVC effector leader sequence and a payload which are no longer in direct contact (e.g. no longer fused, such as following cleavage of the leader sequence from the payload).

The term “treat” or “treating” as used herein encompasses prophylactic treatment (e.g. to prevent onset of a disease) as well as corrective treatment (treatment of a subject already suffering from a disease). Preferably “treat” or “treating” as used herein means corrective treatment. The term “treat” or “treating” encompasses treating both the disease and a symptom thereof. In some embodiments “treat” or “treating” refers to a symptom of a disease.

Therefore, a PVC Needle Complex may be administered to a subject in a therapeutically effective amount or a prophylactically effective amount.

A “therapeutically effective amount” is any amount of the (packaged/laden) PVC Needle Complex, which when administered alone or in combination (e.g. with another therapeutic, administered parallel or in series and acting additively or synergistically) to a subject for treating a disease (or a symptom thereof) is sufficient to effect such treatment of the disease, or symptom thereof.

A “prophylactically effective amount” is any amount of the (packaged/laden) PVC Needle Complex that, when administered alone or in combination (e.g. with another therapeutic, administered parallel or in series and acting additively or synergistically) to a subject inhibits or delays the onset or reoccurrence of a disease (or a symptom thereof). In some embodiments, the prophylactically effective amount prevents the onset or reoccurrence of a disease entirely. “Inhibiting” the onset means either lessening the likelihood of disease onset (or symptom thereof), or preventing the onset entirely.

In a related aspect, there is provided a (packaged) PVC Needle Complex comprising (e.g. that holds/that is packaged with) an effector fusion;
a. wherein said effector fusion comprises (or consists essentially of) a PVC effector leader sequence fused to a payload (or in other words, wherein said effector fusion is formed by a PVC effector leader sequence and a payload);
b. wherein said payload is one or more selected from a polypeptide, a nucleic acid or a combination thereof; and
c. wherein the effector fusion is distinct from a wild-type PVC effector protein.

In other words, one aspect of the invention provides a (packaged) PVC Needle Complex that holds (e.g. is packaged with) a fusion;
a. wherein said fusion comprises (or consists essentially of) a PVC effector leader sequence fused to a payload (or in other words, wherein said fusion is formed by a PVC effector leader sequence and a payload);
b. wherein said payload is one or more selected from a polypeptide, a nucleic acid or a combination thereof (preferably a polypeptide); and
c. wherein the fusion is distinct from a PVC effector protein (e.g. wild-type PVC effector protein).

In a preferable embodiment, the (packaged) PVC Needle Complex is an isolated (e.g. non-natural) PVC Needle Complex.

As explained below, the PVC Needle Complex typically functions in nature to deliver toxigenic PVC effectors to insect targets. By expanding greatly the number and variety of payloads which may be packaged into a PVC Needle Complex, the invention concomitantly expands the number and variety of invertebrates (e.g. pests), such as amoeba, nematodes, helminths and insects, which may be targeted and killed.

In a further aspect of the invention, there is provided a method for suppressing a pest, the method comprising:
a. contacting a pest, or a target area comprising a pest, with a (packaged) PVC Needle Complex comprising (e.g. holding/packaged with) an effector fusion;
b. wherein the effector fusion comprises (or consists essentially of) a PVC effector leader sequence fused to a payload (or in other words, wherein said effector fusion is formed by a PVC effector leader sequence and a payload);
c. wherein said payload is one or more selected from a polypeptide, a nucleic acid or a combination thereof (preferably a polypeptide); and
d. wherein the effector fusion is distinct from a wild-type PVC effector protein.

An aspect of the invention provides a method for suppressing a pest, the method comprising:
a. contacting a pest, or a target area comprising a pest, with a (packaged) PVC Needle Complex holding (e.g. packaged with) a fusion;
b. wherein the fusion comprises (or consists essentially of) a PVC effector leader sequence fused to a payload (or in other words, wherein said fusion is formed by a PVC effector leader sequence and a payload);
c. wherein said payload is one or more selected from a polypeptide, a nucleic acid or a combination thereof (preferably a polypeptide); and
d. wherein the fusion is distinct from a PVC effector protein (e.g. wild-type PVC effector protein).

The terms “PVC Needle Complex holds an effector fusion” and “PVC Needle Complex comprising an effector fusion” means a PVC Needle Complex having a packaged effector fusion.

The term “target area” refers to an area where a pest is present and/or where a pest may be (e.g. is expected to be, or suspected of being) present.

Thus, in one embodiment, a target area may be contacted before, and/or when a pest is present. The target area may be in the vicinity of (e.g. close proximity to) a pest.

Alternatively, the target area may be an area that a user wishes to protect from a pest. For example, a target area may comprise a plant and/or plant product.

The term “suppressing a pest” embraces “pest control”, “inhibiting the growth of a pest”, “inhibiting the proliferation of pest”, and/or “mortality of a pest”.

Examples of such pest include one or more insect(s), mite(s), sowbug(s), pillbug(s), centipede(s), mollusk(s), millipede(s), protist(s), fungus (fungi), helminth(s) and/or bloodborne parasite(s). The pest may be at any stage of development e.g. may be a larvae and/or adult pest (e.g. imago).

The invention may be used to target a variety of agricultural, commercial, home and garden pests.

In one embodiment the pest is an insect, a mite, a sowbug, a pillbug, a centipede, a mollusk and/or a millipede. Suitably the pest may be an insect and/or a mite (preferably insect).

Examples of suitable insects include, an insect of the order Lepidoptera, Coleoptera, Diptera, Blattodea, Hymenoptera, Isoptera, Orthoptera, Thysanura, and/or Dermaptera. In one embodiment an insect of the order Lepidoptera may be one or more of a moth and/or a butterfly. Suitable moths include Manduca Sexta and/or Galleria mel/one/Ia.

In one embodiment an insect of the order Coleoptera may be one or more of a European chafer grub, a northern masked chafer grub, a southern masked chafer grub, a Japanese beetle grub, a June beetle grub, a black vine weevil, a strawberry root weevil, a clay-colored weevil, a Colorado potato beetle, and/or a wireworm. In another embodiment an insect of the order Diptera may be one or more of a leatherjacket (e.g. larvae of a crane fly), an onion maggot, a cabbage maggot, a carrot rust fly maggot, a fungus gnat, and/or a mosquito. In another embodiment an insect of the order Blattodea may be a cockroach, suitably one or more cockroach selected from an American cockroach, and/or a German cockroach.

In one embodiment an insect of the order Hymenoptera may be an ant. Suitably, the ant may be one or more of a carpenter ant, an odorous house ant, a pavement ant, an Argentine ant, a Pharaoh ant, a tawny crazy ant, a harvester ant, a red imported fire ant, a Southern fire ant, a European fire ant, and/or a little fire ant. In another embodiment an insect of the order Hymenoptera may be a yellowjacket.

In one embodiment an insect of the order Isoptera may be a termite. Suitably the termite may be one or more of a damp wood termite, a dry wood termite, and/or a subterranean termite. In another embodiment an insect of the order Orthoptera may be one or more of a cricket, a grasshopper, and/or a locust. In one embodiment an insect of the order Thysanura may be a silverfish. In another embodiment an insect of the order Dermaptera may be an earwig.

Examples of suitable molluscs include a slug and/or a snail.

In one embodiment, the pest is a protist. In one embodiment, said protist is one or more selected from Chaos carolinense, Amoeba proteus, Naegleria fowleri, Dictyostelium discoideum, Entamoeba histolytica, Trichomonas vaginalis, Blastocystis hominis, Leishmania Spp., and Giardia lamblia. In one embodiment, said protist is one or more selected from Fonticula alba, Dictyostelium discoideum, Chlamydomonas reinhardtii, Crytomonas paramedium, Paulinella chromatophora, Nannochloropsis gaditana, and/or Tetrahymena Spp.

In one embodiment, the pest is a fungus. In one embodiment, said fungus is one or more fungus selected from Encephalitozoan cuniculi, Nasema apis, Namema ceranae, Vittaforma carneae, Enterocytosoan bieneusi, Spraguea lophii, Vavra culiculis, Edharzardia aedes, Nematocida parisii, Razella Spp., Parasitella parasitica, Lichteimia ramose, Sporisorium scitamineum, Trametes versicolor, and/or Punctularia strigosozonata.

In one embodiment, said fungus is a Candida spp. Said Candida spp. may be one or more selected from C. albicans, C. ascalaphidarum, C. amphixiae, C. Antarctica, C. argentea, C. atlantica, C. atmosphaerica, C. auris, C. blattae, C. bromeliacearum, C. carpophila, C. carvajalis, C. cerambycidarum, C. chauliodes, C. corydalis, C. dosseyi, C. dubliniensis, C. ergatensis, C. fructus, C. glabrata, C. fermentati, C. guilliermondii, C. haemulonii, C. humilis, C. insectamens, C. insectorum, C. intermedia, C. jeffresii, C. kefyr, C. keroseneae, C. krusei, C. lusitaniae, C. lyxosophila, C. maltose, C. marina, C. membranifaciens, C. mogii, C. oleophila, C. oregonensis, C. parapsilosis, C. quercitrusa, C. rugose, C. sake, C. shehatea, C. temnochilae, C. tenuis, C. theae, C. tolerans, C. tropicalis, C. tsuchiyae, C. sinolaborantium, C. sojae, C. subhashii, C. viswanathii, C. utilis, C. ubatubensis, and/or C. zemplinina. Suitably, said Candida spp. may be C. albicans.

In another embodiment, the pest is a helminth. Said helminth may be one or more selected from the phyla Annelida, Platyhelminthes, Nematoda and/or Acanthocephala. In one embodiment, said helminth is a parasitic flatworm. Said parasitic flatworm may be one or more selected from a Cestoda, a Trematoda and/or a Monogenea. In one embodiment, said helminth is a parasitic nematode. Said parasitic nematode may be one or more selected an ascarid (Ascaris), a filaria, a hookworm, a pinworm (Enterobius), and/or a whipworm (Trichuris trichiura).

In one embodiment, the pest is a bloodborne parasite. Said bloodborne parasite may be one or more selected from Trypanosoma Spp (e.g. Trypanosoma brucei and/or T. cruzi), Babesia Spp (e.g. Babesia microti), Leishmania Spp, Plasmodium Spp (e.g. P. falciparum), and/or Toxoplasma Spp. (e.g. Toxoplasma gondit).

The PVC Needle Complex for pest control is suitably environmentally safe (e.g. an environmentally safe pesticidal composition).

Other advantageous utilities include delivering a payload to a cell, for example, during laboratory research. Such cell may be part of an in vitro cell line, or may be a cell of an animal (e.g. a research animal model). Additionally or alternatively, the cell may be comprised within an ex vivo system, such as an organoid.

Another aspect of the invention provides an in vitro (and/or ex vivo) method for delivering a payload into a cell, the method comprising:
a. contacting a cell with a (packaged) PVC Needle Complex comprising (e.g. holding/packaged with) an effector fusion;
b. wherein the effector fusion comprises (or consists essentially of) a PVC effector leader sequence fused to a payload (or in other words, wherein said effector fusion is formed by a PVC effector leader sequence and a payload);
c. wherein said payload is one or more selected from a polypeptide, a nucleic acid or a combination thereof (preferably a polypeptide); and
d. wherein the effector fusion is distinct from a wild-type PVC effector protein.

An aspect of the invention provides an in vitro (and/or ex vivo) method for delivering a payload into a cell, the method comprising:
a. contacting a cell with a (packaged) PVC Needle Complex holding (e.g. packaged with) a fusion;
b. wherein the fusion comprises (or consists essentially of) a PVC effector leader sequence fused to a payload (or in other words, wherein said fusion is formed by a PVC effector leader sequence and a payload);
c. wherein said payload is one or more selected from a polypeptide, a nucleic acid or a combination thereof (preferably a polypeptide); and
d. wherein the fusion is distinct from a PVC effector protein (e.g. wild-type PVC effector protein).

In one aspect, the invention provides an effector fusion comprising (or consisting essentially of) a PVC effector leader sequence fused to a payload (or in other words, an effector fusion formed by a PVC effector leader sequence and a payload);
a. wherein said payload is one or more selected from a polypeptide, a nucleic acid or a combination thereof; and
b. wherein the effector fusion is distinct from a wild-type PVC effector protein.

An aspect of the invention provides a fusion comprising (or consisting essentially of) a PVC effector leader sequence fused to a payload (or in other words, a fusion formed by a PVC effector leader sequence and a payload);
a. wherein said payload is one or more selected from a polypeptide, a nucleic acid or a combination thereof (preferably a polypeptide); and
b. wherein the fusion is distinct from a PVC effector protein (e.g. wild-type PVC effector protein).

In one embodiment, the fusion/effector fusion is an isolated fusion/effector fusion (e.g. an isolated, non-naturally occurring fusion/effector fusion).

The present invention embraces a nucleic acid comprising a nucleotide sequence which encodes the fusion/effector fusion, and/or an expression vector comprising said nucleic acid.

Also embraced is a host cell comprising said nucleic acid and/or expression vector.

As discussed above, the present inventors have discovered and practically utilised the leader sequence(s) described herein for the first time.

Thus, another aspect of the invention provides an isolated PVC effector leader sequence (e.g. wherein the isolated PVC effector leader sequence is capable of packaging a payload into a PVC Needle Complex).

In a related aspect there is provided an isolated nucleic acid comprising a nucleotide sequence which encodes a PVC effector leader sequence.

The isolated PVC effector leader sequence may be recombinant, synthetic, and/or purified.

The isolated nucleic encoding a PVC effector leader sequence may be recombinant, synthetic, and/or purified.

Further details on the background of the invention, and terminology used herein, is provided below.

Photorhabdus is a bacterium of the genus Enterobacteriacae, represented by three formally recognized (to date) species—namely P. luminescens, P. asymbiotica, and P. temperata. Important strains include P. asymbiotica subsp. australis, and P. luminescens subsp laumondii. Currently available genome sequences are available on GenBank (Photorhabdus asymbiotica ATCC43949 complete genome—GenBank Accession Number: FM162591.1; Photorhabdus laumondii subsp. laumondii strain TT01 chromosome, complete genome—GenBank Accession number: CP024901.1).

Reference to “Photorhabdus luminescens subsp. laumondii” may be used interchangeably with “Photorhabdus luminescens subsp. laumondii TT01”, “Photorhabdus laumondii subsp. laumondiistrain TT01” and “P. luminescens TT01” herein.

The genome sequence for a further strain of P. asymbiotica, namely P. asymbiotica Kingscliff, is described in Wilkinson et. al. (FEMS Microbiology Letters, Volume 309, Issue 2, August 2010, Pages 136-143), incorporated herein by reference. Further genome sequences are described in Thanwisai et. al. (PLoS ONE 7(9): e43835), incorporated herein by reference.

Each of these species comprise at least one operon known as a Photorhabdus Virulence Cassette (PVC) operon, encoding a PVC Needle Complex, which may be referred to as a “nanosyringe” herein. Given that Photorhabdus is typically found in nature as an insecticidal bacterium following regurgitation from a (symbiont) entomopathogenic Heterorhabditis sp. nematode (e.g. in order to avoid competition for food and resources from insects), it is understood that the PVC Needle Complex functions in nature to suppress insects. Indeed, it has been shown that an isolated PVC Needle Complex (holding/packaged with a natural effector toxin, such as Pnf) can be used to kill insect larvae—see Example 2. The Photorhabdus Virulence Cassettes represent one of at least four well-characterised toxin delivery systems of Photorhabdus. Other major classes of Photorhabdus protein insecticidal toxins include the “Toxin Complexes” (Tcs), the “binary PirAB toxins”, and the “makes caterpillars floppy” (Mcf) toxins.

The term “Photorhabdus Virulence Cassette” (PVC) (used synonymously with the term “PVC operon” herein) means a discrete operon of a Photorhabdus genome comprising genes encoding for polypeptide subunits which, when expressed, assemble to provide the macromolecular PVC Needle Complex. The molecular architecture of these cassettes have been well characterized and described, for example in The Molecular Biology of Photorhabdus Bacteria (Springer International Publishing AG 2017, ISBN: 978-3-319-52714-7, Chapter 10, pages 159-177), incorporated herein by reference. A PVC (operon) typically comprises around sixteen genes (pvc1-pvc16) encoding structural proteins which assemble to provide a “PVC Needle Complex”, which are typically followed by one or more genes at the 3′ end which encode PVC effector genes, having toxic activity (and typically being homologues of typical T3SS-like effectors). A Photorhabdus genome typically comprises a plurality of such cassettes (e.g. at least four), which are often associated with different effector payloads, or even a plurality of effector payloads.

Three classes of PVC structural operons (Classes I, II and III) have been observed in the genomes of Photorhabdus, and members of other genera. PVCs within each class are similar in terms of the number and type of genes encoding structural proteins they contain (see FIG. 1(B)). In more detail, Class I PVCs (which may be referred to as a “prototypical PVC” herein) comprise 16 conserved genes (pvc1-16). Class II lack pvc13 host cell binding fibres and pvc3, which (without wishing to be bound by theory) the inventors believe may be a minor specialised sheath subunit that attaches pvc13 fibre proteins onto the PVC Needle Complex (nanosyringe). As such, it is believed this class may be “non-specific”, injecting payloads into multiple (potential any) cell types. Class III is similar to Class I, but has an additional Pvc0 gene at the start of the operon (of unknown function) and two additional genes encoded between pvc13 and pvc14 that resemble “invasion” type protein genes. This class is typically seen in the human clinical isolate strains of Photorhabdus—the inventors have shown that optimal transcription of PVC Class III may occur when the strain (harboring the PVC operon encoding a PVC Class III operon) is grown at 37° C. and exposed to human serum, suggesting this class may be a mammalian adapted version of a PVC Needle Complex.

An example cassette (PVC) is shown in FIG. 1(D), which shows a map of the model “Class I” PVC operon of Photorhabdus asymbiotica ATCC43949 (obtainable from the ATCC, accession number: ATCC 43949), said operon being associated with the downstream effector gene “PAU_03332” (encoding a Pnf protein effector, e.g. SEQ ID NO.: 32). This model operon is referred to as Pa^{ATCC43949} PVCpnf. This operon comprises sixteen structural genes (pvc1-16), and two genes (3′ end) encoding effectors (in this case the pvc17/Rhs-like, encoding an Rhs-like effector, and pvc21, encoding a Pnf effector). Said genes pvc1-16 correspond to genes PAU_03353 to PAU_03338 of the sequence of GenBank accession no. FM162591.1, and are represented by the sequence of SEQ ID NO.: 93.

An example PVC operon (e.g. encoding the structural genes, but not a/the PVC effector) is provided in SEQ ID NO: 93 (which is encodes the operon shown schematically in FIG. 1(D)), with other examples being SEQ ID NO: 94 and in SEQ ID NO: 95. These sequences begin at the ATG start codon of the first structural gene (pvc1) of the PVC cassette/operon, and end at the TAA stop codon of the final structural gene (pvc16).

A PVC Needle Complex from any one of Classes I-III may be used for a variety of applications. However, PVC Needle Complexes of a certain class may be particularly suitable for delivery to a defined cell type. For example, a PVC Needle Complex for delivery of a payload to a mammalian cell may suitably be a member of Class III. A PVC Needle Complex for delivery of a payload to an insect cell (e.g. to an insect) may suitably be a member of Class I (such as P. asymbiotica PVCpnf, encoded by SEQ ID NO.: 93, e.g. as expressed in E. coli from a cosmid clone).

Thus, as will be understood by the skilled person, the term “PVC Needle Complex” (used synonymously with the terms “PVC Needle Complex delivery system” and “nanosyringe” herein) means a macromolecular protein complex comprising polypeptide subunits encoded by a PVC (operon) of a Photorhabdus bacterium. A PVC Needle Complex is assembled in a nanosyringe structure, having a physical structure (superficially) similar to the antibacterial R-type pyocins (see FIG. 3). Functional and molecular studies have shown that a PVC Needle Complex becomes packaged (loaded) with a PVC effector protein(s) (i.e. the PVC effector proteins are packaged therein, or thereon), the packaged PVC Needle Complex is released from the bacterium, and then injects the PVC effector into a target cell such that the PVC effector protein may exert toxicity.

The term “PVC Needle Complex” preferably encompasses PVC Needle Complex-like structures/complexes, encoded by operon(s) comprising genes which are homologous to genes of a Photorhabdus PVC operon. PVC-like elements are not restricted to Photorhabdus, and a well characterized homologous operon (to a PVC operon) is present on the pADAP plasmid of the insect pathogenic bacteria Serratia entomophila. Furthermore, an analogous, and (at least partially) homologous, PVC-like ‘injectosome’ Needle Complex system is employed by the bacterium Pseudoalteromonas luteoviolacea (e.g. used to control the metamorphosis of the marine worm Hydroides elegans). Structures exist in other Enterobacteriaceae (such as Yersinia Spp.) which are encoded by operons having homology to a PVC operon, and may be used with a leader sequence described herein. Each of these (PVC-like) structures are embraced by the term “PVC Needle Complex” as used herein.

Thus, a PVC Needle Complex is a “nanosyringe” complex, with the polypeptide encoded by the effector gene being packaged (loaded) within, or at the end (tip) of, the PVC Needle Complex, thus representing a “payload” or “warhead” of the PVC Needle Complex. The present inventors have demonstrated that the PVC Needle Complex itself (with the payload still loaded) is freely released (e.g. secreted) from Photorhabdus cells, before interacting with the membrane of a target cell and injecting the payload into the cell's cytosol. Indeed, the inventors have successfully expressed and loaded PVC Needle Complexes in heterologous expression systems, before isolating/purifying the PVC Needle Complexes and using them to suppress (e.g. kill) insect larvae (see Example 2). Thus, the PVC Needle Complexes act as long-range protein delivery systems.

In one embodiment, the PVC Needle Complex is encoded by a sequence having at least 75% sequence identity (preferably at least 85% sequence identify; more preferably at least 95% sequence identity) to a sequence selected from SEQ ID NO.: 93, SEQ ID NO.: 94, and SEQ ID NO.: 95 (for example, SEQ ID NO.: 93).

In one embodiment, the PVC Needle Complex is encoded by a sequence selected from SEQ ID NO.: 93, SEQ ID NO.: 94, and SEQ ID NO.: 95 (for example, SEQ ID NO.: 93).

Leader/signal sequences are typically peptides, often of 10-30 amino acids long present at the N-terminus of the majority of (newly) expressed proteins that are destined towards the secretory pathway (e.g. for directing said proteins to a protein-conducting channel on the cell membrane). Many proteins require a signal sequence for Golgi or endoplasmic reticulum entry.

The term “leader sequence” (used interchangeably with the terms “leader peptide”, “signal sequence”, “targeting signal”, “localization signal”, “localization sequence”, and “transit peptide” herein), used in the context of a “PVC effector leader sequence” herein, means a polypeptide sequence which functions to direct the PVC effector into the interior, or the end (tip), of a PVC Needle Complex—as such, the leader sequence functions to package a PVC effector into a PVC Needle Complex. The PVC Needle Complex can subsequently deliver (e.g. inject) the PVC effector into a target cell. The PVC Needle Complex may be an assembled PVC Needle Complex. The term “PVC Needle Complex” may refer to a fragment of a PVC Needle Complex (e.g. wherein the leader sequence contacts said fragment, and optionally the PVC Needle Complex assembles around the leader sequence-payload ‘effector fusion’).

A PVC leader sequence is typically present in the N-terminus (characterized by or encompassed within the first 50 amino acids) of a PVC effector or homologue thereof. However, the invention embraces leader sequences of PVC effectors and PVC effector homologues, which may be found in regions other than the N-terminal region of such PVC effectors/homologues (e.g. in the C-terminal region).

In one embodiment, the leader sequence comprises (or consists essentially of) amino acid residues 1-50 of a PVC effector (e.g. PVC effector protein). Reference to “amino acid residues 1-50” embraces “amino acid residues 2-50”, wherein the N-terminal methionine is omitted e.g. has been cleaved. The leader sequence may be a fragment of the N-terminal 50 amino acids of a PVC effector (e.g. a fragment comprising or consisting essentially of ≤45, ≤35, ≤25, or ≤15 amino acids), with the proviso that the fragment is capable of packaging a payload into a PVC Needle Complex.

In one embodiment, a leader sequence (e.g. isolated leader sequence) of the invention comprises (or consists essentially of) an amino acid sequence having at least 50%, 55%, 60%, 65%, 70%, 75%, 80%, 85%, 90%, 95% or 100% sequence identity to one or more sequence selected from SEQ ID NO.: 47-SEQ ID NO.: 92 (preferably SEQ ID NO.: 50, SEQ ID NO.: 68, SEQ ID NO.: 71, SEQ ID NO.: 76, SEQ ID NO.: 78, or SEQ ID NO.: 92)—e.g. with the proviso that the leader sequence is capable of packaging a payload into a PVC Needle Complex. In a preferable embodiment, a leader sequence comprises (or consists essentially of) an amino acid sequence having at least 60% sequence identity to one or more sequence selected from SEQ ID NO.: 47-SEQ ID NO.: 92 (preferably SEQ ID NO.: 50, SEQ ID NO.: 68, SEQ ID NO.: 71, SEQ ID NO.: 76, SEQ ID NO.: 78, or SEQ ID NO.: 92)—e.g. with the proviso that the leader sequence is capable of packaging a payload into a PVC Needle Complex. In a more preferable embodiment, a leader sequence comprises (or consists essentially of) an amino acid sequence of one or more selected from SEQ ID NO.: 47-SEQ ID NO.: 92 (preferably SEQ ID NO.: 50, SEQ ID NO.: 68, SEQ ID NO.: 71, SEQ ID NO.: 76, SEQ ID NO.: 78, or SEQ ID NO.: 92). In one embodiment, a leader sequence comprises (or consists essentially of) an amino acid sequence selected from SEQ ID NO.: 47-SEQ ID NO.: 92 (preferably SEQ ID NO.: 50, SEQ ID NO.: 68, SEQ ID NO.: 71, SEQ ID NO.: 76, SEQ ID NO.: 78, or SEQ ID NO.: 92).

In one embodiment, a leader sequence comprises (or consists essentially of) an amino acid sequence selected from SEQ ID NO.: 50, SEQ ID NO.: 68, SEQ ID NO.: 71, SEQ ID NO.: 76, SEQ ID NO.: 78, and SEQ ID NO.: 92.

In one embodiment, the leader sequence comprises (or consists essentially of) an amino acid sequence of SEQ ID NO.: 50. In one embodiment, the leader sequence comprises (or consists essentially of) an amino acid sequence of SEQ ID NO.: 68. In one embodiment, the leader sequence comprises (or consists essentially of) an amino acid sequence of SEQ ID NO.: 71. In one embodiment, the leader sequence comprises (or consists essentially of) an amino acid sequence of SEQ ID NO.: 76. In one embodiment, the leader sequence comprises (or consists essentially of) an amino acid sequence of SEQ ID NO.: 78. In one embodiment, the leader sequence comprises (or consists essentially of) an amino acid sequence of SEQ ID NO.: 92.

Without wishing to be bound by theory, it is believed that the leader sequences share a “chemical composition consensus”, based on amino acid properties. More particularly, the leader sequences comprise similar charge patterns, the pattern comprising 2× negatively charged regions, each followed by a positively charged region (e.g. [−ve] [+ve] [−ve] [+ve])—see FIG. 9. This is consistent with leader sequences of toxins of the type 2 secretion system, which comprise a charge/property pattern of [+ve] [Hydrophobic] [+ve] [C]. A further theory posits that the leader sequences share a typical “helix-turn-helix” structure. Another theory is that the leader sequences form a structure recognised by an ATPase enzyme (e.g. encoded by the gene PAU_03339 (pvc15) in the model operon of FIG. 1(D)) present in the interior, or at the end (e.g. tip), of a PVC Needle Complex.

The term “PVC effector” (used synonymously with the term “PVC operon-encoded effector”, and “PVC effector protein”) means an effector polypeptide encoded by a Photorhabdus PVC operon, more particularly (and typically) found shortly downstream (3′) of the structural genes of said operon (preferably shortly or immediately downstream of pvc16, and typically within 5 kb). The term “PVC effector” preferably embraces homologues thereof. Thus, the leader sequence may also be from a polypeptide encoded by a gene which is a homologue of gene encoding a PVC effector—see Table 1 for examples of such homologues. Indeed, identification of PVC effectors is aided by detecting homology of a gene downstream of pvc16 with a known toxin polypeptide (e.g. a gene which encodes said toxin polypeptide). As will be understood by the skilled person, the term “homologue” preferably means a gene that descended from the same ancestral gene, and shares similar function—such gene (or polypeptide encoded thereby) is homologous to a gene encoding the PVC effector. A homologue may be from the genome of a Photorhabdus species or from a species other than a Photorhabdus species. Examples of suitable homologues are outlined in Table 1.

The present inventors have elucidated and characterised, in detail, genes that encode PVC effectors of these PVC Needle Complexes in the three most common (best characterised) strains of Photorhabdus, as well as the P. asymbiotica Thai strain PB68.1. This was conducted based on analysing proximity of genetic linkage to the 3′ end of the PVC structural genes of the operons, and predicted function of the protein sequence of the effector (e.g. a homologue of a known effector/toxin protein). In more detail, the PVC effectors (e.g. genes encoding the PVC effectors) were typically identified as open reading frames (ORF) having homology to genes encoding known toxin polypeptides (e.g. homologues as outlined in Table 1), and being typically present within a distance of 1 kilobase to 5 kilobase (kb) (e.g. within 1 kb) downstream of the final structural gene of a PVC operon (e.g. pvc16) (typically with few or no intervening genes). Typically, there are no “non-toxin-like” ORFs between the end of the operon (encoding the PVC Needle Complex) and the PVC effector gene(s). Although there may be (e.g. one, or two) other small predicted genes present in these regions, these other genes are not assigned as PVC effectors (due to lack of homology to a known effector/toxin gene, as described above).

In order to assign a putative PVC effector gene (e.g. ORF within a distance of 5 kb, for example within 1 kb downstream of the final structural gene of a PVC operon) as a PVC effector gene, the inventors used a combination of BlastP and HHPRED (https://toolkit.tuebingen.mpg.de/#/tools/hhpred). Putative PVC effector genes were assigned as PVC effector genes based on direct homology to known toxin encoding genes, similarity to a toxin protein family, proximity to the PVC operon (e.g. within 1-5 kb downstream of the final structural gene of a PVC operon, pvc16) and/or based on domain similarities of predicted secondary structures to that of known toxins.

Thus, a PVC effector (gene) may be identified (within a Photorhabdus genome) by (i) identifying pvc16 (e.g. via sequence homology to a known pvc16), (ii) identifying an ORF 3′ to pvc16, preferably 55 kb downstream of pvc16), and (iii) confirming said ORF encodes a PVC effector through identification of sequence homology to a known gene encoding a toxin polypeptide (for example, a toxin protein described in the column of Table 1 labelled “Homologue(s)”).

By way of example, the PVC effector gene PAU_03337 (referred to herein as “sepC” due to homology to virulent sep genes) is positioned 325 base pairs (bp) downstream of pvc16 (PAU_03338) of the PVC operon referred to herein as PVCpnf (e.g. of SEQ ID NO. 93), which is found in P. asymbiotica ATCC43949. That is, the start codon of PAU_03337 begins 325 bp downstream of the end of the stop codon of PAU_03338.

This can be illustrated by reference to the P. asymbiotica ATCC43949 complete genome, accessible via GenBank accession no. FM162591.1 (see also e.g. Wilkinson et al, BMC Genomics volume 10, article number: 302 (2009), incorporated herein by reference), in which effector gene PAU_03337 is annotated as being positioned in the genome as follows: complement (3913237 . . . 3914247)—that is, at nucleotide positions 3913237 . . . 3914247; and PAU_03338 is annotated as being positioned in the genome as follows: complement (3914573 . . . 3915454). No other ORF (encoding an effector or otherwise) is found between these two genes.

A further PVC effector gene associated with the PVC operon referred to herein as PVCpnf (e.g. of SEQ ID NO. 93), namely PAU_03332 (referred to herein as “pnf”), is positioned 3535 bp downstream of pvc16 (PAU_03338).

The PVC effector gene PAU_02095 (referred to herein as “Rhs-like toxin effector” due to homology to virulent Rhs toxin genes) is positioned 3961 bp downstream of pvc16 (PAU_02099) of a PVC operon referred to herein as PVC/opT (e.g. of SEQ ID NO. 94), which is found in P. asymbiotica ATCC43949. That is, the start codon of PAU_02095 begins 3961 bp downstream of the end of the stop codon of PAU_02099.

In a further example, the PVC effector of gene PAU_02009 (referred to as “cif” herein due to predicted function as a cell cycle inhibiting factor/ATP/GTP binding protein) is positioned 157 bp downstream of pvc16 (PAU_02008) of the associated PVC operon, referred to herein as PVCcif, found in P. asymbiotica ATCC43949.

In yet further examples: with regard to a PVC operon of P. luminescens TT01 referred to as a PVCunit4 operon herein, PVC effector gene “pvc17” (e.g. “p/u1651”) is positioned 104 bp downstream of pvc16 (gene “plu1655′); and with regard to a PVC operon of Photorhabdus temperata subsp. temperata Meg1 referred to as a PVCcif operon herein, PVC effector gene “CIF toxin effector” (e.g. MEG1DRAFT_03529) is positioned 4216 bp downstream of the relevant pvc16 gene.

These examples, illustrate that a gene encoding a PVC effector is typically positioned within a distance of ≤5 kb downstream of the final gene of a PVC operon (e.g. of pvc16), more typically within a distance of ≤1 kb downstream of the final gene of a PVC operon.

In summary, there exists 46 PVC effectors that have been identified in these four strains (based on currently available sequence data) (see Table 1). The first 50 amino acids of each of these PVC effectors represent (or encompass) their endogenous leader sequence, and the inventors have demonstrated the leader sequences may be cloned and fused to a variety of payloads to be packaged into a PVC Needle Complex—see Examples 3 and 4. Thus, a PVC effector (as translated) comprises at least two principle domains: the leader sequence (amino acids 1 to 50) and the actual effector polypeptide (amino acids 51 to C-terminal amino acid)—the latter of which may be referred to as the “effector” (e.g. “effector portion”) or “payload” herein.

Although the Photorhabdus genome sequence(s) continues to be revised, this consolidated list of PVC effector genes represents a comprehensive description of such effectors, and is based on currently available sequence data of the most common (best characterised) Photorhabdus strains, and provides the skilled person with an understanding of the term “PVC effector” as well as the sequences of these PVC effectors (as well as how to search/mine for further PVC effectors, e.g. in alternative (genome) sequences). As described above, the inventors have found that the PVC effector proteins comprise a leader sequence which is necessary (and sufficient) for directing the PVC effector protein (e.g. payload) to be packaged/loaded into a PVC Needle Complex.

TABLE 1

| | | Accession No. | | |
|---|---|---|---|---|
| Gene | | (polypeptide | | SEQ ID |
| (Locus Tag) | Predicted Function | sequence) | Homologue(s) | NO.: |
| PAK_1985 | Cell cycle inhibiting | WP_036768136 | Cif type III | 1 |
| | factor/ATP/GTP binding protein. | | effector | |
| PAK_1987 | Cytidine deaminase | WP_036768135 | YwqJ family | 2 |
| | toxin-like. | | | |
| PAK_1988 | RHS repeat toxin like/Cholera | WP_036768134 | RHS repeat- | 3 |
| | enterotoxin (A chain) ADP | | protein | |
| | Ribosyltransferase like | | | |
| PAK_2075 | CNF; cytotoxic necrotizing factor | WP_036768069 | CNF1 family | 4 |
| | (zinc metallo-peptidase)/Neurotoxin | (exemplary, not | | |
| | A; botulinum | 100% identical) | | |
| PAK_2077 | LopT cysteine proteinase | WP_036768068 | YopT type III | 5 |
| | (peptidase C58 family). | | effector from | |
| | | | Yersinia. | |
| PAK_2892 | Pvc17; putative nematode | WP_065822933 | n/a | 6 |
| | symbiosis protein | (exemplary, not | | |
| | | 100% identical) | | |
| PAK_2893 | Similar to Type III secretion | WP_065822917 | GogB type III | 7 |
| | protein GogB | (exemplary, not | effector from | |
| | | 100% identical) | Salmonella. | |
| PAK_2894 | RHS-repeat protein. Function | WP_036774164 | RHS-repeat | 8 |
| | unknown. | | protein. | |
| PAK_3525 | CNF/PaTox domain like tyrosine | WP_036768627.1 | PaTox from | 9 |
| | glycosylase UDP-GlcNAc | (exemplary, not | Photorhabdus | |
| | | 100% identical) | asymbiotica | |
| | | | ATCC43949 | |
| PAT_00148 | Cif-like; Cell cycle inhibiting | WP_065823029 | Cif type III | 10 |
| | factor/ATP/GTP binding protein. | | effector | |
| PAT_00149 | YwqJ -Cytidine deaminase | WP_065823017.1 | YwqJ family | 11 |
| | toxin-like. | | | |
| PAT_00150 | RHS repeat toxin like/Cholera | WP_065823018.1 | RHS repeat | 12 |
| | enterotoxin (A chain) ADP | | protein | |
| | Ribosyltransferase like | | | |
| PAT_00152 | Domain similar to Colicin A and | WP_065823019 | n/a | 13 |
| | Glucosly Transferase | | | |
| PAT_02308 | Pvc17; putative nematode | WP_065822933 | n/a | 14 |
| | symbiosis protein | | | |
| PAT_02309 | Similar to Type III secretion | WP_065822917 | GogB type III | 15 |
| | protein GogB | | effector from | |
| | | | Salmonella. | |
| PAT_02310 | RHS-repeat protein. Function | WP_065822916 | RHS repeat | 16 |
| | unknown. | | protein | |
| PAT_02956 | cytotoxic necrotizing factor (zinc | WP_065822174 | CNF1 family | 17 |
| | metallopeptidase)/Neurotoxin | | | |
| | A; botulinum | | | |
| PAT_02957 | LopT cysteine proteinase | WP_065822175 | YopT type III | 18 |
| | (peptidase C58 family). Similar | | effector from | |
| | to YopT type III toxin of Yersinia. | | Yersinia. | |
| PAT_03171 | YwqJ-Cytidine deaminase | WP_065823264 | YwqJ family | 19 |
| | toxin-like. | | | |
| PAT_03172 | Cytotoxic necrotizing factor 1, | WP_065823265 | CNF1 family | 20 |
| | Rho deamidase | | | |
| PAT_03177 | Calmodulin-sensitive adenylate | WP_065823268 | CyaA family | 21 |
| | cyclase | | | |
| PAU_02009 | Cif-like; Cell cycle inhibiting | CAQ84101 | Cif type III | 22 |
| | factor/ATP/GTP binding | | effector | |
| | protein. | | | |
| PAU_02010 | TccC3/RHS repeat toxin like. | CAQ84102 | RHS repeat | 23 |
| | | | protein | |
| PAU_02095 | TccC2/RHS repeat toxin like. | CAQ84187 | RHS repeat | 24 |
| | Neutral metalloproteinase II. | | protein | |
| PAU_02096 | LopT cysteine proteinase | CAQ84188 | YopT type III | 25 |
| | (peptidase C58 family). Similar | | effector from | |
| | to YopT type III toxin of Yersinia. | | Yersinia. | |
| PAU_02097 | RHS-repeat protein. Function | CAQ84189 | RHS repeat | 26 |
| | unknown. | | protein | |
| PAU_02098 | Dermonecrotic toxin; Pasteurella | CAQ84190 | RtxA family | 27 |
| | multocida toxin-like/RtxA-like | | from Vibrio | |
| | (Glucosyltransferase) | | | |
| PAU_2230 | PaTox; tyrosine glycosylase and | CAQ84322 | Type III | 28 |
| | cysteine protease domains. | | effector Ssel | |
| | | | from | |
| | | | Salmonella | |
| PAU_02805 | Pvc17; putative nematode | CAQ84177 | n/a | 29 |
| | symbiosis protein | | | |
| PAU_02806 | Similar to Type III secretion | CAQ84895 | GogB type III | 30 |
| | protein GogB | | effector from | |
| | | | Salmonella. | |
| PAU_02807 | Similar to Type III secretion | CAQ84179 | GogB type III | 31 |
| | protein GogB | | effector from | |
| | | | Salmonella. | |
| PAU_03332 | Pnf; Rho GTPase deaminidase | CAQ85420 | CNF1 family | 32 |
| | and tranglutination. | | from E. coli | |
| PAU_03337 | CyaA-like; Adenylylcyclase toxin | CAQ85425 | Antrax Edema | 33 |
| | (anthrax EF-like). | | Factor, | |
| | | | Pseudomonas | |
| | | | ExoY toxin | |
| Plu1651 | Pvc17. Putative nematode | WP_011145938 | n/a | 34 |
| | symbiosis protein. Induces | | | |
| | endotokia matricida in C. | | | |
| | elegans (experimental) | | | |
| Plu1671 | RHS repeat toxin like. ADP- | WP_011145957 | RHS repeat | 35 |
| | ribosyltransferase | | protein | |
| Plu1672 | RHS repeat toxin like. ADP- | WP_011145958 | RHS repeat | 36 |
| | ribosyltransferase | | protein | |
| Plu1690/PLU_RS08490 | RHS repeat toxin like. Rho | WP_011145974 | RHS repeat | 37 |
| | deamidase | | protein | |
| Plu1691 | RHS repeat toxin like. ADP- | WP_011145975 | RHS repeat | 38 |
| | ribosyltransferase | | protein | |
| Plu1712 | CyaA calmodulin-sensitive | WP_011145994 | CyaA family | 39 |
| | adenylate cyclase like. | | | |
| Plu1713 | Weak similarity to Diphtheria | WP_011145995 | n/a | 40 |
| | toxin catalytic domain. | | | |
| Plu1714 | RHS repeat toxin like. Neutral | WP_041380028 | RHS repeat | 41 |
| | metallo-proteinase II. | | protein | |
| Plu2400 | Dermonecrotic toxin-like; | WP_011146635 | RtxA family | 42 |
| | Pasteurella multocida toxin-like/RtxA- | | from Vibrio | |
| | like Glucosyltransferase. | | | |
| Plu2401 | LopT cysteine proteinase | WP_011146636 | YopT type III | 43 |
| | (peptidase C58 family). Similar | | effector from | |
| | to YopT type III toxin of Yersinia. | | Yersinia. | |
| Plu2514 | Similar to Xenorhabdus Mcf- | WP_011146737 | n/a | 44 |
| | domain/weak acid phosphatase. | | | |
| Plu2515 | Cif-like; Cell cycle inhibiting | WP_011146738 | Cif type III | 45 |
| | factor/ATP/GTP binding protein. | | effector | |
| Plu1649 | N-acyl homoserine lactonase like | WP_133148775 | Weak | 46 |
| | | | similarity to | |
| | | | HvnA of Vibrio | |

The accession numbers provided in Table 1 are provided for exemplary purposes, providing example amino acid sequences of (or having high similarity to) PVC effectors described herein. The sequences of said accession numbers may be accessed through GenBank (https://www.ncbi.nlm.nih.gov/genbank/).

The locus tag (beginning with “PAU” or “Plu”) corresponds to the locus tag assigned to the effector in genome sequences available through GenBank above. Locus tags beginning with “PAT” (referring to strain P. asymbiotica Thai strain P1B68.1) and “PAK” (referring to strain P. asymbiotica Kingscliff) have been assigned by the present inventors upon identification of the PVC effector genes within the genomes of said strains (in a consistent manner with the locus tags of publicly available sequences).

This locus tags may be used herein to refer to the corresponding PVC effector polypeptide.

In one embodiment, the PVC effector is encoded by one or more gene (with the SEQ ID NO. of the encoded PVC effector protein in parentheses) selected from PAK_1985 (SEQ ID NO: 1), PAK_1987 (SEQ ID NO: 2), PAK_1988 (SEQ ID NO: 3), PAK_2075 (SEQ ID NO: 4), PAK_2077 (SEQ ID NO: 5), PAK_2892 (SEQ ID NO: 6), PAK_2893 (SEQ ID NO: 7), PAK_2894 (SEQ ID NO: 8), PAK_3525 (SEQ ID NO: 9), PAT_00148 (SEQ ID NO: 10), PAT_00149 (SEQ ID NO: 11), PAT_00150 (SEQ ID NO: 12), PAT_00152 (SEQ ID NO: 13), PAT_02308 (SEQ ID NO: 14), PAT_02309 (SEQ ID NO: 15), PAT_02310 (SEQ ID NO: 16), PAT_02956 (SEQ ID NO: 17), PAT_02957 (SEQ ID NO: 18), PAT_03171 (SEQ ID NO: 19), PAT_03172 (SEQ ID NO: 20), PAT_03177 (SEQ ID NO: 21), PAU_02009 (SEQ ID NO: 22), PAU_02010 (SEQ ID NO: 23), PAU_02095 (SEQ ID NO: 24), PAU_02096 (SEQ ID NO: 25), PAU_02097 (SEQ ID NO: 26), PAU_02098 (SEQ ID NO: 27), PAU_02230 (SEQ ID NO: 28), PAU_02805 (SEQ ID NO: 29), PAU_02806 (SEQ ID NO: 30), PAU_02807 (SEQ ID NO: 31), PAU_03332 (SEQ ID NO: 32), PAU_03337 (SEQ ID NO: 33), Plu1651 (SEQ ID NO: 34), Plu1671 (SEQ ID NO: 35), Plu1672 (SEQ ID NO: 36), Plu1690 (SEQ ID NO: 37), Plu1691 (SEQ ID NO: 38), Plu1712 (SEQ ID NO: 39), Plu1713 (SEQ ID NO: 40), Plu1714 (SEQ ID NO: 41), Plu2400 (SEQ ID NO: 42), Plu2401 (SEQ ID NO: 43), Plu2514 (SEQ ID NO: 44), Plu2515 (SEQ ID NO: 45), Plu1649 (SEQ ID NO: 46), or a combination thereof.

In one embodiment, the PVC effector is encoded by one or more gene (with the SEQ ID NO. of the encoded PVC effector protein in parentheses) selected from PAU_02009 (SEQ ID NO: 22), PAU_02010 (SEQ ID NO: 23), PAU_02095 (SEQ ID NO: 24), PAU_02096 (SEQ ID NO: 25), PAU_02097 (SEQ ID NO: 26), PAU_02098 (SEQ ID NO: 27), PAU_02230 (SEQ ID NO: 28), PAU_02805 (SEQ ID NO: 29), PAU_02806 (SEQ ID NO: 30), PAU_02807 (SEQ ID NO: 31), PAU_03332 (SEQ ID NO: 32), PAU_03337 (SEQ ID NO: 33), Plu1651 (SEQ ID NO: 34), Plu1671 (SEQ ID NO: 35), Plu1672 (SEQ ID NO: 36), Plu1690 (SEQ ID NO: 37), Plu1691 (SEQ ID NO: 38), Plu1712 (SEQ ID NO: 39), Plu1713 (SEQ ID NO: 40), Plu1714 (SEQ ID NO: 41), Plu2400 (SEQ ID NO: 42), Plu2401 (SEQ ID NO: 43), Plu2514 (SEQ ID NO: 44), Plu2515 (SEQ ID NO: 45), Plu1649 (SEQ ID NO: 46), or a combination thereof. These gene names correspond to the ‘locus tags’ of PVC effector genes in the Photorhabdus genome sequences accessible via GenBank, as described above. The PAT and PAK locus tags were generated by the present inventors, such that terminology is consistent with the PAU and Plu locus tags of publicly available genome sequences.

Thus, the PVC effector may be encoded by one or more gene listed above.

In one embodiment, the PVC effector is encoded by one or more gene (with the SEQ ID NO. of the encoded PVC effector in parentheses) selected from PAK_02075 (SEQ ID NO: 4), PAU_02009 (SEQ ID NO: 22), PAU_02096 (SEQ ID NO: 25), PAU_02806 (SEQ ID NO: 30), PAU_03332 (SEQ ID NO: 32), Plu1651 (SEQ ID NO: 34), Plu1649 (SEQ ID NO: 46), or a combination thereof.

In a preferable embodiment, the PVC effector is encoded by one or more gene (with the SEQ ID NO. of the encoded PVC effector in parentheses) selected from PAU_02806 (SEQ ID NO: 30), PAU_03332 (SEQ ID NO: 32), Plu1651 (SEQ ID NO: 34), Plu1649 (SEQ ID NO: 46), or a combination thereof.

The PVC effector may have a sequence having at least 80% sequence identity (preferably at least 90% sequence identity; more preferably 100% sequence identity) to an amino acid sequence selected from SEQ ID NO: 1-SEQ ID NO: 46. For example, the PVC effector may have a sequence having at least 80% sequence identity (preferably at least 90% sequence identity; more preferably 100% sequence identity) to an amino acid sequence selected from SEQ ID NO: 22-SEQ ID NO: 46.

The present inventors have identified the leader sequences of the gogB1 (PAU_02806) and Pnf (PAU_03332) PVC effectors as being particularly efficient at packaging a (fused) payload into a PVC Needle Complex. In one embodiment, the PVC effector is encoded by PAU_02806 (e.g. has an amino acid sequence of SEQ ID NO: 30). In one embodiment, the PVC effector is encoded by PAU_03332 (e.g. has an amino acid sequence of SEQ ID NO: 32).

In one embodiment, the PVC effector comprises (or consists essentially of) an amino acid sequence of one or more selected from SEQ ID NO: 1-SEQ ID NO: 46 (for example SEQ ID NO: 22-SEQ ID NO: 46), or a combination thereof. For example, the PVC effector may comprise (or consist essentially of) a sequence selected from SEQ ID NO: 4, SEQ ID NO: 22, SEQ ID NO: 25, SEQ ID NO: 30, SEQ ID NO: 32 and SEQ ID NO: 46.

In one embodiment, the PVC effector comprises (or consists essentially of) an amino acid sequence of SEQ ID NO.: 4. In one embodiment, the PVC effector comprises (or consists essentially of) an amino acid sequence of SEQ ID NO. 22. In one embodiment, the PVC effector comprises (or consists essentially of) an amino acid sequence of SEQ ID NO. 25. In one embodiment, the PVC effector comprises (or consists essentially of) an amino acid sequence of SEQ ID NO: 30. In one embodiment, the PVC effector comprises (or consists essentially of) an amino acid sequence of SEQ ID NO: 32. In one embodiment, the PVC effector comprises (or consists essentially of) an amino acid sequence of SEQ ID NO. 46.

The term “packaging” (used synonymously with the terms “trans-packaging” and “loading”) means the directing of a payload, by a leader sequence of the invention (to which the payload is linked/fused), into the interior, or end (tip), of an assembled PVC Needle Complex, such that the PVC Needle Complex is subsequently configured for delivering (e.g. injecting) the payload into a target cell. Thus, the payload may be packaged within a PVC Needle Complex, or may be packaged at the end (or tip) of the PVC Needle Complex (e.g. at least a portion of the payload may be external to the PVC Needle Complex).

The term “payload” (used synonymously with the term “warhead” herein) means a molecule which is packaged into the interior, or end (tip), of an assembled PVC Needle Complex, and subsequently delivered (e.g. injected) into a (target) cell. In wild-type Photorhabdus, the payload is a PVC effector (more particularly, the effector portion of said PVC effector), encoded (as described above) by a gene that is downstream to (3′ to) the structural genes of a PVC operon. For example, see model PVC operon of FIG. 1(D), having effector genes PAU_03337 (listed as PVCpnf 17), encoding an adenylate cyclase effector (e.g. SEQ ID NO.: 33); and PAU_03332 (listed as PVCpnf 21), encoding a Pnf effector (e.g. SEQ ID NO.: 32).

A leader sequence and a payload of the present invention form an “effector fusion” (or simply “fusion”) that is “distinct from a (e.g. wild-type) PVC effector” (e.g. a polypeptide encoded by one of the genes outlined in Table 1). For example, the effector fusion may be a chimaera, formed of a leader sequence from a first PVC effector fused to (an/the effector portion of) a second (different) PVC effector (preferably amino acids 51 to the C-terminal amino acid of said second PVC effector), wherein said first PVC effector and said second PVC effector are different. The effector fusion may be a chimaera, comprising (or consisting essentially of) a leader sequence described herein fused to a non-PVC effector polypeptide. The effector fusion may be a chimaera, comprising (or consisting essentially of) a leader sequence described herein fused to a non-Photorhabdus polypeptide. The effector fusion may be a leader sequence-nucleic acid fusion (preferably conjugate), comprising a leader sequence described herein fused to a nucleic acid.

An effector fusion is not limited to a fusion complex comprising a leader sequence fused to a toxic payload (e.g. the leader could be fused to a therapeutic payload). Thus, the term “effector” as used in the context of “effector fusion” means the payload which is packaged into the PVC Needle Complex (which could provide a variety of effects, including toxigenic and/or therapeutic effects). Thus, the term “effector fusion” may be used interchangeably with the term “fusion” herein.

The term “effector fusion” may be used synonymously with the term “leader sequence-payload fusion”, and/or “leader sequence-payload complex”.

Alternatively or additionally, the payload may be distinct from a PVC effector protein (e.g. distinct from amino acids 51 to the C-terminal amino acid of a PVC effector). For example, the payload may be a polypeptide or nucleic acid that is not found in a wild-type Photorhabdus bacterium.

Analysis of the size (e.g. polypeptide length) and structure of the various natural PVC effector payloads encoded by Photorhabdus, shows that there exists a wide variety of different PVC effector lengths and structures, demonstrating that the applicability of the PVC Needle Complex delivery system of the present invention is not limited by the size or properties of the payload of interest. To summarise, there is no requirement for particular secondary structure, biophysical property, or length of cargoes, confirming that that the PVC Needle Complex can be utilised as a versatile multifunctional delivery vehicle.

The payload may be one or more selected from a polypeptide (e.g. a polypeptide payload), a nucleic acid (e.g. a nucleic acid payload), or a combination thereof. In a preferable embodiment, the payload is a polypeptide.

Examples of polypeptide payloads include an antibody (e.g. an anti-MDM antibody), a nanobody, a peptide vaccine (e.g. a tyrosinase-related protein 2 (TRP2) peptide vaccine), a nuclear factor-KB inhibitor, a T3SS payload (e.g. a T3SS payload which inhibits the NF-kB and/or MAPK pathways), an anti-apoptotic peptide (e.g. BH4), nicotinamide adenine dinucleotide quinone internal oxidoreductase (Ndi1), a PHOX complex subunit, a myotubularin, a nucleic acid (preferably DNA)-modifying enzyme, or a combination thereof.

Examples of suitable nucleic acid-modifying enzymes include a recombinase (e.g. Cre recombinase), a transposase, a Cas enzyme (e.g. Cas9), and/or a Mad7 (preferably Mad7, more preferably Cre recombinase). The payload may be, for example, tBid (SEQ ID NO.: 109) and/or BaxBH3 peptide (aa59-73) (SEQ ID NO.: 111).

Any polypeptide having enzymatic activity may be a payload.

A nucleic acid payload may be conjugated/crosslinked to a leader sequence of the invention. For example, copper-free click chemistry (e.g. strain-promoted alkyne azide cycloaddition (SPAAC)) may be used to crosslink a nucleic acid to a leader sequence. Examples of nucleic acid payloads include a primer, an mRNA, a nucleic acid analogue, an aptamer, a small interfering RNA (siRNA), a microRNA therapeutic inhibitor (antimiR), a microRNA therapeutic mimic (promiR), a long non-coding RNA modulator, a single guide RNA (sgRNA), or a combination thereof.

The leader sequence may be fused directly or indirectly (e.g. by means of a spacer) to the payload. The leader sequence may be fused covalently or non-covalently to the payload. In a preferable embodiment, the leader sequence is covalently fused to the payload. For example, the fusion/effector fusion may be a (recombinant) fusion protein comprising (or consisting essentially of) a PVC effector leader sequence fused to a (polypeptide) payload.

Another aspect of the invention provides an isolated nucleic acid comprising a nucleotide sequence which encodes a PVC effector leader sequence of the invention. Another aspect of the invention provides an isolated nucleic acid comprising a nucleotide sequence which encodes an effector fusion (e.g. fusion) of the invention, and optionally a nucleotide sequence which encodes a PVC Needle Complex.

Another aspect of the invention provides an expression vector comprising: a nucleic acid (preferably an isolated nucleic acid) comprising a nucleotide sequence which encodes a PVC effector leader sequence of the invention. Another aspect of the invention provides an expression vector comprising: a nucleic acid (preferably an isolated nucleic acid) comprising a nucleotide sequence which encodes an effector fusion (e.g. fusion) of the invention, and optionally a nucleotide sequence which encodes a PVC Needle Complex.

Another aspect of the invention provides a host cell comprising an isolated nucleic acid, the isolated nucleic acid comprising a nucleotide sequence which encodes a PVC effector leader sequence of the invention. Another aspect of the invention provides a host cell comprising an isolated nucleic acid, the isolated nucleic acid comprising a nucleotide sequence which encodes an effector fusion (e.g. fusion) of the invention, and optionally a nucleotide sequence which encodes a PVC Needle Complex.

The term “nucleic acid” may be used synonymously with the term “polynucleotide”.

Another aspect of the invention provides a host cell comprising an expression vector, the expression vector comprising a nucleotide sequence which encodes a PVC effector leader sequence of the invention. Another aspect of the invention provides a host cell comprising an expression vector, the expression vector comprising a nucleotide sequence which encodes an effector fusion (e.g. fusion) of the invention, and optionally a nucleotide sequence which encodes a PVC Needle Complex.

Said host cell may be a mammalian cell, an insect cell, a yeast cell, a bacterial cell (e.g. E. coli), or a plant cell. In a preferable embodiment, the host cell is a bacterial cell (preferably E. coli).

In one embodiment, the host cell is a Photorhabdus cell, optionally wherein the Photorhabdus cell comprises a PVC operon operably linked to an inducible promoter (e.g. see Example 3). The PVC operon may be endogenous to the Photorhabdus cell (e.g. the PVC operon may be PVCu4). Suitably, the Photorhabdus cell may be obtainable from the ATCC under accession no. ATCC 29999.

The sequences (e.g. leader sequence and/or nucleic acid sequence) of the present invention include sequences that have been removed from their naturally occurring environment, recombinant or cloned (e.g. DNA) isolates, and chemically synthesized analogues or analogues biologically synthesized by heterologous systems.

The leader sequence(s) and/or polynucleotide(s) of the present invention may be prepared by any means known in the art. For example, large amounts of the leader sequence(s) and/or polynucleotide(s) may be produced by replication and/or expression in a suitable host cell. The natural or synthetic DNA fragments coding for a desired fragment will typically be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured bacterial, insect, mammalian, plant or other eukaryotic cell lines.

The leader sequence(s) and/or polynucleotide(s) of the present invention may also be produced by chemical synthesis, e.g. a polynucleotide by the phosphoramidite method or the tri-ester method, and may be performed on commercial automated oligonucleotide synthesizers. A double-stranded (e.g. DNA) fragment may be obtained from the single stranded product of chemical synthesis either by synthesizing the complementary strand and annealing the strand together under appropriate conditions or by adding the complementary strand using DNA polymerase with an appropriate primer sequence.

When applied to a leader sequence or nucleic acid sequence, the term “isolated” in the context of the present invention denotes that the leader sequence and/or polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | -1 | 5 | | | | | | | | | | | | | | | | | | |
| N | -2 | 0 | 6 | | | | | | | | | | | | | | | | | |
| D | -2 | -2 | 1 | 6 | | | | | | | | | | | | | | | | |
| C | 0 | -3 | -3 | -3 | 9 | | | | | | | | | | | | | | | |
| Q | -1 | 1 | 0 | 0 | -3 | 5 | | | | | | | | | | | | | | |
| E | -1 | 0 | 0 | 2 | -4 | 2 | 5 | | | | | | | | | | | | | |
| G | 0 | -2 | 0 | -1 | -3 | -2 | -2 | 6 | | | | | | | | | | | | |
| H | -2 | 0 | 1 | -1 | -3 | 0 | 0 | -2 | 8 | | | | | | | | | | | |
| I | -1 | -3 | -3 | -3 | -1 | -3 | -3 | -4 | -3 | 4 | | | | | | | | | | |
| L | -1 | -2 | -3 | -4 | -1 | -2 | -3 | -4 | -3 | 2 | 4 | | | | | | | | | |
| K | -1 | 2 | 0 | -1 | -3 | 1 | 1 | -2 | -1 | -3 | -2 | 5 | | | | | | | | |
| M | -1 | -1 | -2 | -3 | -1 | 0 | -2 | -3 | -2 | 1 | 2 | -1 | 5 | | | | | | | |
| F | -2 | -3 | -3 | -3 | -2 | -3 | -3 | -3 | -1 | 0 | 0 | -3 | 0 | 6 | | | | | | |
| P | -1 | -2 | -2 | -1 | -3 | -1 | -1 | -2 | -2 | -3 | -3 | -1 | -2 | -4 | 7 | | | | | |
| S | 1 | -1 | 1 | 0 | -1 | 0 | 0 | 0 | -1 | -2 | -2 | 0 | -1 | -2 | -1 | 4 | | | | |
| T | 0 | -1 | 0 | -1 | -1 | -1 | -1 | -2 | -2 | -1 | -1 | -1 | -1 | -2 | -1 | 1 | 5 | | | |
| W | -3 | -3 | -4 | -4 | -2 | -2 | -3 | -2 | -2 | -3 | -2 | -3 | -1 | 1 | -4 | -3 | -2 | 11 | | |
| Y | -2 | -2 | -2 | -3 | -2 | -1 | -2 | -3 | 2 | -1 | -1 | -2 | -1 | 3 | -3 | -2 | -2 | 2 | 7 | |
| V | 0 | -3 | -3 | -3 | -1 | -2 | -2 | -3 | -3 | 3 | 1 | -2 | 1 | -1 | -2 | -2 | 0 | -3 | -1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine, lysine, histidine
Acidic: glutamic acid, aspartic acid
Polar: glutamine, asparagine
Hydrophobic: leucine, isoleucine, valine
Aromatic: phenylalanine, tryptophan, tyrosine
Small: glycine, alanine, serine, threonine, methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins.

For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an effector” includes a plurality of such effectors and reference to “the effector” includes reference to one or more effectors and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows (A) a schematic representation of one PVC operon layout (gene clusters present in varying regions of the originating genome) encoding a PVC Needle Complex. (B) A schematic representation of Class I, II and III PVC operon layouts. Homologous subunit types amongst the classes are show as having similar shading (in grey scale). (C) An illustration of an assembled PVC Needle Complex. The numbering shown is used to correlate a gene cluster in (A) with the position of the encoded proteins in the structure in (C) (e.g. the cap ‘16’ cluster in A is shown as ‘16’ in the left-most cap region of (B)). (D) A map of the model Class I PaATCC⁴³⁹⁴⁹PVCpnf operon (e.g. encoded by SEQ ID NO.: 93), showing two effector genes in the payload region (Rhs-like adenylate cyclase, and PAU_03332).

FIG. 2 shows an overview of a cloning procedure for preparation of PVC Needle Complex-expressing plasmids, based on overlapping PCR. PCR fragments (having overlapping regions) are provided from template gDNA of P. asymbiotica^{ATCC43}949 (available from the ATCC under accession no. ATCC 43949) with relevant primers targeting the PVC operon.

FIG. 3 shows a transmission electron micrograph of an (in vitro) sample of PVC Needle Complexes (e.g. prepared from cells having the expression vector described above). The PVC Needle Complexes assemble in a distinct ‘nanosyringe’ structure, consistent with its role as a contractile structure. A 3D rendered model of a PVC Needle Complex as derived from high resolution single particle cryo-EM tomography structure is shown in (B).

FIG. 4 shows (A) a transmission electron micrograph of a PVC Needle Complex comprising a Pnf payload following immuno-gold staining with an anti-Pnf (immunogold) antibody, confirming the Pnf-payload toxin is associated with the PVC Needle Complex (referred to as PVCpnf). PVCpnf Needle Complexes were prepared from supernatants of an E. coli cosmid clone, which encodes the PVCpnf operon. Anti-peptide antibodies against the Pnf (TGQKPGNNEWKTGR, SEQ ID NO: 96) epitope were used to localise the payload toxin protein. The Pnf toxin could only be detected at the ends of broken or contracted needle complex, providing evidence that the toxins are contained within the complex (arrows). (B) Western blot analysis confirms that the Pnf protein (toxin) can only be detected using the anti-peptide antibody if the PVC Needle Complex is either chemically or physically disrupted. These preparations were taken from Pa^{ATCC43949} supernatants. The inability to detect Pnf in clarified supernatants confirms all the protein is associated with the PVC Needle Complex enrichment preparations. Lanes 1+5; sonicated samples, 2+6; 1M NaCl treatments, 3+7; 1% SDS treatments 4+8; 1M Urea treatments. Note the PVC Needle Complex appears stable in 1M NaCl.

FIG. 5 shows cryo-SEM image of ex vivo hemocytes (insect macrophage/neutrophil equivalents) from 5th instar Manduca sexta that had been injected with a native (A) or heat inactivated (B) enriched preparation of Pa^{ATCC43949} PVCpnf Needle Complexes (nanosyringes) heterologously produced by an E. coli cosmid clone. Note the abundant linear structures corresponding to PVC Needle Complexes (nanosyringe) (small arrows) and membrane ruffling effect (large arrows), consistent with the mode of action of the Pnf payload toxin, which are absent from the control treatment. Scale bar=50 μm. 25 kV; magnification 40K (A) and 50K (B).

FIG. 6 shows experimental results demonstrating the (toxic) cellular phenotype following contact with a PVC Needle Complex is due to intracellular toxin delivery. (A) A Pnf loaded PVC Needle Complex was injected into insects (Galleria mel/one/Ia insect larvae), showing potent activity within 15 minutes for the given dose (explained in the examples)—note mortality/morbidity is typically associated with the “melanisation” immune response in these dead/dying insects. (B) A control, denatured (via boiling) Pnf loaded PVC Needle Complex injected into animals showed no activity. (C) Purified Pnf (payload), absent the PVC Needle Complex (i.e. Pnf not packaged into the complex), showed no activity against either animals (left) or a HeLa cell line (right). (D) Pnf (payload) delivered into the cytosol of HeLa cells—via ‘BioPorter’ liposomal preparations containing the protein, or by intracellular expression following transfection with an appropriate plasmid (E)—showed potent activity/toxicity, as evidenced by multi-nucleation in the cells. (F)—The effect of PVCpnf+Pnf on the respiration rate of THP1 derived human macrophages as measured by Resazurin plate reader assay. Note the heat denatured and empty PVCpnf nanosyringes showed no strong adverse effect. These same samples were tested by injection into Galleria larvae. The PVCpnf+Pnf samples showed over around 50% mortality within minutes (darkened larvae in the bottom two panels) while the heat denatured and empty PVCpnf injected insects all remained healthy (no darkened larvae in the top two panels).

FIG. 7 shows (in silico) predicted secondary structures of a range of the endogenous payload (toxin) associated with various PVC operons, demonstrating the large variety of structure types. (B) The amino acid length of various payloads (toxins) plotted against predicted isoelectric point.

FIG. 8 shows confirmation that leader sequences (e.g. having 50 amino acids) of the invention are necessary and sufficient for (trans-)packaging payload proteins/peptides into PVC Needle Complexes (nanosyringes) expressed in Photorhabdus. (A) 1-6: Schematic maps of chimeric effector protein expression constructs (trans-expressed in the arabinose-inducible pBAD30 vector), including those expressing Pnf and non-native cre-recombinase and Myc-tags. C-terminal Myc-tag epitopes are shown as black arrows. (B) Western blots using anti-Myc mouse antibody. Samples are from purified PVC(u4) Needle Complexes (nanosyringes) overexpressed from chromosomally engineered P. luminescens TT01 which harbour the trans-packaging expression constructs 1-6 shown in (A). A blank pBAD30 plasmid was used as a negative control and showed no signal. Arrows show correct band sizes for expected products.

FIG. 9 shows an alignment of the leader sequences, demonstrating the presence of a chemical composition consensus amongst the leader sequences, based on amino acid properties. More particularly, the leader sequences comprise similar charge patterns, of 2× negatively charged regions, each followed by a positively charged region [−ve] [+ve] [−ve][+ve].

FIG. 10 shows (A) western blot analysis of PVC Needle Complexes and payloads from particulate preparations (Cesium Chloride gradient and Monolith FPLC preparations, as described in Materials and Methods). In [1] (pBADPVCpnf, in which PVC16 of the nanosyringe is FLAG-tagged providing PVC16::FLAG detectable with AntiFLAG Ab), a signal from the tagged cap protein of “PVCPnf” (PVC Needle Complex with a Pnf payload) can be seen, confirming the presence of PVC Needle Complexes in the purified fraction. In [2](pBADPVCpnf+Cre::Myc, detectable with AntiMyc Ab, the Cre having an N-term fusion of the Pnf leader e.g. SEQ ID NO.: 78), a signal from the Myc-tagged payload protein packaged in abundance, in the same sample as (1), confirming presence of Cre payload in purified PVC Needle Complexes (nanosyringes). In [3] (PVCU4+Cre::Myc, detectable with AntiMyc Ab, the Cre having an N-term fusion of the Pnf leader e.g. SEQ ID NO.: 78), a different PVC Needle Complex chassis (“PVCU4”) purification is probed for Myc-tagged Cre revealing a packaged (packaged Myc-tagged Cre) corresponding band. This is highlighted in the blot for clarity. (B) Transmission electron micrograph of a PVC Needle Complex, shows both wild-type (having a Pnf payload) PVC Needle Complexes and PVC Needle Complexes having an atypical (non-native) recombinase (Cre) payload, in any chassis tested, does not affect morphology of the PVC Needle Complexes, ensuring they are not assembled aberrantly.

FIG. 10 (C) provides additional/complementary data to that of (A). In more detail, (C) provides further proof via Western blot analysis of (trans-)packaging of the Cre recombinase into purified PVCpnf expressed in E. coli. The Western blot demonstrates that for a given amount of Anti-FLAG antibody Western signal (a specific probe for the nanosyringe due the incorporation of PVC16::FLAG), a much higher amount of the Cre payload is detected (using the Anti-Myc tag antibody). The numbers denote 2-fold dilutions. Note, upon dilution, the anti-FLAG signal from the nanosyringe is lost, while the payload remains intense in most lanes. CsCl denotes purification by Caesium Chloride density gradient centrifugation. “Mon” denotes the samples were additionally anion exchanged via “Monolithic” columns. “Post-Elution”, “Interphase”, “Sub-Interph.”, denote the liquid fractions where the signal is detected from the purification process. D—Western blot analysis of Cre trans-packaged into PVCpnf in E. coli. Payloads are probed for their incorporated ‘Myc’ tags (C-terminal fusions) after purification of the nanosyringe-payload complex. Western blot analysis of particle preps confirms that all four leaders could efficiently trans-package the exogenous Cre enzyme. E—A phylogenetic tree, demonstrating the exemplified leader sequences are well distributed throughout and are therefore at or close to maximally sequentially diverse (see Example 4.2).

FIG. 11 shows western blot analysis of PVC Needle Complexes expressed without (1) and with (2) concomitant expression of (Myc-tagged) Pnf from a separate plasmid, probed simultaneously with an anti-FLAG and anti-Myc antibody. In the lanes marked 1, the PVC Needle Complex (nanosyringe) was expressed and purified without the presence of a ‘payload plasmid’ (an expression plasmid encoding a payload protein linked to a leader sequence) within E. coli. This leads to a band corresponding only to the FLAG tag present on the syringe (PVC Needle Complex) itself. For lanes 2, the same approach was undertaken, but using cultures which also included a (separate) plasmid bearing a tagged payload (Myc-Pnf). Bands can be seen which correspond to the FLAG and Myc tags, confirming presence of the Pnf payload (the four lanes within 1 and 2 are simply different purification fractions from Caesium Chloride gradients).

FIG. 12 shows western blot analysis of trans-packing experiments in P. luminescens TT01 PVCu4 over-expression strain. Results demonstrate the trans-packaging of a myc-tagged Pvc17 (Plu1651whole::Myc).

FIG. 13 shows further western blot analysis of trans-packaging experiments in P. luminescens TT01 PVCunit4 over-expression strain (as explained in the Examples). Results demonstrate trans-packing of Myc-tagged Pvc17 (Plu1651::Myc) and a Myc tag alone using the leader of Pnf (PAU_03332 leader), and that the leader is necessary. (A) Lane 1 shows packaging of the leader of fused to a Myc-tag (PAU_03332::Myc); Lane 3 shows a lack of packaging when the leader sequence is absent (Myc only is not packaged); lane 4 shows lack of packaging of HvnA (a natural effector) when the leader sequence is absent; lane 6 shows packaging of Myc-tagged PAU_03332::Plu1649, i.e. a chimaera of the leader from PAU_03332 (i.e. amino acids 1-50 of PAU_03332) and the effector (i.e. amino acids 51-C-terminus) from Plu1649. The high intensity of bands in lanes 1 and 6 demonstrate that the Pnf (PAU_03332) leader is particularly effective at packaging a payload). (B) Lane 1 shows packaging of Plu1651 with a C-terminal Myc tag using an anti-Myc antibody Western blot.

FIG. 14 shows further Western blot analysis demonstrating the very high level of trans-packaging of Myc-tagged Pnf (PAU_03332::Myc) using the PAU_02806 (GogB) leader (second lane, not including the ladder lane). The first lane demonstrates use of the Plu1649 leader for packaging the PAU_03332 effector (Myc-tagged Plu1649::PAU_03332). The band appears weak due to the relative intensity of the band in the second lane. The experiment involved filter sterilisation of 50 mL culture, 8 M final concentration of urea added to break down PVCs. Samples collected from 10 mL supernatant.

FIG. 15 shows further western blot analysis demonstrating trans-packaging of Plu1651 (pvc17) with a C-terminal Myc tag as described in FIG. 13 into PVCunit4 expressed from Photorhabdus. Raw represents particulate preps from supernatants, Be, Be2 and IP represent different “cuts” from a Caesium chloride gradient purification.

FIG. 16 (A) provides a diagrammatic explanation of the mechanism of action of Cre in the mouse organoid experiment (of Example 6), and how the positive control (TAM) facilitates Cre activation. White arrows show the location of cells expressing the tdTom fluorescent reporter gene. B—Demonstration of delivery of active trans-packaged Cre-recombinase into murine bile duct organoids by PVCpnf expressed and purified from E. coli. White circles show the location of groups of cells expressing the fluorescent reporter gene. The upper images show a direct grey scale conversion of an images obtained via light microscopy. The lower image shows a corresponding image with false-colour enhancement of positive cells, which is provided simply to aid identification of the difference between effected cells and surrounding unaffected ones within the former grey scale conversion.

FIG. 17 shows a dot-blot analysis of nanosyringe expression both with a payload (the Cas9-like protein MAD7) and without. Some leaky expression of the IPTG inducible MAD7 is seen before induction (T1) as is common with this expression system. There is no Myc signal from the PVC only sample at any time point as expected, and the MAD7 signal grows throughout the expression over a ˜24 hour period. Strong Myc signal is maintained post purification via ultracentrifugation as described elsewhere, indicating that the protein is incorporated into the nanosyringe chassis system. FLAG signal is robust in the MAD7 sample, and occurs as expected post-induction and persists post-purification, as this promoter system has reduced leaky expression. It is concluded that the nanosyringes and MAD7 are compatible with one another in terms of expression, and that MAD7, the largest protein tested to date, can be packaged in to the nanosyringe system.

FIG. 18 shows western dot-blot analysis confirming trans-packaging of the pro-apoptotic tBid protein domain and BaxBH3 (both having the leader sequence of SEQ ID NO.: 78 fused to the N-term) peptide into purified PVCpnf expressed from E. coli (7 & 8). The nanosyringe with its cognate toxin “Pnf” is shown, as purified by 2 different methods (5 & 6) as a positive control. The blots at the bottom of the panel represent the same examples as in 7 & 8 in the panels above. These blots were made from another purification of the same constructs, demonstrating reproducibility of purification. This experiment demonstrated that “tBid protein domain and BaxBH3 peptide” packed samples (nanosyringes) can be successfully prepared, e.g. for used in the apoptosis delivery assays in Example 9.

FIG. 19 (A) shows TUNEL-stain microscopic analysis from cells exposed to the packaged nanosyringes for 20 minutes only. First (left) bar=DNase I treated cells (+control); Second bar=no DNAse I or nanosyringe treatment (− control); Third bar=cells were exposed to nanosyringes packaged with tBid (via leader sequence of SEQ ID NO.: 78 fused to the N-term); fourth (right) bar=cells were exposed to nanosyringes packaged with Bax_BH3 domain (via leader sequence of SEQ ID NO.: 78 fused to the N-term). B—Representative micrographs as described in Example 9, showing TUNEL staining of PBMC's, following treatment with nanosyringes and controls. PBMCs were treated with tBID, Bax loaded nanosyringes, and the positive (DNase I treated cells) and negative (no DNase I treatment) controls for 20 minutes at room temperature before performing TUNEL staining to determine an apoptotic response. In the original (non-grayscale) micrographs: Cells negative for apoptotic response show blue or light brown staining. Blue staining (Methyl green) or light brown staining indicates healthy cells with absence of apoptotic signal. Dark brown staining indicates cells undergoing apoptosis.

### EXAMPLES

### Materials and Methods

### Cloning

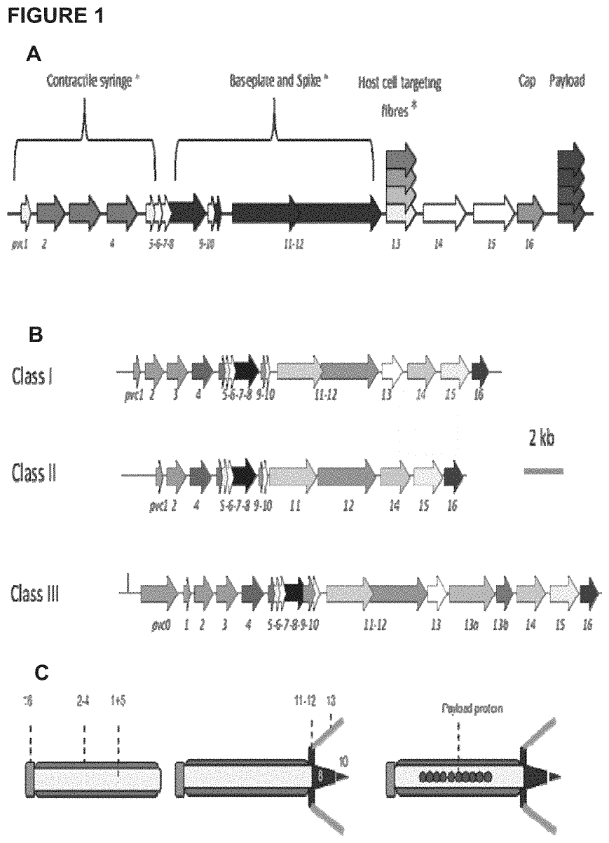

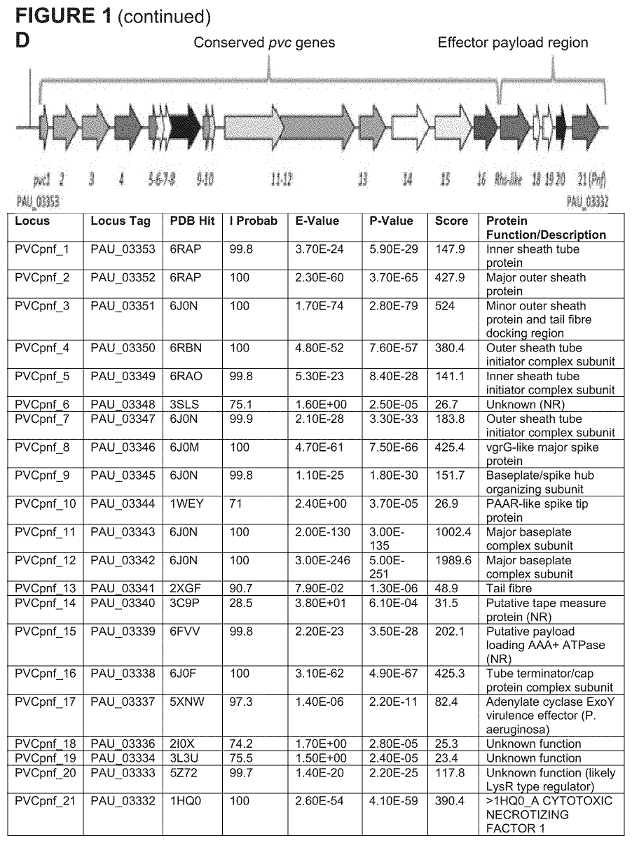

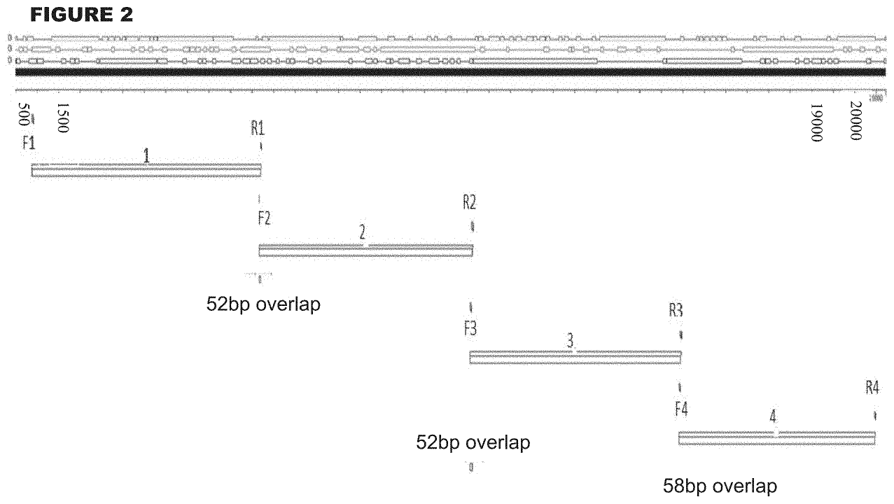

Plasmids encoding PVC Needle Complexes were prepared using standard molecular techniques known in the art. Briefly, genomic DNA from P. asymbiotica^{ATCC43949} (obtainable from the ATCC under accession no. ATCC 43949) was used in PCR (with appropriate primers) to amplify multiple (e.g. four) overlapping regions of the PVC operon. Overlap/extension PCR was employed to prepare a whole operon, and fused (again using overlapping PCR) into an appropriate expression vector as detailed in FIG. 1 (using the primers of SEQ ID NO: 101-SEQ ID NO: 106).

Briefly: four overlapping PVC fragments (generated with primers of SEQ ID NO: 101 (F1) and SEQ ID NO: 105 (R1); SEQ ID NO: 102 (F2) and SEQ ID NO: 106 (R2); SEQ ID NO: 103 (F3) and SEQ ID NO: 107 (R3); and SEQ ID NO: 104 (F4) and SEQ ID NO: 108 (R4), respectively) were made covering the PVC operon (e.g. of SEQ ID NO: 93). The target cloning vector was cut at the required insertion site. These 5 DNA fragments were then assembled by overlapping PCR (using primers of SEQ ID NO: 101 and of SEQ ID NO: 108), and the resulting fragment was ligated into the cloning vector. Products were transformed into laboratory E. coli and recovered with vector marker selection (e.g. due to ampicillin resistance).

The operons are typically operably linked to an inducible promoter (e.g. arabinose inducible, and/or IPTG inducible) as is known in the art. This is generally achieved by cloning into pBAD family plasmids (inducible via arabinose) (Invitrogen, catalog number: V43001) and pVTRa (inducible via IPTG) (Biomedal, S.L.) vectors (although any combination of compatible expression vector systems should suffice).

A PVC Needle Complex can be expressed independently of the payload (toxin), and vice versa. Separate expression vectors (e.g. having differing inducible promoters) may harbour the PVC Needle Complex and the payload, respectively.

Expression (e.g. laboratory scale expression)/Purification of PVC Needle Complexes in E. coli

A typical process to purify a PVC Needle Complex from a 1 L culture of an E. coli expression strain (transformed with an appropriate expression vector/cosmid) is as follows:
1—An overnight culture of the bacteria (transformed with PVC Needle Complex expression vector) is prepared by picking a colony from a plate and inoculating 100 mL of LB media.
   The culture is grown at 37° C. with shaking.
      a. Typically, the media may be routinely supplemented with 0.2% d-Glucose to aid repression of the genetic constructs for optimal cell health.
      b. The media is also supplemented with the relevant antibiotics for maintenance of the expression (PVC Needle Complex) vector. If a payload vector is also being used, the relevant antibiotic for that vector is also supplied.
   2—The next day, a 1 L flask is inoculated via dilution in a 1:100 ratio from the overnight culture. The media for the 1 L flask is identical to the overnight media but typically does not contain glucose.
3—Cultures are grown to approximately mid-to-late exponential (an OD600 nm of ˜0.8) at which point the plasmids are induced.
   a. For the PVC Needle Complex (nanosyringe) plasmid, typically 0.2% arabinose is added to induce expression. For the payload plasmid (plasmid encoding for the payload, such as Pnf), IPTG concentrations may typically be optimised on a per-protein basis, and a typical starting figure of 0.1 mM is preferable.
4—The cultures are returned to the incubator post-induction and cultured at 18° C. until the following day.
5—Cultures are harvested by centrifugation in appropriate centrifuges/bottles/rotors at 5000×g for 30 mins.
6—Cell pellets are then lysed to release PVC Needle Complexes (nanosyringes).
   a. The following lysis methods may be used:
      (i) Lysozyme incubation overnight. (ii) Sonication with a needle sonicator (with or without first treating with lysozyme. (iii) Cell disruptor/homogenisers.
7—Optionally, DNAse, and protease inhibitors can be added to the lysate.
8—Cell debris is removed by centrifugation at 50,000×g, 4° C., for 20 minutes in a high speed centrifuge.
9—Concentrate the lysate through a 100,000 kDa MWCO centrifugation column to reduce volumes and remove small proteins. Once the volume is down to a manageable volume, centrifuge several times replacing the retentate solution with an appropriate sample buffer such as TM (20 mM Tris-HCl, 8 mM MgCl₂, pH 7.4) to dialyse.

A subsequent process for purification via Caesium Chloride density gradient is as follows:
1. Prepare CsCl density solutions as follows:
   (a) 1.7 g/mL CsCl in H₂O; (B) 1.5 g/mL CsCl in H₂O; (C) 1.45 g/mL CsCl in H₂O
2. Gradients (from bottom-to-top of the tube) are then set up in ultracentrifuge tubes like so:
   (1) (bottom of tube)—2 mL density, 1.7 CsCl; (2)—3 ml density, 1.5 CsCl; (3)—3 mL density, 1.45 CsCl; (4) (top of tube)—sample in TM buffer. Suitably, apply each density carefully to side of tube so as not to blend the boundary with the previous density layer.
3. Balanced tubes are then subjected to ultracentrifugation at 35,000 RPM in an SW40Ti swinging bucket rotor, equivalent to 155,000×g, for 2 hours, 4° C.
4. The correct gradient fraction will be the region just above a ‘blue-ish-white’ halo that appears. Fractions are extracted via puncturing the tube with a syringe and needle.
5. PVC Needle Complexes of good purity can be obtained in this manner, and stored in buffer at 4° C. Suitably, dialyse back in to TM buffer to remote the CsCl.

Following, or in place of CsCl gradient purification, PVCs can be extracted via Monolith anion exchange chromatography, as follows (note all steps can be performed manually with a peristaltic pump or syringe apparatus, or via F/HPLC):
1. Unless already done, dialyse the sample extract into the binding mobile phase (typically TM buffer) with a low concentration of salt (20 mM NaCl).
2. Equilibrate the column according to the manufacturer's guidelines, briefly:
   a. At least 5 Column Volumes (CV) of dH₂O;
   b. At least 5 CV of binding buffer (TM, with low salt);
   c. At least 5 CV of elution buffer (TM with high salt, >=1M NaCl);
   d. At least 10 CV of binding buffer once more.
3. Apply the sample to the column at a low flow rate (1-2 mL/min)
4. Wash the column with up to 200 mM NaCl-containing TM buffer.
5. Elute with 1M NaCl-containing TM buffer (alternatively, use a gradient elution if using an FPLC machine).
6. PVC Needle Complexes are present in the elution fractions. If a fraction collector is used, subsequent SDS-PAGE or similar may be needed to identify the correct fraction.

The column (of e.g. step 2) was of the CIMmultus™ Quaternary Amine anion exchange columns (BIA Separations d.o.o.). For example, the CIMmultus™ QA-1, which is a monolithic column with 1.3 μm channel size and a column volume of 1 mL.

Alternatively, a DEAE (a weak anion exchanger) column may be used.

Alternatively, for use with a Photorhabdus expression system, PVC Needle Complexes can be purified from supernatants as well as/instead of cell pellets, with the following additions/modifications:
1. Following cell harvest from the standard protocol above, supernatants are transferred to a pyrex bottle, and can optionally be concentrated via 100,000 MWCO columns if necessary.
   a. DNAse (0.25 U/mL) and protease inhibitors can optionally be added.
2. NaCl is added to a final concentration of 0.5M, and 80 g/L of PEG6000 is also added. The solution is mixed at 4° C. overnight.
3. The solution is centrifuged to pellet the PEG6000 at 8000×g, 4° C. for 30 mins.
4. The pellet is resuspended in a small volume (˜5 mL) of TM buffer (or similar) and incubated for 2 hours at room temperature, shaking.
5. Pellet by centrifugation at 13,000×g for 10 mins, and collect the supernatant to a new tube. Proceed with purification method of choice.

Other methods for purifying PVC Needle Complexes have been described elsewhere, for example in Yang et al (J Bacteriol. 2006 March; 188(6): 2254-2261), incorporated herein by reference.

Construction of an arabinose inducible over-expression strains for P. luminescens TT01 PVCunit4 (chassis encoded by genes Plu1667-plu1652)

Photorhabdus strains overexpressing a PVC Needle Complex were prepared using chromosomal recombineering to place a PVC (operon) of choice (operon encoding PVCunit4 Needle Complex was used here, as an example) under the control of an arabinose inducible transcription promoter. The recombineered strains are then genetically transformed with effector expression plasmids (e.g. based on the arabinose inducible expression vector pBAD30) to facilitate PVC Needle Complex over-expression, PVC effector expression, PVC effector trans-packaging, and secretion of the whole complex simply through the addition of the arabinose sugar.

Recombinant Photorhabdus PVC over-expression strain construction

The promoter region of PVCunit4 was amplified using primers PVCpromF (5′-TATCATATGTCTACAACTCCAGAACAAATTGCTG-3′, SEQ ID NO: 97) and PVCpromR (5′-ATCTCTAGAACAGATATTCCAGCCAGC-3′, SEQ ID NO: 98) using genomic DNA from P. luminescens strain DJC (aka strain TT01) as a template. A suitable P. luminescens strain is obtainable from the ATCC under accession no. ATCC 29999. The PCR product was digested with NdeI and XbaI and introduced by ligation into the suicide vector pCEP (ThermoFisher, catalog number: V04450), using E. coli DH5α λ-pir (Biomedal S.L.) as the carrier strain. The resulting plasmid was transferred to the E. coli donor strain S17.1 λ-pir (Biomedal S.L.) for conjugation into Photorhabdus. Briefly, overnight cultures of the donor strain and a rifampicin resistant (RifR) isolate of P. luminescens DJC were diluted in LB supplemented with 10 mM MgSO₄ and grown to mid-exponential (OD600 ˜0.5). Then, 3 ml of each culture were harvested, washed twice and re-suspended in 100 μl of LB supplemented with 10 mM MgSO₄. 80 μl of P. luminescens DJC RifR were mixed with 20 μl of the donor bacteria (resulting in a recipient to donor ratio of 4:1) and placed in the centre of an LB agar plate supplemented with 0.1% pyruvate and 10 mM MgSO₄. The plate was incubated overnight at 30° C. and the resulting growth was harvested in 1.5 ml LB. Aliquots were plated on plates containing rifampicin (50 μg/ml) and chloramphenicol (25 μg/ml) to select for trans-conjugants and the plates were incubated at 30° C. for 3 days. Possible transconjugants were re-streaked and confirmed by PCR using primers ParaINF (5′-GGCGTCACACTTTGCTATG-3′, SEQ ID NO: 99) and tPVCpR (5′-TCGGTGGCAGTAAATTGTCC-3′, SEQ ID NO: 100).

PVC Needle Complex over-expression and purification from Photorhabdus

Overnight cultures of P. luminescens DJC PVCunit4::pCEP were diluted in 2×250 ml LB supplemented with chloramphenicol (25 μg/ml) and incubated at 28° C., 180 rpm. After 2-3 h, arabinose (0.2%) was added and the cultures were returned to the incubator for another 26 h. The cells were pelleted by centrifugation (7000 g for 30 min) and the supernatant was collected. DNAse I was added to the supernatant at a concentration of 0.25 U/ml to degrade any extracellular DNA. Following an incubation of 30 min at room temperature, polyethylene glycol 8000 (8%) and NaCl (0.5 M) were added to precipitate the proteins. The supernatants were incubated overnight at 4° C., stirring. The precipitated proteins were then collected by centrifugation at 8000 g for 30 min at 4° C. The pellets were re-suspended in 8 ml TM buffer (20 mM TrisHCl, 20 mM MgCl2, pH7.4) and incubated at room temperature for 2 h with gentle shaking. Any remaining debris was removed by centrifugation at 13000 g for 10 min and the supernatant containing PVC Needle Complexes was applied to a CsCl density gradient and centrifuged at 35000 rpm for 2 h in a Beckman coulter Optima L-90K or XPN-80K ultracentrifuge. The CsCl density gradient was made by layering TM buffer containing CsCl at p=1.7 (2 ml), 1.5 (3 ml), and 1.45 (3 ml) from the bottom of the tube, respectively. The fraction containing PVC Needle Complexes was collected and Ultracel-100K devices (Amicon) were used to remove the CsCl and exchange the buffer for TMS (20 mM TrisHCl, 8 mM MgSO4, pH7.4). The PVC Needle Complexes were further purified using a CIMmultus™ quarternary amine 2 μm pore anion exchange column (BIAseparations). The column was washed with TMS buffer containing 200 mM NaCl and the PVC Needle Complexes were eluted in TMS containing 1 M NaCl. The NaCl was removed by buffer exchange using an Ultracel-100K device and the sample was applied to a CIMmultus™ DEAE 2 μm pore column (BIA separations) for a final purification. The column was washed in TMS containing 200 mM NaCl and the sample was eluted in TMS containing 500 mM NaCl.

It is possible to perform this with and without lysis (e.g. because the PVC Needle Complexes appear to be secreted from live cell, and can be collected in supernatant) of the cells (to release the PVC Needle Complexes).

### Transmission Electron Microscopy

For transmission electron microscopy (TEM) pioloform-covered 300-mesh copper grids that were coated with a fine layer of carbon were used as substrates for the protein fractions. A preferred aqueous negative stain is 3% methylamine tungstate. The coated grids were exposed to UV light for 16 h immediately prior to use to ensure adequate wetting of the substrate. A 10 μl drop was applied to the TEM grid, and the protein was allowed to settle for 5 min. Liquid was absorbed with filter paper from the edge of the grid and replaced immediately with 10 μl of filtered negative stain. The drop was partially removed with filter paper, and the grids were allowed to air dry thoroughly before they were viewed with a JEOL 1200EX transmission electron microscope (JEOL, Tokyo, Japan) operating at 80 kV.

BioPORTER assay and actin stress fibre analysis.

For BioPORTER assays (Genlantis), 80 μl of purified wild-type and mutant Pnf proteins (500 μg ml-1), or PBS as a negative control, were added to one BioPORTER tube (Genlantis) and re-suspended in 920 μl of DMEM. The samples were added to HeLa cells grown in 6-well plates and incubated for 4 h. BioPORTER/protein or PBS mixes were replaced by fresh complete medium and the cells were incubated for 20-48 h. To visualize cell morphology and actin cytoskeleton, cells were fixed for 15 min in 4% PBS-formaldehyde, permeabilized with 0.1% Triton X-100 and stained with Tetramethylrhodamine B isothiocyanate (TRITC)-phalloidin (Sigma) and DAPI dihydrochloride (Sigma). Images were acquired with a LSM510 confocal microscope (Leica).

### Example 1

### Cloning and Expression of PVC Needle Complexes

The inventors have successfully excised (cloned) the required expression genes from the host bacterium, Photorhabdus (e.g. which are comprised within SEQ ID NO: 93, SEQ ID NO.:94 and/or SEQ ID NO:95), and have devised a reliable, scalable expression system in laboratory E. coli as explained above. It has been demonstrated that trans-expression on separate plasmids enables incorporation of payloads (e.g. Pnf) into the syringes, creating a multi-plasmid (modular) platform.

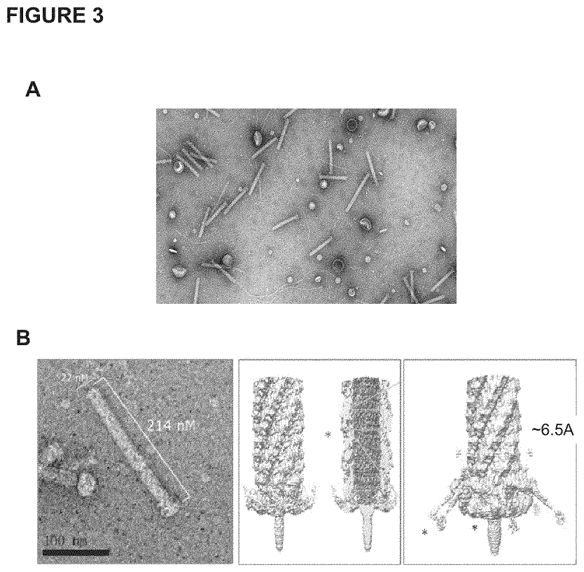

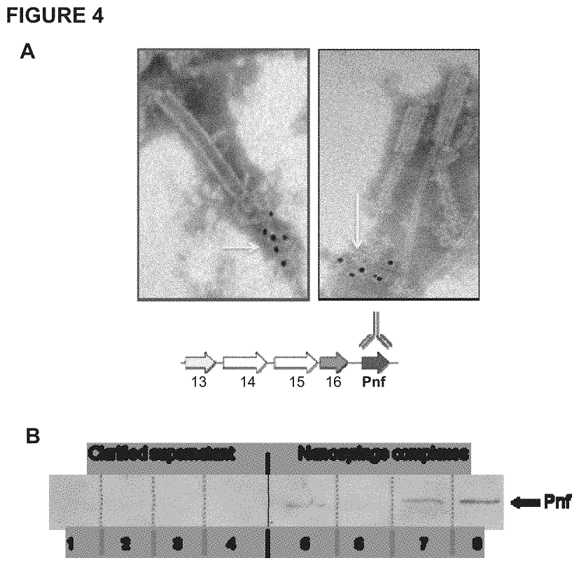

Following purification from E. coli, electron microscopy analysis demonstrated that the purified PVC Needle Complexes retained the correct ‘nanosyringe’ structure (see FIG. 3). Furthermore, PVC Needle Complexes remained correctly associated with the payload (e.g. Pnf) following purification (see FIG. 4), demonstrating that the inventors have successfully prepared the PVC Needle Complexes (nanosyringes) having the correct structure for payload delivery to cells.

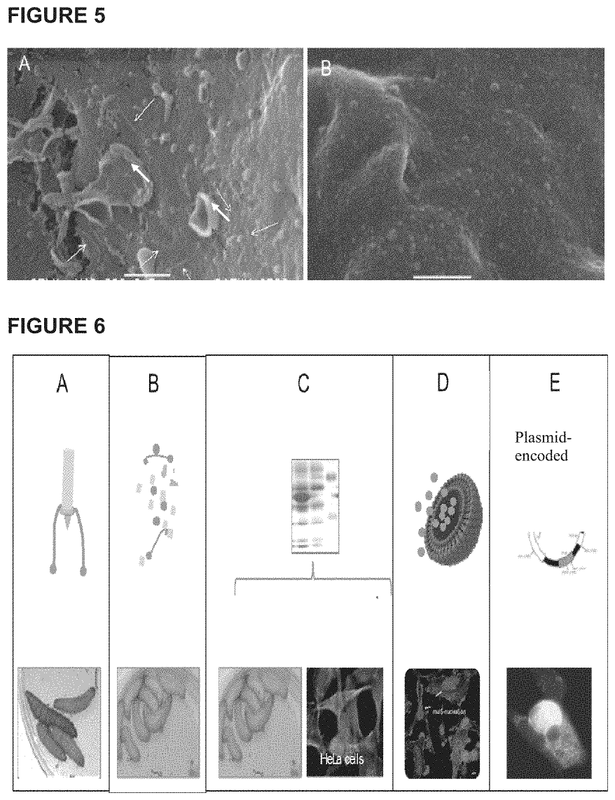

Furthermore, electron microscopy analysis demonstrated that the purified complexes appropriately localise to the cell surface of cells, and PVC Needle Complexes with a Pnf payload (PVCpnt) induces a phenotype (ruffling) consistent with the postulated mechanism of the effector (PVC)—see FIG. 5.

### Example 2

### 2.1 Demonstrating PVC Needle Complexes Exert Effect Via Intracellular Delivery of Effector

The polypeptide Pnf was identified as a PVC effector as follows. This was identified within the Photorhabdus asymbiotica ATCC43949 complete genome—GenBank Accession Number: FM162591.1.

The final gene of the PVC operon (P. asymbiotica ATCC43949 PVCpnf operon, which has a sequence of SEQ ID NO: 93) was identified, namely pvc16 (e.g. PAU_03338). The position of the pvc16 genes of a PVC locus is illustrated in FIGS. 1(A), (B) and (D). ORFs shortly 3′ of pvc16 (e.g. within about 5 kb downstream of pvc16) were identified—one such ORF (PAU_03332) being 3535 bp downstream of pvc16. The predicted function of the polypeptide (having a sequence of SEQ ID NO.: 32) encoded by this putative effector ORF was obtained by a combination of BlastP and HHPRED (https://toolkit.tuebingen.mpg.de/#/tools/hhpred). This ORF could then be assigned as a PVC effector based on direct homology to a known bacterial toxin (e.g. of the CNF1 family from E. coli).

A Pnf loaded PVC Needle Complex was then prepared according to Example 1.

The inventors have demonstrated that these packaged (e.g. laden) PVC Needle Complexes exert cellular effects consistent with the provenance of the cargoes they carry. By way of example, cells and whole insect animals exposed to PVC Needle Complexes loaded with the cytoskeleton toxin Pnf undergo cell death in a manner consistent with cytoskeleton toxicity.

Injection experiments (injection into the insect larvae) were performed by injection of 10 μl of supernatant, provided following centrifugation (pelleting) of an overnight culture (typically 1 L) of a culture of E. coli harbouring a cosmid clone encoding the PVC Needle Complex with Pnf (PVCPnf)—e.g. a PVC encoded by SEQ ID NO.: 93, packaged with a PVC effector of SEQ ID NO.: 32.

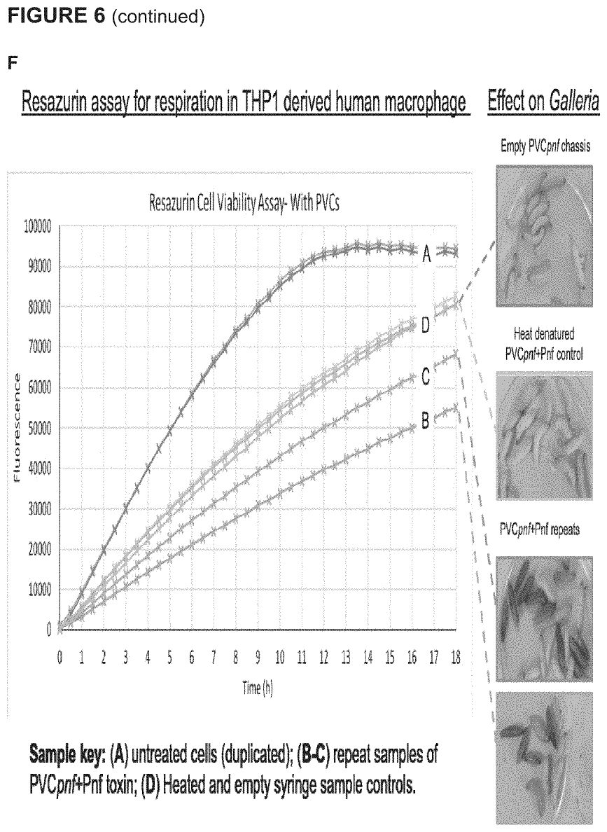

Demonstrating that the PVC Needle Complexes are responsible for the phenotype due to intracellular delivery (e.g. injection) of the Pnf payload, the toxic effect could only be reconstituted when the same protein (Pnf) is provided with another route to access the cell cytosol (transfection and expression of an expression plasmid, or conductance via liposomal preparations containing the protein)—see FIG. 6. Conversely, denatured (via boiling) PVC Needle Complex preparations, toxin proteins overlaid on tissue culture cells or toxin proteins injected into whole animals showed no activity.

2.2. Evidence of Delivery of the Toxic Effector Enzyme Pnf into Cultured Human Macrophages

To complement the data outlined above, the inventors conducted additional experimentation providing further evidence of delivery of the toxic effector enzyme Pnf into cultured human macrophages.

Concept: The inventors tested PVCpnf expressed and purified from E. coli, (trans-)packaged with the native Pnf toxin on cultured human THP1 derived macrophages. Unlike the lethal effect of the Pnf toxin in insect models, previous liposome mediated Pnf protein transfection experiments indicated a subtler phenotype in human Hela cells. In those experiments the cells showed actin stress fibre formation at 24 h and multinucleation at 48 h. The inventors therefore tested the effect of the purified PVCpnf (the nanosyringe) holding/packaged with the Pnf PVC effector on macrophage respiration rate using a Resazurin colourimetric assay.

### Methods:

Background behind Resazurin assays. The blue compound resazurin was explored for use in assays to determine the activity of PVCs on macrophages (MO). Resazurin is metabolically reduced in cell mitochondria, producing a pink and highly fluorescent compound, resorufin. The effect of PVCs on macrophage metabolism can be determined by introducing resazurin into the culture media. The number of macrophages affected by PVCs can be inferred by comparing the fluorescence measured to that of the cell density optimisation curve (see Czekanska, Methods in Molecular Biology, 2011, 740, 27-32, incorporated herein by reference).

Optimisation of use of Resazurin for THP1 derived macrophages. The metabolism of macrophages over 18 h was assessed at different seed densities to determine the optimum cell density for use of this assay with PVCs. A 30 mL culture of THP-1 cells was pelleted at 1000 rpm for 4 min, before resuspension in 2 mL of RPMI media (also containing 10% FBS (v/v) and 2 mM L-glutamine). Cells were counted using a cell haemocytometer, then diluted in media to a density of 2×10⁶ cells mL⁻¹. THP-1 cells were then activated with phorbol 12-myristate-13-acetate (PMA) immediately before plating. 200 μL of the cells were plated in quadruplicate in a 96-well plate, and a 2-fold serial dilution was performed until reaching a final cell density of 1.5625×10³ cell mL⁻¹. 125 μL of the starting cell dilution was also plated in quadruplicate on the same plate, for a 5-fold serial dilution, until reaching a cell density of 0.32×10³ cells mL⁻¹. Four blank wells were also prepared, containing RPMI and PMA. The plate was incubated at 37° C. with 5% CO₂ for 48 h. Media was aspirated from the wells and replaced with fresh RPMI, and the macrophages were incubated for a further 24 h. A resazurin tablet (VWR) was dissolved in RPMI (12.5 mg/mL), and 10 μL added to each well in quick succession (well concentration of 1.25 mg/mL). The fluorescence produced was measured on a plate reader every 30 min for 18 h (excitation: 530-570 nm, emission: 580-620 nm, maintained at 37° C. and 5% CO₂). The optimum cell density over time was then determined for use with PVCs.

Use of assay for PVC testing. THP-1 cells, diluted to 1.25×10⁵ mL⁻¹, were activated and seeded in a 96-well plate, where wells contained 100 μL of cells at a final well density of 1.25×10⁴ cells mL⁻¹. Blank wells were also prepared in quadruplicate, containing cells without PVC samples, as well as wells containing media and PMA only. The plate was incubated for 48 h at 37° C. with 5% CO₂. The media was then replaced with fresh RPMI, before addition of 10 μL of each PVC sample. The plate was incubated for a further 24 h, before the addition of 10 μL resazurin (12.5 mg/mL) to each well, and the fluorescence was measured every 30 min for 18 h (excitation: 530-570 nm, emission: 580-620 nm, maintained at 37° C. and 5% CO₂).

Results: FIG. 6F shows that challenge with PVCpnf+Pnf did indeed lower the respiration rate of the macrophage, while heat denatured or empty PVCpnf nanosyringes had no strong adverse effect. Nevertheless, control cells with no sample addition still showed the best respiration rates. The effects on macrophage were correlated with insect injection toxicity assays. In this case the two PVCpnf+Pnf preparations showed lethality to over half the insect cohort, while the heat denatured and empty PVCpnf injected insects all remained healthy.

### Example 3

Demonstrating that a Leader Sequence is Responsible for Payload Packaging into PVC Needle Complexes

Surprisingly, the inventors have found that the provision of a ‘leader’ peptide sequence, preferably on the N-terminus of a payload (toxin) protein, can direct the payload to the PVC complex and allow for (e.g. trigger) the packaging of the payload into the PVC Needle Complex. The inventors have demonstrated that amino acid residues 1-50 of a PVC effector protein is/comprises a leader sequence.

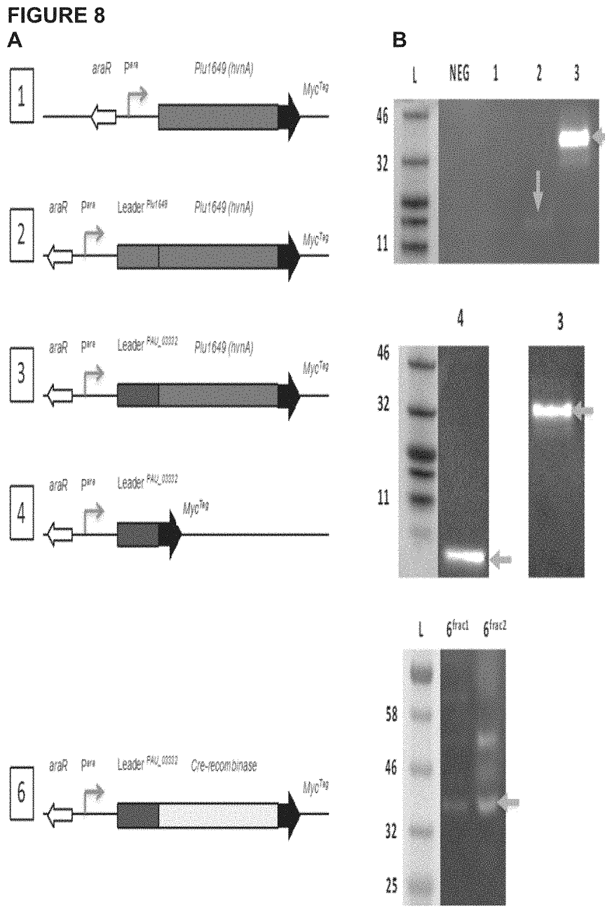

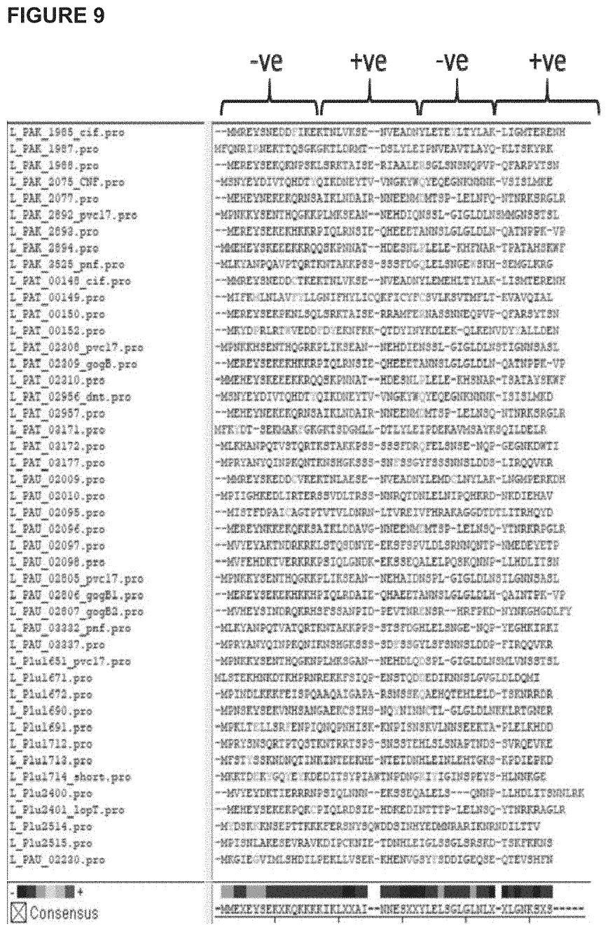

To demonstrate this, an expression construct (overexpression in chromosomally engineered P. luminescens TT01) was prepared, in which the leader sequence (the N-terminal amino acid residues 1-50) was ablated such that the payload expressed by Plu1649 (referred to as “hvnA” in the figure, and having a sequence of SEQ ID NO.: 46) (Myc-tagged for detection purposes) was absent a leader sequence (see FIG. 8A—construct 1). Following expression (of both the payload and PVC Needle Complex) and isolation of the PVC Needle Complex (and running the components thereof, which includes any packaged payload, on a gel), no (Myc-tagged) Plu1649 (“hvnA”) was detectable within the PVC Needle Complex via western blot analysis, demonstrating that the payload (absent the leader sequence) was not packaged into the complex (see FIG. 8B, lane 1), and thus not associated with the isolated complex. Successful packaging was seen, however, for hvnA which did retain the leader sequence, see lane 2 (note that the band appears weak, due to the relative intensity of the band of lane 3).

Surprisingly, hvnA having a leader sequence from a different (non-hvnA) PVC effector (i.e. corresponding to the N-terminal amino acid residues 1-50 from the PAU_03332 effector) (see FIG. 8A, construct 3) was correctly packaged into the complex and remained associated with the PVC Needle Complex upon isolation/purification, as demonstrated by Western blot detection of the Myc-tagged hvnA (see FIG. 8B, lane 3). Thus, the inventors have demonstrated the surprising ability of the ‘PAU_03332’ leader sequence (which is associated with a different payload, Pnf) for packaging of a hvnA payload (i.e. a different payload to that of PAU_03332). This demonstrates the ability to swap the leader sequences of the PVC effector, allowing use of an optimal leader sequence (having optimal packaging activity) for packaging.

### Example 4

4.1 Demonstrating that a Leader Sequence Directs Packaging (into PVC Needle Complexes) of Atypical/Exogenous Payloads

In an unexpected technical effect of the invention, the inventors have found that fusing a leader sequence described herein to exogenous (non-Photorhabdus) polypeptides (preferably at the N-terminus) allows for packaging of said exogenous polypeptides into a PVC Needle Complex, with the exogenous polypeptides remaining associated with the PVC Needle Complex upon isolation/purification. By way of example, see FIG. 8B (lane 4) demonstrating that a non-Photorhabdus ‘Myc’ polypeptide (<10 kDa) is packaged into the PVC Needle Complex when fused to a leader sequence, and lane 6, demonstrating a much larger non-Photorhabdus ‘Cre-recombinase’ polypeptide (>32 kDa) can likewise be appropriately packaged into PVC Needle Complex when fused to a leader polypeptide of the invention.

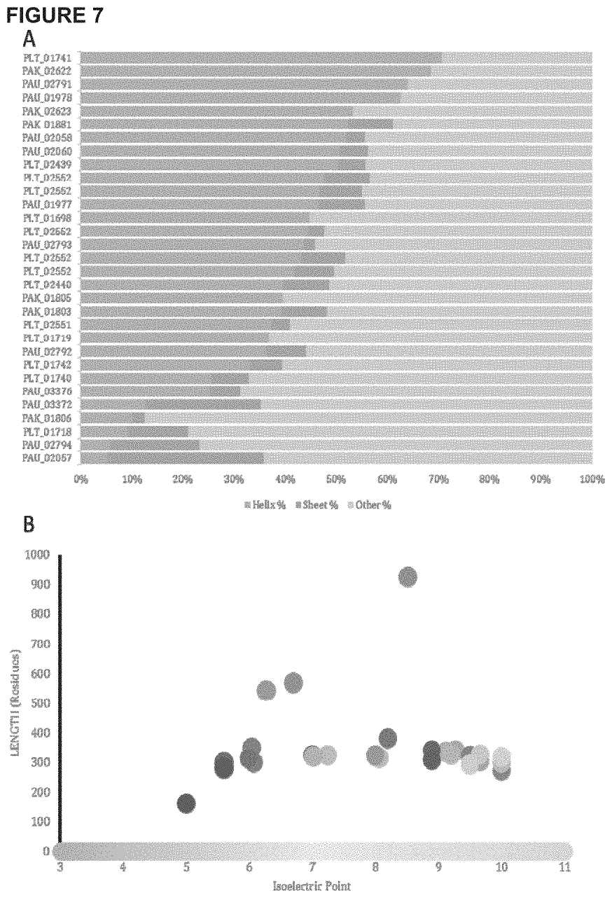

The inventors performed in-depth analysis of the size (e.g. polypeptide length) and structure of the various natural PVC effector payloads encoded by Photorhabdus (see FIG. 7), which show a wide variety of different lengths and structure, demonstrating that the applicability of the PVC Needle Complex (nanosyringe) delivery system of the present invention is not limited by the size or properties of the payload protein of interest. To summarise, there is no requirement for particular secondary structure, biophysical property, or length of cargoes, confirming that that the PVC Needle Complex (nanosyringe) chassis can be utilised as a versatile multifunctional delivery vehicle.

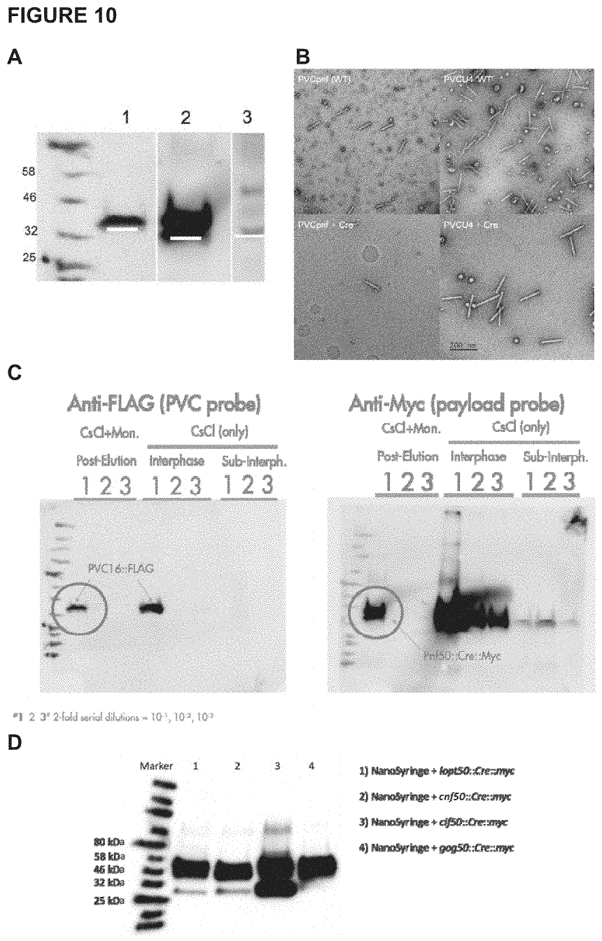

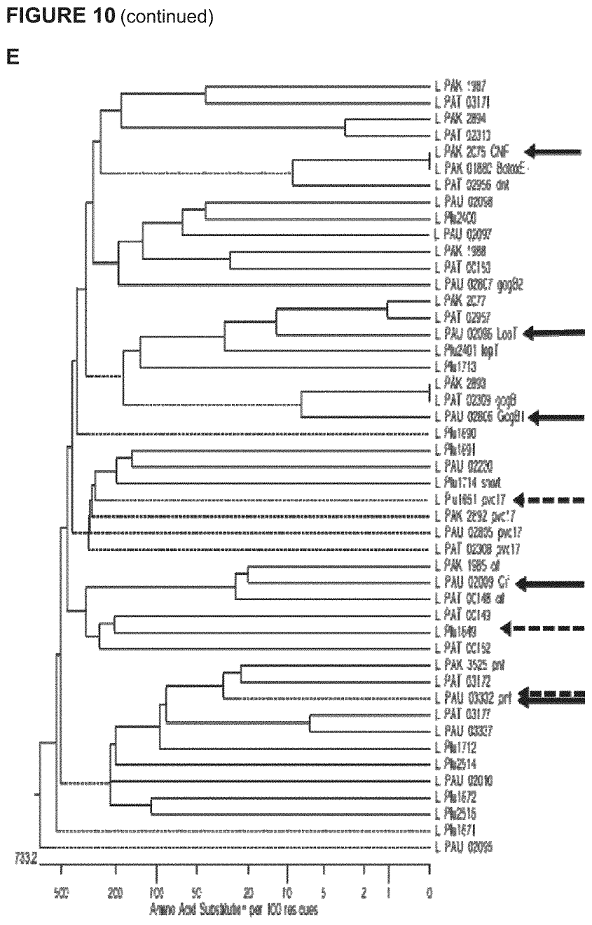

Furthermore, this packaging of exogenous polypeptides is independent of the chosen PVC Needle Complex chassis e.g. has been accomplished using both a “PVCpnf” chassis (SEQ ID NO.: 93) and a “PVCU4” (e.g. PVCunit4) chassis (endogenous to the Photorhabdus overexpression strain) (see FIG. 10A). Importantly, the inventors have demonstrated that packaging exogenous payloads in either chassis does not affect morphology of the PVC Needle Complexes, ensuring they are not assembled aberrantly (see FIG. 10B).

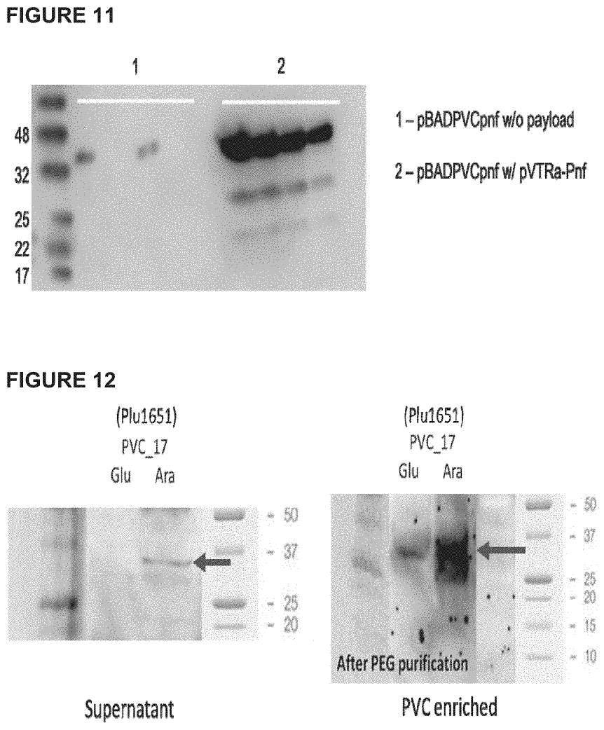

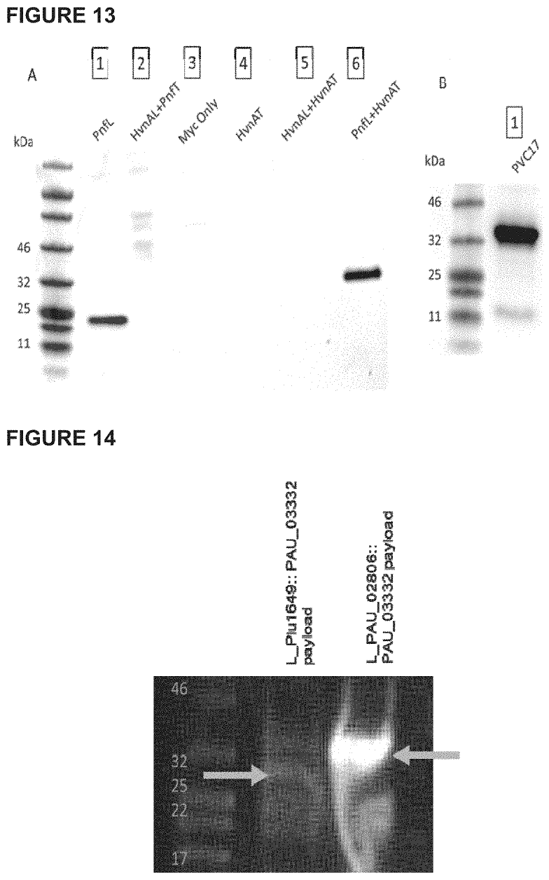

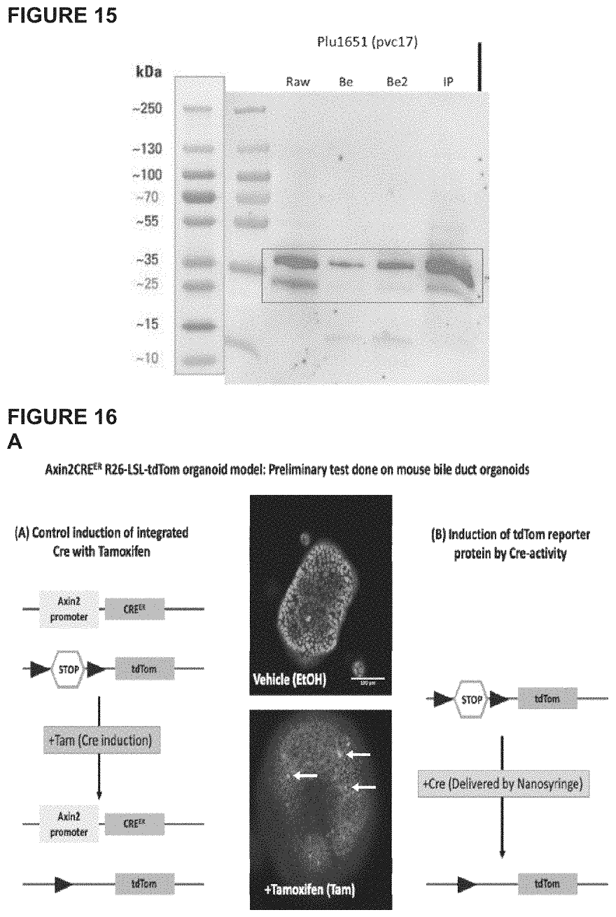

In data shown herein, payload proteins are supplied in ‘trans’ on separate genetic constructs. The leader sequences are surprisingly sufficient to target these separately synthesised proteins for packaging into the PVC Needle Complex vehicle (see FIG. 11). This applies in E. coli when the chassis (PVC) genes themselves are also present on a plasmid, as well as with chassis genes being integrated into the chromosome, as is the case in Photorhabdus, the host organism.

Further exemplification of trans-packaging of high levels of the Cre site specific recombinase into the PVCpnf nanosyringe expressed in E. coli is provided in FIG. 10(C). In more detail, the inventors constructed a laboratory E. coli expression strain harbouring (i) the arabinose inducible expression plasmid for the P. asymbiotica ATCC43949 PVCpnf operon e.g. of SEQ ID NO.: 93 (with a C-terminal FLAG tag on Pvc16, e.g. immediately 3′ to SEQ ID NO.: 93) and (ii) a second IPTG inducible expression plasmid containing the Cre recombinase with a N-terminal fusion of the natural Pnf effector 50 amino acid leader sequence (e.g. leader of SEQ ID NO.: 78) and a C-terminal Myc-TAG epitope. The PVC operon and effector (Cre+leader sequence) were co-induced for 24 hours and the chimeric nanosyringes purified. Western blot analysis was used to confirm the presence of the FLAG-tagged Pvc16 cap protein (and therefore the nanosyringe chassis) and the trans-packaged Myc-tagged Cre recombinase post purification.

4.2 Trans-packaging using additional leaders demonstrating functionality of a larger, diverse sequence space

Complementing the data outlined in Example 3, FIG. 10D demonstrates (trans-) packaging of Cre into PVCpnf (in E. coli) using the following four additional leader sequences (thus demonstrating the functionality of a larger sequence space):
Lane 1: the leader of PAU_02096 (leader sequence=SEQ ID NO.: 71), experiment referred to as “NanoSyringe+lopt50::cre::Myc in FIG. 10D;
Lane 2: the leader of PAK_02075 (leader sequence=SEQ ID NO.: 50), experiment referred to as “NanoSyringe+cnf50::cre::Myc in FIG. 10D;
Lane 3: the leader of PAU_02009 (leader sequence=SEQ ID NO.: 68), experiment referred to as “NanoSyringe+cif50::cre::Myc in FIG. 10D; and
Lane 4: the leader of PAU_02806 (leader sequence=SEQ ID NO.: 76), experiment referred to as “NanoSyringe+gog50::cre::Myc in FIG. 10D.

These results also demonstrate the utility of leader sequences showing greater sequence diversity for (trans-)packaging a payload. Indeed, to provide further validation, the inventors performed a CLUSTALW sequence comparison of a panel of leader sequences to determine diversity. PVC effectors are identified as proteins encoding recognisable toxin-like domains that are encoded immediately downstream of the pvc16 structural gene. Each PVC operon can encode just a single effector, or several different effector genes in tandem array. A phylogenetic tree is shown in FIG. 10E, with the identities of leader sequences exemplified herein for packaging payload proteins into the nanosyringe complexes being elaborated by either the P. asymbiotica ATCC43949 PVCpnf operon (solid arrows) or the P. luminescens TT01 PVCunit4 operons (dashed arrows) or both.

As can be seen from the tree of FIG. 10E, the exemplified leader sequences are well distributed throughout and are therefore at or close to maximally sequentially diverse.

### Example 5

### Tail Fibre/Binding Domain Modification

PVC Needle Complexes are known to comprise tail fibres (see the 3D rendered PVC structure, left most asterix of the rightmost image) which are believed to allow for cell-type specific targeting of the PVC complexes. The inventors have successfully demonstrated that modification of a tail fibre region to incorporate non-natural amino acids (e.g. a substitution of an amino acid in the wild-type sequence for an alternative amino acid of the 20 standard amino acids) does not affect expression of tail fibres.

### Example 6

Demonstrating Delivery of an Active (Exogenous) Enzyme/Payload into Ex Vivo Murine Organoids with a Leader Sequence-Packaged PVC Needle Complex

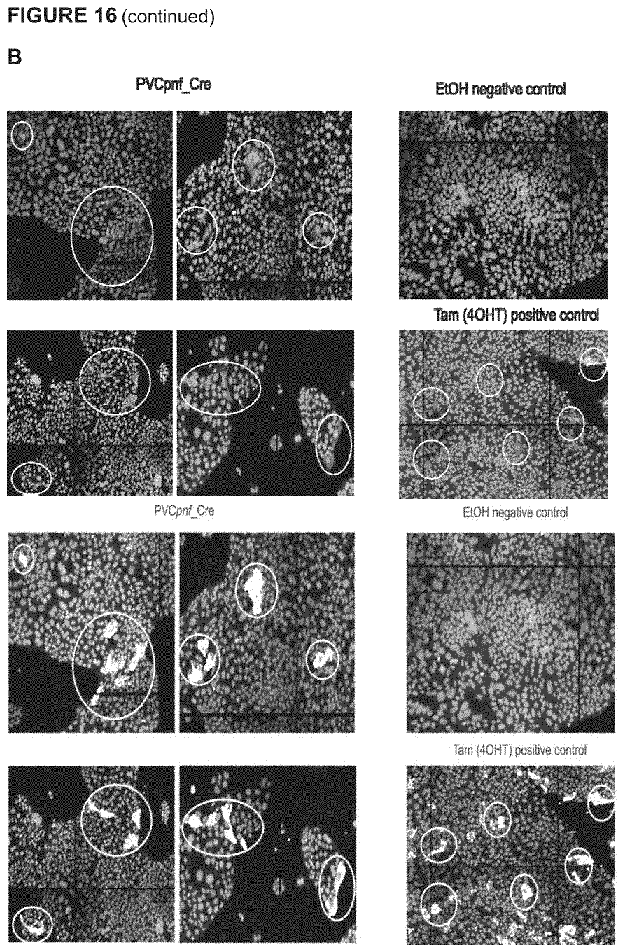

Concept: Obtaining data for the delivery of an exogenous functional enzyme to a mammalian tissue. The inventors have demonstrated the delivery of a trans-packaged bacteriophage derived recombinase protein known as “Cre” into ex vivo mouse bile duct organoids. The organoids are derived from a mouse line in which the expression of a chromosomally encoded red fluorescent protein (RFP) reporter is normally prevented by a stop signal flanked by loxP recognition sites for the Cre-recombinase. If the recombinase is present, the stop signal is recombined out and the cells then go on to express the reporter protein. The general principle behind this experimental demonstration is summarised in FIG. 16A.

Method: The Bile Duct organoid preparation: murine primary bile ducts were isolated and expanded as organoids in matrigel using “BD expansion media” for 12 passages following Huch et al (Regen Med. 2013 Jul.; 8(4):385-7. PMID: 23826690; DOI:10.2217/rme.13.39) protocol. Cells were then plated in 2D and cultured in BD expansion media. Mouse Genotype: LSL-Tom reporter in Rosa26 locus+Axin2CreRT (inducible upon 40HT treatment). Cells were cultured in uncoated polystyrene plates at a seeding density: of 10,000 cells/well. Nanosyringes were prepared as 30% volume syringe preparation in PBS+70% culture media. Total volume of 100 μl per well. The positive control represented 500 nM 40HT (in ethanol) at 1:1000 (v/v) as positive control for the recombination. The negative control represents 1:1000 (v/v) ethanol dilution only. Cells were seeded and grown for 48 h, nanosyringes added and then cultured for another 24 h before fixing (4% PFA fixation 15 min RT) and staining for microscopic examination. Staining: Primary antibody Anti-RFP (1:1000) from Rockland. Secondary Anti-Rabbit 568 (used at 1:500 v/v). Samples were visualized on a laser-confocal microscope.

Result: FIG. 16B includes representative micrographs from these experiments demonstrating signal for the RFP protein could be detected in a number of cells when treated with the Cre loaded PVCpnf nanosyringe. As these are ex vivo organoids, rather than simple cell monolayers, some stochasticity in the number of cells that are dosed is expected, and this is even observed in the positive control, which is a small molecule inducer (rather than a large protein complex). It is anticipated that, as these are organoids, there will be some level of cellular differentiation present which may alter the binding characteristics of the nanosyringes. A further interesting observation from this preliminary run, is that while information on total amounts of nanosyringes applied to the system is not yet available, the inventors demonstrate that the TAM small molecule inducer does not appear to have appreciably greater tissue penetration than the nanosyringes, suggesting their ability to distribute is not majorly hampered by their size.

Additional interpretation: To summarise, the inventors have demonstrated the ability to deliver (e.g. dose) exogenous enzymes to a cellular target. Moreover, this “nanosyringe+Cre” experiment is a promising proof of concept for a biotechnology tool/aide, by demonstrating the ability to provide a DNA change leading to a transformed cell. This experiment therefore demonstrates the use of exogenous payloads (a protein of viruses rather than bacteria), and nucleic acid modifying enzymes in particular. It is evident that the Cre enzyme is delivered in a functional manner and is capable of traversing the cellular interior to the nucleus to affect its DNA modifying changes.

### Example 7

Trans-Packaging of MAD7 Site Specific Recombinase (Exogenous Payload) into the PVCpnf Nanosyringe Expressed in E. coli

Concept: As with the Cre data (of Example 6), and other examples of packaged payloads provided herein, the inventors have demonstrated packaging of the Cas-like enzyme MAD7 into a nanosyringe via a leader sequence. This is the largest exogenous example (MAD7=147.9 kDa) of a payload described herein.

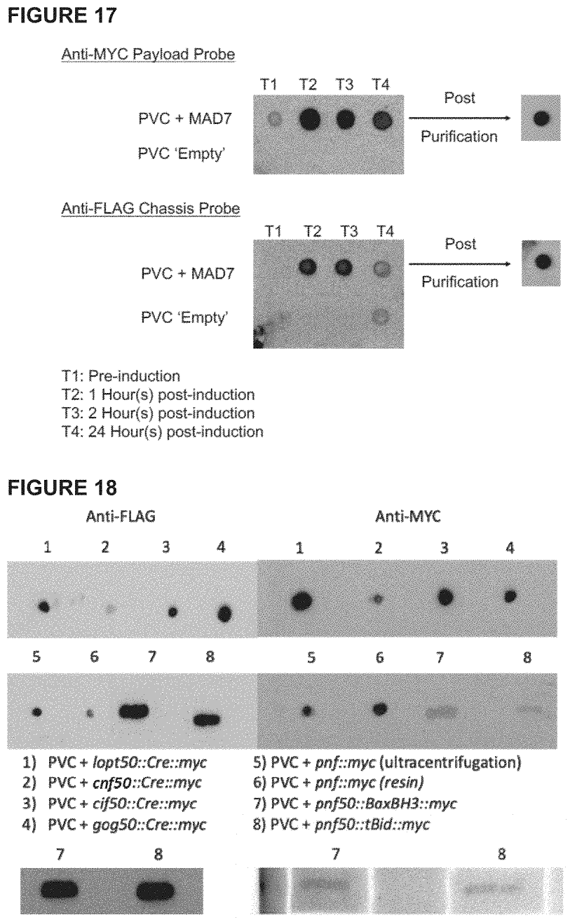

Methods: Briefly, the chassis genes and the MAD7 gene (the latter being tagged with a C-terminal Myc tag for detection, and a leader sequence for nanosyringe incorporation described herein), were expressed (upon induction) simultaneously in E. coli. Upon harvesting and purification of the nanosyringe complex, payload packaging was probed via dot blot analysis (e.g. for detection of the Myc tag). The purification method described herein (using ultracentrifugation) can be employed to select for (e.g. exceedingly) high molecular weight protein complexes/biological matter, enabling recovery of the nanosyringes and any cargo (payload) they carry. ‘Loose’/unpackaged payload remains in solution and is not subject to sufficient centrifugal force and as such is lost during purification, unless contained within the much larger nanosyringe ‘shell’ (that is, when successfully packaged). Successful packaging of MAD7 is demonstrated by FIG. 17.

### Example 8

Trans-Packaging of Apoptosis Inducing Payloads into PVCpnf, Expressed in E. coli

Using the E. coli PVCpnf leader::payload::Myc trans-packaging system described in FIG. 10C (PVCpnf leader=SEQ ID NO.: 78), the inventors demonstrated the ability to trans-package at least two pro-apoptotic human derived protein sequences or peptides (e.g. the sequences of SEQ ID NO.: 109 and SEQ ID NO.: 111). The Pnf effector protein leader sequence (e.g. SEQ ID NO.: 78) was fused to the N-terminus, and a Myc epitope tag was fused to the C-terminus. Western dot blot analysis (similar to that of Example 7) confirmed the presence of these human derived proteins in purified nanosyringes (FIG. 18).

### Example 9

Demonstration of the induction of apoptosis in cultured ex vivo human cells by nanosyringe delivery of (trans-)packaged pro-apoptotic human polypeptides

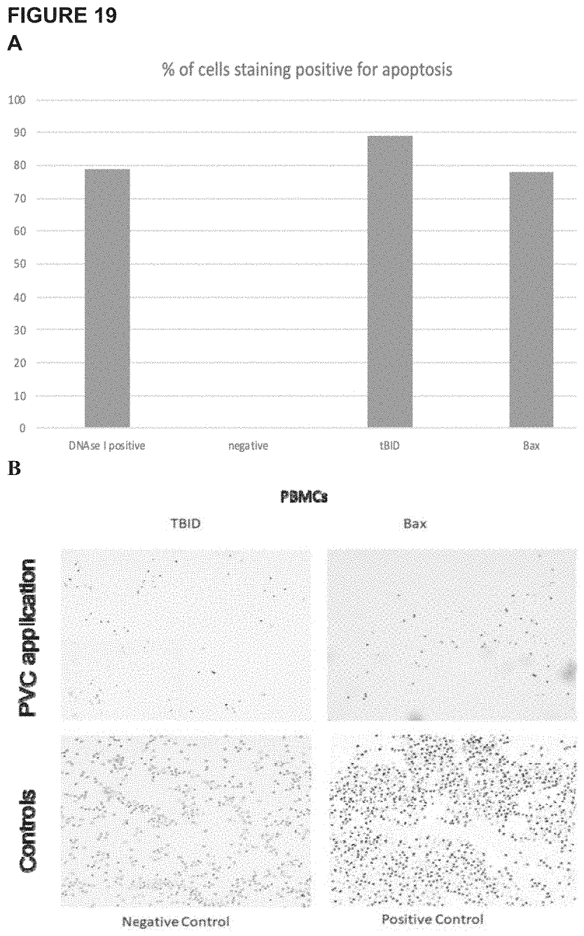

A preliminary test has confirmed the ability to use the PVCpnf nanosyringe, produced in E. coli, to deliver trans-packaged human protein sequences (e.g. packaged according to Example 8) and induce apoptosis in ex vivo circulating PBMC cells from human donors. The assay is a TUNEL-stain microscopic analysis from cells exposed to the packaged nanosyringes for 20 minutes only. Results are shown in FIG. 19A, demonstrating (via successful induction of apoptosis) delivery of tBid p15 fragment and BaxBH3 domain.
tBid p15 fragment (SEQ ID NO: 109) is part of the normal human apoptosis regulation pathway. Cellular effects: a pro-apoptotic member of the Bcl-2 family. The C-terminal part of Bid (tBid) translocates to the mitochondria, where it induces the release of cytochrome c. Bid is normally cleaved by caspase 8 from its latent cytosolic full-length pro-Bid form.
BaxBH3 (aa59-73) (SEQ ID NO: 111) is a minimal BH3 domain synthetic peptide, comprising critical 15 residues of the defined Bax BH3 domain. Cellular effects: these 15 residues contain sufficient information to bind to, and functionally antagonize, Bcl-xL and to induce specifically Bax/Bak. Appears to abrogate Bak/Bcl-2 interactions—freeing up pro-apoptosis factors.

A more detailed test of the delivery of pro-apoptotic human peptides into ex vivo Peripheral Blood Mononuclear Cells (PBMCs) is now described. The aim of this study was to investigate whether the pro-apoptotic peptide loaded PVC nanosyringes could induce apoptosis in ex vivo human Peripheral Blood Mononuclear Cells. The nanosyringes were first assessed for any immediate cell toxicity using Trypan blue dye exclusion assays and then for apoptosis response by using the TUNEL assay.

Trypan Blue Exclusion Test for cell viability: Trypan blue is a Diazo dye commonly used to selectively colour dead tissue or cells, hence, dead cells are shown as a distinctive blue colour under a microscope while live cells or tissues with intact cell membranes remain uncoloured. Since live cells are excluded from staining, this staining method is also described as a Dye Exclusion Method. Trypan blue is commonly used for assessment of tissue or cell viability. A suitable number of cells (2×10⁵) were exposed to the nanosyringes and empty nanosyringe for 20 minutes. A suitable volume of cells (30 μL) were added to an equal volume of 0.4% Trypan blue and the number of viable (unstained) and dead (stained) cells counted using a hemocytometer. Each compound was tested at 3 concentrations. Blood cells from two independent human donors was tested for each compound at each concentration and each sample was tested in duplicate.

Treatment and preparation of cells for microscopy: The viability of Peripheral Blood Mononuclear Cells (PBMCs) from two independent healthy human donors was determined after 20-minute treatment with the two chimeric nanosyringes (e.g. loaded with the exogenous pro-apoptotic peptides) at 3 test concentrations in 2 independent tests. PBMCs were harvested by centrifugation and resuspended in media at 1×10⁶ cells/ml. Cells were fixed in 2.5% formalin and incubated for 20 mins at room temperature. Poly-L-lysine coated slides were prepared by spraying with 70% ethanol and leaving to air dry. Cells were centrifuged for 30 seconds. Supernatant was removed and cells were resuspended in 200 μl dH₂O. 5 μl of cell suspension was added to each slide/fixation. Two fixations were performed per slide to allow staining to be performed in duplicate. Cell suspension was left to air dry.

Results of PBMC cell viability assay: The Trypan blue viability assays confirmed that the PVC preparations were not immediately toxic in themselves to PBMCs taken from healthy human donors (Table 2). Nanosyringe treatment showed >60% viability indicating low toxicity at maximum dose concentration (Table 2). The inventors then moved on to test the ability of the chimeric nanosyringes to induce apoptosis.

TABLE 2
Viability of Peripheral Blood Mononuclear Cells
(PBMCs) from two independent human blood donors
after exposure to each compound for 20 minutes at 3 test
concentrations (v/v dilutions). PBMC controls are untreated.

| | Neat | | 1/10 | | 1/100 | |
|---|---|---|---|---|---|---|
| | Well 1 | Well 2 | Well 1 | Well 2 | Well 1 | Well 2 |
| Blood donor | | | | | | |
| sample 1 | | | | | | |
| TBID | 60 | 70 | 75 | 75 | 80 | 80 |
| Bax | 78 | 82 | 80 | 80 | 83 | 87 |
| PBMC Controls | 93 | 97 | | | | |
| Blood donor | | | | | | |
| sample 2 | | | | | | |
| TBID | 70 | 72 | 81 | 81 | 84 | 84 |
| Bax | 80 | 80 | 84 | 88 | 87 | 87 |

| | | |
|---|---|---|
| PBMC Controls | 95 | 95 |

Testing for chimeric nanosyringe induced apoptosis using the TUNEL assay: The TUNEL assay was then used to identify apoptotic nuclei in single cell suspensions fixed on slides. In the assay Terminal deoxynucleotidyl Transferase (TdT) binds to the exposed 3′-OH ends of DNA fragments which are generated in response to apoptotic signal factors. This in turn catalyses the addition of biotin-labelled deoxynucleotides which can be detected using a streptavidin-horseradish peroxide (HRP) conjugate. Diamineobenzidine (DAB) reacts with the HRP-labelled sample to generate an insoluble brown substrate at the site of DNA fragmentation. Methyl green counterstaining enables the visualisation of normal and apoptotic cells.

The induction of apoptosis following exposure of human PMBCs to the nanosyringes was determined. A TUNEL assay kit (Abcam) was used for detection of apoptotic cells. The assay was performed following the manufacturer's instructions. Briefly, slides were covered with 100 μL proteinase K solution or 5 minutes, slides were rinsed with 1×TRIS buffer saline (TBS). The treatment of nanosyringes or the DNase I positive kit control was performed for 20 minutes at room temperature. Slides were rinsed with TBS. Slides were then incubated with TdT equilibrium buffer for 30 minutes before the addition of TdT labelling reaction mix. Slides were incubated at 37° for 19 minutes. Slides were then washed with TBS before application of the stop buffer and incubation at room temperature for 5 minutes. Slides were washed again with TBS before addition of the blocking buffer for 10 minutes at room temperature. Detection was performed by application of the conjugate to the samples for 30 minutes. Slides were rinsed with TBS before application of the DAB solution for 15 minutes. Slides were rinsed with dH₂O followed by counterstaining with methyl green. Slides were dehydrated in 100% ethanol followed by xylene and mounted with a glass cover slip. All staining was performed in duplicate. An apoptosis endpoint, indicative of positive staining in the apoptosis detection assay is represented by dark brown (DAB) signal. Lighter shades of brown and/or shades of blue/green to green/brown indicate a non-reactive negative cell for apoptosis.

Analysis was performed by selecting 5 random sections of cells on the slide, positive stained cells (dark drown) and negative stain cells (blue or light brown) were counted and the percentage of cells showing apoptotic bodies was determined.

To generate a positive control, slides were treated with 1 μg/μl DNase I (the kit positive control) for 20 minutes at room temperature following the proteinase K treatment step detailed below. The DNase I treatment fragments DNA in normal cells to generate free 3′OH groups identical to those generated during apoptosis. A negative control was generated by substituting DNase I with dH₂O in the reaction mix during the treatment stage.

Results of PMBC apoptosis assays: TUNEL staining using PBMCs was performed following treatment with the intact tBID and Bax loaded nanosyringes, with appropriate positive and negative kit controls. Treatment was performed for 20 mins to determine if the nanosyringes elicited an apoptotic signal. A positive control (DNase I treatment) and negative control (no DNase I treatment) was included. Results showed both nanosyringes, containing either tBID or Bax, showed strong apoptotic signals (89% and 78% positive, respectively) on the PBMCs. The positive control showed a strong apoptotic signal (79%), whereas the negative control showed no apoptotic signal (100% negative). Also observed was a significant loss of the numbers of attached cells in the nanosyringe treated samples, presumably indicative of a rapid and comprehensive apoptosis response, and a failure to be retained after washing. Note this effect is even more pronounced than the kit positive control suggesting a more rapid response. Representative micrographs are shown in FIG. 19B.

Conclusion: It is concluded that the tBID and Bax loaded nanosyringes are able to rapidly induce extensive apoptosis in human Peripheral Blood Mononuclear Cells. Furthermore, Trypan Blue dye exclusion assays have confirmed that these chimeric nanosyringes do not cause rapid lethal lysis or extensive membrane damage to the cells.

### Example 10

### Exemplification of Practical Utility of Leader Sequences and PVC Needle Complexes—Intracellular Delivery of Atypical (Non-Photorhabdus) Payload

(1) An anti-MDM (p53 inhibitor) antibody is linked to a leader sequence described herein, and expressed together with a PVC Needle Complex for packaging therein. Isolated PVC Needle Complex (comprising the antibody payload) is contacted with a tumour for intracellular delivery of the antibody (said tumour cells being characterised by having high MDM-suppression of p53 activity for MDM inhibition). The tumour is suppressed by the activity of the anti-MDM antibody.

(2) A PVC Needle Complex is used to (intracellularly) deliver anti-tumour peptide vaccine to activate the MHC-I dependent cytotoxic T-cell lymphocyte (CTL) response. A tyrosinase-related protein 2 (TRP2) peptide vaccine is delivered for enhancing cross-presentation to CTLs occurs and antitumor effects against TRP2-expressing tumours. The tumour is suppressed by the activity of the peptide vaccine.

(3) A PVC Needle Complex is used to (intracellularly) deliver a nuclear factor-KB inhibitors (which are used for the control of inflammatory disorders, such as rheumatoid arthritis) to a cell. The cell subsequently demonstrates a reduced expression of pro-inflammatory cytokines.

(4) A PVC Needle Complex is used to (intracellularly) deliver a T3SS payload (which inhibits NF-kB and MAPK pathways). This is completed with an isolated (purified) PVC Needle Complex, without any need for the PVC Needle Complex to remain associated with the bacterial cell from which it derives.

(5) A PVC Needle Complex is used to (intracellularly) deliver, to a cell, anti-apoptotic peptides including BH4, the Bcl-xL-protein, and/or a peptide inhibitor of c-Jun N-terminal kinase (which can protect the heart and brain against ischemic injuries (a restriction in blood supply to tissues, causing a shortage of oxygen and glucose needed for cellular metabolism)). For example, Jun-kinase inhibition via a 20 amino-acid binding motif of the JUN kinase is sufficient. A release of e.g. cytochrome c in the cell is inhibited.

(6) A PVC Needle Complex is used to (intracellularly) deliver nicotinamide adenine dinucleotide quinone internal oxidoreductase (Ndi1), the single-subunit yeast analog of complex I (which provides significant cardioprotective effects) to complex I-deficient mutant cells. The Ndi1 protein is correctly targeted to the matrix side of the inner mitochondrial membranes, and restores the NADH oxidase activity to the complex I-deficient cells.

(7) A PVC Needle Complex is used to deliver one of two of the essential subunits of the PHOX complex (which are used in enzyme replacement therapy to restore production of ROS in chronic granulomatous disease) to a chronic granulomatous disease cell. A restoration in production of ROS is observed.

(8) A PVC Needle Complex is used to (intracellularly) deliver (e.g. intramuscularly) a myotubularin (which is used for improving local and distant muscle performance in X-linked myotubular myopathy patients). Myotubularin—dephosphorylation of phosphatidylinositol 3-phosphate and phosphatidylinositol (3,5)-bi-phosphate is observed.

(9) A PVC Needle Complex is used to (intracellularly) deliver a recombinase “Cre” (which is capable of excising defined genetic cassettes) into a mouse cell line, in which the genome has loxP recombination sites flanking a stop signal upstream of an mCherry gene. The Cre payload excises the recombination sites, and removes the stop signal, allowing for expression of the mCherry gene in the cell.

(10) A PVC Needle Complex is used to (intracellularly) deliver a ˜15 kDa nanobody (antibody fragment) with affinity for an intracellular component. A nanobody-intracellular complex is detected.

(11) A PVC Needle Complex is used to intracellularly deliver (e.g. into insect cells) an atypical (non-Photorhabdus) polypeptide toxin for insect crop pests and animal parasites. Suppression of the pests is observed.

(12) A PVC Needle Complex is used to (intracellularly) deliver a nuclease (e.g. Cas9 and/or Mad7) into a target cell comprising a guide RNA. The nuclease performs site-directed gene inactivation

All publications mentioned in the above specification are herein incorporated by reference.

Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

SEQUENCES
Where an initial Met amino acid residue or a corresponding initial
codon is indicated in any of the following SEQ ID NOs, said
residue/codon may be optional.

(PAK 1985)
SEQ ID NO: 1
MMREYSNEDDFIKEKTNLVKSENVEADNYLETEYLTYLAKLIGMTERENHHLNSIKLIDDIIELHNDRKGNKLL
WNDNWQDKIIDRDLQSIFKKIDEMVSEFGGLEAYKDIVGENPYDPTEPVCGYSAQNIFKLMTEGEYAVDPVK
MAKTGKINGNQFAEKLEHLNSSNNYVALINDHRLGHMFLVDIPSTNRERVGYIYQSDLGDGALPALKIADWL
KSRGKESINVNKLKKFLNDEFTMLPDNEQKGLIAEIFDLNKDIDSVKSGKIKKDKAVDIYLREYDINDFISNIEKL
KTKLA
(PAK 1987)
SEQ ID NO: 2
MFQNRIRNEKTTQSGKGKTLDRMTDSLYLEIPNVEAVTLAYQKLTSKYRKFDNKTKLILDSSDEFSQLKSEK
QRKGFSKSGLKNNGVSDRKFIYTKNALKNFAAHAGYEHNGHYEDEFVNFKDNNKNLAKGKLFPGISLIERR
KLSIVKNKEGKWEHKETDEAEAYKVTDIEKFISGVRSMYLQGNTFLHAKTEALIRKHIANNENILPTMAGIAGL
HAEVQALNNLFISGDKGTKKREKWKYIRNMLESSIFTQRLTTGQAGKDFAACHNCSGILSSPVNVITGKVES
AGDNFLSTLSRYKTSQESPI
(PAK 1988)
SEQ ID NO: 3
MEREYSEKQKNPSKLSRKTAISERIAALERSGLSNSNQPVPQFARPYTSNRPVVNINPGRSSIAVATANSTS
PVNIPTPAPASPDKLLPSTSCDTTSSILIVGKYNLELTSQGKIVVFRGDNRTPEQIVAAGGFYPWSKQDVGKI
KKELIDEFIEIGPSAHMMGHVRSPNKNYVSTGMNMDSGGFGEQSNYLYKMEIPGLKPQDMNERTLGEKIRQ
DKRGINYPHFLMSHLTLAESEFVAMIPARSEELTFITPIPLSYITSYRKRGTNTWLPMPLKK
(PAK 2075)
SEQ ID NO: 4
MSNYEYDIVTQHDTYQIKDNEYTVVNGKYWQYEQEGNKNNNKVSISLMKENQNDPVWITSDIKEISLYIIENL
FSYHKFSAELQHTLKNAVKAVFNEYSEIKYSELLHNINNIFNLFFIKIYNTSDIDTAINILTAKIEIYDKLEKINQDK
TDSNNTNVDIWEELGINAEEPLLKIYRQAFSTGDIDDEVYSDALLTFMSDGNLELGDKEKSDYNQRIKDKTDL
FESYKKGIEKVASLITTNNINPGIPITYPETEKSINIGDDLLLAQLAKEEIALKKQNRTEYSQQDIFELQTLQAAK
YHLLILSSLGALLYQIAPNVEKMTKGHGDYRDIIFSQEQAESLFKKHNIQYDTNHVLSQESKHIEMEGCIILTAA
IIYRMRKENATVEQALNYSTLETIKLFENDKKKLNPFNTNNVKPAGYFSFIDFKKRDKFDSQYNFNEQFNVYK
NKYSHYESISFSKLILSSPAAQLTAEEIVNPPEEAFLYSVEQGMGNVAMIKMYQGNWLVISTIQGGVKAKKYS
RQQVDSNPTLRAMSKPNALFLIERKMETGMGILMPNMMVNTGKRLFPTGYERAKTLSGFAETSRYKNSYN
AFWNDYYGITSGMNVGISFTGSPKFNFYKEENLLSVTATIIQQGLNDIAIKSKQALDITSGWHIAATILIPFYNVI
YKSTTDSEYELTGEDIGSIVFDTANVLLVVATLGMSLTESMAAKVTQTTLRLRQAGLTGRALITAVVRTLPEH
GIITLRQSSGIILGGLIDLIEPLPIRSTLTLTYRGVINAVGAMRNSIKLEKSFADIFGKSTRGLGKLKNEWKVSNL
PLEEIVPHSNGGEIYKGIYSIRPTNPETAVKQNFYIKEAGANYQVKWDDANHTWRVVNPTYPEQFSYWPAV
KLDKNGHVWTHADVSNKFLILEQSKRIDQELEAAHSNINNDNILDAFIHINTAFKDCERYDIDKLSDITDTLTHF
FEKSLKPGDKKAIFSTEIMSIQQAWIREVILPLQNNSSISIEKINAIKTELPYLLRKTFPIESQLPNQLVANKIALAI
EEIPNTRIPKYTSGNISKTVQYTSLLENNHVDIPPVGITITGNDTFINQVTRVLSEIDEIPSGNIVIQELEKQGLNI
QPPTMNDIVREKNGQFYANNSAGSHIAFDPENHLIGTEEKLIDEPWRTREPAIALYHEMLHIYYNRYPTWFT
SIDNKVIDQKVSGGFSLLEESRIVGTKYYVNDKNTLFDFNDSDYLLENNSALLTENRFRAEYAIFKNKSEYVIR
PYSGKGDSQIPLTKTKININESHRNVMGVGSGKPEKMPNESATDYRNRVREWRKANKQPEADIGTGDMRK
TKAEARVKLLKENYPQFEPQKIELGGAFQLWTVPNEPANKLMLSSHGYFFSDSAATQVPAGKTIQFLGPHG
KTLLEAPENPLYSPFDVTLGNSGFTVQPYATIESGNKAGLGSVKIGDKTFTVNDIQNIATDDVENYLLATGVE
ANASNHGKVRNYGIKYYEKMPDEEVKAAIWKNRADETSTHKYDALLVSPEAGNRKKLSDIFALMKTDERMS
KYDEITFVACREELNRINMKSIHDTGLGGGYEPKLEPTVILSRRRREATFTADGAIIYSIIAVNLHHNFITEEIVG
IAPFLFINN
(PAK 2077)
SEQ ID NO: 5
MEHEYNEKEKQRNSAIKLNDAIRNNEENMDMTSPLELNFQNTNRKSRGLRERFSATLQRNLPGHSMLDRE
LTTDGQKNQESRFSPGMIMDRLMHFGVRTRLGKVRNSASKYGGQVTFKFAQTKGTFLDQIMKHKDTSGGV
CESISAHWISAHAKGDSIFNQLYVGGKKGKFHIDTLFSIKQLQMDGYLDDEQSTMTEYWLGTQGMQPNIQR
NDDTDEHSSKVVGETGNRGTKDLLHAILDTGDKGSGYKKISFLGKMAGHTVAAYVDDQKGVTFFDPNFGE
FSFPDKTSFSHWFTDDFWPKSWYSLEIGLGQEFEVFNYAPEAP
(PAK 2892)
SEQ ID NO: 6
MPNKKYSENTHQGKKPLMKSEANNEHDIQNSSLGIGLDLNSMMGNSSTSLSHIQDYSFWKENISEYYKWM
VVVKAHLKQLDWTLKSMDSPESAGTNIAKNTGTTALQTLLNTGGSIAGAAIGGAIGSAIAPGVGTIAGMGIGA
LAGTGLNYLNDTVIEKLNEKLEIAYPYPKTRNMIFDINNYDKNPIIKAIKKKTNKDNLKVTAGSSLTSQLVGKVT
SPIKFPAYKLADLAIALAGLSSDKARHILDFTDSIREVLNESHSDAVAFMRKNYGDNAMGLAGLSSRIK
(PAK 2893)
SEQ ID NO: 7
MEREYSEKEKHKKRPIQLRNSIEQHEEETANNSLGLGLDLNQATNPPKVPKDNYNEENGDLFYGLANQRG
RYIKSVNPNFDPDKINSSPMIIDVYNNNVSNTILNKYPLDKLVKLSGNPQKYANNIKVENSLQQDVASSKRGW
YPLWNDYFKTGNENKKFNIADIYKETRNQYGSDYYHTWHTPTGAAPKLLWKRGSKLGIEMAASNEKTKIHF
VLDGLNIQEVVNKQKGSTPLEQGRGESITASELRYAYRNRERLAGKIHFYENDQETVAPWEKSPELWQNYI
PKNKNQNESSTPQRNNGTLYRLGGPFRKLRASLRKRS
(PAK 2894)
SEQ ID NO: 8
MMEHEYSKEEEKKRQQSKPNNATHDESNLPLELEKHFNARTPATAHSKWFTYENDTEVELTTERIKEIFSN
KQPKIIIAGDGHNKPPFQYAKNIPDVNSSFDAGTLQLYIEATDEQINENNPEYIPKEFMAKPGLFTNKNRRAEI
VGWEDSELSNAMKEMFELSDKSTREKLTPEETSSFYKLHETAIRHFFRPEFNQLRDEFFEILAKAGSNRELD
KIALEMIGFTSGTWRDEYINPTLAEKIAKHAAEKENHTFVVSIGDAHLSENPMQEYLNKRRNGGEFKHQIIFT
RDKRPILPDNMKTGNKNS
(PAK 3525)
SEQ ID NO: 9
MLKYANPQAVPTQRTKNTAKKPSSSSSFDGQLELSNGEWSKHSEMGLKRGGLINSIRRRIARNGNIGRFNE
LIDSEAKKWPSEPVDKNIHMIWIGTRNISEKNIKLSIDTAKKNPDYNTSIIYDSGISGHEGARNFMLEKFEGSN
VNXSLAFPKGIGVMREYAPEAGKATAFPNTPIAVTKNNPIINKTLDLAVGNYQRGEKNVLKLAGPDVFTQALY
QEIPGLNSKVLNAQLDQFELAKRQALGLPLEKPKSFADEKLTSVEKEKINRPYQSMRGLSGHVMNGADHS
WAVDTEVLGH
(PAT 00148)
SEQ ID NO: 10
MMREYSNEDDCTKEKTNLVKSENVEADNYLEMEHLTYLAKLISMTERENHHLNSIKLIDDIIELHNDRKGNKL
LWNDNWQDKIIDRDLQSIFKKIDEMVSEFGGLEAYKDIVGESPYDPTEPVCGYSAQNIFKLMTEGEYAVDPV
KMAKTGKINGNQFAEKLEHLNSSNNYVALINDHRLGHMFLVDIPSTNRERVGYIYQSDLGDGALPALKIADW
LKSRGKESINVNKLKKFLNDEFTMLPENEQKGLIAEIFDLNKDIDSVKSGKIKKDKAVDIYLREYDINDFISNVE
KLKTKLA
(PAT 00149)
SEQ ID NO: 11
MIFKMLNLAVFYLLGNIFHYLICQKFICYFCSVLKSVTMFLTKVAVQIALYLNILPTMAGIAGLHAEVQALNNLFI
SGDRGTEKRENWKYIRNMLESTIFTQRLTAGQAGKDFAACHNCSGILSSPVNVITGKVESAGGNFFINIISI
(PAT 00150)
SEQ ID NO: 12
MEREYSEKPKNLSQLSRKTAISERRAMFERNASSNNEQPVPQFARSYTSNRSVVNINPGRSSIAVVTANST
SPVNISTPAAASPDKLLPSTSCDTTSSTLTVGKYKLELTSQGKVVVFRGDNRTPEQIVAAGGFGEQSNYLYK
MEIPGLKPQDMNERTLGEKIRQDSRGN
(PAT 00152)
SEQ ID NO: 13
MKYDPRLRTVWEDDFDYEKNFKKQTDYINYKDLEKQLKENVDYYALLDENEAIIFLKELGCDIKSFLNDTAFP
VTDVLSNFAGNIKDALGVFKVAKNFKPINIGIFTYIINELKGKGIKAIEYLGKNGERYIKLTDRPGIRKYLNATRY
LINNKKIMEVGIGSVAMEGSIVKGARFGVIYSAAYRSVELMFKSEYDLTNFFVNLSMDMAKIIVATIIAKSTVAA
ATSFVVTAALSTTAIAIGVFIIGALVVWGLMWLDDEFKISETIIRRLKEHKVKTPISTYHSDQIFNAWGRYYRG
(PAT 02308)
SEQ ID NO: 14
MPNKKHSENTHQGRKPLIKSEANNEHDIENSSLGIGLDLNSTIGNNSASLSQIQDYSFWKENISEYYKWMVV
VKAHLKQLDWTLKSMDSSESAGTNIAKNIGTTALQTLLNTGGSIAGGAIGGAIGSAIAPGVGTIAGMGIGALA
GTGLNYLNDTVIEKLNEKLEIAYPYPKTRNMIFDINNYDKNPIIKAIKKKTNKDNLKVTAGSSLTSQLVGKVTSP
IKFPAYKLSDLAISHNRALAGLSSDKARHILDFTDSIREVLNESHSDAVAFMRKNYGDNAMGLSGLSSRIKGE
KLTLATLARTRNKIENRINSINKQTLKLSSKNSNE
(PAT 02309)
SEQ ID NO: 15
MEREYSEKEKHKKRPIQLRNSIEQHEEETANNSLGLGLDLNQATNPPKVPKDNYNEENGDLFYGLATQRGR
YIKSVNPNFDPDKINSSPMIIDVYNNNVSNTILNKYPLDKLVKLSGNPQKYANNIKVENNLQQDVASSKRGWY
PLWNDYFKIGNENKKFNIADIYKETRNQYGSDYYHTWHTPTGAAPKLLWKRGSKLGIEMAASNEKTKIHFVL
DGLNIQEVVNKQKGSTPLEQGRGESITASELRYAYRNRERLAGKIHFYENDQETVAPWEKSPELWQNYIPK
NKNQNESSTPQRNNGALYRLGGPFRKLRASLRKRS
(PAT 02310)
SEQ ID NO: 16
MMEHEYSKEEEKKRQQSKPNNATHDESNLPLELEKHSNARTSATAYSKWFTYENDMEVELTTERVREIFS
NKQPKIIIAGDGHNKPPFQYTKNIPDVNSSFDAGTLQLYIEATDEQINENNPEYIPKEFMAKPGLFTNKNRRA
EIVGWEDSELSNAMKEMFELSDKSTREKLTPEETSSFYKLHETAIRHFFRPEFNQLRDEFFEILAKAGSNRE
LDKIALEMIGFTSGTWRDEYINPTLAEKIAKHAAEKENHTFVVSIGDAHLSENPMQEYLNKRRNGGEFKHQII
FTRDKRPILPDNMKTGKKNS
(PAT 02956)
SEQ ID NO: 17
MSNYEYDIVTQHDTYQIKDNEYTVVNGKYWQYEQEGNKNNNKISISLMKDNQNDPVWITSDIKEISLYIIENL
FSYHKFSAELQHTLKNAVKAVFNEYSEIKYSELLHNINNIFNLFFIKTYNTSDINTAINILTAKIEIYDKLEKINQD
KTDLNNTKVDIWEELGINAEEPLLKIYRQAFSTGDIDDEVYSDALLTFMSDGNLKLGDKEKSDYNQRIKDKTD
LFESYKKGIEKVASLITTNNINPGIPITYPETEKSINIGDDLLLAQLAKEEIALKKQNRTEYSQQDIFELQTLQAA
KYHLLILSSLGALLYQIAPNVEKMTKGHGDYRDIIFSQEQAESLFKKHNIQYDTNHVLSQESKHIEMEGCIILTA
AIlYRMRKENATVEQALNYSTLETIKLFENDKKKLNPFNTNNVKPAGYFSFIDFKKRDKFDSQYNFNEQFNVY
KNKYSHYESISFSKLILSSPAAQLTAEEIVNPPEETFLYSVEQGMGNVAMIKMYQGNWLVVSTIQGGVKARK
YSQQQVDSQPTLRAMSRPNALFLIERKIMIGIGIFMENQIVNTGKRLFPTGYERAKTLSGFAETSRYKNSYNA
FWNDYYGITSGMNVGISFTGSPKFNFYKEENLLSVTATIIQQGLNDIAIKSKQALDITSGWHIAATILIPFYNVIY
KSTTDSEYELTGEDIGSIVFDTANVLLVVATLGMSLTESMAAKVTQTTLRLRQAGLTGRALITAVVRTLPEHGI
ITLRQSSGIILGGLIDLIEPLPIRSTLTLTYRGVISAVGAMRNSIKLEKSFADIFGKSTRGLGKLKHEWKVSNLPL
EEIVPHSNGGEIYKGIYSIRHTNPETAVKQNFYIKEAGANYQVKWDDANHTWRVVNPTYPEQFSYWPAVKL
DKNGHWWTHADISNKFLILEKSKRIDQELEAAHSNINNDNILDAFIHINTAFKDCERYDIDKLSDITDTLTHFFE
KSLKPGDKKAIFSTEIMSIQQAWIREVILPLQNNSSISIEKINAIKTELPYLLRKTFPIESQLPNQLVANKIALAIEE
IPNTRIPKYTSGNISKTVQYTSLLENNHVDIPPVGITITGNDTFINQVTRVLSEIDEIPSGNIVIQELEKQGLNIQP
PTMNDIVREKNGQFYANNSAGSHIAFDPENHLIGTEEKLIDEPWRTREPAIALYHEMLHIYYNRYPTWFTSID
NKVIDQKVSGGFSLLEESRIVGTKYYVNDKDTLFDFNDSDYLLENNSALLTENRFRAEYAIFKNKSEYVIRPY
SGKGDSQIPLTKTKININESHRNVMGVGSGKPEKMPNESATDYRNRVREWRKANKQPEADIGTGDMRKTK
AEARVKLLKENYPQFEPQKIELGGAFQLWTVPNEPANKLMLSSHGYFFSDSAATQVPAGKTIQFLGPHGKT
LLEAPENPLNSPFDVTLGNSGFTVQPYATIESGNKAGLGSVKIGDKTFTVNDIQNIATDDVENYLLATGVEAN
ASNHGKVRNYGIKYYEKMPDEEVKAAIWKNRADETSTHKYDALLVSPEAGNRKKLSDIFALMKTDERMSKY
DEITFVACREELNRINMKSIHDTGLGGGYEPKLEPTVILSRRRREATFTADGAIIYSIIAVNLHHNFITEEIVGIA
PFLFIDN
(PAT 02957)
SEQ ID NO: 18
MEHEYNEKEKQRNSAIKLNDAIRNNEENMDMTSPLELNSQNTNRKSRGLRERFSATLQRNLPGHSMLDRE
LTTDGQKNQESRFSPGMIMDRLMHFGVRTRLGKVRNSASKYGGQVTFKFAQTKGTFLDQIMKHKDTSGGV
CESISAHWISAHAKGDSIFNQLYVGGQKGKFHIDTLFSIKQLQMDGYLDDEQSTMTEYWLGTQGMQPNIQR
NDDTDEHSSKVVGETGTKGTKDLLHAILDTGDKGSGYKKISFLGKMAGHTVAAYVDDQKGVTFFDPNFGEF
SFPDKTSFSHWFTDDFWPKSWYSLEIGLGQEFEVFNYAPKEP
(PAT 03171)
SEQ ID NO: 19
MFKYDTSEKMAKFGKGKTSDGMLLDTLYLEIPDEKAVMSAYKSQILDELRNFSEKTHSFFSGKKPLYSKKYL
ANLAAHAGYVHVTDYNSIGNYKDGFVNFKDNSRNLAEGKLFPGIRLIKRPKLSIVRDKETERWKKQESDEAD
AYEITDIESFISGVRDMYSRANVDLHPVIESLIRNHIVNNDHVLPTMAGIAGLHAEVQALNNLLILADGRAGKIV
GGRKIEEYMQDMLKSFIFTQRLTTKQAGNDFAACHNCSGILSVPANVITGKVASAGSNFSLILSRYKNSQES
PI
(PAT 03172)
SEQ ID NO: 20
MLKHANPQTVSTQRTKSTAKKPSSSSSFDRQFELSNSENQPGEGNKDWTIKGWRQRFADRSLNKGHISPL
MNKGLLVGSEEALINVPVVAHRYDSSHQLTDAGPLKADSHSNNLDPFYGVVTGFRGDQVTSSESGSGSIG
GHWGKNTLDSNITGINVVNGASGTVGIRIALKDIQHGAPVIVTSGALSGCTMVYAVKNGYFFAYHTGQKPGD
KEWKTGRQGVVATYRSHQALSPDSEPMAVGEQNNDLVNIFASYDQGIITYMGKPGVIIDNTAENVGVFNYD
EVKLEKPDIRAGYSYALLAKDDKGKVNVKVLSEDVIVPLGNKGKTIKAINSLKKRLL
(PAT 03177)
SEQ ID NO: 21
MPRYANYQINPKQNTKNSHGKSSSSNFSSGYFSSSNNSLDDSLIRQQVKREFIWEGHMKEIEEASRLGNFA
VSFRAAGGPTLRALGKGAAAKGHDILEKTIKPGSINKAYPKDEASNVIKKVQEAGIEGYVGHWDKKTGRLLGI
YMSSGHGLSDEQVNGKIYPIDLNNLEASLSALKTKENWAALPFTGDYDMHDMISFTGQPHSVPSNSSEERK
HDRINRLVARSDPNRPFGDIEHNVIRHGAQVSYPAFAMDKEKEEIKKHGGIVKAVAEPGEFPVAIVSKGKWTI
ANNIDELNQFYNSIGAKMKVSWKPGAENPGFVSNPQRPGMARFSRKR
(PAU 02009)
SEQ ID NO: 22
MMREYSKEDDCVKEKTNLAESENVEADNYLEMDCLNYLAKLNGMPERKDHSLNSTKLIDDIIKLHNDRKGN
KLLWNDNWQDKIIDRDLESIFKKIDEMVSEFGGIEIYKDIVGENPYDPTEPVCGYSAQNIFKLMTEGEHAVDP
VKMAQTGKINGNEFAEKLEQLNSSNNYVALINDHRLGHMFLVDIPSTNREKVGYIYQSDLGDGALPALKIAD
WLKSRGKESINVNKLKKFLSNEFTMLSESEQKELIAEIFDINKDIANVKLGKIKKDKAVDVYLREYDLNDFISNI
EKLKTKLV
(PAU 02010)
SEQ ID NO: 23
MPIIGHKEDLIRTERSSVDLTRSSNNRQTDNLELNIPQHKRDNKDIEHAVIYGFSQHRGPEMQKAFADNKNP
VTIDEYNAGLGIMGELSLSDYFRISQDLKENRLPELNEKNIQNHSLKYFDAMGVNMKSADPNVKEEAKEQQ
RAYTRSWGFYMMENKEKLDIQSKINNLIPKKKSFFSKSPGEDEYKKLDEFILKNSNGSNLTIPKQRKILMKFA
SAKNAVDVTKNLSGEEQTWLKDIIATAFFRQTSKLGMSWFIEQLASPDFRFVIVGFNGEELTTDQIRSNKPW
KHGNRRKEGASEYAEPITFSEIRHAHRKGYDSKINFIKK
(PAU 02095)
SEQ ID NO: 24
MISTFDPAICAGTPTVTVLDNRNLTVREIVFHRAKAGGDTDTLITRHQYDLRGNLTQSLDPRLYDLMQKDNT
VQPNFYWQHDLLGRVLHTVSIDAGGTVTLSDIEDRPALNVNAMGVVKTWQYEANSLPGRLLSVSEQSANE
AVPRVIEHFIWAGNSQAEKDLNLAGQYMRHYDTAGLDQLNSLSLTGAHLSQSLQLLKDDQMPDWAGDNES
VWQNKLKNEVHTTQSTTDATGAPLTQTDAKENMQRLAYNVTGQLKSSWLTLNGQLEQIIVKSLAYSESGQK
IREEHGNGVVTKYSYEPDTQRLINITTQRSKGHVFSEKLLQDLLYEYDPVGNIVSILNRAEATHFWRNQKVSP
RNTYTYDSLYQLIQSTGREMADIGQQNNKMPTPLVPLSSDDKVYTTYTRTYSYDRGNNLTKIQHRAPASHNI
YTTEITVSNRSNRAVLSHNGLTPREVDAQFDASGHQISLPTGQNLSWNQRGELQQATTINRDNSATDREW
YRYNAGSARILKVSEQQTGNSTQQQQVTYLPGLELRTTKSGTNTTEDLQVITMVETERTQVRILHWSAGKP
NDIANNQVRYSYDNLIESNVMELDTKGKIISQEEYYPYGGTAIWTARNQIEASYKTVRYSGKERDKTGLYYY
RHRYYQPWLGRWLSADPAGTVDGLNLYRMVKNNPIRYQDESGTNANDKAQAIFKEGKKIAINQLKIASNFL
KDSKNSENALEIYRIFFGGHQDIEQLPQWKKRIDSVIYGLDKLKTTKHVHYQQDKSGSSSTVADLNVDEYKK
WSEGNKSIYVNVYADALKRVYEDPLLGREHVAHIAIHELSHGVLRTQDHKYIGVLSSPGSHDLTDLLSILMPP
ANEQDRTEKQRRATGARKALENADSFTLSARYLYYTAQDPNFLSSLRKAHRDFNNKKTDRLIIRPPERR
(PAU 02096)
SEQ ID NO: 25
MEREYNKKEKQKKSAIKLDDAVGNNEENMDMTSPLELNSQYTNRKRPGLRERFSATLQRNLPGHSMLDRE
LTTDGQKNQESRFSPGMIMDRIMHLGVRTRLGKVRNSASKYGGQVTFKFAQTKGTFLDQIMKHKDTSGGV
CESISAHWISAHAKGESIFDQLYVGGQKGKFHIDTLFSIKQLQMDGYLDDEQSTMTEYWLGTQGIQPNRQK
NDNMNEHSSKIVGETGTRGTKDLLRAILDTGDKGSGYKKISFLGKMAGHTVAAYVDDQKGVTFFDPNFGEF
NFPDKVSFSHWFTDDFWPKSWYSLEIGLGQEFEVFNYEPKEP
(PAU 02097)
SEQ ID NO: 26
MVYEYAKTNDRKRKLSTQSDNYEEKSFSPVLDLSRNNQNTPNMEDEYETPQNFINRTGREKLFRAIRMVAS
NKRDPITKDQVSVPPDGNLFTELKDKHLDRAAEYKKLKTWPTHASIIATSPSANTPIAQHVSGDDALSPYIST
GDKPGAVQNTVRNWNGIGPASERRLRPEKTWSPIIEIDVNKLPDTTKIFDLNKPNNTFFSTTNSDIAQNAFAD
KEVLISPEIPGLAITRVINDPEEIKQIANLNPSQSLIEKKNTIPEEKIIFEEKKSVPIHDSDADIPSSSFVFPKRKKP
RNIRSRTDS
(PAU 02098)
SEQ ID NO: 27
MVFEHDKTVERKRKPSIQLGNDKEKSSEQALELPQSKQNNPLLHDLITSNNLRKEAAVFAKQIGPSYQGILD
GLEHLHNLSGNEQLTAGFELHRRITRYLEEHPDSKRNAALRRTQTQLGDLMFTGTLQEVRHPLLEMAETRP
AMASQIYQIARDEAKGNTPGLTDLMVRVWKEDPYLAAKSGYQGKIPNDLPFEPKFHVELGDQFGEFKTWLD
TAQNQGLLTHTRLDEQNKQVHLGYSYNELLDMTGGVESVKMAVYFLKEAAKQAEPGSAKSQEAILLNRFA
NPAYLTQLEQGRLAQMEAIYHSSHNTDVAAWDQQFSPDALTQFNHQLDNSVDLNSQLSFLLKDRQGLLIGE
SHGSDLNGLRFVEEQMDALKAHGVTVIGLEHLRSDLAQPLIDKFLTSENEPMPAELAAMLKTKHLSVNLFEQ
ARSKQMKIIALDNNSTTRPAEGEHSLMYRAGAANNVAVERLQQLPAEEKFVAIYGNAHLQSHEGIDHFLPGI
THRLGLPALKVDENNRFTAQADNINQRKCYDDVVEVSRIQLTS
(PAU 02230)
SEQ ID NO: 28
MKGIEGVIMLSHDILPEKLLVSEKKHENVGSYFSDDIGEQSEQTEVSHFNLSLDDAFDIYADISIENQQELKNK
DNNTNIWSSLGRGDDDHNLKKIINDAFKEKLPQLMEYRRKGYNVIGLDKEGIKKLEGMLKAVPPEIQQPTMK
NLYSAAQELLNTLKQHPLLPENQDMIQQSNLVIRNLSDALEAINAVSKVNQVEVWVEEVHKTNKAQSDRLIAA
TLEELFFKVKDKRLPGSNDDYCQQEREETERKIKDLLLYDGYQLTAEHFKFGRLRKSLLAESRVTRLKLAEY
LEKKSVGILTAARDAKMYAMKILLAQTRNNGFNAKDLINAGQVNDRLLSFQQYARHIRAVDGEIDGIILSNPLV
VACIKETNDEPAHIKIARAILPVSEELGTVSKVLRETKEKVQPSKPKEELNHPHQDVWVNRGDELWKYIKKTS
WNIKETSVHVTQMVGYEASKTASRAKHKLKESSYSESINGAVKGTALLLLDEIQQAENRIRQIPQFAWDVQE
AVEQHSSVIQRTAYPDELPELSELLNEQLKHEEARWQAVKKQSRDKLQELIAPITRLAQEKWAQDLYFQLG
EELRKERQDRWKDIQQFDEIMAEAVGQFAEMARELDSEAVRLAEHGHSGGKELQEKVAKWLRDLSKLKGK
VKAGVAKITGTSLDNFSRSGMLARGMSEWAEDLKQSYLQETLQEGSAVAAELFERTLMEVVEENRTHFAK
ESDPEAERFLKRLALALKHAAENTTVYPPTPEEILAGSRSLPEDIRHWAEKKVVSGAISAAFRGGFKLVTGTF
SLPVRVVIRGAKTGGTLYRGVRAINRSVRLGQGPATQVKSKFINQELSKTAFRLTLSLSPLVAWGMAASITA
GRLYNEKDYPEKIIKNIVIDLPEELLWIGGYAGINAAIRAHAEKAIQQAIQHALDEQADKLALRINKEIAGKSADV
NVEIIPQETSVSPAETAQSTPEPLSDFASTSQLTMPELIDIQDNNSAQQPKVRRKRDVSVESEISIDNLNIINA
NTREDKVNSEIKSELRSELKRFENSDANSPMSDVERAIFIDLFLYKNKYEVSESQQDYKNTWLKFRRELESQ
ENKEIKEYLRFRSIIEAYEIYDKKRLDDDTIPEAGTIIKEVIDFFQKLKKENPITFMKLAEAMVKFQYYYEEEDEN
EDRYFKMAEIYYFLNKTENEKKSKTFHLDIIDKYPNENNRLLDEFFLNKNNNNPDLDEIIYKLQSMQEKYRES
YEMLSKVENIHQVLSDDSKNEENIFLDNRIIAAQVFDGSINISLQDKKKWLNRYDQIRNEEGSDGWKLMHIES
ILINLRRINTAINLTAMKSESALLLIDKLLNFQKKARENILHISETPHEDFTSYSQFKTRKELGNDDSKYYAQFD
NYKDNHDAEKEAKEILSQVVARASLSFSELFDKVESIKLFSFVYKNRDGGAPLAAPGRTVVIKFPGKDTGGL
VISNLFLRNHVKRISTKEMEDLKPLTEGMYTRATQHRSLGSYYHIGSQSEHTNALEILSGMNKEELKTHLKK
QGIWFGEPALFSNEYPKQENTGHLENTTLKNAIIGVSTIQNNAAANYLRSTMYESTGWEKLGDRFIPFYEIGR
RKHYDREYEINSEQLTLDIITSIAIAYPAARGIVATIRSSAIPSILKSGLRGSALFKSLSLELGKMGFNASKVFGG
AVYELIEPYPINSHLNRHNVFNKVKDTAWEFHTDVGLKGGGLKDFIDRFTKEPKEITISGYKFKRIKYNQENF
DTMQRMALDYAYNPDSKGKIAQAQQAYKTGKEDYNAPQYDNFNGLSLDKKIERYISPDTDATTKGVLAGK
MNESIKDINAFQTAKDAQSWKKSANKANKVVLTPQNLYLKGKPSECLPESVLMGWALQSSQDAKLSKMLM
GIYSSNDITSNPLYKSLKELHANGNASKFNASATSISNINVSNLATSETKLFPTEISSVRVDAPKHTMLISKIKN
RENKIKYVFYDPNYGMAYFDKHSDMAAFFQKKMQQYDFPDDSVSFHPLDYSNVSDIKISGRNLNEIIDGEIP
LLYKQEGVQLEGITPRDGIYRVPPKNTLGVQETKHYIIVNNDIYQVEWDQTNNTWRVFDPSNTNRSRPTVPV
KQDTNGEWFKHSETGLKGGGPIDDIRKYIARKSAIKIFNQSINYSATKWPPEPIDKNIHMIWIGTKNISEKNIKL
SIDTAKKNPDYNTSIIYDSGISGHEGAKKFMLEKFQDSNVNIIDFRKKSYFSQLKQEPSFAYYEQVIAENKYAQ
ASDILRLLVLKYEGGIYKDIDDIQVKGFGSLTFPKGIGVMREYAPEAGKATAFPNTPIAVTKNNPIINKTLDLAV
SNYQRGEKNVLKLAGPDVFTQALYQEIPGLDSKVLNAQLYQLELAKRQALGVPLEKPKNFADEQLTSAEKE
KINRPYQSIRGLSGYVENGADHSWAVDTNIPSTSTQTSTIVTPLAPKTEMLPPVPSSSTKSSTSAPVLQEKIS
YNLATDIDATDYLNQLKQKTNINNKISSPAGQCESLMKPVSDFMRENGFTDIRYRGMFIWNNATEQIPMNHF
VVVGKKVGKDYVFDVSAHQFENKGMPDLNGPLILAAEDWAKKYRGATTRKLIYYSDFKNASTATNTYNALP
RELVLESMEGKTFITSPNWYQTFKRTHNIHPEVTVSDPATFSLNYSVNPTAENLSPPPPPPIPSHGQVPKTV
TPPPPPMRSPLSLSQPLERLPANKTKPIGFNPGENKASFSKLEEAGKHYYKDDKSRQAAPVNTMSDFDNRY
LSHTTEAPAPSNVAHLAPGNIYNTKVTAKGAEKPAYDIYISKDGESLITSSSYKVDDITTDSKFGKPLPYSEIM
FNSLKKSGVDPKNLKRSVQASIENKVTQDVISAIGTRIQRGQVIRVSPTENPDAFYTLLGTDNCKATLHMLNQ
HAEEFGHKWTSIEFKGTGYLVMNIGTSTQTSTIVTPPPMPGTSQLVQ
(PAU 02805)
SEQ ID NO: 29
MPNKKYSENTHQGKKPLIKSEANNEHAIDNSPLGIGLDLNSILGNNSASLSQIHDYSFWKENISEYYKWMW
VKAHLKQLDWTLKSMDSPESAGANIAKNIGTTTLQTLLNTGGSIAGGAIGGAIGSAIAPGVGTIAGMGIGALA
GTGLNYLNDTAIEKLNEKLEIAYPYPKTRNMIFDINNYDKNPLIKAIKKKTKKDNLKVMAGSSLTSQLLGRITPI
KIPAYKLADLAVSHHRALAGLSSDKARHILDFTNSIREVLNESHSDAVAFMRKNYGDNAMGLSGLSSKIKGD
KLTLDTLARTRNKIENRINSINKQTLKLSSKNSNE
(PAU 02806)
SEQ ID NO: 30
MEREYSEKEKHKKHPIQLRDAIEQHAEETANNSLGLGLDLHQAINTPKVPKDNYNEENGDLFYGLAAQRGR
YIKSVNPNFDPDKTNSSPMVIDVYNNHVSNTILNKYPLDKLGKLYGNPQKYAKDIKVTNSLQQDVAASKRGW
YPLWNDYFKAGNENKKFNIADIYKETRNQYGSDYYHTWHEPTGAAPKLLWKRGSKLGIAMAASNEKTKIHF
VLDGLNIQEVVNKQKGSTPLEQGRGESITASELRYAYRNRERLAGKIHFYENDQETIAPWEKSPELWQNYIP
KNKSQNESSTPQRNNGALYRLGGPFRKLRASLRKRS
(PAU 02807)
SEQ ID NO: 31
MVHEYSINDRQKRHSFSSANPIDPEVTNRENSRHRFPKDNYNKGHGDLFYGLAPERGKYIKEANPKFDPNN
PENAAMIIDVYNDEISRVILNNNANKISTNRLLNFIYNFRKNRLENLMKNPEKYAKDIKVKDNLRENISPKKIEK
YPLWNDYFEAGIRNKKFNIAEIFKETASQYNSDYYHAWHIGGNSAPRLLWKRGSKLGIEIAASNQRTKIHFIL
DGLKIEDWNKTKGPAPLKAGPGESITASELRYAYRNRARLAGRIHFYENGKETIAPWDKDPELWQKYTPKN
RSGMEL
(PAU 03332)
SEQ ID NO: 32
MLKYANPQTVATQRTKNTAKKPPSSTSFDGHLELSNGENQPYEGHKIRKIKGLRQHLADRSLNKGHISPLM
NKGLLVGSKDVSIDIPVIAHRYDSSHQLTDAEPLKADSHSNHLDPFYGVIAGFRGDQVTSSESGSGSIGVHW
GKNTLDSNIMGVNVVNGASGTVGIRIALKDIQHGSPVIVTSGALSGCTMVYSVKNGYFFAYHTGQKPGNNE
WKTGRQGVVATYLSHQALSPDSEPMTVGEQNNDLVNIFANYDQSVITYMGKPGVLIDKMAENVGVFNYDEI
KPEKPAIRAGYSYALLAKDDKGKVNVKVLSEDVIVSSGKQGNTVKAINSLKKRLL
(PAU 03337)
SEQ ID NO: 33
MPRYANYQINPKQNIKNSHGKSSSSDFSSGYLSFSNNSLDDPFIRQQVKREFIWEGHMKEIEEASRLGNFA
VSFRAAGGPTLRALGKGAAAKGHDILEKTIKPGSINKAYPKDEASDVIKKVQEAGIEGYVGHWDKKTGRLLGI
YMSSGHGLSDEQVNGKIYPIDLNNLEASLSALKAKENWAALPFTGDYDMHDMISFTGQPHSVPSNSSEERK
HDRINRLVARSDSNRPFGDIEHNVIRHGAQVSYPAFAMDKEKEEIKKHGGIVKAVAEPGEFPVAIVSKGKWTI
ANNIDELNQFYNSIGAKMKVSWKPGAENPGFVSNPQRPGMARFSRKR
(Plu1651)
SEQ ID NO: 34
MPNKKYSENTHQGKNPLMKSGANNEHDLQDSPLGIGLDLNSMLVNSSTSLSQIQDYSFWKENISEYYKWM
VVVESHLKQLDWTLKSMDSPESAGTNVAKNMGVTALQSLLNTGSSIAGGAIGGAIGSAIAPGVGTIAGAGIG
ALAGTGLNYLNDTAMSKLSKKLEIAHPYPKTRNMILDINNYDKNPIIKAIKKNVNKDNLKVTAGSSLTSKLVGT
VTSPIKFPAYKFAELAVSHHRALEGLSDDKARHILDFTNSIREVLKESHSDAVAFMRKNYGDNAMGLSGFSS
KIKREKLTLNTLAKTKNEIENRINSINKQTLKVSSRSRNE
(Plu1671)
SEQ ID NO: 35
MLSTEKHNKDTKHPRNREKKFSIQPENSTQDDEDIKNNSLGVGLDLDQMIRNTSSTLTNAPQKPEDGYYYHI
SRGNNLQSFLQNGFKPQGSPGPTLSEEDFSRRKIGIIKLIYSIIATTINKNRKAKKISKDNFLMPQEFWHEFKN
FYQNIPTQTNIDDQLLKKSITESIDKLDQNKFMEKHSDRKQTIINNEREAILQQDERINEIISSRAKMIQQREAE
NTEGYIYLAPHKNTLLEYMKHLQEEKNLFLILAVKEDIFTEKGLEQDPQEPHGAVRYKGALSTEELNFVNQE
GQICAIPASIGEMDYGDFILNQQQVIDFCKK
(Plu1672)
SEQ ID NO: 36
MPINDLKKKFEISPQAAQAIGAPARSNSSKQAEHQTEHLELDTSKNRRDRKDLNAQATPNQQHTKKLETEV
NNGGNKSKAQAHTPDLVMKKESSVTPNTRKSPNEKIKAEDIFHRYKDRFSPSDRELPFEIMNEITNNGIAFS
SEKAPESHLDKVKDKKFTLRHYTSGNGQEKPTFNEIGSNFNLVNEGIKTLKRTQGSNTNEDDWNRLGNTAF
TFFLLAIDGEVSDRKFLSNTTHFAEIDIENPAELKELGLDETEFFASPDLLHEKNLSQAPAVKGKLSDLKSLLL
KQSGIKPVQLQSLGAKGILERIDSKFNGSLEIKIPGNVKVKEWKKVEK
(Plu1690)
SEQ ID NO: 37
MPNSKYSEKVNHSANGAEKCSIHSNQYNINNCTLGLGLDLNKKLRTGNERNIEGAQPFIPFPSKQKQYSTSP
IAMADILNESALTSQPIITDLINPQKIKMSDGVKNILNNKEGGGDLVFKALQIKPSDETLPFNALKIVDTYQEEM
PNKDMSISAYWAPQGGYVDIPAQPDISRHPQYVFTPNFSGCSFVVDKMNEDTLRVRHVQGGQEDVEYNN
QNIDHGMGMITAMEFRDYGYHEADDKVIENTYGFAFLKFNQEKKQWQLHYQKIAAAPNIINIKTKSSWLPFS
KPSIEADTFTFKNMKVPGYSRKNINNN
(Plu1691)
SEQ ID NO: 38
MPKLTELLSRFENPIQNQPNHISKKNPISNSKVLNNSEEKTAPLELKHDDSKIKSQVSIPNLVKKNEKPAASNT
PNNSHEKVKAEDIFNRFKSKFDPYDRELPFDIMNKITNNEIKFSSEKSKDDYLAKVKDKKFTLRHYTAGTGQE
KPTFDEISSNFNLVNKGIKTLNRTQGSNTNEDDWNRLGNTAFTFYLLAIDGEVSNRKFLSNTTHFAEINIEDS
EELKELGLDQAEFFASPDLLHEKNLSQAPAVKGKLSDLKSLLLKRSGISSVQLGRLDAKAILKSIDNEFGNSL
EIKIPGNVKVNKWNKI
(Plu1712)
SEQ ID NO: 39
MPRYSNSQRTPTQSTKNTRRTSPSSNSSTEHLSLSNAPTNDSSVRQEVKEKFIWEGHWEGHMEAIEKASIL
GNFAVSFRAAGKPTLEALGKGAAAKGHDILEKTIKPGSIEKAYPENEASDVIKKVREAGIEGYVGHWNKETG
RLEGIYMSSGHGLPNGQVNGKIYPIDLNNLEASLAPLKEKKNWAALPFTGDYDMHDMISFTTQPHSVPSNS
SEEKKIIDRINEYIAKSDSNRPFEDIEHNVIRHGPQVSYPAFAMDKEKKEIKERGGIVKAVAEPGEFPVAIVSK
GKWTIANNINELEQFYNSIGAKMKASWKPGAGNPGFVSNPQKPGMARFSRKK
(Plu1713)
SEQ ID NO: 40
MFSTYSSKNDNQTINKINTEEKHENTETDNHLEINLEHTGKSKPDIEPKDVTTGTINAGTLLYKTTAIPEFLDN
AKSLGLAEYEKRHKDIQDYLNLGKAEDAEKLKNKSQWAGQYFALEKSYDEYANEAPDSYNNLLKNAGKDLL
ENTEEVKVFLYTFKVTKDIKVLKPHNNSNSYYVGDTEGWEKAKEIMNDVQSQSEKNDNPFPELKNLEDKNF
LLEELGEKGYAWMGPLHAKEGAEKGTEFSYELAISPNLLRQHLTLESEELLGTYKNRYGYWDKK
(Plu1714)
SEQ ID NO: 41
MKKTDEKYGQYEYKDEDITSYPIAWTNPDNGKIYIGINSPEYSHLNNKGESELNLAKIISTIIHESLHASSHQH
KGLQSQTDTGADNLNYDEYVTDYFAREVYKQILPDKDYVANCFTKGLGGENKIWGGNIVEFMIQ
(Plu2400)
SEQ ID NO: 42
MVYEYDKTIERRRNPSIQLNNNEKSSEQALELSQNNPLLHDLITSNNLRKEAAVFAKRIGPSYQEILDELEHL
HHLSGNEQLAAGFELHRRITHYLEEHPDSKRNTALRRTQTQFGDLMFTGTLQKIRHSLLEMAETRPEMASHI
YQIAREEVKGNTPGLTDLMVRVWKEDPYLAAKTGYQGKIPNDLPFEPKFHVELGAQFDDFKKWLDTAQSK
ELLTHTRLDEQNKQVHLGYSYNELLDMTGVESVQMAVYFLKEAAKQAEPGSTKSQEDILLHRFANPTYLAQ
LEHSRLAQIEAIYHSSHDTDVTAWDQQFASDALTQFNHQLNNTVDLNSQLSLLLKDRQGLLIGESHGSDLNG
LRFVEEQMEVLKAHGVTVIGLEHLRSDLAQPLIDKFLASGNEPMPAELAALLKTKHLSANLFEQARSKQMKII
ALDNNSTTRPTVEGTQHGLMYRAGAANNVAVERLRQLPAGEKFVAIYGNAHLQSHEGIDHFLPGITHRLGL
PALKVDENNRFTAQVDNINQRKRYDDVVELPRIQLTS
(Plu2401)
SEQ ID NO: 43
MEHEYSEKEKPQKCPIQLRDSIEHDKEDINTTTPLELNSQYTNRKRAGLRERFSTTLQRNLPGHSMLDRELT
TDGMKNQESRFSPAMIMDRMMHFGVRTRLGKVRNSASKHGGQVTFKFAQTKGTFLDQIMKHKDTSGGVC
ESISAHWISAHAKGESIFDQLYVGGQKGKFHIDSLVSIKQLQMDSYLDDEQSTMTEYWLGTQGIQPIMQKND
VDEHSSKVVGQTGNKGTTDLLRAILDTGDKGSGYKKISFLGKMAGHTVAAYVDDQKGVIFFDPNFGEFSFP
SITSFSRWFTDDFWPKSWYNLEIGLGQQFEVFNYELKKS
(Plu2514)
SEQ ID NO: 44
MYDSKKKNSEPTTKKKFERSNYSQWDDSINHYEDMNRARIKNRNDILTTVDYFGEKKKTMHTFEYQSDIKH
DTNFNNKNKSLFESFAASFVLQNPSFFSGVIDKLSKKLFNIISKIDERNNFQKKLYDFIEKDTSPEGQFGRFTL
GKNEILNVLQVKSDTPQLFVKKMLLIKSLGAFIIDFSSKDIGNYDFIFDGKGREVNDIIEKNRPTNLFKVRGRTN
IKSSQHRSDIGILDTPTFDSLTEEQKSFLTIPELTKRRPLFRTFTHELDAEDKRVVESVFVNRTFDCDSPLIGS
VSGSTSCVLVAADILFPDMTMVERKKLAIATFAFLVGGGYHSATEVFDVAYPGLDLNKEIEELIENNPIQENA
GVATLRQLIGNSGF
(Plu2515)
SEQ ID NO: 45
MPISNLAKESEVRAVKDIPCKNIETDNHLEIGLSSGLSRSKDTSKFKKNSINTIKLIDDIIALHNDPKGNKLLWN
DNWQDKIINRDLANIFEKIDESVSELGGLEMYQEMVGVNPYDPTEPVCGLSAQNIFKLMTEGEHAVDPVEM
AQTGKIDGNEFAESVDQLSSAKNYVALVNDRRLGHMFLIDIPSNDQETVGYIYQSDLGQGALPPLKIADWLN
SRGKDAVSLNKLKKLLSREFNLLSDDEKRALISETLDIHKDVSNVELDRIKRDRGVDIYLTEYDVNNFYENIET
LKSKLSNYDKKLSKPK
(Plu1649)
SEQ ID NO: 46
MLANVLPNLASFLKYEKETPLFFIEDGFNFQNLNPGRVPLIKTPEQRKAGDTQSPAFLCSGVILRGTIHSNDY
KFWQPSPSSIKSGGVSFSYLRKDAKFKRLAYGYKNGFIIFPEHIAPEDRVDFSVLCAFPIDGYTNERANQGC
GENITKAKDKGKSCQEQNVTNSDDWIKNYRKVNSQDFFQCGFNVTKDVNNPAIAFYQMLESIKKLPRTPNT
PPKQNEIRISTWEESDPNKLPIEALFYSENSGLADAQKDQRDYKNATGKFLPIVKMLLPRTLNEDALFKFNIK
DQVINP

Leader Sequences (e.q. with SEQ ID NO: 47 -
92 correspondinq to amino acids 1-50 of
SEQ ID NO: 1 - SEQ ID NO: 46, respectively)

| Sequence | |
|---|---|
| ID | Amino acid sequence |
| SEQ ID | MMREYSNEDDFIKEKTNLVKSENVE |
| NO: 47 | ADNYLETEYLTYLAKLIGMTERENH |
| SEQ ID | MFQNRIRNEKTTQSGKGKTLDRMTD |
| NO: 48 | SLYLEIPNVEAVTLAYQKLTSKYRK |
| SEQ ID | MEREYSEKQKNPSKLSRKTAISERIA |
| NO: 49 | ALERSGLSNSNQPVPQFARPYTSN |
| SEQ ID | MSNYEYDIVTQHDTYQIKDNEYTVVN |
| NO: 50 | GKYWQYEQEGNKNNNKVSISLMKE |
| SEQ ID | MEHEYNEKEKQRNSAIKLNDAIRNNE |
| NO: 51 | ENMDMTSPLELNFQNTNRKSRGLR |
| SEQ ID | MPNKKYSENTHQGKKPLMKSEANN |
| NO: 52 | EHDIQNSSLGIGLDLNSMMGNSSTSL |
| SEQ ID | MEREYSEKEKHKKRPIQLRNSIEQHE |
| NO: 53 | EETANNSLGLGLDLNQATNPPKVP |
| SEQ ID | MMEHEYSKEEEKKRQQSKPNNATH |
| NO: 54 | DESNLPLELEKHFNARTPATAHSKWF |
| SEQ ID | MLKYANPQAVPTQRTKNTAKKPSSS |
| NO: 55 | SSFDGQLELSNGEWSKHSEMGLKRG |
| SEQ ID | MMREYSNEDDCTKEKTNLVKSENVE |
| NO: 56 | ADNYLEMEHLTYLAKLISMTERENH |
| SEQ ID | MIFKMLNLAVFYLLGNIFHYLICQKFIC |
| NO: 57 | YFCSVLKSVTMFLTKVAVQIAL |
| SEQ ID | MEREYSEKPKNLSQLSRKTAISERRA |
| NO: 58 | MFERNASSNNEQPVPQFARSYTSN |
| SEQ ID | MKYDPRLRTVWEDDFDYEKNFKKQ |
| NO: 59 | TDYINYKDLEKQLKENVDYYALLDEN |
| SEQ ID | MPNKKHSENTHQGRKPLIKSEANNE |
| NO: 60 | HDIENSSLGIGLDLNSTIGNNSASL |
| SEQ ID | MEREYSEKEKHKKRPIQLRNSIEQHE |
| NO: 61 | EETANNSLGLGLDLNQATNPPKVP |
| SEQ ID | MMEHEYSKEEEKKRQQSKPNNATH |
| NO: 62 | DESNLPLELEKHSNARTSATAYSKWF |
| SEQ ID | MSNYEYDIVTQHDTYQIKDNEYTVVN |
| NO: 63 | GKYWQYEQEGNKNNNKISISLMKD |
| SEQ ID | MEHEYNEKEKQRNSAIKLNDAIRNNE |
| NO: 64 | ENMDMTSPLELNSQNTNRKSRGLR |
| SEQ ID | MFKYDTSEKMAKFGKGKTSDGMLLD |
| NO: 65 | TLYLEIPDEKAVMSAYKSQILDELR |
| SEQ ID | MLKHANPQTVSTQRTKSTAKKPSSS |
| NO: 66 | SSFDRQFELSNSENQPGEGNKDWTI |
| SEQ ID | MPRYANYQINPKQNTKNSHGKSSSS |
| NO: 67 | NFSSGYFSSSNNSLDDSLIRQQVKR |
| SEQ ID | MREYSKEDDCVKEKTNLAESENVEA |
| NO: 68 | DNYLEMDCLNYLAKLNGMPERKDHS |
| SEQ ID | MPIIGHKEDLIRTERSSVDLTRSSNN |
| NO: 69 | RQTDNLELNIPQHKRDNKDIEHAV |
| SEQ ID | MISTFDPAICAGTPTVTVLDNRNLTVREIVF |
| NO: 70 | HRAKAGGDTDTLITRHQYD |
| SEQ ID | MEREYNKKEKQKKSAIKLDDAVGNNEEN |
| NO: 71 | MDMTSPLELNSQYTNRKRPGLR |
| SEQ ID | MVYEYAKTNDRKRKLSTQSDNYEEKSFS |
| NO: 72 | PVLDLSRNNQNTPNMEDEYETP |
| SEQ ID | MVFEHDKTVERKRKPSIQLGNDKEKSSEQ |
| NO: 73 | ALELPQSKQNNPLLHDLITSN |
| SEQ ID | MKGIEGVIMLSHDILPEKLLVSEKKHENVG |
| NO: 74 | SYFSDDIGEQSEQTEVSHFN |
| SEQ ID | MPNKKYSENTHQGKKPLIKSEANNEHAID |
| NO: 75 | NSPLGIGLDLNSILGNNSASL |
| SEQ ID | MEREYSEKEKHKKHPIQLRDAIEQHAEET |
| NO: 76 | ANNSLGLGLDLHQAINTPKVP |
| SEQ ID | MVHEYSINDRQKRHSFSSANPIDPEVTNR |
| NO: 77 | ENSRHRFPKDNYNKGHGDLFY |
| SEQ ID | MLKYANPQTVATQRTKNTAKKPPSSTSFD |
| NO: 78 | GHLELSNGENQPYEGHKIRKI |
| SEQ ID | MPRYANYQINPKQNIKNSHGKSSSSDFSS |
| NO: 79 | GYLSFSNNSLDDPFIRQQVKR |
| SEQ ID | MPNKKYSENTHQGKNPLMKSGANNEHDL |
| NO: 80 | QDSPLGIGLDLNSMLVNSSTSL |
| SEQ ID | MLSTEKHNKDTKHPRNREKKFSIQPENST |
| NO: 81 | QDDEDIKNNSLGVGLDLDQMI |
| SEQ ID | MPINDLKKKFEISPQAAQAIGAPARSNSSK |
| NO: 82 | QAEHQTEHLELDTSKNRRDR |
| SEQ ID | MPNSKYSEKVNHSANGAEKCSIHSNQYNI |
| NO: 83 | NNCTLGLGLDLNKKLRTGNER |
| SEQ ID | MPKLTELLSRFENPIQNQPNHISKKNPISN |
| NO: 84 | SKVLNNSEEKTAPLELKHDD |
| SEQ ID | MPRYSNSQRTPTQSTKNTRRTSPSSNSS |
| NO: 85 | TEHLSLSNAPTNDSSVRQEVKE |
| SEQ ID | MFSTYSSKNDNQTINKINTEEKHENTETD |
| NO: 86 | NHLEINLEHTGKSKPDIEPKD |
| SEQ ID | MKKTDEKYGQYEYKDEDITSYPIAWTNPD |
| NO: 87 | NGKIYIGINSPEYSHLNNKGE |
| SEQ ID | MVYEYDKTIERRRNPSIQLNNNEKSSEQA |
| NO: 88 | LELSQNNPLLHDLITSNNLRK |
| SEQ ID | MEHEYSEKEKPQKCPIQLRDSIEHDKEDIN |
| NO: 89 | TTTPLELNSQYTNRKRAGLR |
| SEQ ID | MYDSKKKNSEPTTKKKFERSNYSQWDDS |
| NO: 90 | INHYEDMNRARIKNRNDILTTV |
| SEQ ID | MPISNLAKESEVRAVKDIPCKNIETDNHLEI |
| NO: 91 | GLSSGLSRSKDTSKFKKNS |
| SEQ ID | MLANVLPNLASFLKYEKETPLFFIEDGFNF |
| NO: 92 | QNLNPGRVPLIKTPEQRKAG |

(Photorhabdus asymbiotica strain ATCC43949 PVCPnf operon, pvc1 -
pvc16; e.g. corresponding to genes PAU 03353 to PAU 03338 of the
sequence of GenBank accession no. FM 162591.1)
SEQ ID NO: 93
ATGTCTACAAGTACATCTCAAATTGCGGTTGAATATCCTATTCCTGTCTATCGCTTTATTGTTTCTGTCGGAGA
TGAGAAAATTCCATTTAATAGTGTTTCAGGATTAGATATTAGTTATGACACCATTGAATACCGAGATGGTGTTG
GTAATTGGTTCAAAATGCCGGGTCAGAGTCAGAGCACTAATATCACCTTGCGTAAAGGCGTTTTCCCGGGGA
AAACAGAACTGTTTGATTGGATTAACTCTATTCAGCTTAATCAGGTAGAGAAAAAGGATATTACCATCAGTTTA
ACTAATGATGCAGGTACCGAATTATTAATGACCTGGAATGTTTCTAATGCTTTTCCCACTTCATTGACTTCACC
TTCATTTGATGCCACCAGTAATGATATTGCAGTACAGGAAATTACGCTGATGGCAGATCGGGTGATTATGCAG
GCTGTTTGAAGCATTGATATTTAATCATCTCATATAAGGGAACTTTTATGACAACCGTTACCAGTTATCCTGGC
GTTTATATTGAAGAATTAAATAGCCTGGCCTTGTCAGTTTCAAATAGCGCCACAGCGGTTCCTGTTTTTGCTGT
GGACGAACAAAACCAATATATTAGTGAAGATAATGCAATCCGTATTAATTCGTGGATGGATTATCTTAATCTGA
TTGGCAATTTTAATAATGAAGACAAATTAGATGTTTCTGTGCGTGCTTATTTTGCCAATGGAGGTGGATATTGT
TATCTCGTCAAAACAACGAGTTTAGAAAAAATTATTCCAACCTTGGATGATGTAACCTTATTGGTTGCTGCGG
GCGAAGATATTAAAACGACAGTAGATGTTTTATGTCAGCCAGGAAAAGGGTTATTCGCAGTCTTTGATGGCCC
TGAAACAGAGTTGACTATCAACGGTGCGGAAGAGGCAAAACAAGCCTATACCGCCACACCATTCGCTGCGGT
TTATTATCCTTGGTTGAAAGCGGATTGGGCTAACATAGATATTCCACCCAGTGCAGTGATGGCGGGAGTTTAT
GCATCGGTGGATTTATCCCGTGGTGTATGGAAAGCGCCTGCCAATGTTGCGTTGAAAGGGGGCCTGGAACC
TAAATTTTTAGTCACGGATGAATTGCAGGGTGAATATAACACTGGCCGCGCTATCAATATGATTCGTAATTTGA
GTAACACAGGTACTACGGTTTGGGGTGCAAGAACCCTGGAAGATAAAGACAATTGGCGTTATGTTCCAGTGC
GACGCTTGTTTAATTCTGTGGAGCGGGATATCAAGCGTGCCATGAGCTTTGCTATGTTCGAGCCTAATAATCA
GCCTACTTGGGAGCGGGTACGGGCGGCGATTAGCAACTACCTTTATAGCCTGTGGCAACAGGGGGGATTAG
UTGGCAGCAAAGAAGAAGACGCTTATTTTGTGCAAATTGGTAAAGGTATAACGATGACACAGGAGCAGATTG
ATGCAGGGCAAATGATTGTTAAAGTCGGTTTGGCTGCTGTACGGCCTGCGGAATTTATCATTCTCCAGTTTAC
GCAAGATGTAGAACAGCGTTAATCATATGATTATGAGGAGTTATCATGTCTGCTATTCTGAAAGCGCCTGGCG
TTTATATTGAAGAAGACGCTTCCCTAGGGTTGTGTGTGAGTAAGAGGGGGAGTGGGGTGGGTGTTTTTATGGG
AAAATTTACTCCGACAGTGGTTGATTCAATCCAAGTCTGTACCCGTATCAGCAACTGGCTTGAATTCACTTCC
TGTTTTTGGGTAGGTGGAAGAGTTGAGATTGTTGTGGAATGTAAGAGTGAATGTGAATGTGAATGTGAAAGTTA
GGAGTATATTGAGAGAATGAATTTATGTGGAGGTGTGGAAGGATTGGGAGTGTATTTTGAAAATGGGGGAGGA
GCTTGCTATATCTACCCATTAAATGATGCTGAAGATGAATTGGTTCTGGCGGCCATACCAGAAGTCATTGAAC
AGAAAGGTGATATTACTCTGTTGGTTTGCCCGGAACTCGATCTGGATTACAAAACTAAGATCTATGGCGCAGT
GAGCTCACTGTTGAATGATAACAAAGTGGGCTATTTCCTGATTGCGGATAGCAATGATGGAGAATCTGTGTCA
GGAGTATGGAATAGTGGTAAGGGGGGGGGGTATTATGGGGAGTTGGAAAGTAAGGTAAAATTTTGGAGGTTGG
CTGGGGATAAGGACATTCGTATCAGCGGTTATCAGGATGATGATGAAACACATAAACCGAAAAACTTGGATG
AGCTCAGGACAATCAACGAGGCGTTGGCACAGGATATTGATGCAAGATTGCTCGAGGAGAAACAACGTGCT
GTCATCATTCCGCCAAGTGCTGCCATTGCGGGCATTTATTGCCAAACGGATAATCGTCGCGGTGTTTGGAAA
GCGCCAGCCAACGTTGCGCTCACAGGGATCGGGAGTTTGCTTGATAAGGTAGACGATGAACGGCAGGGAGA
GATGAATGAGAAGGGAATGAATGTGATGGGTTGATTTAGGGAGGGTGGTTTTATGGTGTGGGGAGGGGGTAG
TTGTGTGGACGCTGCCAACATCAGCTGGCGTTATATTCCTGTTCGTCGCCTGTTCAATTCCGTTGAACGAGAT
ATCCGCCAGGCGCTGCGCGCTGTGTTGTTTGAAACTAATAGTCAGCCTACCTGGGTACGTGCTAAGGCTGC
GGTTGATGAATATGTTTATAGGGTTTGGGAGAAAAATGGATTGATGGGTGGTGGGGGGGAAGAAGGTTATTTT
GTGCAAATTGGTCAGGATATCACCATGTCCGAGGCTGATATTAAACAGGGTAAGATGATCATGACTGTTGGTT
TGGCAGCAGTGCGGCCAGCTGAGTTCATCATTCTGCAATTTACGCAGGATGTTGTTCAGTAATCTCCATGACT
AAACGCCAGGCACTGTATTGACAGTGCCTACTCTAACCATCTTGGAGGAGGTGATGATGATGGAGAGACTCC
AACCGGGTGTGACTTTAACAGAAAGTATAATCACGATGGGTCAGCAAGAGATACCCAGTGCTGTGCCGGTGT
TTATTGGTTACACCGTTCGTTATCCGGAACAATCGGAAGCATCAGTCCGTATCGACAGTTTGGCCGAGTATAC
GAGGGTGTTTGGTGAGGAGGATGTGATGATGTTTGGTGTGAGGGAGTATTTTGATAATGGGGGGGAAGAGGG
ATTTGTTTTAGGGGTGAAGGAGAATATGGGATGAGTGGAGATGAGGAGAGGTGAAGGGGAAAATGTGATAGG
GGGATTGGGGTGTGGTAGGGTTAGGGAAGGGATTGGTGGGGATAGTGAGATTAGAGTGATTTTGGTAGGGGA
TATGGCTCGGCTTAATGACAGTGATATTGATGACTCCTCAACCCAGGTAAGCCTGTGGTCCCAAGGCTGGGA
GGCGCTGCTGCAATTGAGTCAGGTTAGGCCCAACCTCTTTGTGCTGTTAGATGCGCCGGATAATGTTGAACA
GGCGCAGAAGTGTATGACAACGCTATCGTCAGATTATCGTCAATGGGGGGCAGCATATTGGCCTCGTCTGG
AAAGTAGGTATGAGAAAGAAATATGTGGGAAGGAGAATGAATGTGAGGGAATTTTGGAGGGGAGTGTTGTGTG
ACCCACAGCCGCGGTCGCAGCGGTAATTCAACGCACGGATAACGACGCGGGTGTTTGGAAAGCACCGGCC
AATATTGCCTTATCCCAGGTTATTCGACCTGTTAAATCTTATCTTCAGGGAAGTGTACTGTTTAACAGCAGCG
GCACTTCGCTCAATGTGATCCGCAGTTTCCCAGGTAAGGGCATACGGGTATGGGGATGCCGCACTCTGGAA
AACACGGATAATACGCAGTGGCGCTATCTGCAAACACGTCGGCTGGTTTCCTATGTAACAGCGCATTTGACC
CAATTGGCTCGCATGTATGTCTTTGAGCCAAATAATGAACTTACCTGGATGAAGTTAAAAGGACAAAGTTACA
ACTGGTTACGGCAATTATGGTTGCAGGGTGGCTTGTATGGTTCACAGGAGGATGAGGCATTTAACATTCTGT
TAGGCGTAAACGAGACGATGACTGAGGATGATGTTCGTGCAGGAAAAATGATCATGAAAGTTGAGTTGGCTG
TGTTGTTTCCTGCCGAATTTATTGAGATCAGTTTGGTGTTTAATACCCAAACAGAGGCGCTGTCTTAAGAAGG
AAAAAGTAGGATGAAGGATTATTAGAGAGGGGTGGTATGGGATGGTTTTATGGGGAGTTTTATTTTTAAGGGGA
TTGGGGATGGGGTGGATATTGGTTTTGAGGGTATGTGTGGGGTTAGTGGGGAAGTAGAGGTGAGTGAGTAGA
GTGAGGGAGGAGAAAATGCCCGTAATAACTATTTAGCTGAGAAAATCCAACACGGTACGTTGACTTTGGAAC
GGGGCGTGATGACAGTCTCGCCATTGACCTGGATGTTTGATCGGGTATTGAGTGGTGAAAAAATCGCTTATG
CCGATGTGGTGGTGATGCTACTGAATGAAAATTCACTGCCATTGTCCAGTTGGACGTTGAGCAATGCGCTGC
CGGTACGCTGGCAAACCAGCGACTTTGACGCTAACAGCAATGCCATATTGGTGAATACCCTTGAATTGCGTT
ACCAGGATATGCGCTGGCTTGGAGTCAAAATATGACAGTAGAAATCAGAGAGTTACTTATCCAGGCAAAGGT
AGTGCCATCAACACGACCGACTGAATCAGAACGGCAAAACCATTCTTTGATACAGGAAAGTCTGGATGAGGC
GACTTGGGTGGAAACGATAAAACGCGAAGTGTTGGCCGCATTACGCGATGAGGAAGGGTGGCGTCCATGAG
TCTGATTGAACGTGGTTTAGCTAAGCTGACAATTAATGCTTATAAGGATAGGGAAGGGAAGATACGGGCAGG
AACGTTGCAGGCCATGTATAACCCTGACTCCTTGCAACTGGATTACCAAACGGATTATCAGCAATCCCAAGC
GATTAATAGGGAAAAGGAAAGTAGGATTTATGTAGAGGGGAAGGGGGGAGGGTTATGAGTTGAATTAATTTTT
GATGCCACGATGCCGGGTAACAAAACCCCCATTGAAGAGCAGCTCATGCAGCTCAAGCAACTGTGCAGTGT
GGATGCAACCAGTAACGAGACGCGATTCCTGCAAGTTAAATGGGGCAAAATGCGTTGGGAAAGTCGGGGTT
ACTTTGCTGGCAGGGCCAAGAGTTTGTCTGTGAATTACACTTTGTTTGATCGTGATGCGACTCCCTTGAGGGT
ACGGGTAATATTGGCATTAGTGGCTGATGAAAGTCTGGTGTTGCAGGAGACTGAACAAAATCTGCAATCTCC
GGCAAAAATCGCATTACGCATACAGGATGGGGTATCTCTGGCTCTGATGGCAGCCAGTACGGCATCAACATT
GTCAGGCGGTGTGGATTATCTGACGCTGGCCTGGCAAAACGGTCTGGATAATCTCAATGGGTTCGTTCCGG
GTGAAATATTGCAGGCCACCAGGGGAGACGAATCATGAGCCACCAACTGAAAATTATTGCAGATGGTAAGGC
ACTGTCACTTTTGGCCGCGGTAGATGTGGACACCTGTTATCGGGTTAACAGTATACCTTCTGCGACATTGAAA
CTGAGCGTACCGGATAGGCCACTCTCTTCTTTCAGTCAGACGGATGTTCAGACAGAACTGGCCCACTGTCAG
GTAGGGAAAACCCTGCGTCTGGAATTGATTGATGGTAGCAAAAAATGGGTGCTGTTTAATGGTCTTATTACCC
GTAAGGCTCTGAGAATTAAGAATAAGCAATTATTGCTCACTCTGGTTGTCAAGCATCGGTTGCAACTGATGGT
GGATACCCAGCATTCACAGCTGTTTAAAGACAAAAGCGAAAAAGCGATCTTAAGCACGCTATTGAATCAGACC
GGAATCAATGCTCGCTTCGGAAAGATAGCGGCGTTAGATCAAAAGCATGAACAGATGGTGCAATTTCGTTGT
TCAGACTGGCATTTTCTGTTGTGCCGACTGTCGGCAACCGGTGCATGGTTGTTACCTGCCATAGAAGACGTT
CAGTTTGTTCAACCTGATGCTCTGAAATCAAACTCAGCCTATACCTTGAAGAGCAGGGGGGATGAGAACAAA
GACATCGTTGTCAAGGATGCTTACTGGCAGTTTGACAATCAAATCAACCCCGCTTTGCTGGAAGTCAGTGGC
TGGGATATCAGTAAGCAGCAGGTACAATCAGGCGGTCGCTACGGAAAAATCGCGTTGGGTAAGGCGGCACT
CTCTCCTGATGGATTGGCATCCCTTAATAAAACGGGTTGGGACATTTGTTATAGCAGTCCGTTAACAACCCAG
GAAAGCGGTTATCTGGCACAGGGATTATTGCTTAACCAGCGCATTTCTGGGGTGACAGGAGAATTTTGCTC
AAAGGAGATGGGCGTTACCAGTTGGGAGACAACATTCAGCTGACTGGATTTGGTTCACAGTTAGATGGTACG
GCAAGCATTACTGAGGTTCGCCACCGTCTTAATCGGCGAATTGATTGGGAAACCACGGTGAGCATTGGTTTA
CAACATGAATATTTGCCGATATTACCTGATGCTCCCGAACTACATATTGCGACAGTAGCGAAATATCAGCAGG
ACAGTGCGGTGTTAAACCGTATCCCCATTATTCTGCCGGTACTGAATCGTCCCAATGAATTTTTGTGGGCCAG
ATTGGGGAAACCTTATGCTAGCCATGAAAGCGGTTTCTGTTTTTACCCAGAGCCAGGTGACGAAGTTATTATT
GGTTTTTTTGAAAATGATCCGCGTTATCCAGTTATTTTAGGTGCTATGCATAATCCGAAAAATAAGGCCCCTTT
TGAACCAACCCAAGATAATAGGGAAAAAGTATTGATCGTTAAAAAAGGTGAAGCGCAACAACAATTAGTCATT
GATGGCAAAGAGAAAATGATCCGAATTAATGCGGGTGAAAATCAAATAATGCTTCAGCAAGATAAAGACATTT
CTCTGTCAACGAAAAAAGAATTAACACTGAAAGCGCAGACAATGAATGCCACGATGGATAAATCATTGGCAAT
GTCCGGGAAAAACAGTGTTGAAATCAAAGGCGCAAAAATTAATCTTACCCAATGAAAGGTGACGATGAATGG
AAAATCAAATACTGACACAACTCTATGGTCGTGGTTGGGCTTTTCCTCCGGTCTTTTCCCTTGAAAAGGGGGT
AGAGATGGCTGAAGGGGCGGAAGATGTGAGACAAAGTTTGCAGATTCTGTTTAGTACTGAGCCGGGGGAAC
GTCTTATGCGTGAAAATTATGGCTGCGGATTAAATGATTTTATGTTTGAAAATATCCGCAATGAACTTATTGCT
GAAATTGAATCCCATATCCATGACAACGTATTACGATATGAACCCCGGGCTGATATGACTGATATTCAGGTTC
GTCAATCCCCTGGCATGGGGAATACTTTGCAAGTGCAGGTCATGTATCGCCTGAGAGGGAGTGATATCAATC
AACAAATCCAGGGAGTACTTGCACTGAGTGAAGGCCGGGTGACGGAGGTAGTATGAGTGAAGCGATTGTGG
TGGATGGTGACGTGTTACAGTTTGATCCCAACTTTGGCAATCGGCAGGTGACGGTTCCCAGCCCAGGAAAAA
TTAGCGGCACAGGACATGCGCAGGTAAGTGGAAAAAAAGTGTGTATTCTGGGGGATGAGAAACAGGTCAGG
GTTTCTGCAACCTATATTACAACAACACATACTACGCCGGGAACAGGAACCATTACTATCAGTGCTCTGGATG
CTGGCCAGCAGGCCCTTCAGTGTACCAGTGGGGCGGCTTTAATTATCAAGGGGCAGCAATTTACGGCGATG
TTTACGCCTGAATTGCCAGCCATGAATAATACAGTGACTCCGCCACAACCGGATGTTACGACACCTTCATCAG
GAAAAGGACGTTTTATCACTCAACAAAATTTTGCTACCGTAAATTAGAGTATTGACTGAATTAAATAGAATTAA
CGAAGGTGTAAATAATTATTTATTTGCTGACGAATCGCTGTGACAAATAAACACAGGTGATGTTATGGAATTAA
ATGAGTTAACTAACAAATTGTCAAATTTGGTGCCAATGACCGATTTTAAATTAGATAATCGAGCCAGTTTGCAA
TTGCTTAAATATATTGAAGCGTATACGAAGATAATACCCTTTAATTCTGGCGATAAATATTGGAATGACTTTTTC
TTTATGTCAGGAAATACGCCAGAGAAACTTGCAAAATTATATCAGAAAGAAATAGAACCCAATGGGGAGTTAT
TACCTCAGCAGGCTTTTTTGTTGGCGGTTTTGCGTTTATTGGAAACACCAATATCCTTATTAAATGTATTACCT
GCTGCTCATCGTGAGCTCTATTATCGGGAGCTTTTAGGCTTGTCTTCCCATGCGGCACAGCCTGATCAGGTT
GCTTTATCTATGGAACTGAATTCGACAGTGATGGAACAGCTGCTCCCTGAAGGAACCCTGTTTGAGGCTGGT
CAGGATGAACAAGGCAATGCATTGCAATATGCCCTGGATGCCAGTTTGCTGGCTAATCGTGGATATATCAGT
GACTTGCGCTGGTTACGGAATGACGGGGAAAAGCAATGGGTTACTTCTGCTCCATGGGATTTACAGGCACAG
GTGTCACTGCCGTCTGATGGGATACGATTATTTGGTAAGACAAATAGTGATCAGCAGGTATTTGGTGGGGTG
TTGATAACGTCATCACTTCTGGCGATGGAAGCGGGGATAAGGAAGATCATTGTTACTTTTGAGCAGGAGATG
AACACCCAAGAACTGGTGGCACAGGTCAGCAGTGGAAATCAATGGCTAACATTGACGTCTGAGGTAAATAAG
AAAGAGGTCACACTGACACTGTCAGACAAAGAACCGGCAATCAGTGCGCCAGAGGATCTGGATAATCTCTTT
TTCACGCAACCGGTACTCAGGCTACAGGGAAAGGATAGTCAGGCACTGCCGGAGGTGACGGGTATCAGCGT
TTCGGAAAAGGATGATACTAAGGATACCTCTTTTGAGATGTATCACTTAACACCATTTGGTTATAGCAGTGATA
TAGAGCCATTGGAGGAAAATCCAGCGTTATATTTAGGCTTTACTGATGTAAAGCCAGGGCAAACACTGGCGC
TGTATTGGAAATTAAAATCCCCGCAGCAACCAACCGTTTCCTGGTATTACCTGGATCAACATAATCAATGGGC
TGAATTGGATTCATGGGTCAGTGATGGAACCCAGAATCTGTATCAGGATGGTACTTGGCACGTTGAGTTGCC
TGTGGATGCATCCAATCAGGCAGAGCAGATGCCAGTTGGACGCTATTGGTTGCGGGCAGTGGTGGAGGTAC
CCGCTCATGAGGGGGCGTTGGGGAAGGCTCCTTGGCTATATGGTCTAATCTATAACGCCATGACGGCAACC
TTGGTTAATGTAGATAGCATCAGTGACAGCCATTTCTTAACCCCTTTGCCTGCCAGCAGCATACAGCGGCCC
GTTGAACCCATCATTGTGTTGGCATCGGTCAACCAGCCTTGGGCATCATGGGGTGGACGTATACCTGAATCC
TACAGTGCCTTTTTTGAACGGATAGCTCAAAACCTGTCTCATCGAAACCGGTCCTTAACCTGGGGAAATATGG
TGACATTACTCAAAGAGCGTTATGTCAGCATCTTTGATGTTAAGTATCCAGGTAATGATGAACTCACCAGAGT
GCCAGCATTGGAGCAGCAGCAACTAACAGTGATTCCAGCAAACCGGTACAACGATAGCGATGATTCTCTGCG
TCCGGTACTGAATCCTGCTCGTCTGCAAGAGATGGCTGATTGGTTGCAGCAGAAAGACTCTCCCTGGGCCTC
TATTGAGGTCAGGAATCCAGAATACTTGGATGTGAAAATCCATTACGAGGTGATTTTTAAACCTGATGTGAAC
GAAGATTTTGGCTATCGCCAGCTACAGCAGCAACTGTGTGAGGTGTATATGCCTTGGAGCATAGATGAGCAG
CGGCCCGTTGTATTGAATAACAGCATTAATTATTTCCAGTTGTTAGCCACTATTCAACAGCAACCGCTGGTTG
AGCGAGTCACTCGTCTGACACTACATCGGGCTGATTCTTCTGATGAGAGTGATGGTACAGCATCTGTGGAAG
CCAAAGATAATGAAGTGCTTATTTTAGTCTGGGAAGAGGACGATAATCTGCAATACCGAGGAAATGACTATGA
GTAATCAGGATGCACTGTTTCATAGCGTTAAAGACGATATTCACTTTGATACCTTGCTGGAACAAGCTCATCA
GGGATTGAAAAACAGGCTGAAAAACTGTGGAGTGATACGGCAGAGCATGATCCGGGTATGAGATTTTTGGA
GGGAATCAGTTACGGTGTGTCAGATTTGGCTTACCGACATACATTACCCCTGAAAGATTTACTGACTCCGGC
GGGGGATGAGGAGGAGGAAGAGGGAATTTTTGGTGGGGAATTTGGGGGGGATAATAGAGTGAGTTGTGGGG
CGGTGACAGCGGATGATTATCGCAAGGCATTGTTAGATCTACACAGCAGCGACAGCCTGGATGGTACTCAG
GAGGATGAGGGGGATTTTGTGTTGGGGAGTGTGGAAGTGGTGGGTGAAGGGGAAAAAGAGGGTTATAGGTAT
TGGTATGATGCAACCAAGAGGGAATATAGCTTTGTCAACAGTGAAGGGGCTAAAGAGTTTACCTTGCGGGGG
AATTACTGGTTGTATCTGGAACCAACCCGTTGGACTCAGGGTAATATTGCCGCTGCTACCAGACAACTGACA
GAATTTTTGAGTAAAAATGGGAATATTGGTGAATGTGTGAGGAAGATTATGTGGGTAGAAGGGGTTGATGTGG
GAGTGTTGGTGGATGTTGAAGTGGATGATGATGTAGGTGGAGAGGATGTGGGGGGTATTTTTGGGGGGGTGT
ATAGCACCGCAGAGCAGTATCTGATGCCTGGAGCACAGCGTTACCGTACGGAAGTACTGCAAAATGCTGGG
ATGAGCAATGATCAAATCTTCGAAGGTCCATTATTGGAACATGGCTGGATACCAGAGCTGCCGGCAGCCCGT
GATTATACTCAAAGGCTCACTCTCAATCTTAGCCGGTTGGTAAATAGTCTGCTTGAGATTGAGGGCATTAAAC
ATGTGAATCGTCTTCGTCTGGATGATAGCTTCGATAAAACTGCTATTGAACCCGTTAAGGGGGATACCTGGTC
GTGGTCGATCAAAGAGGGCTATTATCCACGTCTTTGGGGAGAAGACCCACTTAACCAATTGGCGCAACAAAA
TGGCCCGCTTAGGGTGATAGCCAAAGGAGGGATTAGCGTCAGTGTGAGTAAAGAGCAAATCCAGGCCAGTT
TACCCAGTCAATCACTGATTCAAAATGAGCCGGTAATATTGGCTTACGGCCAGCACCGTGACGTTGGCAGCT
ATTATCCCGTCAGTGATACTTTGCCGCCTTGCTATGGACTACAACATTCTTTGTCTGAAAGTGAACACTTATTG
CCACTTCATCAATTTATGTTGCCATTTGAACAATTATTGGCCTGTGGTTGTCAACAGATAGCCATGCTCCCGC
GGTTACTGGCTTTTCAGCGCGAAGGTTATGAGGTTTGGGGTGATCAGTGGCCCTTTAAGTCAGGCTCAGTGA
ATGATGACGCCCATCAAGATTATGCCCCTGCATTAAAGGATTTGTTAGGACAGATTGCGCTGGATAGTGATCA
TGAATTGGATATTATTAATTACTTGCTGGGTTACTTTGGCACACAGCGGGCACCGCGTACCTTTACGACACAA
CTUGATGATTTTCGTGCGGTCCAACAGGGTTATCTGGCCCAGCAACCGACATTGACTTACCACCGCTCCAAT
ATTCGTATCGATCAGGTATCGTCGCTACAAAAACGTATTGCTGCTCGCATGGGGCTGGGCGGTGAGTTGTTT
AAACCTCAACCGGATCTGAGCCAACTGCCTTTTTATTTGATTGAACATCGAGCGTTGCTGCCAGTCAAACCCA
ATAGTCAGTTTGATAAGGAACAGAAACCAGCCTCGGTGACAGAGGAGGGGGGCAGCCAAACAGGTCAACAT
TATGTGGTCATTGAACAGAAGGGCATTGATGGCAAGCTGACACAGGGGCAAGTGATCAATTTAATTCTGTAT
GAAGGAGAGCAGGGAGAAACCCAATTTACGATACGCGGTCAGATGGTATTCAAAACCGAGGGGGATAAGTT
TTGGTTGGATGTGAATAATAGTGCGCAACTGGAATATAATCTGGCGCGGGTAATGACAGCAGCCAAGGCGAG
TAAACTCTTTTGGCAAAACAGCCCGGTATGGATGGAGGATATGGGCTATCGTCTGGCCTATGCTAGTGACCA
ATCCTCATTGCCTGTGAATCAACGGCGCTTGACCCGCACAGTGCAAACTCCATTCCCGCCGATGGTTGTTGT
AGGTAGCGAAATCACCCTGTTAAAGCAGGTGGGGATAGTCAATTTAAAAAAAGCGGAGTCAGAAAAACTTTAT
GCAAAAGTTGTTAGCTTTGATCGCATTGAAGGGACCTTGATTATTGAGCGTTTGGGTAATTCCACTCTGGCTT
TTCCTACCTCGGAAGAGGCGTGGCGGTATAGTTGGTATTTTTCGGGGGAGAAATATGAAAGGACTGACCGCT
TTTCATTTGTGATTAGCGTAGTAGTGAACAGTGACTTAATTAAATTGCCCGGTGTTGATCCCTATAAATTGGAA
GAATGGGTGAAAGAAACGATTCTTACCGAATTTCCAGCTCATATTTCTATGATTATCCATTGGATGGATCGGG
AAGCCTTTTTAAATTTCGCCAATACCTATCAGCGTTGGCAAAATAATGGTACGCCACTGGGGGATGCGGCTTA
TTCCATTCTAGAAAGTTTGACACTTGGTAAATTGCCATCTGCCTTAAAAGGTGTTGGCACAATGCGTATTGCC
ACATCTAGTCAAAGAGAAGAAGTGGTGGGTAGTAATGGTGATCAATGGAATACAGATGGAATAACCCAGAAT
GAATTATTCTATGTTCCTAAAGAGAGCTAGGAAAAATAAATATCTGCCACTAATGATGTTGAATTAAATATGTTT
TCTGGAGTTAATCATGAACGAAACTCGTTATAATGCAACTGTACAAGAACAACAAACATTATCTAATCCAAAAG
CTGTTGGACCTGACATCGATAAATTAAAGGATAAATTTAAAGAGGGCAGTATTCCCCTGCAAACCGATTTCAA
TGAGTTAATTGATATTGCCGATATTGGACGTAAAGCCTGTGGTCAAGCGCCACAACAAAATGGCCCAGGAGA
AGGATTGAAATTGGCTGATGACGGTACGCTTAATTTAAAAATAGGCACTTTTTCCAATAAAGACTTTTCTCCAT
TAATATTAAAAGATGATGTTTTATCTGTAGATCTTGGTAGTGGTCTGACTAATGAAACCAATGGAATCTGTGTC
GGTCAGGGCGATGGTATTACAGTTAACACTAGCAATGTAGCTGTAAAACAAGGTAACGGAATTAGCGTTACTA
GTAGTGGTGGTGTTGCCGTTAAAGTTAGTGCTAATAAGGGACTTAGCGTTGATAGTAGTGGTGTTGCAGTTAA
AGTTAATACTGATAAGGGAATTAGCGTTGATGGTAATGGTGTTGCAGTTAAAGTTAATACTAGTAAAGGAATTA
GCGTTGATAATACAGGTGTTGCAGTTATAGCTAATGCTAGTAAGGGAATTAGCGTTGATGGTAGTGGTGTTGC
AGTTATAGCTAATACTAGTAAAGGAATTAGCGTTGATGGTAGTGGTGTTGCAGTTATAGCTAATACTAGTAAA
GGAATTAGCGTTGATAATACAGGTGTTGCAGTTATAGCTAATGCTAGTAAGGGAATTAGCGTTGATGGTAGTG
GTGTTGCAGTTATAGCTAATACTAGTAAAGGAATTAGCGTTGATGGTAGTGGTGTTGCAGTTATAGCTAATAC
TAGTAAAGGAATTAGCGTTGATAGTAGTGGTGTTGCAGTTAAAGTTAAAGCTAATGGCGGAATTAAAGTAGAT
GCTAATGGTGTTGCAATTGATCCTAATAATGTACTCCCCAAGGGAGTGATTGTAATGTTCTCTGGCAGTACTG
CACCAACTGGTTGGGCGTTATGTGATGGCAATAATGGTACACCAAATTTAATCGATCGATTTATTTTAGGTGG
GAAAGGGACTGATATTAATGGAGTGAGTACTAATACAGCTTCAGGTACTAAAAATAGTAAGTTATTCGATTTCA
GTTCTGATGAAGCTACATTAACTATTGATGGTAAAACACTGGGGAGAGCATTATCGTTACAGCAAATACCTAA
TCATGCACACTTTAGTGGAATAATTATGGATACAGAGAAAGTTAATTATTATGGAAGTAAAAAAATCACAACAA
ATGTGTGGGGTGTAACAACAGGAGATAATACTTCAGTACGATATATTTATAAGTCATCAGGTGTACTTGACTC
TAACAATAATGTCTCCAACAGTACCTTAGGCGGAAACAGTCTGCAGACGCACGATCATGATATTAAGATAACG
GGCACAGGAAAACATTCTCACAAAAACAAAGTAACAGTCCCTTATTATATTCTGGCTTTCATCATAAAGCTTTA
ATATATATGAAAAATTGAAAATATAAATTATCCATTAATAATAAAGAGGATATTAGCATGACTTCGGAGCCAAAT
CTGTTAAACCGGATTACAATTACTATTGAAGCTAATAATCAACAAGTAGCTAGAAAAGTATTGCATGGCTCCTT
GCTTAATCAAGCTAATATAAATAAATTATTTAATTCATACTTTAATGAATATGAAATTAATAGGGGTGTTTATTTA
GAAACATTAATCCTGAATCTTGGTACGATAAATTTCCATGATTTTAATTCATTGTTTCCTACTCTCCTAAAAGCT
GCATTGAATAAAGAATTCAGTCAATATCAGATAAACAACCATAGGGAAGAAATGCTATTTAATGAGACAATATC
AAATCAAGCTACTGATAAGTCTTACATATTTGGCGATAACAAATTAATTGATGCAGAGAATTTCATTCACTTTTT
ATATCAAAAGCATTCCACATTAAATCTAGTAGAAGCAATGGGAAATAATGGTATTGAAAAATTAACAAATCAGT
TAACACAAATAGAAAATAAATTTGCGTTATTATTGGCAAAAAGTTGTTTGTCTGAGGAAGGCTTAAAACGACTC
TTGGCTATCAAACAACCCGATTTATTAATCGCTATCAATCGCAGATTATCTGAAAGAATAAATAGACCACAATA
TCAGGAGAAGCTTGTTTCCTGCGGACAACTGATATTTAGTGCTCTGGGATATATACAACAGTACAATATACAG
GAAATTCCTAAACCGGATGAAAAAGTTATTGCACGCATAACAACTGAACTTAATAATAATGGTTTGCTTAATAC
AATACCTATTATTACACTATTTCGTCAGAGTGGGATTAACGATTCATCACTAAATGATTGGCTAAAGAAAATCT
GGCAGGTGAGATCAATTTCACAGTTATGCAGAAAGTATCTTTCTGCTAAGGAATACCAATATCTGTCAGAACA
TTTTGTTTCAAAGAGCGTCGATAAAAATAGATATGATGAAGAGCCCGTAAATCAGAGCATATTATCAAGGTTG
AATAATAATTCCATTAAAGAAGGAAATAATCACAGTCAACTCTGTACTCTCAGTAGACTATATTCTGAACCCGT
TGTATTACCTGAACAAACCATTCTACGTCAGGTTAGTAATACAGTAGATCAGAGCATATTATCAAGGTTGAATA
ATGCCTCCATTAAAGAAGGAAATAACCAAAGTCAACTTCGCACTCTCAGTAGACTATATTCTGAGCCCGTTGC
ATTACCTGAACAAACCATTCCACGTCAGGTTAGTAATACAGGTATATTAATTCTATGGCCAATGCTACCTACAC
TATTTAACCAGCTTGGTCTACTTGAGAAAAAGAAATTTATCCATCGTCAGGCCCAGTTTAATGCCGTTGATTTT
CTTGATTACCTGATTTGGGGAACCGAAGATGTGAAAGTGGAACGAAAGGTTTTGAATAATGTTCTATGTGGGT
TAATGGCTGATGAAATTACTGAACCAATGCCTATTGAACCAGAAAAACAATGGATAATAATTCAATGGCTGGA
CGCTATTATCTCCCAACTTTCTGGCTGGAAAAAGTTAAGTCGTAATGACGTCCGTCAATTATTTCTACAACGAC
CAGGAGAATTACTGATCAATGAACAGGAAATTAAAATCACAATACAGCAACAACCATTTGATGCTCTGTTAACT
GATTGGCCGTGGCCAATGAATATGGCTTGTTTTAGCTGGTTGAGTCAACCATTAACCATTAUGTGGTTATAAC
GATTGAGGAGAATGAGTTAGTGTGAGTAAAAAATATGAATATATGGGGTGTTTTTTATGATTGATTGAATGAGG
ATAAGGAGGGTGATGTATGGTTTTTATTTAGGGAAGTGGAAGGAATAGATGTGGGTGTTGAAGAGGATTTTTAT
TGTGTAGAAAGTCAGCGAAGTGAGCTCCTGGATGAGTTTCTGCTCACTGAGGCGGAAGTGGTGACCAGGCT
GGATAAGCCACTTGGTAAACCTCATTGGATAAATGATGATTATCTGGCGATATCGCAAAAGGGCAATGTAAGC
GTAATGGGAGGGTGGAGATTAATGGATGTGATGGAAGGGTTTGAAGTGAGTGATTTTGAGGGGGATGTTTTAG
TATTAGGGTTATTGGGGGATTTTGATAGGGGGTATTATGGAGTGTTTTGGGTGATTGAAGGGGGAGAAGAGGG
TGGATTAGGTTGTTTTGGGGTGGGATTGGAAGTGTTTTGGGAGTGGGGGGTGGAGAAAGAGGTAGAGGAAGG
GAGTTTTGTGGAGGGGGGAGGTTTGATGGGTTGGGAGGTATTATGGATGGATAGTAGTGAAAAAAGGGTGGG
GTGGGTGGAGAGTGGGTTTATTAGTGAGAGGGGGGTATATGAGTTTTTAGTGGGGGATGAGTAGATTATGGGG
GCTTTAGAACATTGTGCTGAGTGGTTAACACCGACAGGGATTGGCTGTTATCCTGAAGGATTAAAACAAGTAC
TGGGTAACGTATTGTTATCTGACAACGATAATATTAGACCGATTGTCTTATTACGGGGAATGGCCGGCAGTGC
CAGAGCTTATACCATTACTAATATGATGGCTTCAGAAGGGAAGCAAACACTGCTGGTAGATATATCCAAACTT
GGTGATAGGGATGAAAAAAAGATTATTGTTGAGATAAAGGATATTTTGGGGGAAAGGGGGATGGATGGAGGAT
GTTTATTATTAGGGAATTTTTGGTTGTTAGTGGAAGAGAATAAAGAAGTATTGGAGTGGGTGTGAGAGTTATTG
AATCAACCTGAATTAAGAATTGTTTGCCTGATTGAGCCTTATTCCCCATTGGTATGGCTGAAAAAGATACCGG
TATTAGTGATTGAGATGGGAGTTTTAAGGGGTGGGGAAAAAGGGAGATTGTTAATTGGGAGGTTAGGGGATAA
TTGTTGGGAGGATATTGATAGGATAAGTTTAAGGGAGGGTTAGAGTTTTAAGGGAGAAAGGGTGGGATTGATTT
TGCAAGAGGCCCAGCTTTATCAACAGCAGCGAGATCCGCTGGATATCTTGCAGCAATGCGATATACGCCAGG
CATTAAATTTGCGTGCTCAACAAAATTTCGGTCAATTGGCACAGCGGATTATTCCTAAGCGCTCATTAAAGGA
TTTATTGGTATCCGATGAGATTGCTCAGCAGTTACGGGAAATACTCATAGCAATTAAGTATCGGGAACAGGTT
GTGGGGGGAGGGTTTAAAGATAAAATTGGGTATGGGAGTGGTATGAGGGGGGTGTTTTATGGTGATTGAGGG
ACTGGAAAAACCATGGCAGCAGAAGTGATTGCTGACCACATTGGCGTTGACTTAATAAAAGTGGATTTATCTA
GAGTAGTGAATAAATAGATGGGTGAAAGAGAAAAAAAGTTATGGGGTATTTTGGATTTGGGGGAAGAGGATGG
AGGGGTATTATTCTTTGATGAAGCTGACGCACTGTTTGGTAAACGCAGTGAAACTAAAGATTCCCAGGACAGA
GATGGGAATATTGAAGTTTGTTAGTTATTAGAGGGGGTGGAGAATTAGGGGGGTGTGGTGATTTTATGGAGGA
ATAATGGTGGTGATTTAGAGAGTGGTTTTAATGGTGGTTTTAGTTTGATTAGGGGTTTTAGTTAGGGGGATGAA
AAAATGGGTAAAAAAATGTGGGAGGAAATTTGGGGTAGAAATATAAAAATATGGGAAGATATGGATTTTAAGGA
ATTAGCTCAACGAACAAGCGTGACTGGCGCGAATATCCGCAATATTGCTTTATTGTCTTCATTCTTTGCTTCA
GAGCAGGGGAATGATGAAGTCAGTAATGAAAATATTGAAATTGCATTGAAGCGTGAATTAGCTAAAGTCGGA
GGATTAAGATTTTAAAAGTTATGAGAATGAAAGTATTGAAATATTAAATAAATTTATTAGGAAAAAGTTATGAGG
ATATAATTTAAGAGAGGTTTTTTATGTTAAAGAGGGAAAGTATTATTGATGTGAATAAGGGAATGGATGGGATG
GTGGGGGGATATGTGAATGAAGATATTGGGATTGGTTTTGATGTAGGTGAATTGGATAGTATGGAATGTGATGG
GATGGTAAGTATCTTTCTTTATGACATTCATGAAGATTTACAGCTTCGCTCGGCAGAATCAAGAGGGTTTGAT
GTTTATGCCGGGAGGTTATTGCCTGGTTGGGTAAATATTAAATGTAACTATCTGATTACCTATTGGGAAGCTT
CTAAGCCAGCGACTGATGCCAGCAGTCCGGATAGCCAACCTGATAACCAGGCAATACAAGTGATGTCACAAG
TATTAAATGCCTTGATTAATAATCGTCAATTGGCAGGTATTCCTGGTGCTTATACTCAGGTTGTACCGCCTAAA
GAGAGTTTAAATAGCCTGGGGAATTTCTGGCAATCACTGGGTAATCGCCCACGGCTTTCTCTCAATTATTCAG
TGACAGTACCTGTTAGCCTAAACGATGGTCAGGATAGCGCGACTCCGGTTACCGCGGTTTCTTCTACAGTGG
AACAAACGGCATCGCTCAGTCAAGAAGTGGTTAGTCATGCTTTACGCGAATTACTCATTACGGAATTAGGAG
GAGGAGAGGATAACCGGTTGGTACTGAGTAAAGTTGAATTATCCGCAGTGAAAGAGACGATGACTCAAGACA
GTCCGGCTCAGATGATTATATTGTTGTCTGTTTCAGGCATTACACGACAGGAATATTTGAAGGAAATTGATAAT
ATCTTTGATCGTTGGGTAAATAATGCTGAAGTTATTACCACTATTGATGATTGTGGGATTAGAATTGAAAGTAT
AACGAAAGATAATCTTGTAGGAATTTAA
(Photorhabdus asymbiotica strain ATCC43949 PVCIopT operon, pvc1 -
pvc16; e.g. correspondinq to genes PAU 02112 to PAU 02099 of the sequence
of GenBank accession no. FM 162591.1)
SEQ ID NO: 94
ATGGCCACAACCACAGTTGACTATCCAATACCGGCTTATCGATTTGTTGTCTCCGTTGGTGATGAACAAATCC
CTTTTAACAGCGTTTCGGGGCTGGATATTACTTATGATGTCATCGAGTATAAAGATGGCACCGGTAATTATTAT
AAAATGCCGGGTCAACGTCAGTTAATCAATATTACACTGCGTAAAGGGGTATTCCCTGGCGACACTAAACTTT
TTGATTGGCTTAATTCCATTCAGCTTAATCAGGTTGAGAAAAAAGATGTTTCAATTAGCTTGACCAACGAAGTT
GGAACTGAAATTTTAATGACCTGGAGCGTAGCCAATGCATTCCCAACCTCATTAACATCTCCTTCTTTTGATG
CCACCAGCAATGATATCGCTGTTCAAGAAATAAAACTGACTGCCGATCGAGTCACTATTCAGGCAGCTTAAAG
CATCACGATGATTGATATATCAGACGGGACAAAATGATCCTCAAAATTTGGCACAACGGCTACCCGTCCAACT
AAATTTACCCTCTTACAGTTCACGCAAAATATCGCACAATACAATTGGAGGCAATATGCCAACAACAACTTATC
CCGGCGTTTATATTGAAGAAGACGCCTCACTGTCACTTTCCGTTCGCTCAAGTGCAACGGCGGTGCCCGTTT
TTACCGTTGAAGATGACAGTCAACTTCATACTCCTACCAGAGTGAATAGTTGGTTAGAATATCTGACAAAAAAA
GCAGATAAAAAATTCAATTCTACCGACAAACTTGATATCGCATTGCGCGCTTATTTTATTAACGGCGGCGGAT
ATGGTTATCTCGTCAAAGCGGGTGAATTAACAAATCAAATTCCAAAACTTAACGATGTCACATTACTGGTCGC
GGCTGGAGAAAATATCAAAGATGCTGTGAGTACACTTTGTCAACCGGGCAAAGGCTTATTTGCCATTCTGGAT
GGCCCAACCGAAGAGTTAAAGTCTGATGGCAAATCCAGAGATCCGTATGATCAAAGCCCTTTTGCCGCCGTT
TATTACCCCTGGCTAGTTGCTGATTGGGCAGACAATATTCCGCCAAGCGCGGCCATTGCCGGTATCTATTGT
TCAGTTGACCGTACCCGCGGTGTCTGGAAAGCCCCAGCAAATGTCATATTACAAGGCGGGGTGAAACCGAA
GTTTAAAGTCACCGATGACTTACAAGGTATTTACAACACCGGTAAAGCCATCAATATGATCCGTGAATTTCCG
AATACCGGTGTCACCATCTGGGGCGCCCGCACACTTAAGGACGAAGATAACTGGCGTTACATCCCAGTTCG
CCGCCTGTTTAACAGTGCAGAGCGAGACATTAAAAATGCCATGAGTTTCGCGGTCTTTGAACCTAACAGCCA
ACCCACCTGGAAAGCTGTACACCGAGCTATTGATAATTATCTCTATGCCCTTTGGCAACAAGGAGGGCTAGC
AGGAAACAAAGCTGAACAAGCTTACTTTGTGCAAATTGGTAAAGGGATAACCATGACCGATGATGATATCAAG
CAAGGGAAAATGATTGTTAAAGTGGGTATGGCCGCAGTGCGCCCGGCTGAATTTATCATCCTTCAATTTTCAC
AAAATGTAGCACAGTAACCGTACTGAGGCGCGGTTTAACACCGCGTCCATTCAGTCTATTGAATGGAGGAGA
CAATAATGATAACGGAGATAAAACAGCCGGGCGTCACCATCACGGAAAATTCGATATCCCCGAAATCAGATA
ATGAATTTATCGGCGTCCCCGTTTTTATTGGCCATACCGAAAAAAATTCAAGCCATAAAACGGCTGTTAAACTA
AATAGCCTGATGGACTTTACCCAAGCTTTCGGTGCATCAGGATTAACCTATTATTCAGTACGCCACTTTTTTGA
AAATGGTGGACAGCAAGCTTATATCTTGTCACTGGGGATTAATCAACAGCTAAAAGATTTTCAATCATTGATTA
CCGCCCTGCAATGGAACTGGGTAAAACAAGCCATTGCCGCAGAAAACGAAATCACATTGATTGTTGTGCCTG
ATATTACCCGTTTTAATGATCTCAGCGCTCAAAAAAGCCTTTGGCTACAACTCTGGCAATCAATACTTGAACTG
TGTAAAAGTCGGCGTGGCATCATGGGATTACTGGACGCGCCTGATGATCCAACATTAGCAACTGAGTGTTTA
AAACAATTCTCTTCCACTGATCGCCAATGGGGCGCCGTATACTGGCCAAGGCTAAAAAGTACCTACCAAGAA
AACGGTACATACATTGTACTTTCACCTACTGCTGCGGTCGCCGCCGTTATGCAACGCAATGACAGTCAGAAA
GGCATATGGACTGCTCCCGCCAATGTGGCTTTAGCCAACGTCATCGGTCCGGTACGTTCTTACATTGAAGCT
GGAACCTTGCTGAATCAAGAAGGCACTTCGTTGAATCTGGTGCGTAGCTTCCCCGGCAAAGGCATTAAAATC
TGGGGCTGCCGCACTCTGGATAACATACCTCATTCTCCCTGGCGTTATATCCAAATTCGCCGTTTGGTTTCCT
ATATCGAAGCTCATATAACCCAACTTGGCCGCGCCTTTGTCTTTGAACCCAACAACGCCATCACCTGGATGAA
ATTTAAAGGTCAGGCCCACAACTGGCTACGTCAATTATGGCTAAAAGGTGGATTACGGGGCACTCAGGAAGA
TCAAGCATTTGAGGTGTTACTGGGTGTTAATGAATCCATGAGTGAAACGGATATCTTGGCCGGAAAAATGATC
ATGAAAATCAGGCTGGCGCTGTTAATTCCGGCAGAATTTATTGAGCTGAGTCTGACGTTTGATATCCGTAACA
ATACCGTACCTAGCTAATCTAAACAGGGGAAAAACATGTACAACTTATACACCCCGTCAGTATCTCACCGTTT
TATCGCCAGTTTTCTGTTTAACAACATTCCCAGCCCACTTGATATCGCCTTTCAGCGTATATCTGGCCTGAGC
CGAGAACTGCAAACCACCCAACATAGCCAAGGTGGAGAAAACGCCAGAAACGTCTGGTTATCCGAGAAGAT
CCAACATGGCAGCCTGGTGCTGGAGCGCGGTGTTATGACCATCACTCCCCTCACCTTGGTTTTTGATCGCGT
GCTGCGCGGTGAAAAAGCCGTGTATGCCGATGTTGTCATCATGCTACTGAATGAAAATGCGTTACCCGTGGC
GAGCTGGACAGTCAGTAACGCGCTACCGGTTCGTTGGTCCACCAGCGACTTTGATGCTAATAGCAACACCGT
ACTGGTGAGTTCTCTGGAATTACGTTATCAGGATATGCGCTGGTTAGGAGTAAAAGCATGACGGTAGAAATTA
AAGAACTGATTATTCAGGCTAAAGTCACCGATTCTACGAGTGATCAACTCGCCCCAAGAACATTAGCCCAAGA
AAAGCTGGATAACGCCCGTTTGATTGACATAGTGAAACGGGAAGTGTTAGAGGCATTACGTGAAGGAGGCCA
TCATGAGTTTAATTGAACGTGGTTTATCCAGACTCACCCTAACCGCTTTTAAAGACCGAGAAGGTAAAGTTTC
CGTGGGTCGCTTACAAGCCATGTATAACCCCGATACGATCCAGCTTGACTACCAAACCCGCTACCAACAGGA
TGAAAGTGTTAATCGTGCCAGCCAAAGCAGCCGTTATGTATTATCCCAACCCGCCGGATTATCCTTAGTTCTG
CTGTTTGATGCCTCGATGCCCGATAATAACATGCCGATAGAAACCCAGCTTGCGACCCTGAAATCCCTGTGT
GCGATTGATGCCAGCACCAAAGTACCCCACTTCCTTAAAATCAAATGGGGCAAAATGCGCTGGGAAAACAAA
GGTTATTTCGCCTGCCGAGCCAGTAGCCTGGCCGTCAACTATACCCTGTTTGACCGGGATGCCACACCATTG
CGGGCCAGCGCCACTCTATCTCTGGTAGCGGACGAAAGCTTTATTATTCAAGCTACCGAACGGCAGTTAAAA
TCACCGCCGGCCACTGCGGTTAGCGTAACTGATATGCTCTCCCTGCCTTTGATTGCTTTAGATGCTGGAGCG
TCTCTGGCTGGTGGCATTGATTATCTCTCGCTGGCCTGGCAAAACGGTCTGGATAATCTTGATGACTTTACCC
CCGGACAAACACTGCAAGCGCGGGGGGATGCATGAAGATACCCATGATAACCCTCAAAATAGGTGGCAAAA
CGCTCAATCAATTGACTGTCATCAGTCTGACAATAAACCATCAAATCAATGGCATTCCCTCGACCAACATCAC
CTTGGGGATCGCTGGCGATGCGAGCCATATTTTCGACACCAAAGCCCAAGCTGAACTGGCAAGTTGTCGCC
CCAATAATGAACTCACCCTACAGATCCAAAAAACCGTGGTGTTTAAAGGGAGCATCGTTCGACAAGCACTTGA
ACTGAAAGGTCAAGACAGCATCATTACCCTGACAGCAAAACATCCACTACAAAAGTTAACTCATAGCCTCCAT
TCACAATTATTCAGTCAACAGAGTGATGAAGCGATTATCAGGAAATTATTCAATCAGGCGGGTATCCAAACAA
CGATAAAGCAGGCTCCTCAACTTAAAACCGTTCATGAACAAATGGTGCAATTTCGTTGCAATGACTGGGCATT
CCTAAAAAGCCGATTGATTGCCACTAATACCTGGCTGTTGCCCGGCAATGAATCGGTTACTTTGATAACACCT
AAGGCCCTGAATCAATCGACAGTGCATACTCTTCATCGACAGGCCAGTGCTGAAGATATTGTGTTATTTGCAG
CGGATCTCCAATGGAATAACCAATATAGCCCTAAAACGGTGAGTGTACGTGCCTGGGATATTGCTCAACAAA
AGCTTTCCCCAGCAATTAATACCCAAAACAGTCAGCTTGGCAGTCATAAATTGGCCGTGGACAGTATCGCCG
CACTGGCTGATAAAGAGTGGCAATGGGCTTACAGCTATCCATTAGATAATGAACAAGCCAAACACCTTGCTCA
AGGCATTATGAATAACCTGCGAAGCCATAATATATCTGGCAGTTTTGAAATCGAAGGTAATCACCGTTATCAA
CCGGGGGATGTCTTGGCGTTAAATGGTTTTGGTCAGGGGATGGACGGTCAAGGGATTATCACCGGAGTCAG
TCAGATAATTAATCAGCGGCAAGGCTGGCACACCCTATTAACCTTAGGCATGTTACCCGATGTAGAACCGCC
GGTGCCTCAGGTGAAAGAGTTGCATATCGGTATCGTGGAAAAATACCAGCAAGACCGCCAATCACTAAGCCG
TATCCCAGTCAGAATACCCGCATTAAACTTGACCAAAGGTGTCCTTTTTGCCCGGCTAGGTAAACCTTATGCC
AGTCATGAAAGCGGATTTGCTTTTATCCCGAACCGGGAGATGAAGTGATTATCGGATTCTTTGAATGTGATC
CTCGTTTTCCAGTGATATTAGGTTCCATGCATAATCCGAAAAATAAACCACCGTTAGAACCCAGTGAAAAAAAT
CCGGTGAAAACTTTAGTTATCAAGCAAGGGGATAAACAACAAGCATTAATATTCGATAATAAAGAAAACACGG
TGGCACTTAATAGCGGCGAAAATAAAGTCTCTCTGCAACAGGATAAAAACATTACGCTCAATTCAACTAAAAA
TCTCATCACTCAGGCCCAAGAAATTAATATACAAGCGGAAAAATCTCTGTCAGCCACAGGAAAATCTGGCGTC
GATATTAAGGGCGCGAAAATTAACTTAACCCAGTAATGAGGTATTGAAATGACAAGCCAAATATTAGCCAATA
TTTACGGTTGCGGCTGGAAATTTCCGCCACAGTTTTCTATTGAAACTGGCGTAGAAATGGCCGAAGGTGCCG
AAAACGTTCGCCAAAGTATGAAAATCCTTTTTTTAACTGAACCCGGTGAACGAATTATGCGTGAAGATTATGG
TTGTGGTCTGAATGATTACATGTTTGAAAATATCAGTGATGAATTATTATCGGAGATTCAAACCCGCATTGAAG
AACGAGTATTGCGCTATGAACCCCGTGCTGAAATCACAGATATCCAAGTAACTCAGAAAACAGACTCACCGA
ATACTTTACATATTCAAGTGACCTATGCCCTGAGAGGCAGCCAAATCAGTCAACAGCTTGAAGGGGTTCTTGA
GATCAACGAAGGTCAGGCAAAGGTGAGTCTATGAGCAAACAACTCATTATTGATGGCGACAGCCTGCTATTC
GAGCCATTATTCGGCAACCGGCAGGTCACTATTTTGATGCCAGCGACCATCAGAGGCAGCGGACACGCGCA
AATCCAAGGCAGAAAGATAGCGATTGTCGGCGATGAAAAAAAGGTACAACTTCAAGCGCAATACATTACCCC
AAGCCACCCGGTACCTGGCATAGGCACAGTTACCATTGCTCAATTAGATACCAGCCAGCAAGTCAACTTTTG
CCACAGCCCTGCCACAGTGATAGTTGTCGGGCAGCAATTTACCGCTCGATTTACCCCATCACAGCCGGCAAT
TAATCCGTCAACCGGGCCAGATGTCACAACACCCAGTATGGGCAAAGGCCGTTTTATTGCCAGTCAACATAC
TATCAACGCCGGATAAATAACTCTGCAAAATCATTATTCAATAACGTTCCTATTCTGCAATAGCTATCAGCAAT
ATATTCAAATAACAGGTGGTATAATATGGGACTCACCGAATTAAAAAATAAACTCTCTGCTATCGTACTCGATA
CGGATTTTAAACTTGATGAAAGAAGTACACTGGATATTTTAAACTGGCTACAAGAATATGCTAAAAAAATCCCT
TTCAATCAAGAGAAAAAACAGTTCTGGGATAGTTTCTATTTTATTCAGGAAAATAGTCCTGAGAAATTAGCCGA
TCTTTACCAAAACGTTAATAAAACGAATGGCCATTTACCGGCCCATCAAGCTTTTGTTTTAGCCTTTTTAAAAC
TTTTAGAAACCACCAAAGTATTATTTAATACTTTTCCGGCACGACATCGTGATCTTTATTACCGGGAATTATTA
GGTCTAAAACCCAGAAATGCCCAAGCAGATAGTGTTGCTTTAGGCATTACCTTAAATACAGATAACACAGAAC
ATCTTATTCCTAAAGGAACCTTGTTCGATGCCGGGCAGGACAGGGCCGGAAATCCGCTACAATACGCATCAA
ATGCAGATTTACTGGCGAATCAAGGAAAATTGAGCGATCTGCGTTGGTGTCGAAAAGATAATGATAGCTGGC
AATCTGCAATACTACTGAACCACTCAGATAATATTGAATTACCTGAAAACAGTATTCGACTTTTTAGTCCAACG
CCGGATGATATTCCCGTTTTATCCGGTTATTTGATAACTTCGTCTTTATTTGCTATGCCAACGGGGGAACGCA
GTATTACATTGACTTTAGCAGATAATTGGCATGGTGATATTAAGCACATCACCGCTAAAATCAGTTCGGGAGA
TCACTGGCTTTCACTATCAGTAAAAAAAGAACAAGACAATAGTATTCACTATCTTAAACTTTATTTATCAACCAA
TGATGACCCCATCGGTCCTCCTGATGCTTTGGATAATATAGCGTTTGATGTACCGGTATTAAAGCTGGGCACT
GTTCAGGGACCTATACTACCCAAGATTACGGGTATTGAAATTAGCATTAACGGCAACAGTAATGTACATTATT
CCTCTGATAACGGTATTGAAAAAATAGATGCAGCTAGTTTTCCCTTTGGACAATCACCGTCACCAGGTTCCGG
TTTTAATCTGATTGCCCCTGAATGGTATGGTACAGAAAGCGCCAAAATTACTCTTACTCCTCAATGGACTGGA
TTACCCAAAGAGGGGTTTAAAGAGTGGTATCAAGGATATAGTTCTACCCCCGAAAATAATGCATTTAAAGTAC
AGGCTTATTTAATCACACCTCAAAAGAGAGAAAAATTTAATGAAGCTCAGTCATTATTTAATGAAAGTAAAGAC
AAGAAACCACAAGGAAAAAGCCTAACTTTTACCTTACCTGCAATGGATTATTCCTTTGCAAACAGCCCATCAT
CTAATAACTGGCCCGCATCAATACGCATAGAACTAACCGAACAGGATTTTATGCATGCCCAATATTGGCAAAA
TCCTACGGGTAAAAAACAGCCCTATACCCCCAAAATGAACACATTACAAATTCAGTTCAGTGCCAAAGTTAAA
CCCGAACAATTTTCCGTTTATTCTCTCACGCCTTTTGGTTGGGGAAAAACAGGAGAAAATAGAACATCATTAA
CCCATGATACATTCTATTTAGGTTTTACCGATGTATTACCAGGACAAACTTTATCCCTGTACTGGCAGTTAGAA
GGTATTAAAAAGCTCCCTTTATCCTGGTCTTATCTGAATCAAGAAAATACCTGGAGTCCATTGGATAATCAGGT
GCATGACCAAACCCACAACCTATTTGATCGAGGAATCTGGCGTACCTCATTGCCACATGATGCTTCAAACCAA
GCCTCTCAAATGCCAAAAGGACAATATTGGGTGAAGGCACACATTTTACAAACGAATCAAGCAACCCTGACT
GATCTGTATTGGTATCGAAAAGATAATGATGTCTGGAAATCCGCAACACCTCTTAGCCTTTCAAATAACATGAA
ATTACCCGCAAACGGTATTCAGATTTTTAGCCCAACATCTCATGATGTTCCAGTTCGATACGGCTACCTAATTA
CTTCATCTTTATTCTCATTCCTCAAGAAAGGACGCAATATCACATTAATTTTAGCAGGAGATAGCTGGGAGGG
TAATCCTGAAAACATCACCGCTAAAATCAGTTCAGGAAATCACTGGTTAACACTATCCGTCGAATATCTGAGT
AATACTAATAGTCTTAAGTTGCAATTATCAGATAATAATAATGATCCCATCAGCCCCCCTAATGCTCTGGATAA
TATGACGTTTGACACGCCATTGTTAAAACTAGAAGCCACTCAGGATTTCACTTTGCCCTGGATTTATAAGGTAT
GCGTTAATAGCAACAATATACTCTCTACCTCTGACAGCTCAGATGCAGCGATTACTCGTTTCCCCTTTGGCCA
ATCACCATCGTTGGGTTCCAGCTTTAGTCCGAAAATCGTTTTCCCGGAATGGTTTGAATCTGAATACGCATCA
GACACCACGATCACGATTACCCCTCAATGGGTTAACCTGCCCACAGAAAACTTTTCATCGTGGTATGACGGA
TATATTAATAAACCTGCCGATAATAGCGTATTTAAAATAGAGGGTTATTTACTTACTCATTATCAGGGAAAAATC
AAACTCACAGAAGCTGAGACAGGAAGCGAAACCCAAGCATTATTCAATGGAAACAATGCACCACAAGGAAAA
AGCCTGACTTTCACTTTACCTAATAGGTATAACTTCTATCCGCGCAACCATCAGTCAATGAAGATAGAAATAAA
ACTCGTTAAACAAGACTTTATGCACACTCAACATAAGAGCAATCCCACAGGCAAAAAACCACCCTATACCCCG
CAAATCAGTGCCTTACAGGTGGAATTCAATGCTACAGCTTTCCATCGAAAATTCTCCGTTTATCCTCTCACGC
CTTTGGCTGGGGCAAAACAGGAGAAAATAGCACACCATTAATTUATGATACATTTTATTTAGGGTTGAGGGA
TATATGAGGAGAGGAAAGTTTCTCTGTATTGGGAGGTAAAGGGGGTTAAAGAGGTAGGTTTGTGTTGGTTTT
ATCTAAGTGAAGAAAATAGCTGGAAATCATTAAATAGATCAACTTACAACCAAACCCACAACCTGTTTGAATCA
GCAGAACAAAGTATCCTATTACCACGGGATGCTTCAAACCAAGCCTCTCAAATGCCATTAGGACGGTATTGG
CTGAAAGCACAGATAGAACAGGAGAAAAAACAGATAAAGATAGCGCTTCCTGATTATTATCCAAGAATCAGG
GGGCTGTTGTATAACGCTACCATCGCCACTTTAATCAACGCTGAAGCTGTTGAGCAATCTCACCTTATCAACG
GATTGGCTGCTAACAACATTAAACAACCGGTTAACTCATCCGTTGCCATCAACGAAGTTATTCAACCCTGGAC
ATCCTGGAACGGTCGCCCAAAAGAAACCGAGTCAGCATTCCTGGCACGAGTTCCTGCCCGGCTCTCTCATC
GTAAGGGAGTGGTAAGGTGGGGTAAGATTGGGAGTTTATTAAAAGAGAATTTTAGTAGGTTATTGGATGTGAA
ATACCCTTCTGTCAGTGAATTAACCAAAATTCCAGCGCCAGAAAAGCGACAATTAACCATCATCCCCGACAAC
CGCTATAAAGATAATGATGATTCACTACGCCCAGTATTGAACCAAGCCAGACTGACCGAGATGGTCGAATGG
TTAGATCGATTAAGTAGCCCTTGGACAACTATTGAAATTAAAAATCCCACATATGTTAACGTTCTGATCCACTA
TGAACTGATATTTACCTCGGATGTTAACCCCGATTATGGCCTCCATCAGCTACAACAAGAACTCAGTCGAAAA
TATATGCCGTGGGGAGAAAATGCAGCTATTGGCGTAACACCCGGTAATCGTATTGACTACTTCCAGTTATTAG
CCTCAATTCAACAATCACCGCTGGTTGAACGGGTCACCAACTTAACGTTAAAAAAAGGCAGCCAGCCTACCG
TAAGTGAAAGTATAGAAGGGGGGGATGATGAAGTAGTGATTTTAGTGTGGTGATAAAAAGTTGGGGAAGGTAA
GGAATTAACAAATGAATAATCGAGATATGCTATTTCCTATCATTAAAGACGATATTACCTTTGATTCTTTATTCG
CCCAGGCAAAAGCCGTTATTGAACAACAATCGGGGCAGCTCTGGAATAATACAGGTGAAAATGATCCCGGCA
TTACTTTATTAGAAGCCTGTTGTTATGGCGCATCCGATCTGGCCTATCGCCACACATTGCCACTGCGAGATTT
GGTTAGTGGTGAAGAAAATGAAGGAATAGATGATGGGATTTTTGGGAAAGAATTGGTGGAGAAGAAATAGTG
ACCTGCGGCCCAATTACCGCGGAAGATTACCGTCGAGCTTTGTTAGATTTGCGTAGTGATAACACCGTTGAA
GGTTATTTTTTGTTTAATGATGGAGAGGTGATTGGTGAAGGGGAAAATGAAGGGTATTGATATTGGTATAAGAA
AGAAAAAGGGGAATAGAGTTTTAGTGAAGAGGAATAGAGGGAAGAATTAGAGTTAAGAGTGAGAGGAAAGTAT
TGGGTGTATTTAGTTGGGAGTGGGAAAAGGGAGGTGGATAAGAGGGTGGGTGAAGAAAGAGTGAAGATTTTTG
TGAAAGATAACCGAAACTTAGGAGAATCGGTCAGTAAAATTATTTGGCTAGAACCCATTAAACTGTCATTGAA
AATTGATATTCAGCTTGATGATGACGCCAAAGATATTGCTGATATATTTGCTAAAGTTTATATGATTGCAGAAC
AAATGGTGCTTGAAAAACCATTACGTTATACCACTCAAGCGATGAAAGAACTGGGTTACAGTCAGGAACAAAT
ATTTGAAGGCCCTTATTTACACCACGGTTGGATACCGAAATTACCTCAAACCAAAGATTATACTCACCCTACC
GTATTAAATCTCAGTCCTTTAATTAATCAGTTACTGGCTATCAAAGGGGTGAAACATATTACCCAATTTACATT
GGATAAGCCTGATAAAAAAATTTCTAAGTTACCAAATGATAATTGGTCTTGGGAAATCGCTCCGGGATATTAC
CCAAAACTATGGGGAGATACTCCATTAGAATTAATTACCTCACCAACAAGCCCACTCACCATCACGGCAAAAG
GGGGAATTAAAATTGCTATTACTAAACAACAGATAGAAAAAAACATAATGACAGAACCACTAATTAATACACAG
CCAGAATTATTGAACTGGGGTAAACATCGCAAAGTCCTGGATTACTATCCGATAAGCAATAAATTACCCGCTT
GGTATGGATTAGAAAGTAATAGGGAAGAAGAGGTAGAGTTGGATGAATTTATGGTGGGTTTTGAAGAAATGGTA
GGGAATAAGTGGGGTGAAGTTGGTTTATTGGGAAGAGTATTAGGTTTTAAAGAAGGAGGAAATAGGGTAGATG
GGATTGAATGGGGTTTTAAAGAAAATAGGGTTGGTGAAGATGTTGATAAGGAGATAGTATGTAATTTAAAGAAT
AATGCTACGAAAATCGATAATAATGCCGATGACTACGACAAGGAACTCGTTATTCTAGATTATTTGTTAAGATA
TTTTGGGGGTGAATGTGGAATGGGAGGAGTATGAGGAGAGGGAGGAGAATGATGATTAAGAGAAGGTGAGAGT
AAAAAAGATTTTGTATGTAGTGAGGGGGAATATGTGGGTGAAGAGGGAAAAGTGAGTTATGAGGGTAAGAATAT
TCGGATTGATAAAGTATCAGCACTGCAAAAACGTATCGCTGCCCGATTAGGTCTGGGAGGAGAATGTTTCAA
AGCAGAGCCTGACTTAGCTCACCTTCCTTTCTACCTCATTGAACATCGTAGGCTCTTACCAGTAAAACCTGAT
ATAAAATTCTATATTGAGCAACAACCTAATTCTCTGGAAATTGAAAATGATAAATTAAAAATCACACAGAAAGAT
TCAGCGGGTCGGTTACTGCAAGGTCAAGTTATTAACCTGGAATTTCGTGAGGGCTATGATGAATTTACATTGC
TAAACTTAATGATAACTGAAGTGACAAGAGATACATTCACCATTAGCATTAATAATAGCCGTGATCTCAGAGAC
AATCTGGACAAAGTGCAACACGCGTTTGAACAAACGAATAATCTGAGCTGGCACAATAGCTTAATATGGATGG
AAGATATGGATTATCAATTGGTTTATGCCAATGGAGAACAACTGGAAAAAGCGGAAAATGAACGATGGATTAC
CATTAACAATCAAAGTGCTTTCCCTGCTATGATCGGAGAGAATGATGAAATCACACTAAAAATTCAATCCGATT
ATGAACTTAAAACCAAAGTCGTGCGGCTTGATTATAACAACAAAAAAATTCTGATTATAAAAGATGCGACATCA
ATAAATAATTTTGGGGGAAAAAGAGAAGGATGATATTATTGTTGGTGTTGTGTAAAAGAGAATGGGTAGGGATA
TTCGGATGAATATAAATATGAACTTACTTATATTGATACAGATTCTACAAAAGAAAATGAGTGCTGGATTACTAT
GAGGGATGGAAATAATTTGTTTTGTGGTGATATGATGGGAGAGAATGAGGAAATTATATTGAAAGGTAAGGGTA
ATTATGAGTTTAAAACGCACGTAGTAAAATTTGATCGTATTAATAGACAAATATTACTTAGGAAAAATACAGAC
GTGGAAAATAATTTTGGATGAGAAAAGAAGAGATGGGAGTATGGGTGGGATTTGTGTGGTGAAAAATATGGGG
AAAGTGAGGATTTTTGATTTGTTGTGAGTGGAGTAGTGAATGGAGAATTAATTGAGAGGGGGAGAGTGGATGT
CTATAAATTAGAGTCTTGGGTAAAAACTGAGATTTTATCTGAATTACCCGCGCATATCTCACTCGTTATTCATT
GGCTATCATCGGAAGAATTCGAAAAATTTGCCAGTACTTATAAAGTTTGGCAAAATAATGGCGCTCCTTTAGG
TGATCACGCATATAAAATTCTAGAAACATTAACACTTGGGAAAAAACCTTCTACTTCAGCAAGAAGGTCCAGC
AGCTATATAGAAGCACAGTAATAATTCTTACAGAACATTAACCCATATTTATCTTATAATATCAAACATCATAAA
AACAATCTTCAGCTCATTATAATGACATATTTCATACTCAGGTTTCTTCATATCTGTTAATTACAAAGAGAATAT
TAATATGATCTCAGCACCAAATCTGTTAAATCGGATTATCATTACTATTGAAGCGAATAACGCACAGGCAGCTA
AAAAAGTATTGCATGGCTCCCTGCTTAATCAATCCAGTATAAACAAACTCTTTGATTCATACTTTAACCAATAT
GTTGTAACAGACTATCTACCTGAAGACACTCACCCTGAATCTTGGCGAAATACGATTAAATAGTTTTAATTC
ACAGTTTGTTATTCGGCTTAATACTATTCTGAGTCAAGCATTGAGCCAATATCAGGTAAATAATCAAACTGATA
TTGAGAAATTTATTTATTACTTATATCGAAAAGATTCTATATTAAACCCAATAGAGGAAATCAATAATCGTGAAA
TTACTGACATCAATATTAAGCAATTAATTAACCAATTACCCCAGATACAAAACAATTGGACACTATTATTGGCA
AAAAGCTGTTTATCCACACATAGCCTGAAAAAACTCCTGGCTATCAAAAAAACAGCTTTATTAACCGCCATTAA
TCGTAAATTATCTGAAAAGATCAATATATCACCCTATCAGCAGGAATCGGTTTCCACCTGGCAATTGATACTGA
ATGCGCTGAAATATATACAGCGACATAATACACAGGAAATACCTGAACCCGATGCGAAAGTCATATCACTCAT
TACAACGGAACTCAATGACAATGCCATTAATACAGCACCAATTATTGCATTATTTCGCCAAGTTATAACCAACC
ATTCCCCACTGAATAAGTGGCTGGAACAACTGTGGCAAACAAAGCGAATTTCACAGTTATGTAAAAAACAGCT
GTCAATTGAAGAATACCAACATCTATCGGAGCGCTTTATTGCCAAACACGGGAATAAAAATAAATCTGATAAA
AAATCATCCATGACTTCCGAACCGCTGTTATTACCTGAACACCCTCCACCACGTCAGGTCAATAATGCTGGAA
TATTAGTTCTGTGGCCGATGTTACCTACTCTATTTAACCAATTCGGCCTGTTTGAAAAACAAAAATTTATTCATC
GTCAAGCTCAATTTAGGGCTGTTAATCTACTTGATTATCTCATTTGGGGAAACGAAGAAACACAGACAGAACG
AAAAATATTGAATTGCGTTCTGTGTGGGTTAATTGCCGATGAGGACACGGAATCAATCCCTATTGAGCCAGAA
AAACAACAGGTAATAGAACAATGGTTAGATGCAGTTATCAGTCAACTTCCTGCCTGGAAAAAATTAAGCCGCA
ATGATAGCCGCCAATTGTTTTTACAACGCCCGGGGGAATTGCTGACAAATGAGCAGGAAATCAAAATTACGG
TACAACCTCAACCATTCGATGCACTGTTAAATAACTGGCCCTGGCCGTTAAATATCGCCAAACTTCCCTGGCT
GGATCGCCCTTTATTAATCAACTGGTAAAACATTGACAAGGTTTATATGAAAGAACATCAATATAGAATAGTCG
ATCTACGCTGGATTTATTCCCATTTGGAGCGCATCGATCTGCTGTTACAACGTCACTATTACCAAAAGAGAGA
CAAATACGATTCATTGCCAGAAAGTTTTTGCTTGAAGAAGATGAATTAGAACAACGTCTAGCAAAACCGTTG
GGTATTCCTCATTGGCTAACAGCAAATACCGGCGCTGGTGATACAGAAACAGAAAATCATTCTGCTTCCGGC
ACATTATCACTGCTAGTCACGCGTTTTAAACTCACTGAATTTGAACGTGATGTGTTATTGCTAGGTTTATTACC
GCATTTTGACAACCGCTATCATGCGTTATTTGCTACTCTGCACGGTAACAGTAAAAAACAGTGGCCCAGTTTT
GATTTAGCGATTGAATTATTTAGCCAACATCAAAGTAACTGGCAATTATTTCAACACCACTTTTTACCGCAAGC
TCCATTAATCAATCACCATTTATTACGACTCAATAACCAAGAGGAACCCATTTGGCTACAAACTCAATTTTTAA
CTCACAATGCAGTCTGGTCTTTTTTATCCGGTCAGCGCGTCATTTTACCTCCCTTAATATCCTGCGCTTACTG
GCATATTCCAACCTCACAGACTTGGTATCCACCAATCCTTGGTCATGCATTTGAAAAAATATTGCTGAATGAAA
CGGACGAAATACGCCCGCTGGTGGTTCTTAAAGGAAAACAGGACAGCGCCAGAGAACTGGCAGTCAGTAAT
ATTATGGGAATTCACGGCATTAACACTTTAACGTTCGATTTATTTCACCTGCCAGATGAAGAGTGCACCACCT
CAATACTCAATCTGCTAATAGATGCAATACGAGAAACCCGGCTACATAATGCCTGTTTATTAATCCGTAACTTT
TCTTTGCTGGCAGAGGAAAAGAGAATATCGCATAGAGAATTATCAGCTCTACTGAATCAACCCAAATTACGTG
TGGTTTGTCTGGCAGAGTCAGAAGAATCATTAGCATGGGTTAAACACCTGCCGATAGTGCAAATTAATATGCC
ACCGGCGACGCTGGCAGATAAAAAAACGATGCTGGAAGCCAGTTTGCCAGATAATGTCACTAAAGGAATTAA
TATAACTCAATTATGTCAACGTTTTTCATTTACAGCAGAAACATTACCGTTAATTATCAAGGAAGCTCATCAATA
CCAAATCCTCCGACAACCGGAAGATCAATTGAAAGAATCTGATCTACGTAAGGCATTAAATTGCCGCGCCCA
ACAAAATTTCGGTAAATTAGCCCAGCGTATGACACCAAAACGAAGTTTTAATGATTTGGTTATTTCCGCTGACT
TAACTCAACAGTTGAAAGAAATCATCGCAGCAATTAATTACCGTGACCAAATTCTGGGCGCAGGTTTTCGGGA
AAAAATCAGCTATGGTACTGGTATTAGCGCCCTATTTTACGGTGAATCCGGGACGGGGAAAACCATGGCCGC
AGAAGTGATTGCCAGCTATCTTGGTGTTGATCTGATTAAGGTAGATCTTTCTACCGTGGTGAATAAATACATC
GGTGAAACCGAAAAAAATATCTCCCGTATTTTCGATCTGGCCGAAGCGGATTCCGGGGTGCTGTTTTTCGAT
GAAGCCGATGCCTTATTCGGTAAACGCAGTGAAACCAAAGATGCCCAAGATAGACATGCCAATATTGAAGTTT
CTTATTTATTACAGCGACTAGAAAATTATCCGGGATTAGTGATTTTAGCGACTAACAATCGCAACCATTTGGAT
AGTGCGTTTAATCGCCGCTTTACCTTTATTACCCGCTTTACTTATCCCGATGAAGCATTACGCAAAGCAATGT
GGCAGGCAATTTGGCCTGAACAACTTAAGTTATCAGATCAACTTGATTTTGAGCATTTGGCTAAACAGGCAAA
TCTGACCGGTGCTAATATCAGAAATATTGCCTTATTATCATCAATATTAGCTACAGATAATAATAGTGATCAAAT
TGAAAATAAACATATAGCGCGAGCATTGATACTTGAATTAAATAAAACGGGCCGATTGATTTTTTAATCATTTA
TACCCAATAAATTTCGAGTTGCAGCGCGGCGGCAAGTGAACGAATCCCCAGGAGCATAGATAACTATGTGAC
TGGGGTGAGTGAAAGCAGCCAACAAAGCAGCAACTTGAAGGATGAAGGGTATATAGAATTGGAGTGAATATG
ACAAATATAATTAACCCTAATAATGCGATTCTTGAAGTTAATAACGCATTAAATGATATTTTATCTCAGTATTTA
ACTAATATTGATATCCGCTTTGATCTACCAGAAATAAATTCAATCCCATCAACCCCTACAGTGAGTATATTTCTT
TATGATATACATGAAGACCTACAATTACGTTCTGCTGAACCAAGAAGTTATCATCCTACCACCAGCTCATTATT
GCCGGGATGGGTAAATATTAATTATAACTATTTAATTACTTACTGGCATTCAAGTAATCCATCAAGCGACAGTT
CTACCCCTGATAGTCAACCCAATAATCAAGCGGCACAAGTCATGACTGCTATTTTAAATGCATTGGTTAACAA
CCGACAATTACCTAAAATTCCTGGCGCATATACCAGAGTCATTCCACCTCAAGAAAATCTAAATAGCTTAGGT
AACTTTTGGCAAGCGCTTGGCAATCGCCCTCGCCTTTCTTTATTATATTCAATTACCGCACCGGTAAAACTGC
AAAATATTAAAGATGTCATAAAGCCCATTAGCCAAATTTCCACTTCTGTGGATCAAAAATCAAATCTGGATAAT
TCGCAAATCAACCAAGCCTTATTTAGCAAATTGGGTGCCGATTTAGGTGGCACACAAGATGTTCGTCTTGCTC
TTGCGAAAGTGAATCTGACAACCAAACCTGCTAAAGAAAATAATGAAAATCAAAATAATAAAAATGTAATTATT
GAAGTTTCTGGCATTACCCATTTGGATTATTTACCCAGAATAAAAGGTATTCTTTCAACATGGGTAAATAGTCA
TAGTGCTGTTGTTAGGATAAATGATATTGGTATTATTGTTTCAGAATATAAATATGATAAATTAACAGGCGTTTA
A
(Photorhabdus asymbiotica strain ATCC43949 PVCPaTox operon,
pvc1 - pvc16}
SEQ ID NO: 95
ATGAATACAGCTCAAGAAATTATTAACCGTTTATCGGGGAGAGCCGTTACGCTTGGTTGGGATGTTGTTATTG
CTTATGACCGAAAAAAAATTAACACTCTGTTAGAGCAACAATATGTTGAAAAGGTAAAAAACGGGGAGAACTT
CCCGCTTATCAACTGGGAGAACCAGAGAAAAACACTTCAATTTAAAGATCTTCAATTAGGTGTTCCACTTATTT
CTTTTGAGAATTCAACACTGGAAAATTCAAGGGCGCTTGCCACGATAGAATTTATTTCAGGAGCTATTATTGAA
TTTAGTGACTCCGGGCAAATAATCAACTATAAGAAGATTGAACCTAGTCATGGTTATGGCATGGTGCTGACTA
TCGATCTCATGGCTGGTACAGGTTCAGTAGAAGAACAAGGTCGGGTGATAATAAATCTTAACGAAGGCGCCA
TACTCGATTTGCATGTTATCCAACAACCGCCAGCAGAAGTGGTAGAATTTTTCCGCACTTGGTTGATGGCTAA
TAAAATGACTTATGAATTAGGTAAGCTGGATCTGAGTAGTCAAGCTGGTCTAGTGCCTCGTTCTTTTCGTATTC
GTACTCAGCGGGCGCCTGAAAAAATTCGTAAAGCGACGAGCGATGAAGGAAATGGCGCTGTTTTGTTGTTTG
TTGCCACTAACTATAACCCTACAAGTGGAACTTTACCTGCCAAGGATTATCCGTGGCTAATCCCTGAGGAATA
TTCAGGCGCATTGCTTATCGGTAATAAATGCTTATTTAAAGACATTCTGAAACCGAATCTGGATCAGTTGTTTG
ATAAAGGGGAATGGACATTAAAAGTTCAGCAAACGGATTCTGATCAACTGCTGCATTATCTGGAGGCAAACTC
TGCATATATAACAGATAAGCCTTATATGGCAGACTTTGAAGGAACTCAGGATGGAGTCTGGACAGGACGTTAT
AAATTTGAGACTGGCCGGGGACATTATGGGGTGTATGAAAATGTACGCTTTCCTATCAATGGAATGTTGATGA
AACCGGCTAAAACTGGATTACAGTTATCAATAGATTCACCACAAAGCCATCAATTTAATGTTGATTTCGGAATG
AAGTGGTTCCATTGTGCTAATATAATGTGTGGTTATTCCTGGTTTAACGAGACTTACCCATTTTATCTTGATGG
AAAATCATTTTATCAAGTTCATATTGACCCTGATAAAGAGGTGATTTATTTTACTGGGCCAGATGAAGATATTA
ATATTGTAGGAAATTACAGCCCGCCTGCGTGGTGGCAATCTAAATGGCAAAAACATATCAGTGATGATTTTAC
GGATATTTCCTCGGAAAAATTTAAGCGACTCAGTCAAATAAAATTGCCAGAAATATGCATGTTTGCCGTGAAC
CATTTATTATTTCCTGGTCATAATACTTTGCTGTTGAAAGACGTTTATTTACCGGGTGATATGGTGATTTTCGG
TGATATTAACCCATCACTTACCGCTTTTCGGGTTACGCCATTAAAAGCAACAGTGGTGGCAAAGGGAACCCAA
CAATTTAAAGCCATAGAAACTAATTGATGATTATACCCTTCATCCTTCAAGTTGCTGCTTTGTTGGCTACGTTC
ACTCACCCCAGTCACATAGTTAGCTATGCTCCCGGGGATTCGCTCCCTGGCCGTCGCGATGCATCTTGAAAT
CCATAGGGTATATATTTAATTGGATAAGTCTTTTTTATTTTAACATTATAACCTGATTCTTTTTGGATAAAATTAA
AGGATTATTAACATGTCTATTACACAAGAACAAATCGCTGCTGAATATCCTATTCCTAGTTACCGTTTTATGGT
TTCTATAGGAGATGTGCAAGTCCCTTTTAATAGTGTTTCGGGATTAGATAGGAAATATGAGGTTATTGAATATA
AAGATGGCATTGGTAATTATTATAAAATGCCAGGACAAATACAGAGGGTTGATATTACACTTCGGAAAGGCAT
ATTCTCTGGGAAAAATGATTTATTTAATTGGATTAATTCCATTGAACTCAATCGGGTAGAAAAAAAGGATATTA
CAATTAGTTTAACTAATGATACTGGCAGTAAAGTCTTAATGAGTTGGGTTGTTTCGAACGCCTTTCCGAGCTC
ACTGACGGCCCCTTCATTTGATGCTTCAAGTAATGAAATTGCAGTACAAGAAATTTCATTAGTTGCTGATCGG
GTAACAATTCAGGTTCCCTGATAACTAAAAACTTTAAGGAAAAATAATGTCTGTACAAACAACTTATCCCGGAA
TTTATATTGAAGAAGATGCATCATTGTCTCTATCTATCAATAATAGTCCAACAGCAATCCCTGTTTTTATCGGTA
AATTTTACAACTTGGATGGTTCCTTACCTAAAGTGGGAACATGTTCTAGAATTACCAGTTGGTTAGATTTCACT
AAAAAATTTTCGGTAGCTCCTCCTCAAACCATTTCATTGATCGCGTCGCCAATTGCTGACACACAAGAAAGTG
TACCCAAAGCAGTTCAATATACTTATAAGGCCGAGTTTGAAACCTCAGAAAATCTGGCAAATGGTGCCTATGC
GGTACAACATTATTTCCAGAATGGCGGTGGTATTTGCTATATCATACCTTTAGTTAGCGTGAAAAAAGAGGAT
GCTGCGATTGAGTTAACAAAATTACCTGAATTAATTGAAAGACAACAAGAGATTACGTTAATCGTCTGCCCGG
AGGACGATAAGACGCTCACTGTTGATAGCAGTAAAAAATCGGATGTTTATAACAGCATCAATACATTATTGAG
TAATAAGGTAGGTTATTTTCTCATTGCAGATTCAGATGATGGCAAAGCAGTTCCTGATACGTTGCCGGAAAAA
ACTGCGGTCTATTATCCTGGTTTACTAACTTCTTTTACACAACGCTATGCCCGACCTGCCGATTCTGCTATCAA
AGTGACCGGTATTACAAATATATCAACTCTGGCTGATATTCACACCAACTTGGCCGATGACTACTCAACAGCA
AGTCAGGTTATTAATGATGTTTTGGAAAAAAATAATAAGCTCGCATCGTCTCCCATTATTTTACCTCCCAGCGC
CGCTGTTGCTGGTGCTTATGCCGCTGTTGATGTGAGTCGTGGTGTTTGGAAAGCACCTGCGAATGTGATGTT
AAGTAATGCCACGCCAATCATTAGTATTTCCGATGCGGAACAAGGTGTGATGAACCCATTAGGTATTAATGCT
ATTCGTAGTTTTACTGGTAGAGGTACTTTGATTTGGGGAGCTCGTACTCTGGATAAAACGGATAACTGGCGCT
ATGTTCCTGTACGTCGTTTATTCAATAGCGCAGAGCGAGATATTAAGTTAGCAATGCGTTTTGCAGTTTTTGA
GCCTAACTCCCAACCAATTTGGGAAAAGGTCAAGGCTGCTATCAATAGCTATTTGCAGTCACTTTGGCAGCAA
GGTGCACTGCAAGGCAATAAACCCGATGAAGCCTGGTTTGTACAAATTGGTAAAGGCGTGACCATGACAGAT
GATGATATTAAGAATGGGAGAATGATTATCAAAATCGGCATGGCGGCAGTACGTCCGGCAGAATTCATTATTT
TACAGTTTACGCAGAATATCGCCCAGTAACTTAGGTCTATACCCTATAGATTTCAAGATGCATCGCGGCGGCA
AGGGAGCGAATCCCCGGGAGCATATACCCAATAGATTTCAAGTTGCAGTGCGGCGGCAAGTGAACGCATCC
CCAGGAGCATAGATAACTATGTGACTGGGGTAAGTGAACGCAGCCAACAAAGCAGCAGCTTGAAAGATGAA
GGGTATAGATAACGATGTGACCGGGGTGAGTGAGTGCAGCCAACAAAGAGGCAACTTGAAAGATAACGGGT
ATATTTAATATGGGCGATTTATTGCCCATTTTTGTGAAAGGAAATGAGTTATGTCGCCAACGCTACCCGGTGT
AACGATGACTCAGGCGCAGATAACAGCGTTCGGTGTCAGTACATTAAATATGCCCGTATTCATAGGGTATTGT
ACGAGATTGCCTGCCTTTTCAGCGCCTGTAAAAGTAAACAGTTTAGCTGAAACAGAACAAATAATAGGGAAAG
AAGGGCGTTTGTATGCTCTATTGCGCCACTTTTTCGATAACGATGGGATACAAGCTTTTATTCTGTCGTTAGG
CGCACCTGCTGGGGAAAATGCTAATAGTTGGCTTGAGGCATTACAACAGCCCGATTTGTATGCGGCTGTTGC
AGCAGAGCCGCTAATTACACTTTTAGCCGTCGTTGAGGCAAGTGAACTGAACCAAAAAGAAGGTAATGAGGC
TGTGGAAGCTTGGCGACAGTACTGGAAAGCAGTATTAGCGTTATGTCAGGCACGCAGTGACTTGTTTGCCAT
ATTGGAGGCACCAGATGATACCGCATTAATCAAGCGTAGTTTGCAGGATTTTCATCATAAGGCACGTCAGTTT
GGCGCTCTCTACTGGCCAAGGCTAGAAACATCTTATCAATCCTCTCAGTTAAAAATTTTGTCTCCTATTGGTG
CAGTAGCAGCGGTTATTCAAAGTAATGATGTCCGGCGAGGGGTAGGACATGCACCTGCCAATATAGCGTTAA
AACAGACGATTCGCCCGATAAAGTCCCGCCTGGAATTAGAAGAGTTGTATGAAGAATCGGATGGTTCACTGA
ATCTGATTTGTAGTTTTCCAGCTCGTGGTACTCGTATTTGGGGATGTCGTACGTTGGCGGGTATTGATTCACC
TTGGCGTTATATTCAAACCCGATTATTGACTTCACACGTGGAAAGGCAACTCAGCCAGTTAGGGTGCATGTTG
ATGTTTGAACCTAATAACGCAGTCACTTGGATGAAGTTTAAAGGCCATGCTGGGAATCTATTAAGGCAGCTTT
GGTTACAAGGGGTGCTGTATGGGCAGCGTGAAGATGAAGCCTTTTCCGTTGAAATAGATGAAAACGAAACGA
TGACTCGCCAGGATATTGATGAAGGCAGAATGATTGCTCGTATTCATTTGGCATTGTTAGCACCGGCAGAGTT
TATCGCTGTGAGTTTGAATTTTGATAGTGGGTGAGGGATTGGGAGGAGTAGATAATAAATGGGAATATGTGGAT
GAGAGTAGGAGGAGAGGTTTATAGGGGAGGGGTTTGAGATGGTTTTATTGTTAATTTTGTTTTTAAAGGTTTAG
TTCCTTCTCCCGTAGATATTCGATTTCAACGTGTTTCTGGTTTAGGGCGTGAGTTACAGGTTGAACAGCGCCA
TCAGGGGGGAGAAAACGCACGGAATCATTGGTTGGCTGAACGTATACAGCATAATAGCTTGATATTAGAAAG
AGGGGTTATGGTCGTTACCCCTTTAACACTGATGTTTGATCAGGTGATGCGGGGGGAAACTCTCAATTGGGC
AGATGTGGTAATTATTCTTCTCGATCAGGCTCAACGTCCGATAACAAGTTGGACCTTGAGTCATGCGCTACCG
GTTCGCTGGCAAACAGGAGATTTAGATGCCAACAGTAACCAAGTGCTGATTAACACCTTAGAGCTGCGTTAT
GAAGATATGCGCATTATAGGGGTAAAATTATGACTATCGAAATCCGTGAACTCATTGTTCAAGCCCGTGTTGT
CGGGACTGATACCAAAACAACACGAACCGTTCCTTTATCTATTGTGCAAATGGAAACACTTATAGAACAACGT
CTGGTTGAAAAAGTGAAGCGGGAGATATTAGACGTACTCCGGGAAGAACAAGGTGGTGGGTTATGAGCTTG
CTTGAACGAGGTCTGGCTAAACTCACGATTACGGGTTGGAAGGAGCGTGAGCGTAAACATCAGATTGGTAAA
CTAGAAGCAATGTATAACCCGGAAACACTTCAACTGGATTATCAAACTGATTATCTCCCTGATGTTAGCAATAA
TGAGGTAAGAGTGAGTAAGGGGTAGGTTTTGTGAAAGGGGGGAGGGTTAAGAGTATGGTTGTTATTTGATGGG
AATATGGCTGGTCTTACGACAACCGTCGAGTCCCAAATCACTACCCTCAAATCGCTTTGTTTAGTTAATGCAA
GTAGTGATGAAGGGAATTTTTTGGAAATTAATTGGGGGGGAATGGGTTGGGAAAATAAAAATTATTTTGTTGGT
CGGGCTAGTGGATTGTCTCTGACTTATTTGCGCTTTGATCGTAACGCAACACCATTGCGTGTGAGTGCGCAG
CTCACATTAGTCGCAGATGAAAGCTTTGTGCTCCAGGATAACCAAGCCAAGTTAGATGCGCCGCCGGTATCA
GTAGTTAATGTCCCGGATCTGACTTCATTACCTGCACTGGCGAATATCGCTAGCGTAACCACTATGTTGGGA
GTGGATTATTTAATGTTAGCCCGCACCAATGATATGGATAATTTGGATGATATGCAGCCAGGTCAGACATTGC
GAAGAGGGGAGGGATGATGAGTTTTTTAGATAAGAGTAAGTTGAAGGGATGAGATATGAAAGTGTTGGTTAAG
ATTCAGGGAGTGGAGAAGGAACTCAACGAACTGATAGTAAGCGAATTGAAAATCTCCCGACGTATCAATGCC
ATTCCGCAGGCAGTTGTAAAGCTAAGAGCGAAAGAGAGTGAAAGTGGTGTATATCAGTCTGATGTACAGCGG
ATGTTGAAGAGTTGGGGTGGGGGAGTAAAGGGAGAGGTTGGTATTTTGAATAGGGGGGTATTGAGTGGGGAT
ATTGTGCAGCAAAAAACAGAGTTAGTGTATGCGAAAACACACACTATCAAATTGGTGCTACGCCATGACTTAC
AGGGGATGAGGGGTAATTTTGGTAGGAGAGTGTTTGGGAATAGGGGTGATGGTAAAGTGATAGGGGATGTATT
GAATAGGGGAAGATTAAAGGGGGGATTTTGGGGGAGATGAGATTGGGATATAGATGATGAGGAAGTGGTTGA
GTATGGTTGGAGTGATTGGGAATTTTTGTTGGAAGGGGTGTATGGTAGGAATAGGTGGTTGTTAGGTGAAGAA
GATAAAGATAACACTCAGGGGAAAGTGACCATTATTGCTCCAAATTCTTTGCCCCTGAATGAGCGTTGGACAC
TGCAACATCAGGCTGATCATCAGGCTATCCGGCTTTACAGCACGGAGCTGATGCTGGATAACCGGTTTGATA
CAGCGGAGGCTGTTGTTAGTGCTTGGGATATTGATGATCAGGCATTACTCGTGGCGTGGAAAGAAACCCTTA
GTGAAGTTGGGAAAGATGGGTTAGGGTGAGATAATTTTAGGGAGAGAAATAAAGATTGGAGTGAAGTGTTATT
AAGTTGTGGGGTGTGTAGAAAAGAAGTTGAATTTTTAAGGGGTAGGGAATTAGTGATGGGGGGGTTGAGGGGG
GTTGGTGGTTGAGTGAAGGTTGAAGGGAGTAGTAAGTAGGGTTTAGGGGATGAAGTGATGTTGTGAGGTTTT
GGTGAAAATATGGATGGCTCACAAATACTGACGGGAGTGGATCATCGAATAACGGCAGAAGAAAGTTGGAAA
ACAACCTTACATGTGGGATTAGAACTGCCGTTAAAGGCAGAGTATGTCACTCAGGTTAACGGTGTTCATATCG
GGAAGGTTGGTGATTATGAATGAGATAGGAAAAAATGGGATGGTATTGGTGTTTTGATGGGTGGATTTGGAAG
GAATATTGGGTTGTTTGGGGGATTGGGAAAAGGGTAGGGGAGGGAGGAAAGTGGATTTTGTTTGTATGGTGAA
AGGGGTGATGAAGTGATTGTGAGTTTTTTGGAAGGGGAGGGTGGTTATGGTGTGATTATTGATTGGGTGGATA
ATCCTAAACAACAGACTCCATTGCAAATCAGCAAAGAGAATAATCTCAAAATGTTGATGATTAAGCAGAGCGA
TAAAGATGAGCAACAATTGTTATTTGATAGCCAGCAACAAACAGTCGCGTTAATCGGTAAGAAAAATATCGAG
GTTAAAGGTGAGTATATCAACCTGACTAAATCAAAGGGGACTCGATAATGGCAAATACGCTTATTGGCCAGGT
ATATGGTGAAGGATGGGGTTTTGGGATTAAATTTATTGGTGATAATAAAGAAAGGGGAGATGAAAGAGGGGGT
ATTGTTATGGCTCAAGGGATTGAAGATGTCAGTCAATCGCTGGAAATATTATTTCTTACCGAGCCTGGCGAAC
GAATTATGGGTGAAGATTTTGGTTGTGGTTTAGAAGATTTTGTTTTTGAAAATATTAGTGATAGGGTAATTTGTG
CCATCAAAAATCGTATTCAGCAAGCAATATTACGTTATGAACCTCGCGCATATTTATTGAACGTTGATATTCAA
ACCAAAGAAAACCAACCTGGACATCTGCTCATTCAGATTAATTGGAAATTACGTGGTAGTGATATATCTCAGC
GTTTAGACGGAGTGCTTAGACTCCATTCAGGTCAAGCATTGGAACTGTTATGACCAATTATATTATTATCGAC
GGGGATCTCATTCAAATAAATCCCAAATTTGAGGGTGATCGAACTCTTACGATTAATGGTATTCCTAAAATAAG
CGGGAATGGAGATGCGCAAATTGAAGGAAAAAATATTTGTGTGTCAGGTGATCACTTAACTGTCTCAATTCCA
GCCATTTATATAACCTCCAGACATCCTGTTGCAGGTAGTGGAAAAGTGAAAATTACAAATTTATCTGACGACC
AAGTAGGAGAATTTTGTGTTAGTGGGGATGTTGTGATTATTGAAGGGAGTGAGTTTGAAGGTGAGTTTAGAGG
GGATAAGCCGGCCACTAATCCAAGTAACCAAGATGCAGATAATCCTGCGCCTTCGAATGGGAGTGGGAGATT
TATAGAGTGAGAGAAGTTGGTTAAGGGAGAAAAATAAAAAATTTTGGGGAAGGGGTTAATAAGTATGAATAAG
GGGGGGGGATAAAAAGATGGATGTTGGTGAATTAAATAATAGGTTGATGAATGAGTTAGGAAGGAGGAATTTT
AAGTTAGAAAGAAAGGAGGGATTAAGGGAATTAAAGTGGTTAGAAGGTTATAGAGAAAATATTGGTTTTTATGG
GAATGATGATTATTTCTGGCATCAATTCTGGTTCTTAAAAAATCACACACCAGAAGCGCTCTTTGCTCGTTTGC
AAGGTGAAAGGTTGGGTGATGGAGAATTGGGTGGTGATGAAGGGGTATTGGTGGGGTTTTTAGAAGAGGTTAA
GAGGGGAGGAATGATGGTTGATAGTTTTTGAGGGGGTGATGGGGAATTGTAGTATGAGGAATTGGTAGGGATA
ACGCAGAAAGATGCACAACCTGATCATGTGGCGCTTGGCGTGGTATTAAGTACTGGTATTGCAGAATATTTAT
TACCGACAGGCACATTAGTGGATGGTGGACAAGACAGCAGCGGAAATTCACTGCAATATGCGTTGGATACCG
ATTTATTGGTTAATCCAGGGCAATTAACAGATGTTCGCTACAGCTATTTGGATCATAAGACCTATAAAATCTTC
ATCTTGCAAGATGATAAAGCGAATATCAGTTGGCCCTCTTCAGGCGCTCGTTTATTTGTAGCACCTGAGGGCA
ACGGACAGGAAAAGGCACCTGAACAAAAGTTGGCACTTTACCTGGGATTTGATGATATACAGCCAGGGCAAA
CTCTTCTTTATTTTGGCAATTCATGCATCAACTCCCCTGACATTAAAATGGTTTTATCTGAACGAGATAAATA
ACTGGGTGAAGCTAGATAGTGTCAGAGATAACACGGATGGCTTTTTTATCAGTGGATTATGGCAAGCGATATT
ACCTGATGATGCGGTGAAAATGTATTTTCCAGAGACAACTTCTGTAAAACGCTACTGGATTAAAGCTGAGGTG
GAATCGCTTACTGAATCTGGCGATTTGTGGCAACCGCTATTAGAAGGCATCTTGTATAACGCTCAAACAGCAA
CGCTGGTTGATGCAGACAACACAGATGAAAAGCACTTTCATGATGGGCTGATGCCTTTTAGCGTGCAGCATT
TGGTCAACACCGTTTCAGAGGTAAAAAAAATTGAGCAGCCCTGGTCTTCTTGGGGGGGAACGCCACAGGAA
GACACTACTGATTTCTTCCATCGAGCGGCAACACGTCTTCAGCATCGCCAGCGTGCGTTAACTTGGGATAAC
CAAATTGCCATGTTGAAGGCTGAATTTCCGCGGATTTATGATGTCATCTCACCAAATATCACGTGGATGAACC
AACTTCAGACATCAAATACGCAAACGCTGATCGTTATTCCTGATGTGAACTACAGCGACAACAAGGATCGCTT
ACGGCCACAATTCAGCCCTGCCAGCTTGCGACAAATGAGTGACTGGTTACAGATTCACACTAGCGCATGGGC
GAATCCACAAGTGGAAAATCCAATTTATATTGATGTCTCTGTGACCTATGAGGTGCAATTTAGTGCGGGTGTG
AATCCTGATTATGCCCTCCGGCAATTACAACAATGGTTGAGTTCAATTTATATGCCATGGTATCACGCAGATA
AAAAAGGTGTTGCCGCTGGCGATCAAATCGATTTTTACCAACTGTTTGCAGATATTCAGCGAGTACCTTACGT
GGAGCATGTCAAAACATTGACATTGACCACAAAAGACACCTCATTAACCAATGGCGGGGTTATTAAGGCACA
GCAAAATGAAGTGCTGGTGTTGGTATGGCAACAAGGAGAACAAATTAGGCAGGGAGAATCGAAATGAGGCA
GCATAATGAGTTATTTCCTGTAGTAAAAGACGCGATAAGCTTTGAAAACCTGCAAGCTCAGGGTGAGAAGGTT
ATTAGTGATCAGTCCGGTAACATATGGAGCGATAAAGATAAACATGATCCTGGTATAACATTACTAGACTCTTT
AAGTTACGGTGTTTCGGATTTAGCGTATCGGCACTCATTACCTTTAACCGATTTATTAACCATTGCTGGAAAAG
ATACGCTTTTTCCAGCCGAATTCGGGCCACAGCAGACGCTAACTTGTGGCCCTATAACACTGGATGATTACC
GGCGTGCGTTACTTGATTTACATGGTAATGATGCATTTAAAATATCAGCTAGTGACCCCAGAGACTTTTTGTTT
CAGGATATACAGTTAATTTGTGAGCCAAAAAGTAAGCGTTATAAATACTATTTCAATCCCGAAACGCTTGAATA
TACATTCACGCCACCTTCAGGGGATAAATTTAAAACTTTAACACTACGAGGGAATTATTGGCTTTATTGGATAC
CAACCCGTTGGGCAGGTAAATCAGCTAATTTGCCGTTAGTTAAGCGGGTGATGGAAGATTTTCTCCGTGAAA
ATCGAAATTTGGGGGAAAATGTTGTTCAAGTGACACGGGTGATATCAACGCCTATTTATCCTGAGCTGGTCAT
TGAGCTGGCGGATGATATTACAGATGCGGCATCAGTATTAGCATCAATCTATATGCTATTAGAACAGTGGGC
GATGCCGATGCCTGCTCGCTTTACTACCGAAGCATTACAGGCCAAGGGATTAACAAACGAAGAGATCTTTGA
TGGGCCGTGGTTGCGTCATGGTTGGATACCTCAGTTACCGACCTCTCAAAACTACCATACAGGCATGGTTCT
GAAGATGAATCATCTGATTAACCAATTGCTGGCGGTTGAAGGTATAAAGCGCGTAGTTAGCCTGACGTTGCC
AGAAACAGAATATTTGCATCAGATAAAAGATGATAATTGGTCCTGGCAATTAGATGTTGGTTATTATCCATTAT
TATGGGGAGCTAATCCACTAGAGGTAATTACAGAGAAAAATAACAATTATGTCAAATTGTTCGCAAAAGGTGG
GGTACGATTACAACCTGATCAGAAAAGTGTTGAGCGGTTATTATCACAGGAATCACTCATTAATAATGCTGCA
TCCACGTTACCGGCTGGTAAGGTGCGTGATCTCAAAGCCTATACACCTATAAGCCGCAGGTTGCCTGCCTGT
TATGGTTTGCAGAATACTTTGCAAAAGTTAAAACCTGAACAACGACACTTATATCAGTTCCTATTACCATTGGA
GCAAATGCTTGCTGATGGATGTGCGCGGCTTGCATTTTTGCCACATTTGTTAGCATTTAGGGACCGAAGCGG
AAATATCAGTGATACACTCTGGCCTTTCAAGAATACAGAGGACACAATTGCCCAACAGGTTCATCAGGAATAT
GCCGGTACATTAAAAGCCTTTCAACAGCAGGAAATTAGCCTGTTTGATGATAAAAATAGACCGCATCATGGCA
ATATCAATCGGGAATTAGATATTCTTGATTATCTGCTAGGGTATTTTGGTACACAACGTGCAAAGCGTCCATTA
ACGCAGGATATTCATGATTTTCTGCAAACCCAGCGAGGTTATTTGGCACAGCAGCCGGAGTTGGGTTATCAG
CGTGATAATATCCGTATTGATCGAGTTTCAGCTTTACAAAAACGTATAGCAGCCCGAATTGGGCTAGATGGTA
CTATTTTCAAAGAATCGGTTGATTTAAGTAAGTTACCTTTTTATTTGATTGAACATCGTCAGCTTTTACCAAATT
TACCCCATCTTGACTTTCAACATGATCAAACTCCCCAATCTTTTGTGATTTCCGACAACATTGTTAAAGTGAAA
CAAGCGGGAATAGCAGATAAAATCGTTCGTGGACAGCTTATTGATTTTATAGATATTGAAAGCAAATTTACCG
TTCGTGCCCAAATGATTGTCGCTGTAGAGGGAAATGAATTTTCTCTGGATACAAAAAATAGTATTCAACTTGAA
AAGAATCTGCAGTTATTACAATCAGCGTCTGAGAAAAACAATTTACGATGGAGAAATAGCACGGCGTGGTTAG
AGGATATGACGTATCGTATCAATTATACTGACGATCAGGTTATAGACGATAAAACAAAACAATGTCGTTTACAA
AGTAATACTAAATCGCCTTTTCCAGCCTTAATTGCACCAAAAAATAAGATTACGATTATTAAGCAATCTTCTCC
ACTCTCCAGTATTGCTGAATTTACTGATGAACCAGAATTCAAATTAGTTGCAACGGTGACAGAGATTGATCGG
ATTGAAGGGATATTGACTATCGAACGGGATGACAACCAACTCCCTTTCCCGACTAAAGAAGAGAGTAATCAAT
ATATATGGTACATATCTGATGAAAACTATATTTCAAGTGATCGTTTCTCTTTTGTGGTGAGCGTCGTGCTGAAT
CGCGGTTTGGTTGAAAGGGAAGATATTGATCAATATAAGCTAGAGGAATGGATAGAGCGTGAAACACTTGCA
GAGTTTCCTGCACATATTTCGTTAATTACTCATTGGCTGGCATCTGAAAATTTCGATGATTTTGCGAAGACATA
TCAACGTTGGCAAAACAATGGGGCGCAGTTAGGGGATGAATCCTACACCATTTTGGAAAAACTGACATTAGG
GCATTTACCAACAGGACTTACTGGCATTAGTAATATGTTTATTGCTACAGAAGCTCAGCGTCTAGAAGTTGTT
GGCGAGAGTGGTAATGAGTGGAATACCCAGGCAATTATTAACAACGAACTATTCTATGTTCCCTCACAGAATA
GTTAATACCGAGTGTTGTGATCAACTTTTATTATAAGCCGGAGGATAAATGGACAACAAAAATAACAAACCTAC
TGATCAAGAGATTCTAAAAACATCACGGGCTGTCGGAGAAATTCCTTCAGCGGATAATTTAAAAAATCGTTTTA
AAGCTCGTTCGATTCCATTAGAGACGGATTTTACTAATCTCATTGACCTTGCTGAAGTTGGACGATTGGCTAT
CGGCCAGTCACCATCGCAGCAAAGTAAAACGCCTGGCACCGGAATGGAATTAACTTCGGATGGTAAATTACA
AGTCAAGGCTGGGGCAGGTGTTGATATCGATAATAATAATCGTATTACTATTAAGTCTGGTCATGGAATTAAG
GTTGATGGAAACGGCATTTCCGTTAAACCAGGTTCGGGTATTAAGGTTGATAGTAATGGTGTAAATGTCAATA
TTGATGATTTTTGGGAGGAAATACGCAATAAAATTATGCCTAAAGGAACCATGCTGCCTATTTATGGCACACC
TAACCCCTCTGCGCTGCCAACAGGATGGGAATGGTGTGATGGTAAAGATGGCAGACCTAATTTAAAAAAAGG
GAAATATAACTTACTATCAGGTCAGTCTTCAGGTACTGATACTTTTTGGGCAGATAATAAGAATGGAGATACA
GAGATCAACGTGTTATTTGTTTACTATATGATTAAGGTTGTGTAATATCTTAAGTAATATGCATTACTCTAAAAT
GAATGATTTATATTTAAGTAACATAATAATTAAGTTGTGTTGTAGGGCTGTTTTTATGAGAAATATAAAAACGGA
GGTAATAATTGGCTTCAAAATATCAGTGATGAAATAGAGTTATTTCGCTTTATAAAAATTTGTTTTATTTCTTTT
AATAATTATTTATAGAAGGTAATGATATGTGCACACAAAAAAACGTGTTAGATAGACTGAAAGATAGAAATATT
ACATTGGGTTGGGATGTTGTTGTTGCATATAACCAAGAAAGTGTTAATAAGTTATTGAAGCAACAATATGTTGA
AAAAGTTTACTCAAATGAACATTTTGTTTTTAAAGATTGGCATGATGATAATAAAACGAAATTTATTGAGGGATT
AACAGTAGGCGCTCCACTAGTTTCATTTGAGGAGGCGTCTTTATCCGATGCTAATGTAAAAGTGACACTTAAC
TTTCTTTCTGGTAGATGGAGAGTTATACAAGCAAATACCGGCACACCAATTGAATGGAAAGAAATTGTTCCTG
GCAGTGGCTATAAAGCAGAATTAGTTGTTCCGCTTAAATCAATAACTGGTAGTGTAAGTAAAAAAGATATCATA
TTAAAATTCAAAGATGCTGTCGTAAAAAAAATAAATTTATTTGACAATCAAGAGCCTGATTTTATTAATTATTTC
AAGCAATCGATCAGTGAGGGAAATTATACTTTAGGGCAACTGGTGACAGACAGCACACCGGGATTAATTCCT
GCTGAATTTCATATTCGTACTCAACCCCATCCAAAAACACGTGAGCGTGGTTCTCAATATGTAGGAAATGGTG
CGGTACTGTTGTTTATTAAAACGCAATATGGCGGAAGTGGAACATTGCCTGTAAATGATTTTGATTGGTTAATT
CCTGATGATCATACTAGCGCATTAGTCATTTCGAGTAAGACCATGATGGGGCAAATATTGCCAAAACAATACA
AAGATAAATTGCCTGGTGATCCTCAGTTTAGCCCACCAAAAAGAGTCAATGATAAACAAGACTCTGCTTATTAT
ATTACGATTACCGATGGTGGATTTGATGGTAATAGCCCTATAGAGAAGTCATGGTTACGTTCTGATTATAGCA
ATGGGATTTGGACTGGTGAACGTGGTAATGCTATTATTGGTGAAAAAGGAAAGCGGATACCACCACGTTTTC
CATACCAAAATTTTGTTATTAAACCTCATGGTGAATCGTTATTTCAAGGATGGGAGAATAAGATAAATTACACT
CAAAAGTGTGCAAGATATTTCCGACATCATAGTAATAGTATAACTTTCGAAGATACTGCATTAATGGATCTCAG
TATTGGTGGACAAGGTAGTATCAATTGCCAGATTGATGGTGAACATTTCTATTTAAAATCAGATGATTTTTCCC
CCAATGTCAGCTATGAACCAACTTCATTCTGGGATAAATTTATCGGTGGGGTGGATGCAAATGTGAAAGATGA
ATTCAGAGATGAATTAGCACAACAGGCAGAAGCAAAGTTAAAACAGGTATTTAATATTGAATTGCCTGAAATC
AGTCTGTTTTCTATTAAACATCTGCTCTTTCCTGGCATGGATGTTATGCAACTTAAACAGGGTTATTTCCCAGG
AGATTTGATTATCTTTGGGGATATTTCACCTAAATTGACCACAATTCAGGTGGCTCCTTTGGAAGCCATGGTT
GCCCTTAAAGAAAATCAAAAATTCACTGTCGTACCTGAAAATAAAAATGTTAGTTGGAAGTTGGATCATAATAG
TGAGGCTATCAATGATCCGGGAAATATTGATGATAAAGGTATTTATACGGCACCGGGCAGAATCAGATCTGG
TTCTGAAGTCATTAAAGTCACTGCAACTGACGGCGATGGAAATCAGGCATCGGCGGCGCTGACGTTGGTTCC
TTCTTCTGTTGCATTAACACCTTCTTTTGCTTTTATCTCTGAAGCAGATAAGAAACCTATATTATTATTGGCGAA
TGTCCTAGACGGAAAAGCAGTAACATGGAATGTGGAAAGCTGTACAGGCAGCCAATGTGGTTCTGTTGATCA
GAATGGGCTTTATACTCCACCAGCAGGGCGTTTTAACGATGGATTTACTTTTGCATCCATCACCGCAACTGCA
AAAGATGGTAGTCAAGCACGAACCATTATTTGTCTAATGGCATCAATGCCAGGACATGGTTTTTACAAGGTTG
AACCTAATTTACGTTTGAATGTGAAAGTAGGGGAAGAAATTATCTTTAAAGCGCAGGCAGATAGCTATAATGG
TGATCCTGATACTTGGGAAATTTTCCCTCCTCGCGGAAAATTAAGTGAACCTGAGTTTGAACCCAATAATGAT
CCTGAAACTAATGATACAATTTTTGGTCATTATAAGGTGACCTATACCGCGCCGACTAATGTTACCTCACCTG
AATTGCTTGTTGTCCATGTATGGGAGAAAAATAGGCATAATGAGAAAAACAAAGGTAAGGCAGGATATGCACT
TATTGAAATTATCCCAGATGATAAATAGAAAATTTATTTAAATAAAAATCACAGCGGGTTTATCTCGCTGTGATT
AAAGTCATCTTTTTTTATAGATTGTTTATCTCTAATAATAATTTTATTTTATAATATAAAGGAAATTAAAATGAATA
ATGAATATAAAAATAACACCGTGAATTGGCGTATTTCACCTGATACGGTAGGAAGTATTGATAATAACGGTTTA
TATACAGCACCTAATCGGGTAAAGAATATCGAATTTGTCCAAGTAATGGCAAGCGATGCTAATAATAATCAAT
CTTCTGCGATTATTACTGTTATTCCCTCTTCTGTTGCGTTAACGCCATCGTTTACTTTTATCTCTGAGGCAAAA
AAAACATCAGTCACTTTTAAAGCGACAGAACTTGAAGGGAAAAAAGTGACATGGAGTATAAATAATTATACCA
GTAATCAGTATGGTTCCATCGATCAAAATGGTATCTACACACCACCGGAAAGTCGTTTTAACGATGGATATAC
TTTTGTATCTATTACAGCAAAAGCGGAAAATGGCGCTGAAGCGCAAGCGCTTATTTGCTTGATGGCCAAAATT
CCAGGGCATGCCTTTTTCGATGTTCAGCCTAATATATGTTTAAGTGTGAAGCCTGGAGAAGAAATCATTTTTA
GAGCTAACGCAGATCGTTATAATGGTGATCCTGATTCCTGGGAAATTTTCCCGTCTCTTGGTAAATTGGGTGA
GCCTGAGTATATAAAAAATAACGATCCAGAAATTCCTATTTATGGATATTATCAAGTGAAATATATTGCGCCAA
CCAATATAAATTCTTCCCAAATACTCGTTGTGCGTACTTGGGAATATGACAAACATGATGAGCATAATCAAGGT
AAAGCAGGATATGCATTCATTGAAATTGTGCCAGAAAATGAGCTTTAATATATATACCCAATAGATTTCGAGCC
GCAGCGCGGCGGCAAGTGAATGAATCCCCAGGAGCATAGATAACGATGTGACTGGGGTGAGTGAACGCAG
CCAACAAAGAGGTAACTTGAAAGATAATGAGTATAAATGACTTTAGTAAGAGAAATTATGGCTTCATTCAGAAC
TATTTATTAGAGTAATTAACTTTATAAAGACATTTAATGGAAAATATAATAGAAAAATTTAATATTAATATTGAAG
TCTCATCTGAAATTATTGGAGAGAGTTTATTAAACTCCCCTTTATTGATGAGTAGAGAAATCAGCAATCAATTA
TCTGAAATATTATTAGATTATAAAGAATATAATATTGCATTGGATAAGTTAGTGTTAAATATAGGAGAAATACCC
TATGAAATATTTGAACAACAATTCTATGGTCGTTTGGGAAAATTATTAAATGAAAAGTTAACAATAATAATAAAT
GATAAATTATTGGTAAAAAACATATCAACCTCGTTATTTCCTGAATGTTTTAGTGAAAAAAGAAACCCATTATTA
AATAGAGTCATAAAAAATTTACCTTCTAATTTGGTTTTTGAAGTTCATTCAATGGTAAAAATAGAATCAGTAAAT
AACAAAAAACAAGCTAATATATTGACATCTTATCTGGCTTATTCTTTTTTTAATAAAAGCAAATTACAACAACAT
TTATTTTCCACTAGTAATAATAAATTAATTGAGAGCTTATACGCACTTTTTCTAACGGATCAGAATCGAATACCT
ACTGCTCATAAAATAGGAAAAGGTGCACTTATACTATCTGCCCTTATTTGGCTTTATTCTAATTCCAATGATTAT
CTGCCCAAACCAGAAAGCACTCTGTTGTTACAAATAGAACAGGATATAAAACAAGGATATTTGCCTTTAACGT
TGTTAATCACTTTCTTCCAGAACAGAAATGGCGGGCGTGTTTTTTGCGATTGGCAGTATGCGTTATGGCAAAT
CGATATCATCAAAAATCACTTAGGCATTAAAATAACATCGAAAGAACCCCATTTACGGGAGAAAATAATGTTAC
AACCAGTTAATGCTTCTGATCGATCCTCTGTGCTGATATCAGACGAAAAATTGACAATACCGTTAACAATTACA
GGTGCGGGATTAGTGCTTCTCTGGCCACTATTAACTCCACTATTTTCGTCTTTTGATTTGTTAGATAAGAAAAG
TTTTTCAGACAATTTGGCACAGGAAATAGCATTTAATTTATTGGAATGGTTAGTCTGGGGAGATGAGATGCTG
TTACATCAGGAATCATCATTATCTTTATTACTCTGCGGAATAGATCACCAAACAATACTGGAGCGCCAGGTTCT
TATTCCTGAGCACAAGGAAAAATTAAATAACTGGTTGCAAGGTATTTGTACTCAACTTTTCTCTTGGAAAAAGC
TAGGGATCGATGATATGCGCCAACTTTTTTTGCAGCGTCAGGCTGCACTTTATTATGAAGATGATGGCCGGTTG
GTTATTAACGGTGCAGCGTGAAGCTTATGATGTATTACTGACTCAAATGCCTTGGCCGTGGCCATTGAATATT
GTGACATTACCTTGGCTAGCTGAGCCGATTAGTATCACTTGGGAAGGTATCTCTGAACCAACGGATTTGTCAT
TTTGGTAATCCAATATCTCATTAGGAACTCTATGCATGTACGATTTATCTGATGATCTTGCCAGACAGAATATT
TCACCGGAATATGAATTGACGGTTTTGCTGTCTCAGACTGCTATATTGGATAAACGAATTCGTTTACGAATTGA
GGAATTAATGCAACAGCAAACAC;TATTGGGAGAAAGTGGACAGACGTCTTTTGATGATATTTGATTTTUATTC
GTTTGGAGTGAAGAAGAAAAATGATGTTATTTGGTGTGAGGGGATGAAAATTGGAGGAAAGAGGATTTTGGTG
CTGAGCCGATCCCATCTCGTAGCCGTCTAGGACAATTAGTTGAACGGTTTGACTTAACTCAATTTGAAATTGA
TTTGATTTTATTGTGGGTGTTGGGTGATGTTGAGAGAGGTTATGTAAGGTTATTTTCTCTTGTTGGGGTAAGTG
GAGGTAATAACAGCAAAAAGCAGATGTTAACGTTGGGATTGGCTTTGGAGTTGCTTTGTCCGAGTGTAGTAG
AGCGCAATGCGCAACGTGCCAGTTTATTACCACAGGCACCGCTTTGGGATTATCGTTTATTTCAGTTGCGCG
GTGATATGTCTGTTTCCTACGATGAAATACCGTTAGCAATCGATAATTCTCTTATGCATTGGTTATTGGGGCAT
GATGGTGTGGGGATTTGTGTTGTGTGGGGGGGTGATTGGGTTGGTGTTGGTGAAGTGGGTGATATTTTGGGTG
ATTTCACCAACCAATTGATAGAACTCTGCCAAATGGAACAAGAGGGGATGCTGACAATAATCGCCGGCGGAG
CCGGAAGTGGCAGCAAAACAAGTGTTGCACGCGCAGCATCACAAGTAGGGCGCTCTGTATTGTTGTTATCGT
TAGCATCAGTGACACTGAGTGAACATGAAACTATTACACTGATAACACTGGCATTACGTGAAGCACAACTAAG
AAATGGGTGTGTTATGTTTGAAGGTTTGGATGAGTTTTGTGAAGGAGGGGGGGGTTTGGAGGTGTGGGTAGGA
AATGGAGTGGGTGGTTGTTGGATTGGGGTGTTTTGTGAATTAGGTAAGGAAGGATGATTATTGGGATTGGATG
CAATTTCACAAGTTGTATTGTCTATGCCAATGCCTTCTTTAATGGTGAAGGCTGCAGCATTAGCTTCAATGATG
AGGAATTATTTTGGAGAGAATTGATTGGATGTTGAAAGTTTAGTGAGATGTTTGGATGGTTGTGGATTGATATTG
AAAAAGGCCCTTAGTGAAGCAGAAATTTATCGCCGACTACGGGGGGAAACGGCTAGTTTGAGATTAGATGAT
GTGGAAATGTGGGTGGGTTTTGGGTTAGAGGAGAATTTTGGAGGTTTAGGAGAGAGAATTAGAGGAGAAGGAA
CCTTTGATGATTTGATCATCAGTGAATCTCAACAGCAACAATTACAAGAAATCCTGGCGGCTATTCGGCAACG
AGATAGGATGGTAGAGGAAGGATTTGGTGGTAAAGTGAGGTATGGGAGGGGTATGAGGAGGGTATTTTTTGG
TGAATGTGGGAGAGGAAAAAGGATGGTAGGAGAAGTGTTAGGTGGTGTTTTAGGTGTGGATTTGATGAAGGT
AGATTTGTGGAGTGTGGTTAAGAAATATATTGGTGAAAGTGAAAAAAATGTGGGTGGTGTTTTTGATTATGGGG
AAGAAGACGCCGGGGTATTGTTCTTTGATGAGGCAGATGCATTGTTTGGCAAACGAAGTGAAACTAAAGATG
CAAAAGATCGTCATGCTAATATTGAAGTTTCCTACCTATTGCAACGCCTTGAAAGTTATCCAGGGCTGGTGAT
ATTAGGGAGGAATTAGGGTAATGATTTAGAGTGAGGATTTAGTGGTGGGGTGAGTTTTTGGGTAGGATTGTGTT
TTCCAGATGTTTCCTTACGGGAACGGATGTGGCGGATTATCTGGCCATCGGGAATTCAATTAGCCGACGACA
TGAGTTTTTGAGGGTTGGGAAAAGGGGGTGAATTAAGGGGGGGGAATATGGGTAATATTGGGGTAGTGGGTA
GTTGGCTGGCAGTAGATGAAGGAAATGAAAAAATTACTATGGCTCATATTGAATGCGCATTACGACGTGAACT
GAGTAAAGTTGGGGGGATTGATTTAGGTTAATTTTTGTTTGTAATGGGGAGAGAAGTATGGTTAAAAATATGAA
ATCAGATGAAACCTTACTGATATTAAATAGTAAAATAGAAGATGCATTAAAAGCGTATTTACCGGGCGAAGAT
GTGGTTATTGGGTTGGATATGTTTGGTAAAAATGAAAATGGAGATTGTGGTAGGGTGTGGGTTTTTGTTTATGA
TATTGAGGAAGATGTGGAATTAGGGGTGGGAGAAGGGGGGGAATAGGTGGGTGGGAGAGGAAATTTTGTGGG
GGGATGTGTCAATGTTCGTTGTAATTATCTTATTTCCTACTGGGAGCCGGAACAGAGCGGAGGGCAGGGATC
GCCAACCATACGTTCTAATAGTCAATCAATGAAGATAATGAACTGTGTATTGAATGCATTAATTAATCATCGTT
CATTTCCTGGTTTACCCAGAACTTATACGAGAGTTCTTCCTCCTAATGAACAATTAAATAGCTTAGGAAACTTT
TGGCAATCATTAGATAATAAGCCTCGACTATGTTTAAGTTATATGGTGACTATTCCTATTCAACTTACCCCGCC
GAGAGAGAAGGTATGTGGTGTGATTAGGTGAAAAAGTGATATTAGTGGAAAAGGATGGGTTAAGTTTTATGTTG
AGGCAGATGAAATTATCCGTCAGGCATTAGTTGATGCCTTAATATCTCAAACAACAGAATCTATGGATACGAT
AACTAGCTGGCTGGCAAAAGTTGTTATTATTTGTCGACCACCAGAAATAATGAATAAACAAATGATTGAACAAA
CTGTGAAATTAATTATTGCTGGAATTACAGAAGAGGGATTAGCTGGAAATATAAAGACAATCACTCAAAAGTG
GGTGGAAGAGAAGACGATTATTGGTGAAATCGACGATGTTTCTCTAGTTATTTCCCAAGTTGACACGACAGC
GTTGTCTGCTGTAACAATACCGACATCTGTTTAA
(Pnf epitope)
SEQ ID NO: 96
TGQKPGNNEWKTGR
(PVCpromF)
SEQ ID NO: 97
TATCATATGTCTACAACTCCAGAACAAATTGCTG
(PVCpromR)
SEQ ID NO: 98
ATCTCTAGAACAGATATTCCAGCCAGC
(ParaINF)
SEQ ID NO: 99
GGCGTCACACTTTGCTATG
(ParaINF)
SEQ ID NO: 100
TCGGTGGCAGTAAATTGTCC
(F1 primer)
SEQ ID NO: 101
ATGTCTACAAGTACATCTCAAATTGCG
(F2 primer)
SEQ ID NO: 102
GACTCCCTTGAGGGTACGG
(F3 primer)
SEQ ID NO: 103
TTCTGATGAGAGTGATGGTAC
(F4 primer)
SEQ ID NO: 104
TGAATAAAGAATTCAGTCAATATC
(R1 primer)
SEQ ID NO: 105
TAGTGGCTGATGAAAGTCTG
(R2 primer)
SEQ ID NO: 106
GGAAGCCAAAGATAATGAAGTG
(R3 primer)
SEQ ID NO: 107
CATTTCTTCCCTATGGTTG
(R4 primer)
SEQ ID NO: 108
TTAAATTCCTACAAGATTATCTTT
(tBid amino acid sequence)
SEQ ID NO: 109
RSSHSRLGRIEADSESQEDIIRNIARHLAQVGDSMDRSIPPGLVNGLALQLRNTSRSEEDRNRDLATAL
EQLLQAYPRDMEKEKTMLVLALLLAKKVASHTPSLLRDVFHTTVNFINQNLRTYVRSLARNGMD
(E. coli Sequence Optimised tBid bases)
SEQ ID NO: 110
CGGTCAAGTCACTCGCGTCTGGGGAGAATCGAGGCTGATAGTGAGAGCCAAGAGGATATCATAA
GAAACATAGCACGCCATTTGGCACAGGTAGGCGATTCTATGGATCGCTCCATCCCGCCTGGACTT
GTCAATGGTCTTGCGCTTCAACTTCGTAACACTTCCCGGTCCGAGGAAGACAGAAATCGGGACCT
TGCGACTGCTCTGGAACAACTGCTTCAAGCATATCCTCGTGACATGGAGAAAGAAAAGACTATGT
TAGTATTAGCTCTTCHTTAGCTAAAAAGGTAGCTTCGCACACTCCAAGTTTATTGCGGGACGTTT
TTCACACCACTGTTAATTTCATCAATCAGAACCTGCGTACTTATGTGAGATCTTTGGCGAGAAATG
GTATGGAT
(BaxBH3 peptide (aa59-73))
SEQ ID NO: 111
LSESLKRIGDELDSN
(E. coli Sequence Optimised BaxBH3 bases)
SEQ ID NO: 112
CTGTCGGAGAGTTTGAAGCGTATAGGTGACGAGCTGGACAGCAAT

1. A method for packaging a payload into a Photorhabdus Virulence Cassettes (PVC) Needle Complex, the method comprising:
a. providing an effector fusion comprising a PVC effector leader sequence fused to a payload, and
b. contacting a PVC Needle Complex with said fusion thereby allowing the leader sequence to package the payload into the Needle Complex;
wherein the payload is one or more selected from a polypeptide, a nucleic acid, or a combination thereof; and
wherein the leader sequence and the payload form an effector fusion that is distinct from a wild-type PVC effector protein.

2. The method according to claim 1, wherein the leader sequence comprises amino acid residues 1-50 of a PVC effector.

3. The method use according to claim 1, wherein the leader sequence comprises an amino acid sequence having at least 60% sequence identity to one or more sequence selected sequence from SEQ ID NO.: 47-SEQ ID NO.: 92.

4. The method according to claim 1, wherein the PVC effector comprises an amino acid sequence of one or more sequence selected from SEQ ID NO.: 1-SEQ ID NO.: 46.

5. The method use according to claim 1, wherein the PVC effector comprises a sequence selected from SEQ ID NO: 4, SEQ ID NO: 22, SEQ ID NO: 25, SEQ ID NO: 30, SEQ ID NO: 32 and SEQ ID NO: 46.

6. The method use according to claim 1, wherein the leader sequence is covalently fused to the payload, preferably at an N-terminus of the payload.

7. (canceled)

8. The method according to claim 1, wherein said contacting occurs within a cell, in a cell lysate, or in a purified cell lysate.

9. The method of claim 1, wherein said method is performed in vitro and/or ex vivo.

10. (canceled)

11. (canceled)

12. A PVC Needle Complex comprising an effector fusion;
a. wherein said effector fusion comprises a PVC effector leader sequence fused to a payload;
b. wherein said payload is one or more selected from a polypeptide, a nucleic acid or a combination thereof; and
c. wherein the effector fusion is distinct from a wild-type PVC effector protein.

13. An effector fusion, comprising a PVC effector leader sequence fused to a payload;
a. wherein said payload is one or more selected from a polypeptide, a nucleic acid or a combination thereof; and
b. wherein the effector fusion is distinct from a wild-type PVC effector protein.

14. (canceled)

15. The PVC Needle Complex according to claim 12, wherein the leader sequence comprises amino acid residues 1-50 of a PVC effector.

16. The PVC Needle Complex according to claim 12, wherein the leader sequence comprises an amino acid sequence having at least 60% sequence identity to one or more sequence selected from SEQ ID NO.: 47-SEQ ID NO.: 92.

17. The PVC Needle Complex according to claim 12, wherein the PVC effector comprises an amino acid sequence of one or more sequence selected from SEQ ID NO.: 1-SEQ ID NO.: 46.

18. The PVC Needle Complex according to claim 12, wherein the PVC effector comprises a sequence selected from SEQ ID NO: 4, SEQ ID NO: 22, SEQ ID NO: 25, SEQ ID NO: 30, SEQ ID NO: 32 and SEQ ID NO: 46.

19. The PVC Needle Complex according to claim 12, wherein the leader sequence is covalently fused to a payload.

20. (canceled)

21. (canceled)

22. (canceled)

23. (canceled)

24. (canceled)

25. (canceled)

26. The effector fusion according to claim 13, wherein the leader sequence comprises amino acid residues 1-50 of a PVC effector

27. The effector fusion according to claim 13, wherein the leader sequence comprises an amino acid sequence having at least 60% sequence identity to one or more sequence selected from SEQ ID NO.: 47-SEQ ID NO.: 92.

28. The effector fusion according to claim 13, wherein the PVC effector comprises an amino acid sequence of one or more sequence selected from SEQ ID NO.: 1-SEQ ID NO.: 46.

29. The effector fusion according to claim 13, wherein the PVC effector comprises a sequence selected from SEQ ID NO: 4, SEQ ID NO: 22, SEQ ID NO: 25, SEQ ID NO: 30, SEQ ID NO: 32 and SEQ ID NO: 46.

30. The effector fusion according to claim 13, wherein the leader sequence is covalently fused to a payload.
